SEQUENCE LISTING (428 sequences)

SEQ ID NO: 1 - protein, 746 aa, Hepatitis C virus
  VARIANT 248: note = X can be any amino acid
  VARIANT 262: note = X can be any amino acid
  VARIANT 270: note = X can be any amino acid
  VARIANT 275: note = X can be any amino acid
  VARIANT 399: note = X can be any amino acid
  VARIANT 408: note = X can be any amino acid
  VARIANT 524: note = X can be any amino acid
  VARIANT 580: note = X can be any amino acid
  VARIANT 641: note = X can be any amino acid
  VARIANT 709: note = X can be any amino acid
  VARIANT 733: note = X can be any amino acid
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREGNASRCWVALTPTVAXRDGSLPTTQLRRHXDLLVGSAXLCSAXYVGDLCGSVFLVGQLFTFSPRRHWTTQDCNCSIYPGHITGHRMAWDMMMNWSPTTALVVAQLLRIPQAILDMIAGAHWGVLAGIAYFSMVGNWAKVLVVLLLFAGVDAETHVTGGSAARATSGXASLFSPGAXQNIQLINTNGSWHINRTALNCNDSLDTGWVAGLFYYHKFNSSGCPERMASCRPLADFDQGWGPISYANGSGPDQRPYCWHYPPKPCGIVPAQQVCGPVYCFTPSPVVVGTTDRLGXPTYNWGENETDVLVLNNTRPPLGNWFGCTWMNSTGFTKVCGAPPCNIGGVGNNTLXCPTDCFRKHPEATYSRCGSGPWLTPRCLVDYPYRLWHYPCTVNYTIFKVRMYVGGVEHRLXAACNWTRGERCDLDDRDRSELSPLLLSTTQWQVLPCSFTTLPALSTGLIHLHQNIVDVQYLYGVGSSXVSWAIKWEYVILLFLLLADARICXCLWMMLLISQAEA

SEQ ID NO: 2 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSTGLYHVTNDCPNSSIVYEAADAILHTPGCVPCVREGNTSRCWVAMTPTVATRDGKLPTTQLRRHIDLLVGSATLCSALYVGDLCGSIFLVGQMFTFSPRRHWTTQDCNCSLYPGHITGHRMAWDMMMNWSPTAALITAQLLRIPQAILDMIAGAHWGVLAGIAYFSMVGNWAKVLVVLLLFAGVDAQTHVTGGRAAHITAGLTSLFSPGPSQKLQLVNTNGSWHINSTALNCNDSLKTGWIAGLLYSYKFNSSGCPERLASCRRLTDFAQGWGPISHANGSGPDERPYCWHYPPRPCGIVPAKSVCGPVYCFTPSPVVVGTTDKSGAPTYNWGENDTDVFVLNNTRPPLGNWFGCTWMNSTGFTKVCGAPPCVIGGAGNNTLRCPTDCFRKHPDATYSRCGSGPWITPRCLVDYPYRLWHYPCTVNYSIFKIRMYLGGVEHRLEAACNWTRGERCDLEDRDRSELSPLLLSTTQWQVLPCSFTTLPALSTGLIHLHQNIVDVQYLYGVGSSVASWAIKWDYVVLLFLLLADARICSCLWMMLLISQAEA

SEQ ID NO: 3 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEA

SEQ ID NO: 4 - protein, 746 aa, Hepatitis C virus
MSTNPKPQRKTKRNTNRRPQDVKFPGGGQIVGGVYLLPRRGPRLGVRAVRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGIYHVTNDCPNSSIVYETADTILHSPGCVPCVREGNASKCWVALAPTVATRDGKLPTTQLRRHIDLLVGSATLCSALYVGDLCGSVFLVGQLFTFSPRRHWTTQDCNCSIYPGHITGHRMAWDMMMNWSPTTALVVAQLLRVPQAILDMIAGAHWGVLAGIAYFSMVGNWAKVLVVLLLFAGVDAETYTTGGSVAQAAFGLTSLFRPGPKQDIQLINTNGSWHINRTALNCNASLDTGWVAGLFYYHKFNSSGCPERMASCRSLADFDQGWGPISYANGSGPEHRPYCWHYPPKPCGIVPAQNVCGPVYCFTPSPVVVGTTNKLGVPTYSWGSNETDVLVLNNTRPPLGNWFGCTWMNSSGFTKVCGAPPCVIGGAGNRTLHCPTDCFRKHPEATYSRCGSGPWITPRCLVHYPYRLWHYPCTVNYTMFKVRMYVGGVEHRLEVACNWTRGERCDLDDRDRSELSPLLLSTTQWQVLPCSFTTLPALTTGLIHLHQNIVDVQYLYGVGSSIVSWAIKWEYVILLFLLLADARICSCLWMMLLISQAEA

SEQ ID NO: 5 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRSWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAFQVRNSSGLYHVTNDCPNSSIVYETADTILHSPGCVPCVREDNTSRCWVAVAPTVATRDGRLPTTQLRRHIDLLVGSATLCSALYVGDLCGSVFLVGQLFTFSPRRHWTTQDCNCSIYPGHITGHRMAWDMMMNWSPTTALVVAQLLRVPQAILDMIAGAHWGVLAGIAYFSMVGNWAKVLVVLLLFASVDAETYTSGGSVARATAGFAGIFNPGAKQDIQLINTNGSWHINRTALNCNASLDTGWVAGLFYYHKFNSSGCPERMASCKPLAHFAQGWGPISYANGSGPDHRPYCWHYPPKPCGIVPAQNVCGPVYCFTPSPVVVGTTNKLGAPTYNWGSNDTDVFILNNTRPPGGNWFGCTWMNSSGFTKVCGAPPCTIGGVGNNTLLCPTDCFRKHPEATYSRCGSGPWVTPRFLVHYPYRLWHYPCTVNYTLFKVRMYVGGVEHRLEVACNWTRGERCDLDDRDRSELSPLLLSTTQWQVLPCSFTTLPALTTGLIHLHQNIVDVQYLYGVGSSLVSWAIKWEYVILLFLLLADARICSCLWMMLLISQAEA

SEQ ID NO: 6 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREGNTSKCWVAVAPTVATRDGKLPTTQLRRHIDLLVGSATLCSALYVGDLCGSVFLVGQLFTFSPRRHWTTQDCNCSIYPGHITGHRMAWDMMMNWSPTAALVTAQLLRVPQAILDMIAGAHWGVLAGIAYFSMAGNWAKVLLVLLLFAGVDAETYTTGGSVARTTRGLASLLQVGPKQDIRLIHTNGSWHINRTALNCNASLDTGWLAGLLYYHKFNSSGCPERMASCRPLADFDQGWGPISYANGSGPEHRPYCWHYPPKPCGIVPAQTVCGPVYCFTPSPVVVGTTNKLGVPTYTWGSNDTDVFVLNNTRPPLGNWFGCTWMNSTGFTKVCGAPPCVIGGVGNNTLHCPTDCFRKHPEATYSRCGSGPWITPRCLVDYPYRLWHYPCTVNYTLFKVRMYVGGVEHRLQAACNWTRGERCDLDDRDRSELSPLLLSTTQWQVLPCSFTTLPALTTGLIHLHQNIVDVQYLYGVGSSIVSWAIKWEYVILLFLLLADARICSCLWMMLLISQAEA

SEQ ID NO: 7 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREDNASRCWVPVAPTVATRDGKLPTTQLRRHIDLLVGSATLCSALYVGDLCGSVFLVSQLFTFSPRRHWTTQDCNCSIYPGHITGHRMAWDMMMNWSPTVALVMAQLLRVPQAILDMIAGAHWGVLAGIAYFSMVGNWAKVLVVLLLFAGVDAQTYVTGGSAARGASGLANLFTPGAKQDIQLINTNGSWHINRTALNCNASLDTGWVAGLFYYHKFNSSGCPERMASCKPLADFDQGWGPIRHANGSGPEHRPYCWHYPPKPCGIVSAQTVCGPVYCFTPSPVVVGTTNRLGVPTYSWGTNDTDVFVLNNTRPPLGNWFGCTWMNSSGFTKVCGAPPCVIGGVGNNTLHCPTDCFRKHPEATYSRCGSGPWITPRCLVHYPYRLWHYPCTVNYTLFKVRMYVGGVEHRLEVACNWTRGERCDLDDRDRSELSPLLLSTTQWQVLPCSFTTLPALTTGLIHLHQNIVDVQYLYGVGSSIVSWAIKWEYVILLFLLLADARICSCLWMMLLISQAEA

SEQ ID NO: 8 - protein, 746 aa, Hepatitis C virus
MSTNPKPQRKTKRNTNRRPQDVKFPGGGQIVGGVYLLPRRGPRLGVRTTRKTSERSEPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETKDTILHSPGCVPCVREGNVSKCWVPVALTVATRDGNLPTTQLRRHIDLLVGSATLCSALYVGDLCGSVFLVGQLFTFSPRRHWTTQDCNCSMYPGHITGHRMAWDMMMNWSPTSALVVAQLLRVPQAILDMIAGAHWGVLAGLAYFSMVGNWAKVLVVLLLFASVDAGTHVTGGSAAHDVSALAGFFRRGAKQNIQLINTNGSWHVNRTALNCNASLDTGWVAGLLYYHRFNSSGCPERMASCRPLADFDQGWGPITNVDGGGSEYRPYCWHYPPKPCGIEPAQNVCGPVYCFTPSPVVVGTTDKVGVPTYNWGENDTDVFVLNNTRPPLGNWFGCTWMNSSGFVKVCGAPPCIIGGAGNKTLHCPTDCFRKHPDATYSRCGSGPWLTPRCLVDYPYRLWHYPCTVNYTLFKIRMYVGGVEHRLVAACNWTYGERCNLDDRDRSELSPLLLSTTQWQVLPCSFTTLPALTTGLIHLHQNIVDVQYLYGVGSSIVSWAIKWEYVILLFLLLADARICSCLWMMLLISQAEA

SEQ ID NO: 9 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREGNASKCWVAVAPTVATRDGTLPTTQLRRHIDLLVGGATLCSALYVGDLCGSVFLVGQLFTFSPRRHWTTQDCNCSIYPGHITGHRMAWDMMMNWSPTTALIVAQLLRVPQAILDMIAGAHWGVLAGIAYFSMVGNWAKVLVVLLLFAGVDAETHVTGGSAAQVTSRVAGFFNPGPKQNVQLINTNGSWHINRTALNCNASLDTGWVAGLFYHYNFNSSGCPERMASCRPLADFDQGWGPISYANGSGPEHRPYCWHYPPKPCGIVPAQNVCGPVYCFTPSPVVVGTTNRLGVPTYNWGSNDTDVFVLNNTRPPLGNWFGCTWMNSSGFTKVCGAPPCVIGGVGNNTLHCPTDCFRKHPEATYSRCGSGPWITPRCLVHYPYRLWHYPCTVNYTLFKVRMYVGGVEHRLEAACNWTRGERCDLDDRDRSELSPLLLSTTQWQVLPCSFTTLPALTTGLIHLHQNIVDVQYLYGVGSSIVSWAIKWEYVILLFLLLADARICSCLWMMLLISQAEA

SEQ ID NO: 10 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRSWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGIYHVTNDCPNSSIVYETADTILHSPGCVPCVREGNASKCWVALAPTVATRDGRLPTTQLRRHIDLLVGSATLCSALYVGDLCGSVFLVGQLFTFSPRRHWTTQDCNCSIYPGHITGHRMAWDMMMNWSPTTALVVAQLLRIPQAILDMIAGAHWGVLAGIAYFSMVGNWAKVLVVLLLFAGVDAHTRVTGGSAARATARLTTLFSPGAKQDIQLINTNGSWHINRTALNCNASLDTGWVAGLFYYHKFNSSGCPERMASCRPLADFDQGWGPISYANGSGPEHRPYCWHYPPKPCGIIPAKTVCGPVYCFTPSPVVVGTTDRSGAPTFNWGDNDTDVLVLNNTRPPLGNWFGCTWMNSSGYTKVCGAPPCIIGGVGNNTLHCPTDCFRKHPEATYSRCGSGPWITPRCLVHYPYRLWHYPCTVNYTLFKVRMYVGGVEHRLEAACNWTRGERCNLDDRDRSELSPLLLSTTQWQVLPCSFTTLPALTTGLIHLHQNIVDVQYLYGVGSSIVSWAIKWEYVILLFLLLADARVCSCLWMMLLISQVEA

SEQ ID NO: 11 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREGNASKCWVAVTPTVATRDGRLPATQLRRHIDLLVGSATLCSALYVGDLCGSIFLVGQLFTFSPRRHWTTQDCNCSIYPGHITGHRMAWDMMMNWSPTTALVVAQLLRVPQAILDMIAGAHWGVLAGIAYFSMVGNWAKVLVVLLLFAGVDAGTHVTGGSAAKDTSGFTSLFRIGARQNIQLINSNGSWHINRTALNCNESLDTGWVAGLLYYHKFNSSGCPERMASCRSLADFDQGWGPISYANGSGPEHRPYCWHYPPKPCGIVPAQSVCGPVYCFTPSPVVVGTTDKSGAPTYNWGCNETDVFVLNNTRPPLGNWFGCTWMNSSGFTKVCGAPPCVIGGVGNNTLHCPTDCFRKHPEATYSRCGSGPWITPRCLVHYAYRLWHYPCTVNYTLFKVRMYVGGVEHRLDVACNWTRGERCNLDDRDRSELSPLLLSTTQWQVLPCSFTTLPALSTGLIHLHQNIVDVQYLYGVGSSIVSWAIKWEYVILLFLLLADARICSCLWMMLLISQAEA

SEQ ID NO: 12 - protein, 746 aa, Hepatitis C virus; identical to SEQ ID NO: 11

SEQ ID NO: 13 - protein, 746 aa, Hepatitis C virus; identical to SEQ ID NO: 11

SEQ ID NO: 14 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADSILHSPGCVPCVREGNASKCWVAVAPTVATRDGKLPATQLRRHIDLLVGSATLCSALYVGDLCGSVFLVSQLFTFSPRRHWTTQDCNCSIYPGHITGHRMAWDMMMNWSPTTALVVSQLLRVPQAILDMIAGAHWGVLAGIAYFSMVGNWAKVLVVLLLFAGVDAETHTTGGSAAYATSGFVGLFRQGAKQNIQLINTNGSWHVNRTALNCNASLDTGWVAGLFYYHKFNSSGCPERLASCKPLANFDQGWGSISYTNGSGPEHRPYCWHYPPKPCGIVPAQNVCGPVYCFTPSPVVVGTTDRLGAPTFNWGENESDVFVLNNTRPPSGNWFGCTWMNSSGFTKVCGAPPCNIGGVGNNTLHCPTDCFRKHPEATYSRCGSGPWVTPRCLVHYPYRLWHYPCTLNYSLFKVRMYVGGIEHRLEVACNWTRGERCNLDDRDRSELSPLLLTTTQWQVLPCSFTTLPALTTGLIHLHQNVVDVQYLYGVGSSIVSWAIKWEYVILLFLLLADARICSCLWMMLLISQAEA

SEQ ID NO: 15 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREGNASKCWVAVAPTVATRDGRLPTTQLRRHIDLLVGSATLCSALYVGDLCGSVFLVGQLFTFSPRHHWTTQDCNCSIYPGHITGHRMAWDMMMNWSPTTALVVAQLLRVPQAILDMIAGAHWGVLAGIAYFSMVGNWAKVLVVLLLFAGVDATTHTTGGAVAHNTRMFTSIFSLGPRQEIQLVNTNGSWHINRTALNCNASLETGWIAGLLYANRFNSSGCPERMASCKPLADFDQGWGPISYANGSGPEHRPYCWHYPPKPCGIVPAQNVCGPVYCFTPSPVVVGTTDRLGTPTYDWGSNDTDVFVLNNTRPPAGNWFGCTWMNSSGYTKVCGAPPCVIGGVSNNTLHCPTDCFRKHPEATYSRCGSGPWITPRCLVHYAYRLWHYPCTVNYTLFKVRMYVGGVEHRLEVACNWTRGERCNLDDRDRSELSPLLLSTTQWQVLPCSFTTLPALTTGLIHLHQNIVDVQYLYGVGSSIVSWAVKWEYVILLFLLLADARICSCLWMMLLISQAEA

SEQ ID NO: 16 - protein, 746 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYGNEGLGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYEVRNVSGVYHVTNDCSNASIVYEAADMIMHTPGCVPCVRENNSSRCWVALTPTLAARNASVPTTTIRRHVDLLVGAAALCSAMYVGDLCGSVFLVAQLFTFSPRRHETVQDCNCSIYPGHVTGHRMAWDMMMNWSPTAALVVSQLLRIPQAVVDMVAGAHWGVLAGLAYYSMVGNWAKVLIVMLLFAGVDGGTYVTGGTMAKNTLGITSLFSPGSSQKIQLVNTNGSWHINRTALNCNDSLNTGFLAALFYVHKFNSSGCPERMASCSPIDAFAQGWGPITYNESHSSDQRPYCWHYAPRPCGIVPAAQVCGPVYCFTPSPVVVGTTDRFGVPTYSWGENETDVLLLNNTRPPQGNWFGCTWMNSTGFTKTCGGPPCNIGGIGNKTLTCPTDCFRKHPEATYTKCGSGPWLTPRCLVHYPYRLWHYPCTVNFTIFKVRMYVGGVEHRLEAACNWTRGERCNLEDRDRSELSPLLLSTTEWQVLPCSFTTLPALSTGLIHLHQNVVDVQYLYGIGSAVVSFAIKWEYVLLLFLLLADARVCACLWMMLLIAQAEA

SEQ ID NO: 17 - protein, 746 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYGNEGMGWAGWLLSPRGSRPSWGPTDPRRRSRNLGKVIDTLTCGFADLMGYIPLVGAPLGGVARALAHGVRVVEDGVNYATGNLPGCSFSIFLLALLSCLTIPASAYEVRNVSGIYHVTNDCSNSSIVYEAADMIMHTPGCVPCVREGNSSRCWVALTPTLAARNASVPTTAIRRHVDLLVGAAAFCSAMYVGDLCGSVFLVSQLFTFSPRRHETIQDCNCSIYPGHVSGHRMAWDMMMNWSPTTALVVSQLLRIPQAIVDMVAGAHWGVLAGLAYYSMVGNWAKVLIVMLLFAGVDGETRVTGGQIARNAYSLTTLFSSGSAQNIQLINTNGSWHINRTALNCNDSLNTGFLAALFYTHKFNASGCPERLASCRPIDKFDQGWGPITYAEQGGQDQRPYCWHYAPKPCGIVSASKVCGPVYCFTPSPVVVGTTDRFGVPTYSWGENETDVLLLNNTRPPQGNWFGCTWMNGTGFTKTCGGPPCNIGGGGNNTLTCPTDCFRKHPAATYTKCGSGPWLTPRCLVDYPYRLWHYPCTANFTIFKVRMYVGGVEHRLDAACNWTRGERCNLEDRDRLELSPLLLSTTEWQVLPCSFTTLPALSTGLIHLHQNIVDVQYLYGIGSAVVSFAIKWDYIVILFLLLADARVCACLWMMLLIAQAEA

SEQ ID NO: 18 - protein, 747 aa, Hepatitis C virus
MSTNPKPQRKTKRNTNRRPQDVKFPGGGQIVGGVYLLPRRGPRLGVRAIRKTSERSQPRGRRQPIPKARRPEGRAWAQPGYPWPLYGNEGMGWAGWLLSPRGSRPSWGPTDPRRRSRNLGKVIDTLTCGLADLMGYIPLVGGPLGGAARALAHGVRVLEDGVNYATGNLPGCSFSIFLLALLSCLTVPASAHEVRNASGVYHVTNDCSNSSIVFEAADLIMHTPGCVPCVREGNSSRCWVALTPTLAARNATIPTTTIRHHVDLLVGAAALCSAMYVGDLCGSVFLVSQLFTFSPRRHATLQDCNCSIYPGHASGHRMAWDMMMNWSPTTALVVSQLLRIPQAVIDMVAGAHWGVLAGLAYYSMAGNWAKVLIVMLLFAGVDGHTLTTGGHAARLTSGFAGLFTPGPSQRIQLINTNGSWHINRTALNCNDSLQTGFLAALFYAHRFNSSGCPERMASCRSIDKFDQGWGPITYAEPTKDPDQRPYCWHYPPQQCGIVPASQVCGPVYCFTPSPVVVGTTDRLGNPTYSWGENDTDVLLLNNTRPPQGNWFGCTWMNSTGFTKTCGAPPCNIGGVGNNTLTCPTDCFRKHPEATYSKCGSGPWLTPRCMVDYPYRLWHYPCTVNFSIFKVRMYVGGVEHRLNAACNWTRGERCNLDDRDRSELSPLLLSTTEWQVLPCSFTTLPALSTGLIHLHQNIVDVQYLYGIGSAVVSFAIKWEYVVLLFLLLADARVCACLWMMLLIAQAEA

SEQ ID NO: 19 - protein, 746 aa, Hepatitis C virus
MSTNPKPQRKTKRNTNRRPQDVKFPGGGQIVGGVYLLPRRGPRLGVRATRKTSERSQPRGRRQPIPKARHPEGRTWAQPGYPWPLYGNEGLGWAGWLLSPRGSRPSWGPTDPRRRSRNLGKVIDTLTCGFADLMGYIPLVGAPLGGVARALAHGVRVLEDGVNYATGNLPGCSFSIFLLALLSCLTIPASAYEVRNVSGVYHVTNDCSNSSIVYEAADVIMHTPGCVPCVQDGNTSRCWVALTPTLAARNASVPVTAIRRHVDLLVGTAAFCSAMYVGDLCGSVFPVSQLFTFSPRRHQTVQDCNCSIYPGHISGHRMAWDMMMNWSPTAALVVSQLLRIPQAIVDMVAGAHWGVLAGLAYYSMVGNWAKVMIVLLLFAGVDGTTHTTGGAAARATQGFTSFFSLGPSQKIQLINTNGSWHINRTALNCNDSLQTGFLAALFYTYRFNASGCPERMASCRPIDKFDQGWGPITYAEPDSSDQRPYCWHYAPRPCGIVPASQVCGPVYCFTPSPVVVGTTDRFGVPTYTWGENETDVLLLNNTRPPLGNWFGCTWMNSTGFTKTCGGPPCNIGGAGNTTLTCPTDCFRKHPEATYTKCGSGPWLTPRCLVDYPYRLWHYPCAVNFTIFKVRMYVGGVEHRLNAACNWTRGERCDLEDRDRSELSPLLLSTTEWQVLPCSFTTLPALSTGLIHLHQNIVDVQYLYGIGSAVIPFAIKWEYVLLLFLLLADARVCACLWMMLLIAQAEA

SEQ ID NO: 20 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGMGWAGWLLSPRGSRPNWGPTDPRRRSRNLGKVIDTLTCGFADLMGYIPLVGAPLGGVARALAHGVRVLEDGVNYATGNLPGCSFSIFLLALLSCLTTPASAYEVRNVSGIYHVTNDCSNSSIVYEAADIIMHTPGCVPCVREKNISRCWVALTPTLAARNISVPTATIRRHVDLLVGTAAFCSAMYVGDLCGSVFLVSQLFTFSPRWHETVQDCNCSLYPGHVSGHRMAWDMMMNWSPTAALVVSQLLRIPQAVVDMVAGAHWGVLAGLAYYSMIGNWAKVLIVMLLFAGADGTTHVTGGVQAHGAYGLASLFNVGPHQKIQLVNTNGSWHINRTALNCNDTLQTGFLAALFYKHRFNASGCPERMASCRPIDKFAQGWGPITYAEPDRLDQRPYCWHYPPRPCGIVPALEVCGPVYCFTPSPVVVGTTDRFGVPTYSWGENETDVLLLNNTRPPQGNWFGCTWMNSTGYTKTCGGPPCNIGGVGNNTLTCPTDCFRKHPEATYTKCGSGPWLTPRCLVHYPYRLWHYPCTVNFTIFKVRMYVGGIEHRLDAACNWTRGERCDLEDRDRSELSPLLLSTTEWQILPCSFTTLPALSTGLIHLHRNIVDVQYLYGIGSAVVSFAIKWEYVLLLFLLLADARVCACLWMMLLIAQAEA

SEQ ID NO: 21 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGLGWAGWLLSPRGSRPSWGPNDPRRRSRNLGKVIDTLTCGFADLMGYIPLVGAPLGGAARALAHGVRVLEDGVNYATGNLPGCSFSIFLLALLSCLTIPASAYEVRNVSGIYHVTNDCSNSSIVYEAADVIMHAPGCVPCVRENNSSRCWVALTPTLAARNASVPTTTLRRHVDLLVGTAAFCSAMYVGDLCGSVFLISQLFTFSPRRHETVQDCNCSIYPGHVSGHRMAWDMMMNWSPTAALVVSQLLRIPQAVMDMVAGAHWGVLAGLAYYSMVGNWAKVLIVMLLFAGVDGHTRVTGGVQGHVTSTLTSLFRPGASQKIQLVNTNGSWHINRTALNCNDSLKTGFLAALFYTHKFNASGCPERMASCRSIDKFDQGWGPITYAQPDNSDQRPYCWHYAPRQCGIVPASQVCGPVYCFTPSPVVVGTTDRFGAPTYNWGDNETDVLLLNNTRPPHGNWFGCTWMNSTGFTKTCGGPPCNIRGVGNNTLTCPTDCFRKHPDATYTKCGSGPWLTPRCLVDYPYRLWHYPCTVNFTIFKVRMYVGGVEHRLDAACNWTRGERCDLEDRDRAELSPLLLSTTEWQILPCSYTTLPALSTGLIHLHQNIVDIQYLYGIGSAVVSIAIKWEYVVLLFLLLADARVCACLWMMLLIAQAEA

SEQ ID NO: 22 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGDEGMGWAGWLLSPRGSRPNWGPTDPRRRSRNLGKVIDTLTCGFADLMGYIPLVGAPLGGVARALAHGVRVLEDGVNYATGNLPGCSFSIFLLALLSCLTTPASAYEVRNVSGIYHVTNDCSNSSIVYEAADIIMHTPGCVPCVREKNTSRCWVALTPTLAARNISVPTTTIRRHVDLLVGTAAFCSAMYVGDLCGSVFLVSQLFTFSPRRHETVQDCNCSLYPGHVSGHRMAWDMMMNWSPTAALVVSQLLRIPQAIVDMVAGAHWGVLAGLAYYSMVGNWAKVLIVMLLFAGADGTTHVTGGVQAHGAYGLASLFNVGPHQKIQLVNTNGSWHINRTALNCNDTLQTGFLAALFYKHRFNASGCPERMASCRPIDKFAQGWGPITYAEPDRLDQRPYCWHYPPRPCGIVPALEVCGPVYCFTPSPVVVGTTDRFGVPTYSWGENETDVLLLNNTRPPQGNWFGCTWMNNTGFTKTCGGPPCNIGGVGNNTLTCPTDCFRKHPEATYTKCGSGPWLTPRCLVDYPYRLWHYPCTVNFTVFKVRMYVGGIEHRLDAACNWTRGERCDLEDRDRSELSPLLLSTTEWQILPCSFTTLPALSTGLIHLHRNIVDVQYLYGIGSAVVSFAIKWEYILLLFLLLADARVCACLWMMLLIAQAEA

SEQ ID NO: 23 - protein, 746 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYGNEGLGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYEVRNVSGVYHVTNDCSNSSIVYEAADMIMHTPGCVPCVREDNSSRCWVALTPTLAARNSSVPTTTIRRHVDLLVGAAAFCSAMYVGDLCGSVFLISQLFTFSPRRYETVQDCNCSLYPGHVSGHRMAWDMMMNWSPTTALVVSQLLRIPQAVVDMVAGAHWGVLAGLAYYSMVGNWAKVLIVMLLFAGVDGNTRVSGGEAAKNTMGFASLFVSGPSQKIQLINTNGSWHINRTALNCDDSLHTGFLAALFYAHKFNSSGCSGRMASCRPIDEFAQGWGPITHGVPDNLDQRPYCWHYAPRPCGIVPASQVCGPVYCFTPSPVVVGTTDRFGAPTYSWGENETDVLLLNNTRPPQGNWFGCTWMNSTGFTKTCGGPPCNIGGVGNNTLTCPTDCFRKHPEATYTKCGSGPWLTPRCMVDYPYRLWHYPCTVNFTIFKVRMYVGGVEHRLDAACNWTRGERCNVEDRDRSELSPLLLSTTEWQILPCSFTTLPALSTGLIHLHQNIVDVQYLYGVGSVVVSVVIRWEYVVLLFLLLADARVCACLWMMLLIAQAEA

SEQ ID NO: 24 - protein, 746 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYGNEGMGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYEVRNVSGMYHVTNDCSNSSIVYEAADMILHAPGCVPCVRENNSSRCWVALTPTLAARNASVPTTAIRRHVDLLVGAAAFCSAMYVGDLCGSVLLVSQIFTFSPRRHETMQDCNCSIYPGHITGHRMAWDMMMNWSPTTALVVSQLLRIPQAIVDMVAGAHWGVLAGLAYYSMVGNWAKVLIVMLLFAGVDGGTYVTGGEAGRRTSGFASIFTPGASQNIQLINTNGSWHINRTALNCNDSLHTGFIAALFYHHKFNASGCPERMASCRPIGEFAQGWGPISYTEPPSSDQRPYCWHYPPRPCGIVPASQVCGPVYCFTPSPVVVGTTDRLGAPTYNWGDNDTDVLLLNNTRPPQGNWFGCTWMNGTGFTKTCGGPPCNIGGVGNNTLTCPTDCFRKHPEATYTKCGSGPWLTPRCIVDYPYRLWHYPCTVNFTITKIRMYVGGVEHRLTAACNWTRGERCDLEDRDRSELSPLLLSTTEWQIMPCSFTTLPALSTGLIHLHQNIVDIQYLYGIGSAAVSFAIRWEYVLLLFLLLADARVCACLWMMLLIAQAEA

SEQ ID NO: 25 - protein, 746 aa, Hepatitis C virus; identical to SEQ ID NO: 24

SEQ ID NO: 26 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRAWAQPGYPWPLYGNEGLGWAGWLLSPRGSRPSWGPTDPRRRSRNLGKVIDTLTCGFADLMGYIPLVGAPLGGTARALAHGVRVLEDGVNYATGNLPGCSFSIFLLALLSCLTIPASAYEVRNVSGVYHVTNDCSNSSIVYETADMIMHTPGCVPCVREDNFTRCWVALTPTLAARNGSVPTTAIRRHVDLLVGAAAFCSAMYVGDLCGSVFLVSQLFTFSPRWHETVQECNCSIYPGHVTGHRMAWDMMMNWSPTTALVVSQLLRIPQAVVDMVAGAHWGVLAGLAYYSMVGNWAKVLIVTLLFAGVDGNTHTIGGKQAQATGGFVAWLARGPSQEIQLINTNGSWHINRTALNCNDSLKTGFIAALFYAHRFNSSGCPERMASCRPIDKFAQGWGPITYAKPDSLDQRPYCWHYAPQPCGIVPASEVCGPVYCFTPSPVVVGTTDRSGVPTYRWGENETDVLLLNNTRPPQGNWFGCTWMNATGFTKTCGGPPCKIGGLGNNTLTCPTDCFRKHPEATYTKCGSGPWLTPRCIVDYPYRLWHYPCTVNFTIFKVRMYVGGIEHRLSAACNWTRGERCDLEDRDRSELSPLLLSTTEWQILPCSFTTLPALSTGLIHLHQNTVDVQYLYGVGSVLVSFAIKWEYILLFFLLLADARVCACLWMMLLIAQAEA

SEQ ID NO: 27 - protein, 746 aa, Hepatitis C virus
MSTNPKPQRKTKRNTNRRPQNVKFPGGGQIVGGVCLLPRRGPRVGVRATRKTSERSQPRGRRQPIPKARRPEGRSWAQPGYPWPLYGNEGCGWAGWLLSPRGSRPSRGPSDPRRRSRNLGKVIDTLTCGFADLMGYIPLVGAPLGGAARALAHGVRVLEDGVNYATGNLPGCSFSIFLLALLSCLTVPASAVEVRNSSGIYHVTNDCPNASVVYETDSLIIHLPGCVPCVREGNASRCWVSLSPTVAAKDPGVPVNEIRRHVDLIVGAAAFCSAMYVGDLCGSIFLVGQLFTLSPRRHWTTQDCNCSIYPGHVTGHRMAWDMMMNWSPTGALVVAQLLRIPQAVLDMIAGAHWGVLAGPAYYSMVGNWAKVLVVLLLFAGVDATTQVTGGTAGRNAYRLASLFSTGPSQNIQLINSNGSWHINRTALNCNDSLHTGWVAALFYSHKFNSSGRPERMASCRPLTAFDQGWGPITYGGKASNDQRPYCWHYAPRPCGIVPAKEVCGPVYCFTPSPVVVGTTDKYGVPTYTWGENETDVLLLNNSRPPIGNWFGCTWMNSTGFTKTCGAPACNVGGSETNTLSCPTDCFRRHPDATYAKCGSGPWLNPRCMVDYPYRLWHYPCTVNYTIFKIRMFVGGIEHRLTAACNWTRGERCDLDDRDRAELSPLLLSTTQWQVLPCSFTTLPALSTGLIHLHQNIVDVQYLYGLSSVVTSWAIRWEYVVLLFLLLADARICACLWMMLLISQVEA

SEQ ID NO: 28 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTYGFADLMGYIPLVGAPLGGAARALAHGVRVLEDGVNYATGNLPGCSFSIFLLALLSCLTVPASAVGVRNSSGVYHVTNDCPNASVVYETDSLIIHLPGCVPCVREGNGSRCWVSLSPTVAAKDPGVPVNEIRRHVDLIAGAAAFCSAMYVGHLCGSIFLVGQLFTLSPRRHWTTQDCNCSIYPGHVTGHRMAWDMMMNWSPTTALVVAQLLRIPQAILDMIAGAHWGVLAGIAYFSMVGNWTKVLVVLLLFAGVDATTIVSGGSAGRSTAGLVGLFSPGARQNIQLINTNGSWHINRTALNCNDTLQTGWVAGLFYTNKFNSSGCPERLASCRPLADFDQGWGPISYTNGSGPDQRPYCWHYPPKPCGIVPAESVCGPVYCFTPSPVVVGTTDRSGAPTYNWGENETDVFVLNNTRPRLGNWFGGTWMNSTGFTKVCGAPPCAIGGVGNNTLYCPTDCFRKHPEATYSRCGSGPWITPRCLIHYPYRLWHYPCTINYTIFKIRMFVGGVEHRLDAACNWTRGERCDLDDRDRAELSPLLLSTTQWQVLPCSFTTLPALSTGLIHLHQNIVDVQYLYGLSSAVTSWVIKWEYVVLLFLLLADARICACLWMMLLISQVEA

SEQ ID NO: 29 - protein, 746 aa, Hepatitis C virus
MSTNPKPQRKTKRNTNRRPQDVKFPGGGQIVGGVYLLPRRGPRVGVRATRKTSERSQPRGRRQPIPKARRPEGRSWAQPGYPWPLYGNEGCGWAGWLLSPRGSRPSWGPSDPRRRSRNLGKVIDTLTCGFADLMGYIPLVGAPLGGAARALAHGVRVLEDGVNYATGNLPGCSFSIFLLALLSCLTVPASAVGVRNSSGVYHVTNDCPNASVVYETENLIMHLPGCVPYVREGNASRCWVSLSPTVAARDSRVPVSEVRRRVDSIVGAAAFCSAMYVGDLCGSIFLVGQIFTFSPRHHWTTQDCNCSIYPGHVTGHRMAWDMMMNWSPTGALVVAQLLRIPQAIVDMIAGAHWGVLAGLAYYSMVGNWAKVVVVLLLFAGVDAETRVTGGAAGHTAFGFASFLAPGAKQKIQLINTNGSWHINRTALNCNESLDTGWLAGLLYYHKFNSSGCPERMASCQPLTAFDQGWGPITHEGNASDDQRPYCWHYALRPCGIVPAKKVCGPVYCFTPSPVVVGTTDRAGVPTYRWGANETDVLLLNNSRPPMGNWFGCTWMNSSGFTKTCGAPACNIGGSGNNTLLCPTDCFRKHPDATYSRCGSGPWLTPRCLVDYPYRLWHYPCTVNYTIFKIRMFVGGVEHRLDAACNWTRGERCDLDDRDRAELSPLLLSTTQWQVLPCSFTTLPALSTGLIHLHQNIVDVQYLYGLSSAVTSWVIKWEYVVLLFLLLADARICACLWMMLLISQVEA

SEQ ID NO: 30 - protein, 750 aa, synthetic construct
  VARIANT 233: note = X can be any amino acid
  VARIANT 249..250: note = X can be any amino acid
  VARIANT 290: note = X can be any amino acid
  VARIANT 378: note = X can be any amino acid
  VARIANT 392: note = X can be any amino acid
  VARIANT 399: note = X can be any amino acid
  VARIANT 445: note = X can be any amino acid
  VARIANT 495: note = X can be any amino acid
  VARIANT 599: note = X can be any amino acid
  VARIANT 628: note = X can be any amino acid
  VARIANT 664: note = X can be any amino acid
  VARIANT 683: note = X can be any amino acid
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPTDPRHRSRNVGKVIDTLTCGFADLMGYIPVVGAPLGGVARALAHGVRVLEDGVNYATGNLPGCSFSIFLLALLSCITVPVSAVEVKNISTSYMVTNDCSNDSITWQLQAAVLHVPGCVPCENDXNTSRCWIPVSPNVAVXXPGALTQGLRTHIDMVVMSATLCSALYVGDLCGGVMLAAQXFIVSPQRHWFVQECNCSIYPGTITGHRMAWDMMMNWSPTATMILAYAMRVPEVIIDIIFGAHWGVMFGLAYFSMQGAWAKVIVILLLXAGVDARTHTVGGSXGRTTSGXAGLFSSGPKQNIQLINTNGSWHINRTALNCNDSLQTGFIASLFYTXNFNSSGCPERLSACRGIEAFRIGWGTLQYEDNVTNPEDMRPYCWHYPPKXCGIVPARSVCGPVYCFTPSPVVVGTTDRLGVPTYTWGENETDVFLLNSTRPPRGSWFGCTWMNSTGFTKTCGAPPCRIRADFNASTDLLCPTDCFRKHPDATYXKCGSGPWLTPRCLVDYPYRLWHYPCTVNXTIFKIRMYVGGVEHRLTAACNFTRGDRCNLEDRDRXQLSPLLHSTTEWAILPCSXSDLPALSTGLLHLHQNIVDVQYMYGLSPALTKYIVRWEWVVLLFLLLADARVCACLWMLILLGQAEA

SEQ ID NO: 31 - protein, 750 aa, Hepatitis C virus
MSTNPKPQRKTKRNTNRRPEDVKFPGGGQIVGGVYLLPRRGPRLGVRTTRKTSERSQPRGRRQPIPKDRRSTGKAWGKPGRPWPLYGNEGLGWAGWLLSPRGSRPSWGPTDPRHRSRNVGKVIDTLTCGFADLMGYIPVVGAPLSGAARAVAHGVRVLEDGVNYATGNLPGFPFSIFLLALLSCITVPVSAAQVKNTSSSYMVTNDCSNDSITWQLEAAVLHVPGCVPCERVGNTSRCWVPVSPNMAVRQPGALTQGLRTHIDMVVMSATFCSALYVGDLCGGVMLAAQVFIVSPQYHWFVQECNCSIYPGTITGHRMAWDMMMNWSPTATMILAYVMRVPEVIIDIVSGAHWGVMFGLAYFSMQGAWAKVIVILLLAAGVDAGTTTVGGAVARSTNVIAGVFSHGPQQNIQLINTNGSWHINRTALNCNDSLNTGFLAALFYTNRFNSSGCPGRLSACRNIEAFRIGWGTLQYEDNVTNPEDMRPYCWHYPPKPCGVVPARSVCGPVYCFTPSPVVVGTTDRRGVPTYTWGENETDVFLLNSTRPPQGSWFGCTWMNSTGFTKTCGAPPCRTRADFNASTDLLCPTDCFRKHPDATYIKCGSGPWLTPKCLVHYPYRLWHYPCTVNFTIFKIRMYVGGVEHRLTAACNFTRGDRCDLEDRDRSQLSPLLHSTTEWAILPCTYSDLPALSTGLLHLHQNIVDVQYMYGLSPAITKYVVRWEWVVLLFLLLADARVCACLWMLILLGQAEA

SEQ ID NO: 32 - protein, 749 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPTDPRHRSRNVGKVIDTLTCGFADLMGYIPVVGAPLGGVARALAHGVRVLEDGVNYATGNLPGCSFSIFLLALLSCITTPVSAAQVKNTSDIYMVTNDCSNDSITWQLQAAALHVPGCVPCERVGNTSRCWIPVSPNVAVRQPGALTQGLRTHIDMVVMSATLCSALYVGDLCGGVMLAAQMFIISPQHHWFVQECNCSIYPGTITGHRMAWDMMMNWSPTTTMILAYVMRVPEVIIDIIGGAHWGVMFGLAYFSMQGAWAKVVVILLLAAGVDAHTRTGSSVGYATSGIVGLFTSGPKQNIQLINTNGSWHINRTALNCNDSLNTGFIVSLFYARNFNSTGCPERLSACRGIEGFRIGWGTLQYEDNVTNPEDMRPYCWHYPPKQCGIVPAGSVCGPVYCFTPSPVVVGTTDRLGVPTYTWGENETDVFLLNSTRPPVGSWFGCTWMNSTGFTKTCGAPPCRIRADFNASTDLLCPTDCFRKHPEATYIKCGSGPWLTPRCLVDYPYRLWHYPCTVNYSIFKIRMYVGGVEHRLTAACNFTRGDRCNLEDRDRSQLTPLLHSTTEWAILPCTYSDLPALSTGLLHLHQNIVDVQYMYGLSPALTKYVVRWEWVVLLFLLLADARVCACVWMLILLGQAEA

SEQ ID NO: 33 - protein, 750 aa, Hepatitis C virus
  VARIANT 458: note = X can be any amino acid
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPTDPRHRSRNVGKVIDTLTCGFADLMGYIPVVGAPLGGVARALAHGVRVLEDGVNYATGNLPGCSFSIFLLALLSCVTVPVSAVQVKNTSETYMVTNDCSNDSITWQLQAAVLHVPGCVPCERTGNTSRCWIPVSPNVAVQQPGALTQGLRTHIDMVVMSATLCSALYVGDLCGGVVLAAQLFIVSPRRHWFVQECNCSIYPGAITGHRMAWDMMMNWSPTTTMVLAYAMRVPEVIIDIISGAHWGVMFGLAYFSMQGAWAKVAVILLLTAGVEARTHTTGSVAGRTTSGFAGIFTSGPKQNIQLINTNGSWHINRTALNCNDSLNTGFMAALFYTKNFNSSGCPERLSXCRNIEAFRIGWGTLQYEDDVTNPEDMRPYCWHYPPKQCGIFPAGSVCGPVYCFTPSPVVVGTTNKLGVPTYTWGENETDVFILNSTRPPRGSWFGCTWMNSTGFTKTCGAPPCRIRADFNASTDLLCPTDCFRKHPEATYIKCGSGPWLTPRCLVDYPYRLWHYPCTVNYSIFKIRMYVGGVEHRLTAACNFSRGDRCNLEDRDRSQLTPLLHSTTEWAILPCSYSDLPALSTGLLHLHQNIVDVQYMYGLTPALTKYVVRWEWVVLLFLLLADARVCACLWMLILLGQAEA

SEQ ID NO: 34 - protein, 750 aa, Hepatitis C virus
MSTNPKPQRKTKRNTSRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPTDPRHRSRNVGKVIDTLTCGFADLMGYIPVVGAPLGGVARALAHGVRVLEDGVNYATGNLPGCSFSIFLLALLSCITVPVSAVQVKNISDSYMVTNDCSNDSITWQLQAAVLHVPGCVPCEKMGNISRCWIPVSPNVAVQRPGALTQGLRAHIDMVVMSATLCSALYVGDLCGGVMLAAQMFIVSPQHHHFVQECNCSIYPGAITGHRMAWDMMMNWSPTATMILAYAMRVPEVIIDIISGAHWGVMFGLAYFSMQGAWAKVVVILLLTAGVDAHTRSIAGSVAHATSGLAGLFTSGAKQNIQLINTNGSWHINRTALNCNDSLNTGFIASLFYTYRFNSSGCPERLSACRGIQAFRIGWGTLRYEDNVTNPEDMRPYCWHYPPKQCGIVSARSVCGPVYCFTPSPVVVGTTDRLGVPTYTWGENETDVFILNSTRPPGGSWFGCTWMNSTGFTKTCGAPPCRIRADFNASMDLLCPTDCFRKHPDATYIKCGSGPWLTPRCLVDYPYRLWHYPCTINYTIFKIRMYVGGVEHRLTAACNFTRGDPCNLEDRDRSQLSPLLHSTTEWAILPCSYSDLPALSTGLLHLHQNIVDVQYMYGLSPALTKYIVRWEWVVLLFLLLADARVCACLWMLILLGQAEA

SEQ ID NO: 35 - protein, 751 aa, Hepatitis C virus
MSTNPKPQRKTQRNTNRRPQDVKFPGGGQIVGGVYLLPRRGPRLGVRTARKTSERSQPRGRRQPIPKDRRSTGKSWGRPGYPWPLYGNEGLGWAGWLLSPRGSRPSWGPTDPRHRSRNVGKVIDTLTCGFADLMGYIPVVGAPLGGVARALAHGVRVLEDGVNYATGNLPGCSFSIFLLALLSCITIPASAVEVKNTSTGYMVTNDCANSSITWQLHAAVLHVPGCVPCERVDNNTSRCWIPVSPNIAVQRPGALTQGLRSHIDIVVMSATLCSALYVGDLCGGVMLAAQMFVVSPEHHWFVQECNCSIYPGTITGHRMAWDMMMNWSPTATMILAYAMRVPEVIIDIIGGAHWGVMFGLAYFSMQGAWAKVVVILLLAAGVDAYTHTVGGAAASTANSIAGLLSRGPRQNLQLINSNGSWHINRTALNCHDSLQTGFITALFYARHFNSSGCPERLAACRNIEAFRVGWGALQYEDNVTNPEDMRPYCWHYPPKQCGIVPARSVCGPVYCFTPSPVVVGTTDKLGVPTYTWGENETDVFLLNSTRPPQGPWFGCTWMNSTGFTKTCGAPPCRTRADFNASTDLLCPTDCFRKHPDATYNKCGSGPWLTPRCLIDYPYRLWHYPCTVNYTTFKIRMYVGGVEHRLMAACNFTRGDSCDLSQRDRGQLSPLLHSTTEWAILPCSFSDLPALSTGLLHLHQNIVDVQYMYGLSPALTKYIVRWEWVVLLFLLLADARVCACIWMLILLGQAEA

SEQ ID NO: 36 - protein, 750 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPNDPRHRSRNVGKVIDTLTCGFADLMGYIPVVGAPLGGVARALAHGVRVLEDGVNFATGNLPGCSFSIFLLALLSCITTPVSAAEVKNISTGYMVTNDCTNDSITWQLQAAVLHVPGCVPCEKVGNTSRCWIPVSPNVAVQQPGALTQGLRTHIDMVVMSATLCSALYVGDLCGGVMLAAQMFIVSPQHHWFVQDCNCSIYPGTITGHRMAWDMMMNWSPTATMILAYAMRVPEVIIDIIGGAHWGVMFGLAYFSMQGAWAKVVVILLLAAGVDAQTHTVGGSTAHNARTLTGMFSLGARQKIQLINTNGSWHINRTALNCNDSLHTGFLASLFYTHSFNSSGCPERMSACRSIEAFRVGWGALQYEDNVTNPEDMRPYCWHYPPRQCGVVSASSVCGPVYCFTPSPVVVGTTDRLGAPTYTWGENETDVFLLNSTRPPQGSWFGCTWMNSTGYTKTCGAPPCRIRADFNASMDLLCPTDCFRKHPDTTYIKCGSGPWLTPRCLIDYPYRLWHYPCTVNYTIFKIRMYVGGVEHRLTAACNFTRGDRCNLEDRDRSQLSPLLHSTTEWAILPCTYSDLPALSTGLLHLHQNIVDVQFMYGLSPALTKYIVRWEWVVLLFLLLADARVCACLWMLILLGQAEA

SEQ ID NO: 37 - protein, 750 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPNDPRHRSRNVGKVIDTLTCGFADLMGYIPVVGAPLGGVARALAHGVRVLEDGVNFATGNLPGCSFSIFLLALLSCITTPVSAAEVKNISTGYMVTNDCTNDSITWQLQAAVLHVPGCVPCEKVGNASQCWIPVSPNVAVQRPGALTQGLRTHIDMVVMSATLCSALYVGDLCGGVMLAAQMFIVSPQHHWFVQDCNCSIYPGTITGHRMAWDMMMNWSPTATMILAYAMRVPEVIIDIISGAHWGVMFGLAYFSMQGAWAKVVVILLLAAGVDARTHTVGGSAAQTTGRLTSLFDMGPRQKIQLVNTNGSWHINRTALNCNDSLHTGFIASLFYTHSFNSSGCPERMSACRSIEAFRVGWGALQYEDNVTNPEDMRPYCWHYPPRQCGVVSAKTVCGPVYCFTPSPVVVGTTDRLGAPTYTWGENETDVFLLNSTRPPLGSWFGCTWMNSSGYTKTCGAPPCRTRADFNASTDLLCPTDCFRKHPDTTYLKCGSGPWLTPRCLIDYPYRLWHYPCTVNYTIFKIRMYVGGVEHRLTAACNFTRGDRCNLEDRDRSQLSPLLHSTTEWAILPCSYSDLPALSTGLLHLHQNIVDVQFMYGLSPALTKYIVRWEWVILLFLLLADARVCACLWMLILLGQAEA

SEQ ID NO: 38 - protein, 750 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGCGWAGWLLSPRGSRPTWGPTDPRHRSRNLGRVIDTITCGFADLMGYIPVVGAPVGGVARALAHGVRVLEDGINYATGNLPGCSFSIFLLALLSCVTVPVSAVEVRNISSSYYATNDCSNNSITWQLTDAVLHLPGCVPCENDNGTLHCWIQVTPNVAVKHRGALTRSLRTHVDMIVMAATACSALYVGDVCGAVMILSQAFMVSPQRHNFTQECNCSIYQGHITGHRMAWDMMLSWSPTLTMILAYAARVPELVLEIIFGGHWGVVFGLAYFSMQGAWAKVIAILLLVAGVDATTYSSGQEAGRTVAGFAGLFTTGAKQNLYLINTNGSWHINRTALNCNDSLQTGFLASLFYTHKFNSSGCPERLSSCRGLDDFRIGWGTLEYETNVTNDGDMRPYCWHYPPRPCGIVPARTVCGPVYCFTPSPVVVGTTDKQGVPTYTWGENETDVFLLNSTRPPRGAWFGCTWMNGTGFTKTCGAPPCRIRKDYNSTIDLLCPTDCFRKHPDATYLKCGAGPWLTPRCLVDYPYRLWHYPCTVNFTIFKARMYVGGVEHRFSAACNFTRGDRCRLEDRDRGQQSPLLHSTTEWAVLPCSFSDLPALSTGLLHLHQNIVDVQYLYGLSPALTRYIVKWEWVILLFLLLADARICACLWMLIILGQAEA

SEQ ID NO: 39 - protein, 750 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGCGWAGWLLSPRGSRPHWGPTDPRHRSRNLGKVIDTITCGFADLMGYIPVIGAPVGGVARALAHGVRVLEDGVNYATGNLPGCSFSIFLLALLSCVTVPVSAVEVRNISSSYYATNDCSNNSITWQLENAVLHLPGCVPCENDNGTLRCWTQVTPNVAVKHRGALTQNLRTHVDVIVVAATVCSALYVGDVCGAVMIASQALIVSPARHNFTQECNCSIYQGRITGHHMAWDMMLNWSPTITMILAYAARIPELVLEVIFGGHWGVMFGLAYFSMQGAWAKVIVILLLVAGVDARHHTTGLQAGKTLARVTSLFSIGAKQNIQLINTNGSWHINRTALNCNDSLQTGFIASLFYVNNINSSGCPERMSSCRELDDFRIGWGTLEYETNVTNDEDMRPYCWHYPPKPCGIVPARTVCGPVYCFTPSPIVVGTTDKQGVPTYSWGENETDVFLLNSTRPPRGSWFGCTWMNGTGFTKTCGAPPCRIRRDYNSTLDLLCPTDCFRKHPDTTYLKCGSGPWLTPKCLVEYPYRLWHYPCTVNFTIFKVRMYVGAVEHRFSAACNFTRGDRCRLEDRDRGQQSPLLHSTTEWAVLPCSFSDLPALSTGLLHLHQNIVDVQYLYGLSPAITRYIVKWEWVVLLFLLLADARVCACLWMLIILGQAEA

SEQ ID NO: 40 - protein, 750 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGCGWAGWLLSPRGSRPTWGPTDPRHRSRNLGRVIDTITCGFADLMGYIPVVGAPVGGVARALAHGVRVLEDGINYATGNLPGCSFSIFLLALLSCCTVPVSAVEVRNISTSYYATNDCSNTSITWQLTNAVLHLPGCVPCENDNGTLRCWIQVTPNVAVKHRGALTHNLRTHVDVIVMAATVCSALYVGDICGAVMIVSQAFIISPERHNFTQECNCSMYQGHITGHRMAWDMMLNWSPTLTMILAYAARVPELVLEVIFGGHWGVVFGLAYFSMQGAWAKVIAILLLVAGVDANTYSSGVTVGHTTSTFANIFSVGPSQKINLINTNGSWHINRTALNCNDSLQTGFLASLFYVRNFNSSGCRERLSSCRRLDDFRIGWGTLEYETNVTNDEDMRPYCWHYPPKPCGIVSARTVCGPVYCFTPSPVVVGTTDRQGVPTYSWGENETDVFLLNSTRPPRGAWFGCTWMNGTGFTKTCGAPPCRIRRDYNSTLDLLCPTDCFRKHPDTTYLKCGAGPWLTPKCLVDYPYRLWHYPCTVNFTIFKVRMYVGGVEHRLSAACNFTRGDRCGLEDRDRGQQSPLLHSTTEWAVLPCSFSDLPALSTGLLHLHQNIVDVQYLYGLSPAITRYIVKWEWVVLLFLLLADARVCACLWMLIILGQAEA

SEQ ID NO: 41 - protein, 750 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGCGWAGWLLSPRGSRPTWGPSDPRHRSRNLGRVIDTITCGFADLMGYIPVVGAPVGGVARALAHGVRVLEDGINYATRNLPGCSFSIFLLALLSCVTVPVSSVEIRNISTSYYATNDCSNNSITWQLTNAVLHLPGCVPCENDNGTLRCWIQVTPNVAVKHRGALTHNLRAHVDVIVMAATVCSALYVGDVCGAVMIVSQALIVSPERHNFTQECNCSIYQGHITGQRMAWDMMLNWSPTLTMILAYAARVPELVLEIVFGGHWGVVFGLAYFSMQGAWAKVIAILLLVAGVDATTYSTGATVGRTVGSFAGLFKLGAQQNVQLINTNGSWHINRTALNCNDSLHTGFMAALFYANKFNSSGCPERLSSCRGLDDFRIGWGTLEYETNVTNVEDMRPYCWHYPPKPCGIVPAQSVCGPVYCFTPSPVVVGTTDRQGVPTYNWGDNETDVFLLNSTRPPRGAWFGCTWMNGTGFTKTCGAPPCRIRKDFNSTLDLLCPTDCFRKHPDATYVKCGAGPWLTPRCLIDYPYRLWHYPCTVNFTIFKVRMYVGGVEHRFSAACNFTRGDRCRLEDRDRGQQSPLLHSTTEWAVLPCSFSDLPALSTGLLHLHQNIVDVQYLYGLSPAVTKYIVKWEWVVLLFLLLADARICACLWMLIILGQAEA

SEQ ID NO: 42 - protein, 750 aa, Hepatitis C virus
  VARIANT 203: note = X can be any amino acid
  VARIANT 295: note = X can be any amino acid
  VARIANT 343: note = X can be any amino acid
  VARIANT 401: note = X can be any amino acid
  VARIANT 426: note = X can be any amino acid
  VARIANT 626: note = X can be any amino acid
MSTDPKPQRKTKRNTNRRPQDVKFPGGGQIVGGVYLLPRRGPRLGVRAARKTSERSQPRGRRQPIPKDRRSPGKSWGKPGYPWPLYGNEGCGWAGWLLSPRGSRPTWGPTDPRHRSRNLGKVIDTITCGFADLMGYIPVVGAPVGGVARALAHGVRVLEDGINYATGNLPGCSFSIFLLALLSCLTVPASAVEVRNISSGYYXTNDCSNSSITWQLTNAVLHLPGCVPCENDNGTLRCWIQVTPNVAVKYRGALTHNLRTHVDMIVMAATVCSALYVGDVCGAVMIVSQAFIMSXERHNFTQECNCSIYQGHITGHRMAWDMMLGWSPTLTMILAYAARVPEXVLEVVFGGHWGVVFGLAYFSMQGAWAKVIAILLLVAGVDAGTYSSGATIGQGTRGLVXLFSAGPSQKISLINTNGSWHINRTXLNCNDSLQTGFIASLFYAKSFNSSGCPERLSSCRGLDDFRIGWGTLEYENNVTNDEDMRPYCWHYPPKPCGIVPARTVCGPVYCFTPSPVVVGTTDKQGVPTYSWGENETDVFLLNSTRPPQGAWFGCTWMNGTGFTKTCGAPPCRIRRDHTSTLDLLCPTDCFRKHPDTTYLKCGAGPWLTPKCLVDYPYRLWHYPCTXNFTIFKVRMYVGGVEHRFSAACNFTRGDRCRLEDRDRGQQSPLLHSTTEWAVLPCSFSDLPALSTGLLHLHQNIVDVQYLYGLSPAITKYIVKWEWVILLFLLLADARVCACLWMLIILGQAEA

SEQ ID NO: 43 - protein, 752 aa, Hepatitis C virus
  VARIANT 431: note = X can be any amino acid
  VARIANT 466: note = X can be any amino acid
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLEWRNTSGLYVLTNDCSNSSIVYEADDVILHTPGCVPCVQDGNTSTCWTPVTPTVAVRYVGATTASIRSHVDLLVGAATMCSALYVGDMCGAVFLVGQAFTFRPRRHQTVQTCNCSLYPGHLSGHRMAWDMMMNWSPAVGMVVAHVLRLPQTLFDIIAGAHWGILAGLAYYSMQGNWAKVAIIMVMFSGVDATTYTTGGSAARGARGLTSLFSVGAKQKLQLVNTNGSWHINSTALNCNXSINTGFIAGLFYYHKFNSTGCPQRLSSCKPITFFXQGWGPLTDANITGPSDDKPYCWHYAPRPCDVVPASSVCGPVYCFTPSPVVVGTTDAKGVPTYTWGENETDVFLLESLRPPSGRWFGCTWMNSTGFVKTCGAPPCNIYGGGGNPHNESDLFCPTDCFRKHPEATYSRCGAGPWLTPRCMVDYPYRLWHYPCTVNFTLFKVRMFVGGFEHRFTAACNWTRGERCDIEDRDRSEQHPLLHSTTELAILPCSFTPMPALSTGLIHLHQNIVDVQYLYGVGSGMVGWALKWEFVILVFLLLADARVCVALWLMLMISQAEA

SEQ ID NO: 44 - protein, 752 aa, Hepatitis C virus
MSTLPKPQRKTKRNTIRRPQDVKFPGGGQIVGGVYVLPRRGPRLGVRATRKTSERSQPRERRQPIPKARRSDGRSWAQPGYPWPLYGNEGCGWAGWLLSPRGSRPSWGPNDPRRRSRNLGKVIDTLTCGFADLMGYIPIVGAPVGGVARALAHGVRALEDGINFATGNLPGCSFSIFLLALLSCLIHPAASLEWRNTSGLYILTNDCPNSSIVYEADDVILHTPGCIPCVQDGNTSTCWTSVSPTVAVRYVGATTASIRSHVDLLVGAATLCSALYVGDMCGAVFLVGQAFTFRPRRHQTVQTCNCSLYPGHLTGHRMAWDMMMNWSPAVGMVVAHVLRMPQTVFDIIAGAHWGILAGLAYYSMQGNWAKVAIIMVMFSGVDAETHTTGGTAARNAFTLTGLFTQGARQKLELINTNGSWHINRTALNCNESLNTGFIAGLFYLHKFNSTGCPERLSSCKPITFFRQGWGSLTDANITGPSDDKPYCWHYAPRPCEVVPALNVCGPVYCFTPSPVVVGTTDRQGVPTYTWGENETDVFLLRSLRPPSGQWFGCTWMNSTGFVKTCGAPPCDIYGGGGNRCNESDLFCPTDCFRKHPEATYSRCGAGPWLTPRCLVDYPYRLWHYPCTVNFTLFKVRMFVGGFEHRFTAACNWTRGERCNIEDRDRSEQHPLLHSTTELAILPCSFTPMPALSTGLIHLHQNIVDVQYLYGVGSGVVGWALRWEFVVLVFLLLADARVCVALWLMLMISQAEA

SEQ ID NO: 45 - protein, 752 aa, Hepatitis C virus
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLVHPAASLEWRNTSGLYVLTNDCSNSSIVYEADDVILHTPGCVPCVQDDNTSTCWTPVTPTVAVRYVGATTASIRSHVDLLVGAATLCSALYVGDMCGAVFLVGQAFTFRPRRHQTVQTCNCSLYPGHVSGHRMAWDMMMNWSPAVGMVVAHILRLPQTLFDILAGAHWGILAGLAYYSMQGNWAKVAIVMIMFSGVDAETYVTGGSVAHSARGLTSLFSMGAKQKLQLVNTNGSWHINSTALNCNESINTGFIAGLFYYHKFNSTGCPQRLSSCKPIISFRQGWGPLTDANITGPSDDRPYCWHYAPRPCSVVPASSVCGPVYCFTPSPVVVGTTDIKGKPTYNWGENETDVFLLESLRPPSGRWFGCAWMNSTGFLKTCGAPPCNIYGGEGDPENETDLFCPTDCFRKHPEATYSRCGAGPWLTPRCMVDYPYRLWHYPCTVNFTLFKVRMFVGGFEHRFTAACNWTRGERCNIEDRDRSEQHPLLHSTTELAILPCSFTPMPALSTGLIHLHQNIVDVQYLYGVGSDMVGWALKWEFVILVFLLLADARVCVALWLMLMVSQAEA

SEQ ID NO: 46 - protein, 752 aa, Hepatitis C virus
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLEWRNTSGLYVLTNDCSNSSIVYEADDVILHTPGCVPCVQDGNTSTCWTPVTPTVAVRYVGATTASIRSHVDLLVGAATMCSALYVGDMCGAVFLVGQAFTFRPRRHQTVQTCNCSLYPGHLSGHRMAWDMMMNWSPAVGMVVAHVLRLPQTLFDIMAGAHWGILAGLAYYSMQGNWAKVAIIMVMFSGVDAHTYTTGGTASRHTQAFAGLFDIGPQQKLQLVNTNGSWHINSTALNCNESINTGFIAGLFYYHKFNSTGCPQRLSSCKPITFFRQGWGPLTDANITGPSDDRPYCWHYAPRPCDIVPASSVCGPVYCFTPSPVVVGTTDARGVPTYTWGENEKDVFLLKSQRPPSGRWFGCSWMNSTGFLKTCGAPPCNIYGGEGNPHNESDLFCPTDCFRKHPETTYSRCGAGPWLTPRCMVDYPYRLWHYPCTVDFRLFKVRMFVGGFEHRFTAACNWTRGERCDIEDRDRSEQHPLLHSTTELAILPCSFTPMPALSTGLIHLHQNIVDVQYLYGVGSGMVGWALKWEFVILVFLLLADARVCVALWLMLMISQTEA

SEQ ID NO: 47 - protein, 752 aa, Hepatitis C virus
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGQNDPRRRSRNLGKVIDTLTCGFADLMGYIPLVGAPVGGVARALAHGVRALEDGINFATGNLPGCSFSIFLLALFSCLIHPAASLEWRNTSGLYVLTNDCSNSSIVYEADDVILHTPGCVPCVQDGNTSTCWTPVTPTVAVRYVGATTASIRSHVDLLVGAATMCSALYVGDMCGAVFLVGQAFTFRPRRHQTVQTCNCSLYPGHLSGHRMAWDMMMNWSPAVGMVVSHVLRLPQTLFDIIAGAHWGILAGLAYYSMQGNWAKVAVIMVMFSGVDAETYITGGSAAHGVSTLTSLFSSGPQQKLQLVKTNGSWHINSTALNCNESINTGFIAGLFYYHKFNSTGCPQRLSSCKPITFFRQGWGSLTDANVTGASADKPYCWHYAPRPCDVVPALNVCGPVYCFTPSPVVVGTTDRKGVPTYNWGENESDVFLLESLRPPSGRWFGCAWMNSTGFLKTCGAPPCNIYGGGGNPNNESHLFCPTDCFRKHPDATYSRCGAGPWLTPRCMVDYPYRLWHYPCTVNFTLFKVRMFVGGFEHRFTAACNWTRGERCNIEDRDRSEQHPLLHSTTELAILPCSFTPMPALSTGLIHLHQNIVDVQYLYGVGSGMVGWALKWEFVILIFLLLADARVCVALWLMLMISQAEA

SEQ ID NO: 48 - protein, 752 aa, Hepatitis C virus
MSTLPKPQRKTKRNTIRRPQDVKFPGGGQIVGGVYVLPRRGPRLGVCATRKTSERSQPRRRRQPIPKARQSGGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLEWRNTSGLYVLTNDCSNSSIVYEADDVILHTPGCVPCVQNDNISTCWTPVTPTVAVRYVGATTASIRSHVDLLVGAATMCSALYVGDMCGAVFLVGQAFTFRPRRHQTVQTCNCSLYPGHLSGHRMAWDMMMNWFPALGMAVAHVLRVPQTLFDIIAGAHWGILAGLAYYSMQGNWAKVAIIMVMFSGVDAVTYTTGGSAAHATRGLTSLFSVGAQQKLQLVNTNGSWHINSTALNCNESINTGFIAGLFYYHRFNSTGCPQRLSSCKPITFFKQGWGPLTDANISGPSDDKPYCWHYAPRPCKVVPASGVCGPVYCFTPSPVVVGTTDAKGVPTYTWGANDTDVFLLESLRPPGGRWFGCTWMNSTGFVKTCGASPCDIYGGGGNSGNESDLFCPTDCFRKHPEATYSRCGAGPWLTPRCMVDYPYRLWHYPCTVNFTLFKVRMFVGGFEHRFTAACNWTRGERCDIEDRDRSEQHPLLHSTTELAILPCSFTPMPALSTGLIHLHQNIVDVQYLYGVGSGMVGWALKWEFVILIFLLLADRRVCVALWLMLMITQAEA

SEQ ID NO: 49 - protein, 752 aa, Hepatitis C virus
MSTLPKPQRKTKRNTVCRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLEWRNVSGLYILTNDCPNSSIVYEADDVILHTPGCIPCVQDGNTSTCWTALTPTVAVRYVGATTASIRSHVDLLVGAATMCSALYVGDMCGAVFLVGQAFTFRPRRHQTVQTCNCSLYPGHLTGHRMAWDMMMNWSPAVGMVVAHVLRLPQTLFDIIAGAHWGILAGLAYYSMQGNWAKVIIIMVMFSGVDATTHVTGGTAGLTAFRLTGLFTVGPQQKLQLVNTNGSWHINRTALNCNDSLNTGFIAGLFRFHKFNSTGCPEMLSSCKPITSFKQGWGPLTDANITIPSDDRPYCWHYPPRSCEVVPALSVCGPVYCFTPSPVVVGTTDAKGVPTYTWGENETDVFLLKSLRPPGGRWFGCTWMNSTGFVQTCGAPPCNIYGGGGDLKNESDLFCPTDCFRKHPEATYSRCGAGPWLTPRCMVDYPYRLWHYPCTVNFTLFKVRMFVGGFEHRFTAACNWTRGERCDIEDRDRSELHPLLHSTTELAILPCSFTPMPALSTGLIHLHQNIVDVQYLYGVGSGMVGWAVKWEFVILVFLLLADARVCVALWLMLMISQAEA

SEQ ID NO: 50 - protein, 752 aa, Hepatitis C virus
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLQWRNTSGLYVLTNDCSNSSIVYEADDVILHTPGCVPCVQDGNTSTCWTPVTPTVAVRYVGATTASIRSHVDLLVGAATMCSALYVGDMCGAVFLVGQAFTFRPRRHQTVQTCNCSLYPGHLSGHRMAWDMMMNWSPAAGMVVAHILRLPQTLFDIIAGAHWGILAGLAYYSMQGNWAKVAIIMVMFSGVDATTYTSGGSVAQQARGLADLFSVGAKQNLQLVNTNGSWHINSTALNCDDSINTGFIAGLFYYHKFNSTGCPQRLSDCKPITFFKQGWGPLTDANITGPSDDKPYCWHYAPRRCGVVPASSVCGPVYCFTPSPVVVGTTDAKGVPTYTWGANETDVFLLESLRPPSGRWFGCAWMNSTGFLKTCGAPPCNIYGGGGNPHNESDLFCPTDCFRKHPEATYSRCGAGPWLTPRCMVDYPYRLWHYPCTVNFTLFKMRTFVGGFEHRFTAACNWTRGERCDIEDRDRSEQHPLLHSTTELAILPCSFTPMPALSTGLIHLHQNIVDVQYLYGVGSGMVGWALKWEFVILVFLLLADARVCVALWLMLMISQAEA

SEQ ID NO: 51 - protein, 752 aa, Hepatitis C virus; identical to SEQ ID NO: 50

SEQ ID NO: 52 - protein, 752 aa, Hepatitis C virus
MSTLPKPQRKTKRNTIRRPQDVKFPGGGQIVGGVYVLPRRGPRLGVCATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLVHPAASLEWRNTSGLYVLTNDCPNSSIVYEADDVILHTPGCIPCVQDGNKSTCWTSVTPTVAVKYVGATTASIRSHVDLLVGAATMCSALYVGDMCGAVFLVGQAFTFRPRRHQTVQTCNCSLYPGHLSGHRMAWDMMMNWSPAVGMVVAHVLRLPQTLFDIIAGAHWGILAGLAYYSMQGNWAKVAIIMVMFSGVDATTYTTGGNAARGASGIVSLFTPGAKQNLQLVNTNGSWHINRTALNCNDSINTGFIAGLIYYHKFNSTGCPQRLSSCKPITFFRQGWGSLTDANITGPSDDKPYCWHYPPRPCDTIRASSVCGPVYCFTPSPVVVGTTDAKGAPTYNWGANETDMFLLQSLRPPSGRWFGCTWMNSTGFTKTCGAPPCNIYGGGGNLNNESDLFCPTDCFRKHPEATYSRCGAGPWLTPRCLVDYPYRLWHYPCTVNFTLFRMRTFVGGFEHRFTAACNWTRGERCNIEDRDRSEQHPLLHSTTELAILPCSFTPMPALSTGLIHLHQNIVDVQYLYGIGSGVVGWALKWEFVILVFLLLADARVCVALWLMLMISQAEA

SEQ ID NO: 53 - protein, 752 aa, Hepatitis C virus
MSTLPKPQRKTKRNTIRRPQDVKFPGGGVIYVGVYVLPRRGPRLGVRATRKTSERSQPRGRRKPIPKARRSEGRSWAQPGYPWPLYGNEGCGWAGWLLSPRGSRPNWAPNDPRRRSRNLGKVIDTLTCGFADLMGYIPLVGAPLGGAARALAHGVRALEDGINFATGNLPGCSFSIFLLALFSCLIHPAASLEWRNTSGLYVLTNDCSNSSIVYEADDVILHTPGCIPCVQDGNTSTCWTPVTPTVAVRYVGATTASIRSHVDLLVGAGTMCSALYVGDMCGPVFLVGQAFTFRPRRHRTVQTCNCSLYPGHLSGQRMAWDMMMNWSPAVGMVVAHILRLPQTLFDVVAGAHWGIIAGLAYYSMQGNWAKVAIIMVMFSGVDASTHVTAGQAARNAYGITSLFSVGAKQNLQLINTNGSWHINRTALNCNESINTGFIAGLFYYHKFNSTGCPQRLSSCKPITFFKQGWGPLTDANITGPSDDKPYCWHYAPRPCGIVPALNVCGPVYCFTPSPVVVGTTDAKGAPTYTWGANKTDVFLLESLRPPSGRWFGCTWMNSTGFVKTCGAPPCNIYGDGRDAQNESDLFCPTDCFRKHPEATYSRCGAGPWLTPRCLVDYPYRLWHYPCTVNFTLFKVRMFVGGFEHRFTAACNWTRGERCDIEDRDRSEQHPLLHSTTELAILPCSFTPMPALSTGLIHLHQNIVDVQYLYGIGSGMVGWALKWEFVILIFLLLADARVCVALWLILTISQAEA

SEQ ID NO: 54 - protein, 754 aa, Hepatitis C virus
MSTLPKPKRQTKRNTLRRPKNVKFPAGGQIVGEVYVLPRRGPQLGVREVRKTSERSQPRGRRQPTPKARPREGRSWAQPGYPWPLYGNEGCGWAGWLLPPRGSRPSWGQNDPRRRSRNLGKVIDTLTCGFADLMGYIPLIGAPVGGVARALAHGVRALEDGVNYATGNLPGCSFSIFLLALFSCLTCPASSLEYRNASGLYLLTNDCSNRSIVYEADDVILHLPGCVPCVETDNNNTSCWTPISPTVAVKHPGVTTASIRNHVNMLVAPPTLCSALYVEDAFGAVSLVGQAFTFRPRQHKTVQTCNCSIYPGHVSGHRMAWDMMMNWSPAIGLVISHLMRLPQTFFDLVVGAHWGVMAGLAYFSMQGNWAKVVIVLIMFSGVDATTHTTGGSAAQATAGFTSFFTRGPSQNLQLVNSNGSWHINSTALNCNDSLNTGFIAGLFYYHKFNSSGCPERMSSCKPITYFNQGWGPLTDANINGPSEDRPYCWHYPPRPCNITKPLNVCGPVYCFTPSPVVVGTTDIKGLPTYRFGVNESDVFLLTSLRPPQGRWFGCVWMNSTGFVKTCGAPPCNIYGGMKDIEANQTHLKCPTDCFRKHHDATFTRCGSGPWLTPRCLVDYPYRLWHYPCTVNFSIFKVRMFVGGHEHRFSAACNWTRGERCDLEDRDRSEQQPLLHSTTDSLILPCSFTPMRRLSTGLIHLHQNIVDVQYLYGVGSAVVGWALKWEFVVLVFLLLADARVCVALWMMLLISQAEA

SEQ ID NO: 55 - protein, 750 aa, Hepatitis C virus
MSTNPKPQRLTKRNTVRRPQNVKFPGGGQIVGGVYLLPRRGPRLGVRGTRKSSERSQPRGRRQRIPKAASSQGKAWGKPGYPWPLYGNEGCGWAGWLLSPRGSRPTWGPTDPRHRSRNLGKVIDTMTCGFADLMGYIPVLGAPLGGVARALAHGVRVLEDGVNYATGNLPGCSFSIFLLALLSCLTVPASAYEVRNSSGVYHLTNDCPNASIVYETDNAILHEPGCVPCVREGNTSRCWEPVAPTLAVRYRGALTDDLRTHIDLVVASATLCSALYVGDICGAIFIASQAVLWKPGGGRIVQDCNCSIYPGHVTGHRMAWDMMQNWAPALSMVAAYAVRVPGVIITTVAGGHWGVLFGLAYFGMAGNWAKVILIMLLMSGVDAETMAVGARAAHTTGALVSLLNPGPSQRLQLINTNGSWHINRTALNCNDSLQTGFIAALFYTHRFNSSGCPERMASCKPLSDFDQGWGPLWYNSTERPSDQRPYCWHYAPSPCGIVPAKDVCGPVYCFTPSPVVVGTTDRRGVPTYTWGENESDVFLLNSTRPPQGSWFGCSWMNTTGFTKTCGGPPCKIRPQGAQSNTSLTCPTDCFRKHPRATYSACGSGPWLTPRCMVHYPYRLWHYPCTVNFTIHKVRLYIGGVEHRLDAACNWTRGERCDLEDRDRVDMSPLLHSTTELAILPCSFVPLPALSTGLIHLHQNIVDAQYLYGLSPAIISWAIRWEWVVLVFLLLADARICACLWMMMLMAQAEA

SEQ ID NO: 56 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 325 aa, Homo sapiens
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 58 - protein, 246 aa, Homo sapiens
HKPSNTKVDKRVELKTPLGDTTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 383 aa, Homo sapiens
PTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 60 - protein, 276 aa, Homo sapiens
VTSTLTIKZSDWLGESMFTCRVDHRGLTFQQNASSMCVPDQDTAIRVFAIPPSFASIFLTKSTKLTCLVTDLTTYBSVTISWTREENGAVKTHTNISESHPNATFSAVGEASICEDBDWSGERFTCTVTHTDLPSPLKQTISRPKGVALHRPBVYLLPPARZZLNLRESATITCLVTGFSPADVFVEWMQRGEPLSPQKYVTSAPMPEPQAPGRYFAHSILTVSEEEWNTGGTYTCVVAHEALPNRVTERTVDKSTGKPTLYNVSLVMSDTAGTCY

SEQ ID NO: 61 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 62 - protein, 222 aa, Homo sapiens
ADPCDSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 63 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 64 - protein, 14 aa, synthetic construct
QYIKANSKFIGITE

SEQ ID NO: 65 - protein, 124 aa, synthetic construct
MIKLKFGVFFTVLLSSAYAHGTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMAN

SEQ ID NO: 66 - protein, 15 aa, synthetic construct
ILMQYIKANSKFIGI

SEQ ID NO: 67 - protein, 7 aa, synthetic construct
VNNESSE

SEQ ID NO: 68 - protein, 17 aa, synthetic construct
PGINGKAIHLVNNESSE

SEQ ID NO: 69 - protein, 6 aa, synthetic construct
PNRDIL

SEQ ID NO: 70 - protein, 7 aa, synthetic construct
FIGITEL

SEQ ID NO: 71 - protein, 6 aa, synthetic construct
SYFPSV

SEQ ID NO: 72 - protein, 20 aa, synthetic construct
NSVDDALINSTKIYSYFPSV

SEQ ID NO: 73 - protein, 20 aa, synthetic construct
IDKISDVSTIVPYIGPALNI

SEQ ID NO: 74 - protein, 15 aa, synthetic construct
QSIALSSLMVAQAIP

SEQ ID NO: 75 - protein, 19 aa, synthetic construct
PVFAGANYAAWAVNVAQVI

SEQ ID NO: 76 - protein, 20 aa, synthetic construct
VHHNTEEIVAQSIALSSLMV

SEQ ID NO: 77 - protein, 20 aa, synthetic construct
QSIALSSLMVAQAIPLVGEL

SEQ ID NO: 78 - protein, 20 aa, synthetic construct
VDIGFAAYNFVESIINLFQV

SEQ ID NO: 79 - protein, 20 aa, synthetic construct
QGESGHDIKITAENTPLPIA

SEQ ID NO: 80 - protein, 20 aa, synthetic construct
GVLLPTIPGKLDVNKSKTHI

SEQ ID NO: 81 - protein, 534 aa, synthetic construct
ADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 82 - protein, 26 aa, synthetic construct
IMQYIKANSKFIGIQSIALSSLMVAQ

SEQ ID NO: 83 - protein, 6 aa, synthetic construct
LEVLFQ

SEQ ID NO: 84 - protein, 17 aa, synthetic construct
AAAAAAAAAAAAAAAAA

SEQ ID NO: 85 - protein, 4 aa, synthetic construct
LVPR

SEQ ID NO: 86 - protein, 4 aa, synthetic construct
  VARIANT 2: note = Wherein the second residue may also be D (Asp)
IEGR

SEQ ID NO: 87 - protein, 5 aa, synthetic construct
DDDDK

SEQ ID NO: 88 - protein, 5 aa, synthetic construct
  REGION 1..5: note = The entire sequence of amino acids 1-5 can be repeated one or more times
GSGGS

SEQ ID NO: 89 - protein, 6 aa, synthetic construct
  REGION 1..6: note = The entire sequence of amino acids 1-6 can be repeated one or more times
GGSGGS

SEQ ID NO: 90 - protein, 4 aa, synthetic construct
  REGION 1..4: note = The entire sequence of amino acids 1-4 can be repeated one or more times
GGGS

SEQ ID NO: 91 - protein, 4 aa, synthetic construct
GGSG

SEQ ID NO: 92 - protein, 5 aa, synthetic construct
GGSGG

SEQ ID NO: 93 - protein, 5 aa, synthetic construct
GSGSG

SEQ ID NO: 94 - protein, 5 aa, synthetic construct
GSGGG

SEQ ID NO: 95 - protein, 5 aa, synthetic construct
GGGSG

SEQ ID NO: 96 - protein, 5 aa, synthetic construct
GSSSG

SEQ ID NO: 97 - protein, 8 aa, synthetic construct
LEVLFQGP

SEQ ID NO: 98 - protein, 7 aa, synthetic construct
ENLYFQS

SEQ ID NO: 99 - protein, 7 aa, synthetic construct
ENLYFQG

SEQ ID NO: 100 - protein, 6 aa, synthetic construct
LVPRGS

SEQ ID NO: 101 - protein, 5 aa, synthetic construct
  VARIANT 2: note = Wherein the second residue may also be D (Asp)
  VARIANT 5: note = X can be any naturally occurring amino acid except R (Arg) or P (Pro)
IEGRX

SEQ ID NO: 102 - protein, 6 aa, synthetic construct
ENLYFQ

SEQ ID NO: 103 - protein, 8 aa, synthetic construct
DYKDDDDK

SEQ ID NO: 104 - protein, 9 aa, synthetic construct
YPYDVPDYA

SEQ ID NO: 105 - protein, 10 aa, synthetic construct
EQKLISEEDL

SEQ ID NO: 106 - DNA, 24 bp, synthetic construct
  source 1..24: mol_type = other DNA
tcgtcgttttgtcgttttgtcgtt

SEQ ID NO: 107 - protein, 6 aa, synthetic construct
  VARIANT 2: note = X is L (Leu) or Q (Gln)
PXGMTS

SEQ ID NO: 108 - protein, 5 aa, synthetic construct
  VARIANT 2: note = X is L (Leu) or Q (Gln)
PXGMT

SEQ ID NO: 109 - protein, 7 aa, synthetic construct
ENLYTQS

SEQ ID NO: 110 - protein, 10 aa, synthetic construct
CGLVPAGSGP

SEQ ID NO: 111 - protein, 12 aa, synthetic construct
SLLKSRMVPNFN

SEQ ID NO: 112 - protein, 12 aa, synthetic construct
SLLIARRMPNFN

SEQ ID NO: 113 - protein, 12 aa, synthetic construct
SKLVQASASGVN

SEQ ID NO: 114 - protein, 12 aa, synthetic construct
SSYLKASDAPDN

SEQ ID NO: 115 - protein, 12 aa, synthetic construct
RPKPQQFFGLMN

SEQ ID NO: 116 - protein, 12 aa, synthetic construct
SLRPLALWRSFN

SEQ ID NO: 117 - protein, 12 aa, synthetic construct
SPQGIAGQRNFN

SEQ ID NO: 118 - protein, 14 aa, synthetic construct
DVDERDVRGFASFL

SEQ ID NO: 119 - protein, 12 aa, synthetic construct
SLPLGLWAPNFN

SEQ ID NO: 120 - protein, 12 aa, synthetic construct
SLLIFRSWANFN

SEQ ID NO: 121 - protein, 12 aa, synthetic construct
SGVVIATVIVIT

SEQ ID NO: 122 - protein, 12 aa, synthetic construct
SLGPQGIWGQFN

SEQ ID NO: 123 - protein, 12 aa, synthetic construct
KKSPGRVVGGSV

SEQ ID NO: 124 - protein, 12 aa, synthetic construct
PQGLLGAPGILG

SEQ ID NO: 125 - protein, 31 aa, synthetic construct
HGPEGLRVGFYESDVMGRGHARLVHVEEPHT

SEQ ID NO: 126 - protein, 12 aa, synthetic construct
GPQGLAGQRGIV

SEQ ID NO: 127 - protein, 12 aa, synthetic construct
GGSGQRGRKALE

SEQ ID NO: 128 - protein, 12 aa, synthetic construct
SLSALLSSDIFN

SEQ ID NO: 129 - protein, 12 aa, synthetic construct
SLPRFKIIGGFN

SEQ ID NO: 130 - protein, 12 aa, synthetic construct
SLLGIAVPGNFN

SEQ ID NO: 131 - protein, 12 aa, synthetic construct
FFKNIVTPRTPP

SEQ ID NO: 132 - protein, 14 aa, synthetic construct
QYIKANSKFIGIFE

SEQ ID NO: 133 - protein, 35 aa, synthetic construct
KSTKVPAAYAAQGYKVLVLNPSVAATLGFGAYMSK

SEQ ID NO: 134 - protein, 35 aa, synthetic construct
  VARIANT 7: note = X is A or V
  VARIANT 10: note = X is A or V
  VARIANT 11: note = X is A or S
  VARIANT 15: note = X is K or N
  VARIANT 31: note = X is A or S
  VARIANT 32: note = X is Y or F
  VARIANT 33: note = X is M or L
  VARIANT 35: note = X is K or R
KSTKVPXAYXXQGYXVLVLNPSVAATLGFGXXXSX

SEQ ID NO: 135 - protein, 35 aa, synthetic construct
KSTKVPVAYAAQGYKVLVLNPSVAATLGFGAYLSK

SEQ ID NO: 136 - protein, 35 aa, synthetic construct
KSTKVPAAYASQGYKVLVLNPSVAATLGFGAYMSK

SEQ ID NO: 137 - protein, 35 aa, synthetic construct
KSTKVPAAYVAQGYNVLVLNPSVAATLGFGSFMSR

SEQ ID NO: 138 - protein, 35 aa, synthetic construct
KSTKVPAAYAAQGYKVLVLNPSVAATLGFGAYMSR

SEQ ID NO: 139 - protein, 35 aa, synthetic construct
KSTKVPAAYASQGYKVLVLNPSVAATLGFGSYMSK

SEQ ID NO: 140 - protein, 10 aa, synthetic construct
  VARIANT 3: note = X is A or V
  VARIANT 4: note = X is A or S
  VARIANT 8: note = X is K or N
AYXXQGYXVL

SEQ ID NO: 141 - protein, 11 aa, synthetic construct
  VARIANT 7: note = X is A or S
  VARIANT 8: note = X is Y or F
  VARIANT 9: note = X is M or L
  VARIANT 11: note = X is K or R
ATLGFGXXXSX

SEQ ID NO: 142 - protein, 10 aa, synthetic construct
AYAAQGYKVL

SEQ ID NO: 143 - protein, 10 aa, synthetic construct
AYVAQGYNVL

SEQ ID NO: 144 - protein, 11 aa, synthetic construct
ATLGFGAYMSK

SEQ ID NO: 145 - protein, 15 aa, synthetic construct
VLVLNPSVAATLGFG

SEQ ID NO: 146 - protein, 30 aa, synthetic construct
PAAYAAQGYKVLVLNPSVAATLGFGAYMSK

SEQ ID NO: 147 - protein, 30 aa, synthetic construct
KSTKVPAAYAAQGYKVLVLNPSVAATLGFG

SEQ ID NO: 148 - protein, 50 aa, synthetic construct
GVYLLPRRGPRLGVRATRKTSERSQPRGRRQPIPKARRSEGRSWAQPGYP

SEQ ID NO: 149 - protein, 50 aa, synthetic construct
  VARIANT 4: note = X is L or V
  VARIANT 16: note = X A or G
  VARIANT 20: note = X is T or S
  VARIANT 32: note = X is P or R
  VARIANT 36: note = X A or D
  VARIANT 37: note = X R or A
  VARIANT 38: note = X is R, Q or S
  VARIANT 39: note = X is S or P
  VARIANT 40: note = X is E, T, or Q
  VARIANT 42: note = X is R or K
  VARIANT 43: note = X is S, T, H, or A
  VARIANT 45: note = X is A or G
  VARIANT 46: note = X is Q or K
GVYXLPRRGPRLGVRXTRKXSERSQPRGRRQXIPKXXXXXGXXWXXPGYP

SEQ ID NO: 150 - protein, 50 aa, synthetic construct
GVYLLPRRGPRLGVRATRKTSERSQPRGRRQPIPKARRPEGRTWAQPGYP

SEQ ID NO: 151 - protein, 50 aa, synthetic construct
GVYLLPRRGPRLGVRATRKTSERSQPRGRRQPIPKARRPEGRAWAQPGYP

SEQ ID NO: 152 - protein, 50 aa, synthetic construct
GVYLLPRRGPRLGVRATRKTSERSQPRGRRQPIPKDRRSTGKSWGKPGYP

SEQ ID NO: 153 - protein, 50 aa, synthetic construct
GVYVLPRRGPRLGVRATRKTSERSQPRGRRQPIPKARRSEGRSWAQPGYP

SEQ ID NO: 154 - protein, 50 aa, synthetic construct
GVYLLPRRGPRLGVRATRKTSERSQPRGRRQPIPKARRPTGRSWGQPGYP

SEQ ID NO: 155 - protein, 50 aa, synthetic construct
GVYLLPRRGPRLGVRATRKTSERSQPRGRRQPIPKARQPTGRHWAQPGYP

SEQ ID NO: 156 - protein, 50 aa, synthetic construct
GVYLLPRRGPRLGVRTTRKSSERSQPRGRRQRIPKAASSQGKAWGKPGYP

SEQ ID NO: 157 - protein, 25 aa, synthetic construct
RRGPRLGVRATRKTSERSQPRGRRQ

SEQ ID NO: 158 - protein, 25 aa, synthetic construct
RRGPRLGVRGTRKSSERSQPRGRRQ

SEQ ID NO: 159 - protein, 24 aa, synthetic construct
RATRKTSERSQPRGRRQPIPKARR

SEQ ID NO: 160 - protein, 24 aa, synthetic construct
RGTRKSSERSQPRGRRQRIPKAAQ

SEQ ID NO: 161 - protein, 24 aa, synthetic construct
RATRKTSERSQPRGRRQPIPKARQ

SEQ ID NO: 162 - protein, 24 aa, synthetic construct
RATRKTSERSQPRGRRQPIPKDRR

SEQ ID NO: 163 - protein, 20 aa, synthetic construct
RRQPIPKARRSEGRSWAQPG

SEQ ID NO: 164 - protein, 20 aa, synthetic construct
RRQPIPKARPSEGRTWAQPG

SEQ ID NO: 165 - protein, 20 aa, synthetic construct
RRQPIPKARRPEGRAWAQPG

SEQ ID NO: 166 - protein, 20 aa, synthetic construct
RRQPIPKDRRSTGKSWGKPG

SEQ ID NO: 167 - protein, 20 aa, synthetic construct
RRQPIPKDRRSTGKSWGKPG

SEQ ID NO: 168 - protein, 20 aa, synthetic construct
RRQPIPKARRPTGRSWGQPG

SEQ ID NO: 169 - protein, 20 aa, synthetic construct
RRQPIPKARQPTGRHWAQPG

SEQ ID NO: 170 - protein, 20 aa, synthetic construct
RRQRIPKAASSQGKAWGKPG

SEQ ID NO: 171 - protein, 23 aa, synthetic construct
  VARIANT 5: note = X is V or C
  VARIANT 6: note = X is A or S
  VARIANT 14: note = X is F or Y
DVVVXXTDALMTGXTGDFDSVID

SEQ ID NO: 172 - protein, 23 aa, synthetic construct
DVVVVATDALMTGYTGDFDSVID

SEQ ID NO: 173 - protein, 23 aa, synthetic construct
DVVVCATDALMTGFTGDFDSVID

SEQ ID NO: 174 - protein, 23 aa, synthetic construct
DVVVCSTDALMTGFTGDFDSVID

SEQ ID NO: 175 - protein, 23 aa, synthetic construct
DVVVCATDALMTGYTGDFDSVID

SEQ ID NO: 176 - protein, 9 aa, synthetic construct
ATDALMTGF

SEQ ID NO: 177 - protein, 9 aa, synthetic construct
STDALMTGF

SEQ ID NO: 178 - protein, 9 aa, synthetic construct
ATDALMTGY

SEQ ID NO: 179 - protein, 9 aa, synthetic construct
GFTGDFDSV

SEQ ID NO: 180 - protein, 9 aa, synthetic construct
GYTGDFDSV

SEQ ID NO: 181 - protein, 15 aa, synthetic construct
ATDALMTGFTGDFDS

SEQ ID NO: 182 - protein, 15 aa, synthetic construct
STDALMTGFTGDFDS

SEQ ID NO: 183 - protein, 15 aa, synthetic construct
ATDALMTGYTGDFDS

SEQ ID NO: 184 - protein, 15 aa, synthetic construct
ALMTGFTGDFDSVID

SEQ ID NO: 185 - protein, 15 aa, synthetic construct
ALMTGYTGDFDSVID

SEQ ID NO: 186 - protein, 23 aa, synthetic construct
DVVVVATDALMTGFTGDFDSVID

SEQ ID NO: 187 - protein, 10 aa, synthetic construct
AYASQGYKVL

SEQ ID NO: 188 - protein, 27 aa, synthetic construct
LEQIKGGRHLIFCHSKKKCDELAAKLT

SEQ ID NO: 189 - protein, 27 aa, synthetic construct
  VARIANT 1: note = X is L or I
  VARIANT 2: note = X is E, A, V, S, OR Q
  VARIANT 3: note = X is Q, T, Y, F, or L
  VARIANT 4: note = X is I or L
  VARIANT 22: note = X is L or I
  VARIANT 24: note = X is A, K, or S
  VARIANT 25: note = X is K, Q, or A
  VARIANT 27: note = X is T, R, or S
XXXXKGGRHLIFCHSKKKCDEXAXXLX

SEQ ID NO: 190 - protein, 27 aa, synthetic construct
LEVIKGGRHLIFCHSKKKCDELAAKLV

SEQ ID NO: 191 - protein, 27 aa, synthetic construct
IETIKGGRHLIFCHSKKKCDELAAKLS

SEQ ID NO: 192 - protein, 27 aa, synthetic construct
LSYIKGGRHLIFCHSKKKCDELAAALR

SEQ ID NO: 193 - protein, 26 aa, synthetic construct
LAFIKGGRHLIFCHSKKKCDELAALR

SEQ ID NO: 194 - protein, 27 aa, synthetic construct
IAQLKGGRHLIFCHSKKKCDEIASKLR

SEQ ID NO: 195 - protein, 27 aa, synthetic construct
IAQLKGGRHLIFCHSKKKCDEIASKLR

SEQ ID NO: 196 - protein, 26 aa, synthetic construct
LELIKGGRHLIFCHSKKKCDELAQLT

SEQ ID NO: 197 - protein, 27 aa, synthetic construct
  VARIANT 2: note = X can be any naturally occurring amino acid
LXLIKGGRHLIFCHSKKKCDELAKQLT

SEQ ID NO: 198 - protein, 27 aa, synthetic construct
LEYIKGGRHLIFCHSKKKCDELAKQLT

SEQ ID NO: 199 - protein, 27 aa, synthetic construct
LQHIKGGRHLIFCHSKKKCDELAGKLT

SEQ ID NO: 200 - protein, 9 aa, synthetic construct
LIFCHSKKK

SEQ ID NO: 201 - protein, 9 aa, synthetic construct
HSKKKCDEL

SEQ ID NO: 202 - protein, 16 aa, synthetic construct
KGGRHLIFCHSKKKCD

SEQ ID NO: 203 - protein, 35 aa, synthetic construct
ILRRHVGPGEGAVQWMNRLIAFASRGNHVSPTHYV

SEQ ID NO: 204 - protein, 35 aa, synthetic construct
  VARIANT 1: note = X is I or V
  VARIANT 2: note = X is L or I
  VARIANT 3: note = X is R or K
  VARIANT 6: note = X is V, I, or T
  VARIANT 8: note = X is P, Q, or T
  VARIANT 9: note = X G, A, or S
  VARIANT 12: note = X is A or V
  VARIANT 13: note = X is V or T
  VARIANT 30: note = X is S or A
  VARIANT 35: note = X is V or I
XXXRHXGXXEGXXQWMNRLIAFASRGNHVXPTHYX

SEQ ID NO: 205 - protein, 35 aa, synthetic construct
ILRRHVGPGEGAVQWMNRLIAFASRGNHVAPTHYV

SEQ ID NO: 206 - protein, 35 aa, synthetic construct
ILRRHVGQGEGAVQWMNRLIAFASRGNHVAPTHYV

SEQ ID NO: 207 - protein, 35 aa, synthetic construct
VLRRHIGPGEGAVQWMNRLIAFASRGNHVSPTHYV

SEQ ID NO: 208 - protein, 35 aa, synthetic construct
ILRRHVGPAEGATQWMNRLIAFASRGNHVSPTHYV

SEQ ID NO: 209 - protein, 35 aa, synthetic construct
IIKRHTGTSEGVTQWMNRLIAFASRGNHVSPTHYI

SEQ ID NO: 210 - protein, 16 aa, synthetic construct
EGAVQWMNRLIAFASR

SEQ ID NO: 211 - protein, 16 aa, synthetic construct
EGATQWMNRLIAFASR

SEQ ID NO: 212 - protein, 16 aa, synthetic construct
EGVTQWMNRLIAFASR

SEQ ID NO: 213 - protein, 9 aa, synthetic construct
WMNRLIAFA

SEQ ID NO: 214 - protein, 15 aa, synthetic construct
RHVGPGEGAVQWMNR

SEQ ID NO: 215 - protein, 15 aa, synthetic construct
RHVGQGEGAVQWMNR

SEQ ID NO: 216 - protein, 15 aa, synthetic construct
RHIGPGEGAVQWMNR

SEQ ID NO: 217 - protein, 15 aa, synthetic construct
RHVGPAEGATQWMNR

SEQ ID NO: 218 - protein, 15 aa, synthetic construct
RHTGTSEGVTQWMNR

SEQ ID NO: 219 - protein, 20 aa, synthetic construct
HVGPGEGAVQWMNRLIAFAS

SEQ ID NO: 220 - protein, 20 aa, synthetic construct
HVGQGEGAVQWMNRLIAFAS

SEQ ID NO: 221 - protein, 20 aa, synthetic construct
HIGPGEGAVQWMNRLIAFAS

SEQ ID NO: 222 - protein, 20 aa, synthetic construct
HVGPAEGATQWMNRLIAFAS

SEQ ID NO: 223 - protein, 20 aa, synthetic construct
HTGTSEGVTQWMNRLIAFAS

SEQ ID NO: 224 - protein, 20 aa, synthetic construct
GAVQWMNRLIAFASRGNHVS

SEQ ID NO: 225 - protein, 20 aa, synthetic construct
GAVQWMNRLIAFASRGNHVA

SEQ ID NO: 226 - protein, 20 aa, synthetic construct
GATQWMNRLIAFASRGNHVS

SEQ ID NO: 227 - protein, 20 aa, synthetic construct
GVTQWMNRLIAFASRGNHVS

SEQ ID NO: 228 - protein, 42 aa, synthetic construct
GTEGEIPFYGKAIPLEQIKGGRHLIFCHSKKKCDELAAKLTG

SEQ ID NO: 229 - protein, 52 aa, synthetic construct
  VARIANT 1: note = X is P, G, or S
  VARIANT 2: note = X is T, N, Q, H, or S
  VARIANT 3: note = X is E, T, or D
  VARIANT 11: note = X is K or R
  VARIANT 15: note = X is L or I
  VARIANT 16: note = X E, A, S, or Q
  VARIANT 17: note = X is Q, T, Y, F, or L
  VARIANT 18: note = X is I or L
  VARIANT 36: note = X is L or I
  VARIANT 38: note = X is A, K, or S
  VARIANT 39: note = X is K, Q, or A
  VARIANT 41: note = X is T, R, or S
  VARIANT 42: note = X is G or S
  VARIANT 43..52: note = Wherein up to 8 copies of K from positions 43-52 may be deleted
XXXGEIPFYGXAIPXXXXKGGRHLIFCHSKKKCDEXAXXLXXKKKKKKKKKK

SEQ ID NO: 230 - protein, 45 aa, synthetic construct
  VARIANT 1: note = X is P, G, or S
  VARIANT 2: note = X is T, N, Q, H, or S
  VARIANT 3: note = X is E, T, or D
  VARIANT 11: note = X is K or R
  VARIANT 15: note = X is L or I
  VARIANT 16: note = X E, A, S, or Q
  VARIANT 17: note = X is Q, T, Y, F, or L
  VARIANT 18: note = X is I or L
  VARIANT 36: note = X is L or I
  VARIANT 38: note = X is A, K, or S
  VARIANT 39: note = X is K, Q, or A
  VARIANT 41: note = X is T, R, or S
  VARIANT 42: note = X is G or S
XXXGEIPFYGXAIPXXXXKGGRHLIFCHSKKKCDEXAXXLXXKKK

SEQ ID NO: 231 - protein, 45 aa, synthetic construct
GTEGEIPFYGKAIPLEQIKGGRHLIFCHSKKKCDELAAKLTGKKK

SEQ ID NO: 232 - protein, 42 aa, synthetic construct
  VARIANT 1: note = X is P, G, or S
  VARIANT 2: note = X is T, N, Q, H, or S
  VARIANT 3: note = X is E, T, or D
  VARIANT 11: note = X is K or R
  VARIANT 15: note = X is L or I
  VARIANT 16: note = X E, A, S, or Q
  VARIANT 17: note = X is Q, T, Y, F, or L
  VARIANT 18: note = X is I or L
  VARIANT 36: note = X is L or I
  VARIANT 38: note = X is A, K, or S
  VARIANT 39: note = X is K, Q, or A
  VARIANT 41: note = X is T, R, or S
  VARIANT 42: note = X is G or S
XXXGEIPFYGXAIPXXXXKGGRHLIFCHSKKKCDEXAXXLXX

SEQ ID NO: 233 - protein, 42 aa, synthetic construct
STTGEIPFYGKAIPLEVIKGGRHLIFCHSKKKCDELAAKLVA

SEQ ID NO: 234 - protein, 42 aa, synthetic construct
SNTGEIPFYGKAIPIETIKGGRHLIFCHSKKKCDELAAKLSG

SEQ ID NO: 235 - protein, 42 aa, synthetic construct
GQEGEIPFYGRAIPLSYIKGGRHLIFCHSKKKCDELAAALRG

SEQ ID NO: 236 - protein, 41 aa, synthetic construct
GHEGEIPFYGKAIPLAFIKGGRHLIFCHSKKKCDELAALRG

SEQ ID NO: 237 - protein, 42 aa, synthetic construct
GSEGEIPFYGKAIPIAQLKGGRHLIFCHSKKKCDEIASKLRG

SEQ ID NO: 238 - protein, 41 aa, synthetic construct
PTTGEIPFYGKAIPLELIKGGRHLIFCHSKKKCDELAQLTS

SEQ ID NO: 239 - protein, 42 aa, synthetic construct
  VARIANT 16: note = X can be any naturally occurring amino acid
PSEGEIPFYGRAIPLXLIKGGRHLIFCHSKKKCDELAKQLTS

SEQ ID NO: 240 - protein, 42 aa, synthetic construct
PTTGEIPFYGKAIPLEYIKGGRHLIFCHSKKKCDELAKQLTS

SEQ ID NO: 241 - protein, 42 aa, synthetic construct
GNDGEIPFYGKAIPLQHIKGGRHLIFCHSKKKCDELAGKLTS

SEQ ID NO: 242 - protein, 11 aa, synthetic construct
  VARIANT 4: note = X is K or R
  VARIANT 8: note = X can be any naturally occurring amino acid
  VARIANT 9: note = X is E, S, A, Q, or V
  VARIANT 10: note = X is L or I
  VARIANT 10: note = X is Q, T, Y, F, I, or H
  VARIANT 11: note = X is I or L
FYGXAIPXXXX

SEQ ID NO: 243 - protein, 11 aa, synthetic construct
FYGKAIPLEQI

SEQ ID NO: 244 - protein, 11 aa, synthetic construct
GRHLIFCHSKK

SEQ ID NO: 245 - protein, 37 aa, synthetic construct
IPFYGKAIPLEQIKGGRHLIFCHSKKKCDELAAKLTG

SEQ ID NO: 246 - protein, 37 aa, synthetic construct
GTEGEIPFYGKAIPLEQIKGGRHLIFCHSKKKCDELA

SEQ ID NO: 247 - protein, 45 aa, synthetic construct
LNAVAYYRGLDVSVIPTSGDVVVVATDALMTGFTGDFDSVIDCNV

SEQ ID NO: 248 - protein, 45 aa, synthetic construct
LNAVAYYRGLDVSVIPTSGDVVVVATDALMTGFTGDFDSVIDKKK

SEQ ID NO: 249 - protein, 55 aa, synthetic construct
  VARIANT 1: note = X is L or V
  VARIANT 2: note = X is N or T
  VARIANT 6: note = X is Y or F
  VARIANT 10: note = X is L or V
  VARIANT 13: note = X is S or A
  VARIANT 14: note = X is V or I
  VARIANT 17: note = X is T or A
  VARIANT 18: note = X is S, Q, or T
  VARIANT 24: note = X is V or C
  VARIANT 25: note = X is A or S
  VARIANT 33: note = X is F or Y
  VARIANT 43: note = X is C or K
  VARIANT 44: note = X is N or K
  VARIANT 45: note = X is V or K
  VARIANT 46..55: note = Wherein up to 8 copies of K from positions 46-55 may be deleted
XXAVAXYRGXDVXXIPXXGDVVVXXTDALMTGXTGDFDSVIDXXXKKKKKKKKKK

SEQ ID NO: 250 - protein, 48 aa, synthetic construct
  VARIANT 1: note = X is L or V
  VARIANT 2: note = X is N or T
  VARIANT 6: note = X is Y or F
  VARIANT 10: note = X is L or V
  VARIANT 13: note = X is S or A
  VARIANT 14: note = X is V or I
  VARIANT 17: note = X is T or A
  VARIANT 18: note = X is S, Q, or T
  VARIANT 24: note = X is V or C
  VARIANT 25: note = X is A or S
  VARIANT 33: note = X is F or Y
  VARIANT 43: note = X is C or K
  VARIANT 44: note = X is N or K
  VARIANT 45: note = X is V or K
XXAVAXYRGXDVXXIPXXGDVVVXXTDALMTGXTGDFDSVIDXXXKKK

SEQ ID NO: 251 - protein, 48 aa, synthetic construct
LNAVAYYRGLDVSVIPTSGDVVVVATDALMTGFTGDFDSVIDCNVKKK

SEQ ID NO: 252 - protein, 45 aa, synthetic construct
  VARIANT 1: note = X is L or V
  VARIANT 2: note = X is N or T
  VARIANT 6: note = X is Y or F
  VARIANT 10: note = X is L or V
  VARIANT 13: note = X is S or A
  VARIANT 14: note = X is V or I
  VARIANT 17: note = X is T or A
  VARIANT 18: note = X is S, Q, or T
  VARIANT 24: note = X is V or C
  VARIANT 25: note = X is A or S
  VARIANT 33: note = X is F or Y
  VARIANT 43: note = X is C or K
  VARIANT 44: note = X is N or K
  VARIANT 45: note = X is V or K
XXAVAXYRGXDVXXIPXXGDVVVXXTDALMTGXTGDFDSVIDXXX

SEQ ID NO: 253 - protein, 42 aa, synthetic construct
LNAVAYYRGLDVSVIPTSGDVVVVATDALMTGFTGDFDSVID

SEQ ID NO: 254 - protein, 48 aa, synthetic construct
ILRRHVGPGEGAVQWMNRLIAFASRGNHVSPTHYVPESDASARVTQIL

SEQ ID NO: 255 - protein, 58 aa, synthetic construct
  VARIANT 1: note = X is I or V
  VARIANT 2: note = X is L or I
  VARIANT 3: note = X is R or K
  VARIANT 6: note = X is V, I, or T
  VARIANT 8: note = X is P, Q, or T
  VARIANT 9: note = X is G, A, or S
  VARIANT 12: note = X is A or V
  VARIANT 13: note = X V or T
  VARIANT 30: note = X S or A
  VARIANT 35: note = X is V or I
  VARIANT 36: note = X iS P, T, A, or Q
  VARIANT 37: note = X is E or D
  VARIANT 38: note = X is S, T, or D
  VARIANT 41: note = X is S or A
  VARIANT 42: note = X is A, Q, R, or K
  VARIANT 43: note = X R or K
  VARIANT 45: note = X is T or M
  VARIANT 46: note = X Q, A, T, or G
  VARIANT 47: note = X is I, L or V
  VARIANT 49..58: note = Wherein up to 8 copies of K from positions 49-58 may be deleted
XXXRHXGXXEGXXQWMNRLIAFASRGNHVXPTHYXXXXDAXXXVXXXLKKKKKKKKKK

SEQ ID NO: 256 - protein, 51 aa, synthetic construct
  VARIANT 1: note = X is I or V
  VARIANT 2: note = X is L or I
  VARIANT 3: note = X is R or K
  VARIANT 6: note = X is V, I, or T
  VARIANT 8: note = X is P, Q, or T
  VARIANT 9: note = X is G, A, or S
  VARIANT 12: note = X is A or V
  VARIANT 13: note = X V or T
  VARIANT 30: note = X S or A
  VARIANT 35: note = X is V or I
  VARIANT 36: note = X iS P, T, A, or Q
  VARIANT 37: note = X is E or D
  VARIANT 38: note = X is S, T, or D
  VARIANT 41: note = X is S or A
  VARIANT 42: note = X is A, Q, R, or K
  VARIANT 43: note = X R, K, or any amino acid residue
  VARIANT 45: note = X is T or M
  VARIANT 46: note = X Q, A, T, or G
  VARIANT 47: note = X is I, L or V
XXXRHXGXXEGXXQWMNRLIAFASRGNHVXPTHYXXXXDAXXXVXXXLKKK

SEQ ID NO: 257 - protein, 51 aa, synthetic construct
ILRRHVGPGEGAVQWMNRLIAFASRGNHVSPTHYVPESDASARVTQILKKK

SEQ ID NO: 258 - protein, 60 aa, synthetic construct
  VARIANT 51..60: note = Wherein up to 8 copies of K from positions 51-60 may be deleted
GVYLLPRRGPRLGVRATRKTSERSQPRGRRQPIPKARRSEGRSWAQPGYPKKKKKKKKKK

SEQ ID NO: 259 - protein, 53 aa, synthetic construct
GVYLLPRRGPRLGVRATRKTSERSQPRGRRQPIPKARRSEGRSWAQPGYPKKK

SEQ ID NO: 260 - protein, 33 aa, synthetic construct
  VARIANT 24: note = Wherein up to 8 copies of K from positions 24-33 may be deleted
DVVVVATDALMTGFTGDFDSVIDKKKKKKKKKK

SEQ ID NO: 261 - protein, 26 aa, synthetic construct
DVVVVATDALMTGFTGDFDSVIDKKK

SEQ ID NO: 262 - protein, 37 aa, synthetic construct
  VARIANT 28..37: note = Wherein up to 8 copies of K from positions 28-37 may be deleted
LEQIKGGRHLIFCHSKKKCDELAAKLTKKKKKKKKKK

SEQ ID NO: 263 - protein, 30 aa, synthetic construct
LEQIKGGRHLIFCHSKKKCDELAAKLTKKK

SEQ ID NO: 264 - protein, 45 aa, synthetic construct
  VARIANT 36..45: note = Wherein up to 8 copies of K from positions 36-45 may be deleted
ILRRHVGPGEGAVQWMNRLIAFASRGNHVSPTHYVKKKKKKKKKK

SEQ ID NO: 265 - protein, 38 aa, synthetic construct
ILRRHVGPGEGAVQWMNRLIAFASRGNHVSPTHYVKKK

SEQ ID NO: 266 - protein, 48 aa, synthetic construct
  VARIANT 1: note = X is I or V
  VARIANT 2: note = X is L or I
  VARIANT 3: note = X is R or K
  VARIANT 6: note = X is V, I, or T
  VARIANT 8: note = X is P, Q, or T
  VARIANT 9: note = X is G, A, or S
  VARIANT 12: note = X is A or V
  VARIANT 13: note = X V or T
  VARIANT 30: note = X S or A
  VARIANT 35: note = X is V or I
  VARIANT 36: note = X iS P, T, A, or Q
  VARIANT 37: note = X is E or D
  VARIANT 38: note = X is S, T, or D
  VARIANT 41: note = X is S or A
  VARIANT 42: note = X is A, Q, R, or K
  VARIANT 43: note = X R, K, or any amino acid residue
  VARIANT 45: note = X is T or M
  VARIANT 46: note = X Q, A, T, or G
  VARIANT 47: note = X is I, L or V
XXXRHXGXXEGXXQWMNRLIAFASRGNHVXPTHYXXXXDAXXXVXXXL

SEQ ID NO: 267 - protein, 48 aa, synthetic construct
ILRRHVGPGEGAVQWMNRLIAFASRGNHVSPTHYVPESDAAARVTAIL

SEQ ID NO: 268 - protein, 48 aa, synthetic construct
ILRRHVGPGEGAVQWMNRLIAFASRGNHVSPTHYVPESDAAARVTQIL

SEQ ID NO: 269 - protein, 48 aa, synthetic construct
ILRRHVGPGEGAVQWMNRLIAFASRGNHVAPTHYVTESDASQRVTQLL

SEQ ID NO: 270 - protein, 48 aa, synthetic construct
ILRRHVGQGEGAVQWMNRLIAFASRGNHVAPTHYVAESDASQRVTQVL

SEQ ID NO: 271 - protein, 48 aa, synthetic construct
ILRRHVGPGEGAVQWMNRLIAFASRGNHVSPTHYVPESDAAARVTALL

SEQ ID NO: 272 - protein, 48 aa, synthetic construct
VLRRHIGPGEGAVQWMNRLIAFASRGNHVSPTHYVPETDASAKVTQLL

SEQ ID NO: 273 - protein, 48 aa, synthetic construct
  VARIANT 43: note = X can be any naturally occurring amino acid
ILRRHVGPAEGATQWMNRLIAFASRGNHVSPTHYVPETDASRXVTTIL

SEQ ID NO: 274 - protein, 48 aa, synthetic construct
IIKRHTGTSEGVTQWMNRLIAFASRGNHVSPTHYIQDDDASKRVMGIL

SEQ ID NO: 275 - protein, 96 aa, synthetic construct
  VARIANT 91: note = X can be any naturally occurring amino acid
IIKRHTGTSEGVTQWMNRLIAFASRGNHVSPTHYIQDDDASKRVMGILILRRHVGPAEGATQWMNRLIAFASRGNHVSPTHYVPETDASRXVTTIL

SEQ ID NO: 276 - protein, 15 aa, synthetic construct
  VARIANT 5: note = X is V or I
  VARIANT 6: note = X P, T, A, or Q
  VARIANT 7: note = X is E or D
  VARIANT 8: note = X is S, T, or D
  VARIANT 11: note = X is S or A
  VARIANT 12: note = X is A, Q, R, or K
  VARIANT 13: note = X is R, K, or any amino acid residue
  VARIANT 15: note = X is T or M
PTHYXXXXDAXXXVX

SEQ ID NO: 277 - protein, 45 aa, synthetic construct
RHVGPGEGAVQWMNRLIAFASRGNHVSPTHYVPESDASARVTQIL

SEQ ID NO: 278 - protein, 45 aa, synthetic construct
ILRRHVGPGEGAVQWMNRLIAFASRGNHVSPTHYVPESDASARVT

SEQ ID NO: 279 - protein, 33 aa, synthetic construct
HSKKKCDELAAKLTGLGLNAVAYYRGLDVSVIP

SEQ ID NO: 280 - protein, 19 aa, synthetic construct
DELAAKLTGLGLNAVAYYR

SEQ ID NO: 281 - protein, 19 aa, synthetic construct
DELAAKLVALGINAVAYYR

SEQ ID NO: 282 - protein, 19 aa, synthetic construct
DELAAKLSGLGLNAVAYYR

SEQ ID NO: 283 - protein, 19 aa, synthetic construct
DELAAALRGMGLNAVAYYR

SEQ ID NO: 284 - protein, 19 aa, synthetic construct
DELAAALRGMGVNAVAYYR

SEQ ID NO: 285 - protein, 19 aa, synthetic construct
DELASKLRGMGLNAVAYYR

SEQ ID NO: 286 - protein, 19 aa, synthetic construct
DELAAKLRGMGLNAVAYYR

SEQ ID NO: 287 - protein, 33 aa, synthetic construct
  VARIANT 11: note = X is A or S
  VARIANT 12: note = X is K or A
  VARIANT 14: note = X is V, S, R, or T
  VARIANT 15: note = X is A or G
  VARIANT 16: note = X is L or M
  VARIANT 18: note = X is I, L, or V
  VARIANT 31: note = X is V or I
HSKKKCDELAXXLXXXGXNAVAYYRGLDVSXIP

SEQ ID NO: 288 - protein, 33 aa, synthetic construct
  VARIANT 14: note = X is V, S, R, or T
  VARIANT 15: note = X is A or G
  VARIANT 16: note = X is L or M
  VARIANT 18: note = X is I, L or V
HSKKKCDELAAKLXXXGXNAVAYYRGLDVSVIP

SEQ ID NO: 289 - protein, 33 aa, synthetic construct
  VARIANT 18: note = X is I, L or V
  VARIANT 31: note = X is V or I
HSKKKCDELAAALRGMGXNAVAYYRGLDVSXIP

SEQ ID NO: 290 - protein, 33 aa, synthetic construct
HSKKKCDELAAKLRGMGLNAVAYYRGLDVSVIP

SEQ ID NO: 291 - protein, 42 aa, synthetic construct
KGGRHLIFCHSKKKCDELAAKLTGLGLNAVAYYRGLDVSVIP

SEQ ID NO: 292 - protein, 42 aa, synthetic construct
  VARIANT 20: note = X is A or S
  VARIANT 21: note = X is K or A
  VARIANT 23: note = X is V, S, R, or T
  VARIANT 24: note = X is A or G
  VARIANT 25: note = X is L or M
  VARIANT 27: note = X is I, L, or V
  VARIANT 40: note = X is V or I
KGGRHLIFCHSKKKCDELAXXLXXXGXNAVAYYRGLDVSXIP

SEQ ID NO: 293 - protein, 42 aa, synthetic construct
  VARIANT 23: note = X is V, S, R, or T
  VARIANT 24: note = X A or G
  VARIANT 25: note = X is L or M
  VARIANT 27: note = X is I L, or V
KGGRHLIFCHSKKKCDELAAKLXXXGXNAVAYYRGLDVSVIP

SEQ ID NO: 294 - protein, 42 aa, synthetic construct
  VARIANT 27: note = X is I, L, or V
  VARIANT 40: note = X is V or I
KGGRHLIFCHSKKKCDELAAALRGMGXNAVAYYRGLDVSXIP

SEQ ID NO: 295 - protein, 85 aa, synthetic construct
KSTKVPAAYAAQGYKVLVLNPSVAATLGFGAYMSKGVYLLPRRGPRLGVRATRKTSERSQPRGRRQPIPKARRSEGRSWAQPGYP

SEQ ID NO: 296 - protein, 85 aa, synthetic construct
GVYLLPRRGPRLGVRATRKTSERSQPRGRRQPIPKARRSEGRSWAQPGYPKSTKVPAAYAAQGYKVLVLNPSVAATLGFGAYMSK

SEQ ID NO: 297 - protein, 27 aa, synthetic construct
LEQIKGGRHLIFCHSKKKCDELAAKLT

SEQ ID NO: 298 - protein, 135 aa, synthetic construct
KSTKVPAAYAAQGYKVLVLNPSVAATLGFGAYMSKGVYLLPRRGPRLGVRATRKTSERSQPRGRRQPIPKARRSEGRSWAQPGYPDVVVVATDALMTGFTGDFDSVIDLEQIKGGRHLIFCHSKKKCDELAAKLT

SEQ ID NO: 299 - protein, 170 aa, synthetic construct
KSTKVPAAYAAQGYKVLVLNPSVAATLGFGAYMSKGVYLLPRRGPRLGVRATRKTSERSQPRGRRQPIPKARRSEGRSWAQPGYPDVVVVATDALMTGFTGDFDSVIDLEQIKGGRHLIFCHSKKKCDELAAKLTILRRHVGPGEGAVQWMNRLIAFASRGNHVSPTHYV

SEQ ID NO: 300 - protein, 45 aa, synthetic construct
  VARIANT 36..45: note = Wherein up to 8 copies of K from positions 36-45 may be deleted
KSTKVPAAYAAQGYKVLVLNPSVAATLGFGAYMSKKKKKKKKKKK

SEQ ID NO: 301 - protein, 52 aa, synthetic construct
  VARIANT 43..52: note = Wherein up to 8 copies of K from positions 43-52 may be deleted
GTEGEIPFYGKAIPLEQIKGGRHLIFCHSKKKCDELAAKLTGKKKKKKKKKK

SEQ ID NO: 302 - protein, 38 aa, synthetic construct
KSTKVPAAYAAQGYKVLVLNPSVAATLGFGAYMSKKKK

SEQ ID NO: 303 - protein, 58 aa, synthetic construct
  VARIANT 49..58: note = Wherein up to 8 copies of K from positions 49-58 may be deleted
ILRRHVGPGEGAVQWMNRLIAFASRGNHVSPTHYVPESDASARVTQILKKKKKKKKKK

SEQ ID NO: 304 - protein, 55 aa, synthetic construct
  VARIANT 46..55: note = Wherein up to 8 copies of K from positions 46-55 may be deleted
LNAVAYYRGLDVSVIPTSGDVVVVATDALMTGFTGDFDSVIDCNVKKKKKKKKKK

SEQ ID NO: 305 - protein, 52 aa, synthetic construct
  VARIANT 43..52: note = Wherein up to 8 copies of K from positions 43-52 may be deleted
LNAVAYYRGLDVSVIPTSGDVVVVATDALMTGFTGDFDSVIDKKKKKKKKKK

SEQ ID NO: 306 - protein, 58 aa, synthetic construct; identical to SEQ ID NO: 303
  VARIANT 49..58: note = Wherein up to 8 copies of K from positions 49-58 may be deleted

SEQ ID NO: 307 - protein, 35 aa, synthetic construct; identical to SEQ ID NO: 203

SEQ ID NO: 308 - protein, 58 aa, synthetic construct; identical to SEQ ID NO: 303
  VARIANT 49..58: note = Wherein up to 8 copies of K from positions 49-58 may be deleted

SEQ ID NO: 309 - protein, 64 aa, synthetic construct
  VARIANT 47: note = X can be any naturally occurring amino acid
PGGGQIVGGVYLLPRRGPRLGVRATRKTSERSQPRGRRQPIPKARRXGRSWAQPGYPWPLYGNE

SEQ ID NO: 310 - protein, 65 aa, synthetic construct
PGGGQIVGGVYLLPRRGPRLGVRATRKTSERSQPRGRRQPIPKARRPEGRTWAQPGYPWPLYGNE

SEQ ID NO: 311 - protein, 65 aa, synthetic construct
PGGGQIVGGVYLLPRRGPRLGVRATRKTSERSQPRGRRQPIPKARRPEGRAWAQPGYPWPLYGNE

SEQ ID NO: 312 - protein, 65 aa, synthetic construct
PGGGQIVGGVYLLPRRGPRLGVRATRKTSERSQPRGRRQPIPKDRRSTGKSWGKPGYPWPLYGNE

SEQ ID NO: 313 - protein, 65 aa, synthetic construct; identical to SEQ ID NO: 312

SEQ ID NO: 314 - protein, 65 aa, synthetic construct
PGGGQIVGGVYVLPRRGPRLGVRATRKTSERSQPRGRRQPIPKARRSEGRSWAQPGYPWPLYGNE

SEQ ID NO: 315 - protein, 65 aa, synthetic construct
PGGGQIVGGVYLLPRRGPRLGVRATRKTSERSQPRGRRQPIPKARRSEGRSWAQPGYPWPLYGNE

SEQ ID NO: 316 - protein, 65 aa, synthetic construct
PGGGQIVGGVYLLPRRGPRLGVRATRKTSERSQPRGRRQPIPKARRPTGRSWGQPGYPWPLYANE

SEQ ID NO: 317 - protein, 65 aa, synthetic construct
PGGGQIVGGVYLLPRRGPRLGVRATRKTSERSQPRGRRQPIPKARQPTGRHWAQPGYPWPLYGNE

SEQ ID NO: 318 - protein, 65 aa, synthetic construct
PGGGQIVGGVYLLPRRGPRLGVRGTRKSSERSQPRGRRQRIPKAASSQGKAWKGPGYPWPLYGNE

SEQ ID NO: 319 - protein, 50 aa, synthetic construct
PTGSGKSTKVPAAYAAQGYKVLVLNPSVAATLGFGAYMSKAHGIDPNIRT

SEQ ID NO: 320 - protein, 50 aa, synthetic construct; identical to SEQ ID NO: 319

SEQ ID NO: 321 - protein, 50 aa, synthetic construct
PTGSGKSTKVPAAYAAQGYKVLVLNPSVAATLGFGAYMSKAHGVDPNIRT

SEQ ID NO: 322 - protein, 50 aa, synthetic construct
PTGSGKSTKVPVAYAAQGYKVLVLNPSVAATLGFGAYLSKAHGINPNIRT

SEQ ID NO: 323 - protein, 50 aa, synthetic construct
PTGSGKSTKVPAAYASQGYKVLVLNPSVAATLGFGAYMSKAHGINPNIRT

SEQ ID NO: 324 - protein, 50 aa, synthetic construct
PTGSGKSTKVPAAYVAQGYNVLVLNPSVAATLGFGSFMSRAYGIDPNIRT

SEQ ID NO: 325 - protein, 51 aa, synthetic construct
PTGSGKSTKVPAAYAAQGYKVLVLNPSVAATLGFGAYMSKAAYGIDPNIRS

SEQ ID NO: 326 - protein, 50 aa, synthetic construct
PTGSGKSTKVPAAYAAQGYKVLVLNPSVAATLGFGAYMSRAYGVDPNIRT

SEQ ID NO: 327 - protein, 50 aa, synthetic construct
PTGSGKSTKVPAAYASQGYKVLVLNPSVAATLGFGSYMSKAHGIDPNIRT

SEQ ID NO: 328 - protein, 50 aa, synthetic construct
PTGSGKSTKVPAAYASQGYKVLVLNPSVAATLGFGAYMSKAYGIDPSVRT

SEQ ID NO: 329 - protein, 50 aa, synthetic construct
  VARIANT 10: note = X can be any naturally occurring amino acid
  VARIANT 24: note = X can be any naturally occurring amino acid
  VARIANT 49: note = X can be any naturally occurring amino acid
NIEEVALGTXGEIPFYGKAIPLEXIKGGRHLIFCHSKKKCDELAAKLTXL

SEQ ID NO: 330 - protein, 49 aa, synthetic construct
NIEEVALSTTGEIPFYGKAIPLEVIKGGRHLIFCHSKKCDELAAKLVAL

SEQ ID NO: 331 - protein, 50 aa, synthetic construct
NIEEVALSNTGEIPFYGKAIPIETIKGGRHLIFCHSKKKCDELAAKLSGL

SEQ ID NO: 332 - protein, 50 aa, synthetic construct
NIEEVALGQEGEIPFYGRAIPLSYIKGGRHLIFCHSKKKCDELAAALRGM

SEQ ID NO: 333 - protein, 50 aa, synthetic construct
NIEEVALGHEGEIPFYGKAIPLAFIKGGRHLIFCHSKKKCDELAAALRGM

SEQ ID NO: 334 - protein, 50 aa, synthetic construct
NIEEVALGSEGEIPFYGKAIPIAQLKGGRHLIFCHSKKKCDEIASKLRGM

SEQ ID NO: 335 - protein, 50 aa, synthetic construct
NIEEVALPTTGEIPFYGKAIPLELIKGGRHLIFCHSKKKCDELAKQLTSL

SEQ ID NO: 336 - protein, 50 aa, synthetic construct
  VARIANT 23: note = X can be any naturally occurring amino acid
NIEEVALPSEGEIPFYGRAIPLXLIKGGRHLIFCHSKKKCDELAKQLTSL

SEQ ID NO: 337 - protein, 50 aa, synthetic construct
NITETALPTTGEIPFYGKAIPLEYIKGGRHLIFCHSKKKCDELAKQLTSL

SEQ ID NO: 338 - protein, 50 aa, synthetic construct
NIEEVALGNDGEIPFYGKAIPLQHIKGGRHLIFCHSKKKCDELAGKLTSL

SEQ ID NO: 339 - protein, 49 aa, synthetic construct
GLNAVAYYRGLDVSVIPTSGDVVVVATDALMTGFTGDFDSVIDCNVAVT

SEQ ID NO: 340 - protein, 49 aa, synthetic construct
GINAVAYYRGLDVSVIPTSGDVVVVATDALMTGFTGDFDSVIDCNTCVT

SEQ ID NO: 341 - protein, 49 aa, synthetic construct
GLNAVAYYRGLDVSVIPTSGDVVVVATDALMTGFTGDFDSVIDCNTCVT

SEQ ID NO: 342 - protein, 49 aa, synthetic construct
GLNAVAYYRGLDVSIIPTQGDVVVVATDALMTGYTGDFDSVIDCNVAVT

SEQ ID NO: 343 - protein, 49 aa, synthetic construct
GVNAVAYYRGLDVSVIPTQGDVVVVATDALMTGYTGDFDSVIDCNVAVT

SEQ ID NO: 344 - protein, 49 aa, synthetic construct
GLNAVAYYRGLDVSVIPTTGDVVVCATDALMTGFTGDFDSVIDCNVAVE

SEQ ID NO: 345 - protein, 49 aa, synthetic construct
GLNAVAYYRGLDVSVIPTSGDVVVCATDALMTGFTGDFDSVIDCNTSVI

SEQ ID NO: 346 - protein, 49 aa, synthetic construct
GVNAVAYYRGLDVAVIPATGDVVVCSTDALMTGFTGDFDSVIDCNTAVT

SEQ ID NO: 347 - protein, 49 aa, synthetic construct
GLNAVAFYRGVDVSVIPTSGDVVVCATDALMTGYTGDFDSVIDCNVAVT

SEQ ID NO: 348 - protein, 49 aa, synthetic construct
GLTAVAYYRGLDVSVIPTSGDVVVVATDALMTGFTGDFDSVIDCNVAVT

SEQ ID NO: 349 - protein, 53 aa, synthetic construct
CAAILRRHVGPGEGAVQWMNRLIAFASRGNHVSPTHYVPESDASARVTQILSS

SEQ ID NO: 350 - protein, 53 aa, synthetic construct
CAAILRRHVGPGEGAVQWMNRLIAFASRGNHVSPTHYVPESDAAARVTAILSS

SEQ ID NO: 351 - protein, 53 aa, synthetic construct
CAAILRRHVGPGEGAVQWMNRLIAFASRGNHVSPTHYVPESDAAARVTQILSS

SEQ ID NO: 352 - protein, 53 aa, synthetic construct
CAAILRRHVGPGEGAVQWMNRLIAFASRGNHVAPTHYVTESDASQRVTQLLGS

SEQ ID NO: 353 - protein, 53 aa, synthetic construct
CAAILRRHVGQGEGAVQWMNRLIAFASRGNHVAPTHYVAESDASQRVTQVLSS

SEQ ID NO: 354 - protein, 53 aa, synthetic construct
CAAILRRHVGPGEGAVQWMNRLIAFASRGNHVSPTHYVPESDAAARVTALLSS

SEQ ID NO: 355 - protein, 53 aa, synthetic construct; identical to SEQ ID NO: 351

SEQ ID NO: 356 - protein, 53 aa, synthetic construct
CAAVLRRHIGPGEGAVQWMNRLIAFASRGNHVSPTHYVPETDASAKVTQLLSS

SEQ ID NO: 357 - protein, 53 aa, synthetic construct
  VARIANT 46: note = X can be any naturally occurring amino acid
CAAILRRHVGPAEGATQWMNRLIAFASRGNHVSPTHYVPETDASRXVTTILSS

SEQ ID NO: 358 - protein, 53 aa, synthetic construct
CAAIIKRHTGTSEGVTQWMNRLIAFASRGNHVSPTHYIQDDDASKRVMGILSS

SEQ ID NO: 359 - protein, 72 aa, synthetic construct
YQVGYLHAPTGSGKSTKVPAAYAAQGYKVLVLNPSVAATLGFGAYMSKAHGIDPNIRTGVRTVTTGAPITYS

SEQ ID NO: 360 - protein, 72 aa, synthetic construct
FQVAHLHAPTGSGKSTKVPAAYAAQGYKVLVLNPSVAATLGFGAYMSKAHGIDPNIRTGVRTITTGSPITYS

SEQ ID NO: 361 - protein, 72 aa, synthetic construct
FQVAHLHAPTGSGKSTKVPAAYAAQGYKVLVLNPSVAATLGFGAYMSKAHGVDPNIRTGVRTITTGAPITYS

SEQ ID NO: 362 - protein, 72 aa, synthetic construct
YQVGYLHAPTGSGKSTKVPVAYAAQGYKVLVLNPSVAATLGFGAYLSKAHGINPNIRTGVRTVTTGEAITYS

SEQ ID NO: 363 - protein, 72 aa, synthetic construct
YQVGYLHAPTGSGKSTKVPAAYASQGYKVLVLNPSVAATLGFGAYMSKAHGINPNIRTGVRTVTTGDSITYS

SEQ ID NO: 364 - protein, 72 aa, synthetic construct
YQVGYLHAPTGSGKSTKVPAAYVAQGYNVLVLNPSVAATLGFGSFMSRAYGIDPNIRTGNRTVTTGAKLTYS

SEQ ID NO: 365 - protein, 71 aa, synthetic construct
  VARIANT 17: note = X can be any naturally occurring amino acid
  VARIANT 31..32: note = X can be any naturally occurring amino acid
GSVTVPHPNIEEVALGXEGEIPFYGKAIPLXXIKGGRHLIFCHSKKKCDELAAKLRGMGLNAVAYYRGLDV

SEQ ID NO: 366 - protein, 71 aa, synthetic construct
GSVTVPHPNIEEVALSTTGEIPFYGKAIPLEVIKGGRHLIFCHSKKKCDELAAKLVALGINAVAYYRGLDV

SEQ ID NO: 367 - protein, 71 aa, synthetic construct
GSVTVPHPNIEEVALSNTGEIPFYGKAIPIETIKGGRHLIFCDSKKKCDELAAKLSGLGLNAVAYYRGLDV

SEQ ID NO: 368 - protein, 71 aa, synthetic construct
GSVTTPHPNIEEVALGQEGEIPFYGRAIPLSYIKGGRHLIFCHSKKKCDELAAALRGMGLNAVAYYRGLDV

SEQ ID NO: 369 - protein, 71 aa, synthetic construct
GTVTTPHSNIEEVALGHEGEIPFYGKAIPLAFIKGGRHLIFCHSKKKCDELAAALRGMGVNAVAYYRGLDV

SEQ ID NO: 370 - protein, 71 aa, synthetic construct
GSITVPHSNIEEVALGSEGEIPFYGKAIPIALLKGGRHLIFCHSKKKCDELASKLRGMGLNAVAYYRGLDV

SEQ ID NO: 371 - protein, 71 aa, synthetic construct
VKFPGGGQIVGGVYLLPRRGPRLGVRATRKTSERSQPRGRRQPIPKARRSEGRSWAQPGYPWPLYGNEGCG

SEQ ID NO: 372 - protein, 71 aa, synthetic construct
VKFPGGGQIVGGVYLLPRRGPRLGVRATRKTSERSQPRGRRQPIPKARRPEGRTWAQPGYPWPLYGNEGCG

SEQ ID NO: 373 - protein, 71 aa, synthetic construct
VKFPGGGQIVGGVYLLPRRGPRLGVRATRKTSERSQPRGRRQPIPKARRPEGRAWAQPGYPWPLYGNEGMG

SEQ ID NO: 374 - protein, 71 aa, synthetic construct
VKFPGGGQIVGGVYLLPRRGPRLGVRATRKTSERSQPRGRRQPIPKDRRSTGKSWGKPGYPWPLYGNEGLG

SEQ ID NO: 375 - protein, 71 aa, synthetic construct
VKFPGGGQIVGGVYLLPRRGPRLGVRATRKTSERSQPRGRRQPIPKDRRSTGKSWGKPGYPWPLYGNEGCG

SEQ ID NO: 376 - protein, 71 aa, synthetic construct
VKFPGGGQIVGGVYVLPRRGPRLGVRATRKTSERSQPRGRRQPIPKARRSEGRSWAQPGYPWPLYGNEGCG

SEQ ID NO: 377 - protein, 70 aa, synthetic construct
PGALVVGVICAAILRRHVGPGEGAVQWMNRLIAFASRGNHVSPTHYVPESDAAARVTQLSSLTITSLLRR

SEQ ID NO: 378 - protein, 71 aa, synthetic construct
PGALVVGVVCAAILRRHVGPGEGAVQWMNRLIAFASRGNHVSPTHYVPESDAAARVTAILSSLTVTQLLRR

SEQ ID NO: 379 - protein, 71 aa, synthetic construct
PGALVVGVVCAAILRRHVGPGEGAVQWMNRLIAFASRGNHVSPTHYVPESDAAARVTQILSSLTITQLLKR

SEQ ID NO: 380 - protein, 71 aa, synthetic construct
PGALVVGVICAAILRRHVGPGEGAVQWMNRLIAFASRGNHVAPTHYVTESDASQRVTQLLGSLTITSLLRR

SEQ ID NO: 381 - protein, 71 aa, synthetic construct
PGALVVGVICAAILRRHVGQGEGAVQWMNRLIAFASRGNHVAPTHYVAESDASQRVTQVLSSLTITSLLRR

SEQ ID NO: 382 - protein, 71 aa, synthetic construct
PGALVVGVICAAILRRHVGPGEGAVQWMNRLIAFASRGNHVSPTHYVPESDAAARVTALLSSLTVTSLLRR

SEQ ID NO: 383 - protein, 93 aa, synthetic construct
  VARIANT 3: note = X can be any naturally occurring amino acid
  VARIANT 17..18: note = X can be any naturally occurring amino acid
  VARIANT 63: note = X can be any naturally occurring amino acid
LGXEGEIPFYGKAIPLXXIKGGRHLIFCHSKKKCDELAAKLRGMGLNAVAYYRGLDVSVIPTXGDVVVVATDALMTGYTGDFDSVIDCNVAVT

SEQ ID NO: 384 - protein, 93 aa, synthetic construct
LSTTGEIPFYGKAIPLEVIKGGRHLIFCHSKKKCDELAAKLVALGINAVAYYRGLDVSVIPTSGDVVVVATDALMTGYTGDFDSVIDCNTCVT

SEQ ID NO: 385 - protein, 93 aa, synthetic construct
LSNTGEIPFYGKAIPIETIKGGRHLIFCHSKKKCDELAAKLSGLGLNAVAYYRGLDVSVIPTSGDVVVVATDALMTGFTGDFDSVIDCNTCVT

SEQ ID NO: 386 - protein, 93 aa, synthetic construct
LGQEGEIPFYGRAIPLSYIKGGRHLIFCHSKKKCDELAAALRGMGLNAVAYYRGLDVSIIPTQGDVVVVATDALMTGYTGDFDSVIDCNVAVT

SEQ ID NO: 387 - protein, 93 aa, synthetic construct
LGHEGEIPFYGKAIPLAFIKGGRHLIFCHSKKKCDELAAALRGMGVNAVAYYRGLDVSVIPTQGDVVVVATDALMTGYTGDFDSVIDCNVAVT

SEQ ID NO: 388 - protein, 93 aa, synthetic construct
LGSEGEIPFYGKAIPIALLKGGRHLIFCHSKKKCDELASKLRGMGLNAVAYYRGLDVSVIPTTGDVVVCATDALMTGFTGDFDSVIDCNVAVE

SEQ ID NO: 389 - protein, 50 aa, synthetic construct; identical to SEQ ID NO: 150

SEQ ID NO: 390 - protein, 50 aa, synthetic construct; identical to SEQ ID NO: 151

SEQ ID NO: 391 - protein, 50 aa, synthetic construct; identical to SEQ ID NO: 151

SEQ ID NO: 392 - protein, 50 aa, synthetic construct; identical to SEQ ID NO: 152

SEQ ID NO: 393 - protein, 50 aa, synthetic construct; identical to SEQ ID NO: 153

SEQ ID NO: 394 - protein, 50 aa, synthetic construct; identical to SEQ ID NO: 148

SEQ ID NO: 395 - protein, 50 aa, synthetic construct; identical to SEQ ID NO: 148

SEQ ID NO: 396 - protein, 50 aa, synthetic construct; identical to SEQ ID NO: 148

SEQ ID NO: 397 - protein, 35 aa, synthetic construct; identical to SEQ ID NO: 133

SEQ ID NO: 398 - protein, 35 aa, synthetic construct; identical to SEQ ID NO: 133

SEQ ID NO: 399 - protein, 35 aa, synthetic construct; identical to SEQ ID NO: 135

SEQ ID NO: 400 - protein, 35 aa, synthetic construct; identical to SEQ ID NO: 136

SEQ ID NO: 401 - protein, 35 aa, synthetic construct; identical to SEQ ID NO: 137

SEQ ID NO: 402 - protein, 35 aa, synthetic construct; identical to SEQ ID NO: 133

SEQ ID NO: 403 - protein, 35 aa, synthetic construct; identical to SEQ ID NO: 133

SEQ ID NO: 404 - protein, 35 aa, synthetic construct; identical to SEQ ID NO: 133

SEQ ID NO: 405 - protein, 42 aa, synthetic construct; identical to SEQ ID NO: 233

SEQ ID NO: 406 - protein, 42 aa, synthetic construct; identical to SEQ ID NO: 234

SEQ ID NO: 407 - protein, 42 aa, synthetic construct; identical to SEQ ID NO: 235

SEQ ID NO: 408 - protein, 42 aa, synthetic construct
GHEGEIPFYGKAIPLAFIKGGRHLIFCHSKKKCDELAAALRG

SEQ ID NO: 409 - protein, 42 aa, synthetic construct
GSEGEIPFYGKAIPIALLKGGRHLIFCHSKKKCDELASKLRG

SEQ ID NO: 410 - protein, 44 aa, synthetic construct
  VARIANT 2: note = X can be any naturally occurring amino acid
  VARIANT 18..19: note = X can be any naturally occurring amino acid
GXEGEGEIPFYGKAIPLXXIKGGRHLIFCHSKKKCDELAAKLRG

SEQ ID NO: 411 - protein, 42 aa, synthetic construct; identical to SEQ ID NO: 228

SEQ ID NO: 412 - protein, 42 aa, synthetic construct
GTEGEIPFYGKAIPLEQIKGGRHLIFCHSKKKCDELAAKLRG

SEQ ID NO: 413 - protein, 42 aa, synthetic construct
KGGRHLIFCHSKKKCDELAAKLVALGINAVAYYRGLDVSVIP

SEQ ID NO: 414 - protein, 42 aa, synthetic construct
KGGRHLIFCHSKKKCDELAAKLSGLGLNAVAYYRGLDVSVIP

SEQ ID NO: 415 - protein, 42 aa, synthetic construct
KGGRHLIFCHSKKKCDELAAALRGMGLNAVAYYRGLDVSIIP

SEQ ID NO: 416 - protein, 42 aa, synthetic construct
KGGRHLIFCHSKKKCDELAAALRGMGVNAVAYYRGLDVSVIP

SEQ ID NO: 417 - protein, 42 aa, synthetic construct
KGGRHLIFCHSKKKCDELASKLRGMGLNAVAYYRGLDVSVIP

SEQ ID NO: 418 - protein, 42 aa, synthetic construct
KGGRHLIFCHSKKKCDELAAKLRGMGLNAVAYYRGLDVSVIP

SEQ ID NO: 419 - protein, 42 aa, synthetic construct; identical to SEQ ID NO: 291

SEQ ID NO: 420 - protein, 42 aa, synthetic construct; identical to SEQ ID NO: 418

SEQ ID NO: 421 - protein, 48 aa, synthetic construct; identical to SEQ ID NO: 267

SEQ ID NO: 422 - protein, 48 aa, synthetic construct; identical to SEQ ID NO: 268

SEQ ID NO: 423 - protein, 48 aa, synthetic construct; identical to SEQ ID NO: 269

SEQ ID NO: 424 - protein, 47 aa, synthetic construct
ILRRHVGQGEGAVQWMNRLIAFASRGNHAPTHYVAESDASQRVTQVL

SEQ ID NO: 425 - protein, 48 aa, synthetic construct; identical to SEQ ID NO: 271

SEQ ID NO: 426 - protein, 47 aa, synthetic construct
ILRRHVGPGEGAVQWMNRLIAFASRGNHVSPTHYVPESDAAARVTQL

SEQ ID NO: 427 - protein, 48 aa, synthetic construct; identical to SEQ ID NO: 254

SEQ ID NO: 428 - protein, 48 aa, synthetic construct; identical to SEQ ID NO: 268

(19) United States
(12) Patent Application Publication
(10) Pub. No.: US 2024/0398938 A1
(43) Pub. Date: Dec. 5, 2024

(54) Hepatitis C Virus Peptide Compositions and Methods of Use Thereof

(71) Applicant: The Governors of the University of Alberta, Edmonton (CA)

(72) Inventors: Michael Houghton, Danville, CA (US); Abdolamir Landi, Edmonton (CA); Michael Logan, Edmonton (CA); John L. Law, Edmonton (CA); Chao Chen, Edmonton (CA); Darren Hockman, Edmonton (CA)

(21) Appl. No.: 18/740,380

(22) Filed: Jun. 11, 2024

Related U.S. Application Data
(63) Continuation of application No. 16/978,486, filed on Sep. 4, 2020, now Pat. No. 12,048,742.
(60) Provisional application No. 62/644,140, filed on Mar. 16, 2018.

(51) Int. Cl. A61K 39/29; A61K 39/00; A61P 31/14; A61P 37/04

(52) U.S. Cl. CPC A61K 39/29; A61P 31/14; A61P 37/04; A61K 2039/55505; C12N 2770/24222; C12N 2770/24234

(57) ABSTRACT

The present disclosure provides an immunogenic composition comprising: a) i) a hepatitis C virus (HCV) heterodimeric polypeptide that includes HCV E1 and E2 polypeptides; ii) an HCV E1 polypeptide; or iii) an HCV E2 polypeptide; b) a polypeptide (also referred to herein as a “T-cell epitope polypeptide” or an “HCV T-cell epitope polypeptide”) comprising T-cell epitopes (e.g., CD4⁺ and CD8⁺ T-cell epitopes that are conserved among some HCV genotypes and that are presented through one or multiple HLA alleles common within the human population) present in an HCV protein other than E1 and E2; and c) a pharmaceutically acceptable excipient. The present disclosure provides a method of inducing an immune response, in an individual, to an HCV polypeptide.

### CROSS-REFERENCE

This application is a continuation of U.S. patent application Ser. No. 16/978,486, filed Sep. 4, 2020, which application is a 371 of international Application no. PCT/CA2019/050320, filed Mar. 14, 2019, which claims the benefit of U.S. Provisional Patent Application No. 62/644,140, filed Mar. 16, 2018, which application is incorporated herein by reference in its entirety.

### INCORPORATION BY REFERENCE OF SEQUENCE LISTING PROVIDED AS A SEQUENCE LISTING XML FILE

A Sequence Listing is provided herewith as a Sequence Listing XML file, “UALB-041CON_SEQ_LIST.xml” created on Jun. 11, 2024 and having a size of 537,852 bytes. The contents of the text file are incorporated by reference herein in their entirety.

### INTRODUCTION

Hepatitis C virus (HCV) is a blood-borne pathogen that is estimated to infect 150-200 million people worldwide. Infection by HCV may be non-symptomatic, and can be cleared by patients, sometimes without medical intervention. However, the majority of patients develop a chronic HCV infection, which may lead to liver inflammation, scarring, and even to liver failure or liver cancer. In the United States alone, over 3 million people have a chronic infection.

The HCV virion contains a positive-sense single stranded RNA genome of about 9.5 kb. The genome encodes a single polyprotein of 3,010 to 3,030 amino acids. The structural proteins comprise a core protein forming the viral nucleocapsid and two envelope glycoproteins, E1 and E2.

A vaccine based on the recombinant envelope glycoproteins (rE1E2) from a single genotype 1a strain (HCV-1) protected chimpanzees from chronic infection following homologous and heterologous genotype 1a (gt1a) viral challenge (reviewed in Houghton, M Immunol Rev 2011). Antisera from the immunized chimpanzees were shown to exhibit in vitro cross-neutralizing activity (Meunier et al. (2011) J. Infect. Dis. 204:1186). A phase I clinical trial was conducted in human volunteers with a similar antigen (Frey et al. (2010) Vaccine 28:6367). Antisera from selected vaccinated individuals were similarly capable of neutralizing chimeric cell culture-derived viruses (HCVcc) expressing the structural proteins of strains representing all 7 major HCV genotypes in vitro (Law et al. (2013) PLoS One 8:e59776) and to be able to compete with the binding of numerous discrete monoclonal antibodies with broad cross-neutralizing activities (Wong et al. (2014) J. Virol. 88:14278).

There is a need in the art for compositions and methods for inducing immune responses to HCV.

### SUMMARY

The present disclosure provides an immunogenic composition comprising: a) i) a hepatitis C virus (HCV) heterodimeric polypeptide that includes HCV E1 and E2 polypeptides; ii) an HCV E1 polypeptide; or iii) an HCV E2 polypeptide; b) a polypeptide (also referred to herein as a “T-cell epitope polypeptide” or an “HCV T-cell epitope polypeptide”) comprising T-cell epitopes (e.g., CD4⁺ and CD8⁺ T-cell epitopes that are conserved among some HCV genotypes and that are presented through one or multiple HLA alleles common within the human population) present in an HCV protein other than E1 and E2; and c) a pharmaceutically acceptable excipient. The present disclosure provides a method of inducing an immune response, in an individual, to an HCV polypeptide. The present disclosure provides an immunogenic composition comprising: a) a polypeptide that comprises one or more T-cell epitopes (e.g., CD4⁺ and CD8⁺ T-cell epitopes that are conserved among some HCV genotypes and that are presented through one or multiple HLA alleles common within the human population) present in an HCV protein other than E1 and E2; and b) a pharmaceutically acceptable excipient.

### BRIEF DESCRIPTION OF THE DRAWINGS

FIG. 1A-1C provide an amino acid sequence alignment of examples of the core-E1-E2 coding regions of a HCV genotype 1 virus, specifically representative HCV 1A, 1B and 1C genotypes. Genbank database sequences for the coding region core-E1-E2 were aligned using Geneious software v5.6.4. Numbering of amino acids is according to strain NP_671941 (H77). Consensus: SEQ ID NO:1; AVI1a129: SEQ ID NO:2; NP_671491 (H77): SEQ ID NO:3; EU155269: SEQ ID NO:4; EU781810: SEQ ID NO:5; EU781771: SEQ ID NO:6; AB250610: SEQ ID NO:7; EU781752: SEQ ID NO:8; EU781759: SEQ ID NO:9; EF407439: SEQ ID NO:10; EF407427: SEQ ID NO:11; EU362905: SEQ ID NO:12; EF407413: SEQ ID NO:13; EU781808: SEQ ID NO:14; EU78170: SEQ ID NO:15; AJ238799 (Con1): SEQ ID NO:16; AAK97744: SEQ ID NO:17; AF139594: SEQ ID NO:18; AF176573: SEQ ID NO:19; BAA19625: SEQ ID NO:20; BAA25076: SEQ ID NO:21; BAC54896: SEQ ID NO:22; BAD91386: SEQ ID NO:23; BAF46764: SEQ ID NO:24; BAG30950: SEQ ID NO:25; CAB41951: SEQ ID NO:26; AAK95832: SEQ ID NO:27; AAT69968: SEQ ID NO:28; and BAA03581: SEQ ID NO:29.

FIG. 2A-2C provide an alignment of amino acid sequences of the core-E1-E2 coding region of representative HCV 2A and HCV2B subtypes. Genbank database sequences for the coding region core-E1-E2 were aligned using Geneious software v5.6.4. The amino acid numbering depicted is in accordance to the common HCV strains: AB047639 (JFH1) and HPCJ8G-J8 (J8) for HCV2A and HCV2B, respectively. AB047639 (JFH1): SEQ ID NO:30; AB047645: SEQ ID NO:31; AF169003: SEQ ID NO:32; AF169005: SEQ ID NO:33; AF238482: SEQ ID NO:34; AY746460: SEQ ID NO:35; HPCPOLP: SEQ ID NO:36; NC_009823: SEQ ID NO:37; HPCJ8G HC-J8: SEQ ID NO:38; AB030907: SEQ ID NO:39; AY232730: SEQ ID NO:40; AY232747: SEQ ID NO:41; and DQ430817: SEQ ID NO:42.

FIG. 3A-3C provide an amino acid sequence alignment of the core-E1-E2 coding region for representative HCV 3A, 3B and 3K genotypes. Genbank database sequences for the coding region core-E1-E2 were aligned using Geneious software v5.6.4. Consensus: SEQ ID NO:43; AVI3a177: SEQ ID NO:44; YP_0014696: SEQ ID NO:45; CAA54244: SEQ ID NO:46; AAC03058: SEQ ID NO:47; AAY29642: SEQ ID NO:48; ABD85062: SEQ ID NO:49; ABD85063: SEQ ID NO:50; ABD97104: SEQ ID NO:51; BAA06044: SEQ ID NO:52; BAA08372: SEQ ID NO:53; and BAA09890: SEQ ID NO:54.

FIG. 4A-4B provide an amino acid sequence of the core-E1-E2 coding region for HCV genotype 7a. Amino acid sequence for the coding region core-E1-E2 of genotype 7a (isolate QC69; Genbank: ABN05226.1; SEQ ID NO:55) is shown according to the numbering scheme of the reference strain, NP_671941 (H77).

FIG. 5A-5C provide amino acid sequences of immunoglobulin Fc regions for GenBank 3S7G_A Homo sapiens IgG1 Fc: SEQ ID NO:56; GenBank AAN76044 Homo sapiens IgG2 Fc: SEQ ID NO: 57; GenBank AAW65947 Homo sapiens IgG3 Fc: SEQ ID NO:58; GenBank AAA52770 Homo sapiens IgD Fc: SEQ ID NO:59; GenBank 0308221A Homo sapiens IgM Fc: SEQ ID NO:60; GenBank P01876 Homo sapiens IgA Fc: SEQ ID NO:61; GenBank IF6A_B Homo sapiens IgE Fc: SEQ ID NO:62; and GenBank P01861 Homo sapiens IgG4 Fc: SEQ ID NO:63.

FIG. 6 provides the solubility of various T-cell epitope polypeptides in an aqueous buffer. TP35-NS3: SEQ ID NO:133; TP35-NS3(Lys)₃: SEQ ID NO:302; TP42: SEQ ID NO:228; TP45 (C-terminal CNV replaced with KKK): SEQ ID NO:248; TP48: SEQ ID NO:254; TP48(Lys)₃: SEQ ID NO:257; and TP50-C: SEQ ID NO:148.

FIG. 7 provides an alignment of amino acid sequences of HCV polypeptides; and shows the positions of TP50-C, conserved MHC class II CD4-specific HCV epitopes, and conserved MHC class I CD8-specific HCV epitopes. Top to bottom: SEQ ID NOs: 309-318.

FIG. 8 provides an alignment of amino acid sequences of HCV polypeptides; and shows the positions of TP35-NS3, conserved MHC class II CD4-specific HCV epitopes, conserved MHC class I CD8-specific HCV epitopes, and MHC class I CD8-specific HCV epitopes with corresponding HLA specificity. Top to bottom: SEQ ID NOs: 319-328.

FIG. 9 provides an alignment of amino acid sequences of HCV polypeptides; and shows the positions of TP27, TP42, conserved MHC class II CD4-specific HCV epitopes, conserved MHC class I CD8-specific HCV epitopes, and MHC class I CD8-specific HCV epitopes with corresponding HLA specificity. Top to bottom: SEQ ID NOs: 329-338.

FIG. 10 provides an alignment of amino acid sequences of HCV polypeptides; and shows the positions of TP23, TP45, conserved MHC class II CD4-specific HCV epitopes, conserved MHC class I CD8-specific HCV epitopes, and MHC class I CD8-specific HCV epitopes with corresponding HLA specificity. Top to bottom: SEQ ID NOs: 339-348.

FIG. 11 provides an alignment of amino acid sequences of HCV polypeptides; and shows the positions of TP35-NS4, TP48, conserved MHC class II CD4-specific HCV epitopes, and conserved MHC class I CD8-specific HCV epitopes. Top to bottom: SEQ ID NOs: 349-358.

FIG. 12 provides the solubility of various T-cell epitope polypeptides. TP45 SEQ ID NO:247; TP45+KKK SEQ ID NO:251; TP23: SEQ ID NO:186; TP23+KKK: SEQ ID NO:261; TP27: SEQ ID NO:188; TP27+KKK: SEQ ID NO:263; TP35-NS4: SEQ ID NO:203; and TP35-NS4+KKK: SEQ ID NO:265.

FIG. 13 provides the solubility of various T-cell epitope polypeptides. TP42: SEQ ID NO:228; TP45+KKK: SEQ ID NO:251; TP48: SEQ ID NO:254.

FIG. 14 provides the solubility of various T-cell epitope polypeptides. TP45+KKK: SEQ ID NO:251; TP48: SEQ ID NO:254.

FIG. 15 provides an alignment of amino acid sequences of a portion of NS3 of HCV-1a, -1b, -2a, -2b, and -3, as well as a consensus sequence; and shows the positions of conserved MHC class II CD4-specific HCV epitopes, and conserved MHC class I CD8-specific HCV epitopes in TP35-NS3. Top to bottom: SEQ ID NOs:359-364. The upper bar (“100% of consensuses”) indicates CD8 epitopes; the lower bar (“100%”) indicates CD4 epitopes.

FIG. 16 provides an alignment of amino acid sequences of a portion of NS3 of HCV-1a, -1b, -2a, -2b, and -3, as well as a consensus sequence; and shows the positions of conserved MHC class II CD4-specific HCV epitopes, and conserved MHC class I CD8-specific HCV epitopes in TP27 and TP42. Top to bottom: SEQ ID NOs:365-370. The bars labeled “100% of 5 consensuses” indicate CD8 epitopes. The bar labeled “100%” indicates CD4 epitopes.

FIG. 17 provides an alignment of amino acid sequences of a portion of NS3 of HCV-1a, -1b, -2a, -2b, and -3, as well as a consensus sequence; and shows the positions of conserved MHC class II CD4-specific HCV epitopes, and conserved MHC class I CD8-specific HCV epitopes in TP50-C. Top to bottom: SEQ ID NOs:371-376. The bars labeled “100% of 5 consensuses” indicate CD8 epitopes. The bars labeled “100%” indicate CD4 epitopes.

FIG. 18 provides an alignment of amino acid sequences of a portion of NS3 of HCV-1a, -1b, -2a, -2b, and -3, as well as a consensus sequence; and shows the positions of conserved MHC class II CD4-specific HCV epitopes, and conserved MHC class I CD8-specific HCV epitopes in TP35-NS4 and TP-48. Top to bottom: SEQ ID NOs:377-382. The bars labeled “100% of 5 consensuses” indicate CD8 epitopes. The bars labeled “100%” indicate CD4 epitopes.

FIG. 19 provides a table showing population coverage (PC) analysis of reported and predicted epitopes.

FIG. 20 depicts T-cell responses to various T-cell epitope polypeptides of the present disclosure in mice.

FIG. 21 provides an alignment of amino acid sequences of T-cell epitope polypeptides designated TP42, TP42-2, TP27, TP33, TP45, and TP23, from HCV-1a, HCV-1b, HCV-2a, HCV-2b, and HCV3. Top to Bottom: SEQ ID NOs:383-388. The bars labeled “100% of 5 co . . . ” or “100% of 5 cons . . . ” indicate CD8 epitopes. The bar labeled “100%” indicates CD4 epitopes.

FIG. 22 provides an alignment of amino acid sequences of TP50-C T-cell epitope polypeptides from HCV-1a, HCV-1b, HCV-2a, HCV-2b, and HCV3; a final consensus sequence; an amino acid sequence of a TP50-C polypeptide on which solubility studies were conducted (“ordered”); and an amino acid sequence that was used for epitope prediction analyses (“for prediction”). Top to Bottom: SEQ ID NOs:389-396.

FIG. 23 provides an alignment of amino acid sequences of TP35-NS3 T-cell epitope polypeptides from HCV-1a, HCV-1b, HCV-2a, HCV-2b, and HCV3; a final consensus sequence; an amino acid sequence of a TP35-NS3 polypeptide on which solubility studies were conducted (“ordered”); and an amino acid sequence that was used for epitope prediction analyses (“for prediction”). Top to Bottom: SEQ ID NOs:397-404.

FIG. 24 provides an alignment of amino acid sequences of TP42 T-cell epitope polypeptides from HCV-1a, HCV-1b, HCV-2a, HCV-2b, and HCV3; a final consensus sequence; an amino acid sequence of a TP42 polypeptide on which solubility studies were conducted (“ordered”); and an amino acid sequence that was used for epitope prediction analyses (“for prediction”). Top to Bottom: SEQ ID NOs:405-412.

FIG. 25 provides an alignment of amino acid sequences of TP42-2 T-cell epitope polypeptides from HCV-1a, HCV-1b, HCV-2a, HCV-2b, and HCV3; a final consensus sequence; an amino acid sequence of a TP42-2 polypeptide on which solubility studies were conducted (“ordered”); and an amino acid sequence that was used for epitope prediction analyses (“for prediction”). Top to Bottom: SEQ ID NOs:413-420.

FIG. 26 provides an alignment of amino acid sequences of TP48 T-cell epitope polypeptides from HCV-1a, HCV-1b, HCV-2a, HCV-2b, and HCV3; a final consensus sequence; an amino acid sequence of a TP48 polypeptide on which solubility studies were conducted (“ordered”); and an amino acid sequence that was used for epitope prediction analyses (“for prediction”). Top to Bottom: SEQ ID NOs:421-428.

FIG. 27 depicts solubility data for a TP33 T-cell epitope polypeptide (SEQ ID NO:279) and a TP42-2 T-cell epitope polypeptide (SEQ ID NO:291).

### DEFINITIONS

The term “hepatitis C virus” (“HCV”), as used herein, refers to any one of a number of different genotypes and isolates of hepatitis C virus. Thus, “HCV” encompasses any of a number of genotypes, subtypes, or quasispecies, of HCV, including, e.g., genotype 1, 2, 3, 4, 6, 7, etc. and subtypes (e.g., 1a, 1b, 2a, 2b, 3a, 4a, 4c, etc.), and quasispecies. Representative HCV genotypes and isolates include: HCV-1, H77, J6, Con1, isolate 1, BK, EC1, EC10, HC-J2, HC-J5; HC-J6, HC-J7, HC-J8, HC-JT, HCT18, HCT27, HCV-476, HCV-KF, “Hunan”, “Japanese”, “Taiwan”, TH, type 1, type 1a, H77 type 1b, type 1c, type 1d, type 1e, type 1f, type 10, type 2, type 2a, type 2b, type 2c, type 2d, type 2f, type 3, type 3a, type 3b, type 3g, type 4, type 4a, type 4c, type 4d, type 4f, type 4h, type 4k, type 5, type 5a, type 6, type 6a, and type 7a.

The terms “individual,” “host,” “subject,” and “patient” are used interchangeably herein, and refer to a mammal, including, but not limited to, non-human primates (e.g., simians), equines (e.g., horses), rodents (e.g., rats; mice), and humans.

As used herein, the term “isolated,” in reference to a polypeptide, refers to a polypeptide that is in an environment different from that in which the polypeptide naturally occurs. An isolated polypeptide can be purified. By “purified” is meant a compound of interest (e.g., a polypeptide) has been separated from components that accompany it in nature. “Purified” can also be used to refer to a polypeptide separated from components that can accompany it during production of the polypeptide (e.g., during synthesis in vitro, etc.). In some embodiments, a polypeptide (or a mixture of polypeptides) is substantially pure when the polypeptide (or mixture of polypeptides) is at least 60% or at least 75% by weight free from organic molecules with which it is naturally associated or with which it is associated during production. In some cases, the polypeptide is from 30% to 60% pure. In some cases, the polypeptide (or mixture of polypeptides) is at least 60%, at least 75%, at least 80%, at least 85%, at least 90%, at least 95%, or at least 99%, by weight, pure. For example, in some cases, an E1 or an E2 polypeptide (or a mixture of E1 and E2 polypeptides, e.g., an E1/E2 heterodimer) is substantially pure when the E1 or E2 polypeptide (or mixture of E1 and E2 polypeptides) is at least 60% or at least 75% by weight free from organic molecules with which the polypeptide(s) is naturally associated or with which it is associated during production. In some cases, the E1 or E2 polypeptide (or mixture of E1 and E2 polypeptides) is at least 60%, at least 75%, at least 80%, at least 85%, at least 90%, at least 95%, or at least 99%, by weight, pure. In some cases, where a composition comprises an E2 polypeptide, the E2 polypeptide is at least 60%, at least 75%, at least 80%, at least 85%, at least 90%, at least 95%, or at least 99%, by weight, pure. In some cases, where a composition comprises an E1/E2 heterodimeric complex polypeptide, the E1/E2 heterodimeric complex polypeptide is at least 60%, at least 75%, at least 80%, at least 85%, at least 90%, at least 95%, or at least 99%, by weight, pure. In some cases, where a composition comprises a T-cell epitope polypeptide, the T-cell epitope polypeptide is at least 60%, at least 75%, at least 80%, at least 85%, at least 90%, at least 95%, or at least 99%, by weight, pure.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. In some cases, a polynucleotide is RNA. In some cases, a polynucleotide is DNA. A “polynucleotide” includes a nucleic acid that is incorporated into a viral vector or a bacterial vector.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The term “polypeptide” includes glycosylated polypeptides.

The term “heterologous” refers to two components that are defined by structures derived from different sources. For example, where “heterologous” is used in the context of a polypeptide, where the polypeptide includes operably linked amino acid sequences that can be derived from one or more different polypeptides, e.g., amino acid sequences that are not operably linked to the polypeptide in nature. As another example, where a composition comprises an HCV E1/E2 heterodimer and a “heterologous” polypeptide, the “heterologous polypeptide is a polypeptide other than HCV E1 or HCV E2. As another example, where a composition comprises an HCV E1 polypeptide and a “heterologous” polypeptide, the “heterologous polypeptide” is a polypeptide other than HCV E1. As another example, where a composition comprises an HCV E2 polypeptide and a “heterologous” polypeptide, the “heterologous polypeptide is a polypeptide other than HCV E2. As another example, where a fusion polypeptide comprises: a) a T-cell epitope polypeptide; and b) a heterologous fusion partner polypeptide, the “heterologous fusion partner polypeptide” is one that is not found associated with the T-cell epitope polypeptide in nature.

A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well known to those of skill in the art. See, e.g., Pearson (1994) Methods Mol. Biol. 24: 307-331, herein incorporated by reference. Examples of groups of amino acids that have side chains with similar chemical properties, and that thus constitute conservative amino acid substitution groups, include: 1) aliphatic side chain-containing amino acids: glycine, alanine, valine, leucine and isoleucine; 2) aliphatic-hydroxyl side chain-containing amino acids: serine and threonine; 3) amino acids with amide-containing side chains: asparagine and glutamine; 4) aromatic side chain-containing amino acids: phenylalanine, tyrosine, and tryptophan; 5) amino acids with basic side chains: lysine, arginine, and histidine; 6) amino acids with acidic side chains: aspartate and glutamate, and 7) amino acids with sulfur-containing side chains: cysteine and methionine. Examples of conservative amino acid substitution groups are: valine-leucine-isoleucine, phenylalanine-tyrosine, lysine-arginine, alanine-valine, glutamate-aspartate, and asparagine-glutamine.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a T-cell epitope” includes a plurality of such epitopes and reference to “the E1/E2 heterodimer” includes reference to one or more E1/E2 heterodimers and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides an immunogenic composition comprising: a) a polypeptide that comprises one or more T-cell epitopes (e.g., CD4⁺ and/or CD8⁺ T-cell epitopes that are conserved between or among two or more HCV genotypes and that are presented through one or multiple HLA alleles common within the human population) present in an HCV protein other than E1 and E2; and b) a pharmaceutically acceptable excipient. The present disclosure provides an immunogenic composition comprising: a) i) a hepatitis C virus (HCV) heterodimeric polypeptide that includes HCV E1 and E2 polypeptides; ii) an HCV E1 polypeptide; or iii) an HCV E2 polypeptide; b) a heterologous polypeptide (also referred to herein as a “T-cell epitope polypeptide” or an “HCV T-cell epitope polypeptide”) comprising T-cell epitopes (e.g., CD4⁺ and/or CD8⁺ T-cell epitopes that are conserved between or among two or more HCV genotypes and that are presented through one or multiple HLA alleles common within the human population) present in an HCV protein other than E1 and E2; and c) a pharmaceutically acceptable excipient. The present disclosure provides a method of inducing an immune response in an individual to an HCV polypeptide.

An immunogenic composition of the present disclosure, when administered to an individual (e.g., an individual in need thereof), induces an immune response to HCV in the individual. In some cases, an immunogenic composition of the present disclosure, when administered to an individual (e.g., an individual in need thereof), induces a protective immune response to HCV in the individual. A protective immune response to HCV can include one or both of: i) an HCV-specific T cell response, which can include an HCV-specific CD8⁺ T cell response and/or an HCV-specific CD4⁺ T cell response; and ii) production of cross-neutralizing antibodies to HCV.

In some cases, an immunogenic composition of the present disclosure comprises, as separate entities (i.e., not in covalent linkage to one another): a) an HCV E1/E2 heterodimer; and b) a T-cell epitope polypeptide comprising a T-cell epitope present in an HCV protein other than E1 and E2. In some cases, an immunogenic composition of the present disclosure comprises, as separate entities (i.e., not in covalent linkage to one another): a) an HCV E2 polypeptide; and b) a T-cell epitope polypeptide comprising a T-cell epitope present in an HCV protein other than E1 and E2. In some cases, an immunogenic composition of the present disclosure comprises, as separate entities (i.e., not in covalent linkage to one another): a) an HCV E1 polypeptide; and b) a T-cell epitope polypeptide comprising a T-cell epitope present in an HCV protein other than E1 and E2.

As noted above, T-cell epitopes that are present in a heterologous polypeptide suitable for inclusion in an immunogenic composition of the present disclosure include CD4⁺ and/or CD8⁺ T-cell epitopes that are conserved between or among two or more HCV genotypes and that are presented through one or multiple HLA alleles common within the human population. Thus, a heterologous polypeptide suitable for inclusion in an immunogenic composition of the present disclosure comprises multiple (e.g., 2, 3, 4, 5, or more than 5) CD4⁺ and/or CD8⁺ T-cell epitopes that are conserved among some HCV genotypes and that are presented through one or multiple HLA alleles common within the human population. In some cases, a T-cell epitope polypeptide includes epitopes that are conserved among a subset of HCV genotypes. For example, in some cases, a T-cell epitope polypeptide includes epitopes that are conserved among at least 3 HCV genotypes. For example, in some cases, a T-cell epitope polypeptide includes epitopes that are conserved among only HCV genotypes 1a, 1b, and 3 (but not genotypes 4, 5, 6, or 7, for example). In some cases, one or more of the epitopes presented by a T-cell epitope polypeptide is/are conserved among all HCV genotypes. A T-cell epitope polypeptide can include: i) one or more epitopes that are conserved among a subset of HCV genotypes; and ii) one or more epitopes that are conserved among all HCV genotypes. For example, a T-cell epitope polypeptide can include: i) one or more epitopes that are conserved among only HCV genotypes 1a, 1b, and 3 (but not genotypes 4, 5, 6, or 7, for example); and ii) one or more epitopes that are conserved among all HCV genotypes.

Suitable T-cell epitope polypeptides are described in detail below. The T-cell epitope polypeptide can be expressed in any suitable host cell, e.g., a bacterial host cell, a yeast host cell, an insect host cell, a mammalian host cell) as a separate polypeptide, then combined with a E1/E2 heterodimer, an E2 polypeptide, or an E1 polypeptide, to form and immunogenic composition. The T-cell epitope polypeptides serve to elicit broad spectrum CD4⁺ and CD8⁺ T cell responses to multiple HCV genotypes because the T-cell epitope polypeptides have been selected to contain a plurality of T cell epitopes that are highly conserved among at least some genotypes of the hepacivirus genus, and which may in some cases be immunodominant. The T-cell epitope polypeptides also contain T cell epitopes presented by various MHC alleles common in the human population. The E1/E2 antigens will also elicit cross-reactive T cell responses; however, the T-cell epitope polypeptides will elicit broader T cell responses that are cross-reactive with multiple HCV genotypes in the general human population. Both neutralizing antibodies and T cell responses are known to be protective against HCV; thus, this combination of antigens, optionally along with a suitable adjuvant (e.g., AS01 or MF59 or Alum/MPL) will optimize the protective effects of a HCV vaccine.

The T-cell epitope polypeptides can be chemically-synthesized, e.g., using standard methods of chemical synthesis of polypeptides. The T-cell epitope polypeptides can also be produced using recombinant means. The T-cell epitope polypeptides may be expressed alone (e.g., without any heterologous polypeptide appended thereto), and then purified conventionally. Alternatively, the T-cell epitope polypeptides can be expressed downstream of, or upstream of, an immunoglobulin (Ig) Fc fragment (or other affinity tag) separated by a protease cleavage site (e.g., a Precision protease cleavage site) and then purified.

Immunogenic Compositions Comprising: A) an HCV E1/E2 Heterodimer, an HCV E2 Polypeptide, or an HCV E1 Polypeptide; and/or B) a Heterologous Polypeptide Comprising a T-Cell Epitope (a “T-Cell Epitope Polypeptide”)

The present disclosure provides an immunogenic composition comprising: a) a T-cell epitope polypeptide (a polypeptide other than an HCV E1 or E2 polypeptide) comprising T-cell epitopes (e.g., CD4⁺ and/or CD8⁺ T-cell epitopes that are conserved between or among two or more HCV genotypes and that are presented through one or multiple HLA alleles common within the human population) present in an HCV protein other than E1 and E2; and b) a pharmaceutically acceptable excipient. In some cases, the immunogenic composition comprises an adjuvant. The present disclosure provides an immunogenic composition comprising: a) i) an HCV heterodimeric polypeptide that includes HCV E1 and E2 polypeptides; ii) an HCV E1 polypeptide; or iii) an HCV E2 polypeptide; b) a T-cell epitope polypeptide (a polypeptide other than an HCV E1 or E2 polypeptide) comprising T-cell epitopes (e.g., CD4⁺ and/or CD8⁺ T-cell epitopes that are conserved between or among two or more HCV genotypes and that are presented through one or multiple HLA alleles common within the human population) present in an HCV protein other than E1 and E2; and c) a pharmaceutically acceptable excipient. In some cases, the immunogenic composition comprises an adjuvant.

As noted above, T-cell epitopes that are present in a T-cell epitope polypeptide suitable for inclusion in an immunogenic composition of the present disclosure include CD4⁺ and/or CD8⁺ T-cell epitopes that are conserved between or among two or more HCV genotypes and that are presented through one or multiple HLA alleles common within the human population. Thus, a T-cell epitope polypeptide suitable for inclusion in an immunogenic composition of the present disclosure comprises multiple (e.g., 2, 3, 4, or 5, or more than 5) CD4⁺ and/or CD8⁺ T-cell epitopes that are conserved between or among two or more HCV genotypes and that are presented through one or multiple HLA alleles common within the human population. In some cases, the immunogenic composition comprises an adjuvant. In some cases, a CD8 epitope present in a T-cell epitope polypeptide of the present disclosure presented through a single HLA allele (a single HLA haplotype). In some cases, a CD4 epitope present in a T-cell epitope polypeptide of the present disclosure presented through multiple different HLA alleles (multiple different HLA haplotypes).

In some cases, an immunogenic composition of the present disclosure, when administered to an individual in need thereof, induces an immune response in the individual to one or more HCV genotypes. In some cases, an immunogenic composition of the present disclosure, when administered to an individual in need thereof, induces an immune response in the individual to one or more HCV genotypes, where the immune response is greater than the immune response induced by administration of a control composition comprising the HCV E1/E2 heterodimer (or E1 polypeptide, or E2 polypeptide) but lacking the heterologous polypeptide.

In some cases, an immunogenic composition of the present disclosure, when administered to an individual in need thereof, increases one or both of: a) the number of antigen-specific proliferating T cells; and b) the number of antigen-specific cytokine-secreting T-cells.

In some cases, an immunogenic composition of the present disclosure, when administered to an individual in need thereof, induces CD8⁺ CTLs specific for HCV, where the number of HCV-specific CD8⁺ CTLs induced is at least 10%, at least 15%, at least 20%, at least 25%, at least 30%, at least 40%, at least 50%, at least 75%, at least 100% (or 2-fold), at least 2.5-fold, at least 5-fold, at least 7.5-fold, at least 10-fold, at least 20-fold, at least 50-fold, or at least 100-fold, or more than 100-fold, higher than the number of HCV-specific CD8⁺ CTLs induced by administration of a control composition (e.g., a composition comprising the HCV E1/E2 heterodimer but lacking the T-cell epitope polypeptide; a composition comprising an E1 polypeptide but lacking the T-cell epitope polypeptide; a composition comprising an E2 polypeptide but lacking the T-cell epitope polypeptide).

In some cases, an immunogenic composition of the present disclosure, when administered to an individual in need thereof, induces CD4⁺ T cells specific for HCV, where the number of HCV-specific CD4⁺ T cells induced is at least 10%, at least 15%, at least 20%, at least 25%, at least 30%, at least 40%, at least 50%, at least 75%, at least 100% (or 2-fold), at least 2.5-fold, at least 5-fold, at least 7.5-fold, at least 10-fold, at least 20-fold, at least 50-fold, or at least 100-fold, or more than 100-fold, higher than the number of HCV-specific CD4⁺ T cells induced by administration of a control composition (e.g., a composition comprising the HCV E1/E2 heterodimer but lacking the T-cell epitope polypeptide; a composition comprising an E1 polypeptide but lacking the T-cell epitope polypeptide; a composition comprising an E2 polypeptide but lacking the T-cell epitope polypeptide).

In some cases, an immunogenic composition of the present disclosure, when administered to an individual in need thereof, induces production of HCV-specific CD4⁺ T cells and CD8⁺ T cells in the individual, where the number of HCV-specific CD4⁺ T cells and/or CD8⁺ T cells is increased, such that the percent of total peripheral CD4⁺ and/or CD8⁺ T cells that is HCV-specific is from 0.01% to 0.05%, from 0.05% to 0.10%, from 0.10% to 0.125%, from 0.125% to 0.25%, from 0.25% to from 0.50%, or 0.5% to 10% (e.g., from 0.5% to 1%, from 1% to 2%, from 2% to 5%, or from 5% to 10%). The number of HCV-specific CD4⁺ T cells and CD8⁺ T cells in a control individual (e.g., an individual not infected with HCV) not treated with the immunogenic composition would be undetectable.

In some cases, an immunogenic composition of the present disclosure, when administered to an individual in need thereof, induces production of HCV NS3-specific CD4⁺ T cells and/or CD8⁺ T cells in the individual, where the number of HCV NS3-specific CD4⁺ T cells and/or CD8⁺ T cells is increased, such that the percent of the total peripheral blood T cells (i.e., the total number of CD4⁺ T cells+CD8⁺ T cells in the peripheral blood) that are HCV NS3-specific CD4⁺ T cells and CD8⁺ T cells is from 0.01% to 10% (e.g., from 0.01% to 0.05%, from 0.05% to 0.1%, from 0.1% to 0.25%, from 0.25% to 0.5%, from 0.5% to 1%, from 1% to 2%, from 2% to 5%, or from 5% to 10%). The number of HCV NS3-specific CD4⁺ T cells and CD8⁺ T cells in a control individual (e.g., an individual not infected with HCV) not treated with the immunogenic composition would be undetectable.

In some cases, an immunogenic composition of the present disclosure, when administered to an individual in need thereof, induces production of HCV NS4-specific CD4⁺ T cells and/or CD8⁺ T cells in the individual, where the number of HCV NS4-specific CD4⁺ T cells and/or CD8⁺ T cells is increased, such that the percent of the total peripheral blood T cells (i.e., the total number of CD4⁺ T cells+CD8⁺ T cells in the peripheral blood) that are HCV NS4-specific CD4⁺ T cells and CD8⁺ T cells is from 0.01% to 10% (e.g., from 0.01% to 0.05%, from 0.05% to 0.1%, from 0.1% to 0.25%, from 0.25% to 0.5%, from 0.5% to 1%, from 1% to 2%, from 2% to 5%, or from 5% to 10%). The number of HCV NS4-specific CD4⁺ T cells and CD8⁺ T cells in a control individual (e.g., an individual not infected with HCV) not treated with the immunogenic composition would be undetectable.

In some cases, an immunogenic composition of the present disclosure, when administered to an individual in need thereof, induces production of HCV core-specific CD4⁺ T cells and/or CD8⁺ T cells in the individual, where the number of HCV core-specific CD4⁺ T cells and/or CD8⁺ T cells is increased, such that the percent of the total peripheral blood T cells (i.e., the total number of CD4⁺ T cells+CD8⁺ T cells in the peripheral blood) that are HCV core-specific CD4⁺ T cells and CD8⁺ T cells is from 0.01% to 10% (e.g., from 0.01% to 0.05%, from 0.05% to 0.1%, from 0.1% to 0.25%, from 0.25% to 0.5%, from 0.5% to 1%, from 1% to 2%, from 2% to 5%, or from 5% to 10%). The number of HCV core-specific CD4⁺ T cells and CD8⁺ T cells in a control individual (e.g., an individual not infected with HCV) not treated with the immunogenic composition would be undetectable.

In some cases, an immunogenic composition of the present disclosure, when administered to an individual in need thereof, increases the number of HCV E1/E2-specific CD4⁺ T cells and CD8⁺ T cells in the individual by at least 10%, at least 15%, at least 20%, at least 25%, at least 30%, at least 40%, at least 50%, at least 75%, at least 100% (or 2-fold), at least 2.5-fold, at least 5-fold, at least 7.5-fold, at least 10-fold, at least 20-fold, at least 50-fold, or at least 100-fold, or more than 100-fold, compared to the number of HCV E1/E2-specific CD4⁺ T cells and CD8⁺ T cells in the individual induced by administration of a control composition comprising the HCV E1/E2 heterodimer but lacking the T-cell epitope polypeptide, or compared to the number of HCV E1/E2-specific CD4⁺ T cells and CD8⁺ T cells in the individual before administration of the immunogenic composition.

In some cases, an immunogenic composition of the present disclosure, when administered to an individual in need thereof, induces helper T lymphocytes (e.g., CD4⁺ T cells) specific for HCV, where the number of HCV-specific helper T lymphocytes induced is at least 10%, at least 15%, at least 20%, at least 25%, at least 30%, at least 40%, at least 50%, at least 75%, at least 100% (or 2-fold), at least 2.5-fold, at least 5-fold, at least 7.5-fold, at least 10-fold, at least 20-fold, at least 50-fold, or at least 100-fold, or more than 100-fold, higher than the number of HCV-specific helper T cells induced by administration of a control composition comprising the HCV E1/E2 heterodimer but lacking the T-cell epitope polypeptide, or compared to the number of HCV-specific CD4⁺ T cells in the individual before administration of the immunogenic composition.

In some cases, an immunogenic composition of the present disclosure, when administered to an individual in need thereof, induces antibody specific for HCV, where the level of HCV-specific antibody induced is at least as high as the level of HCV-specific antibody induced by administration of a control composition comprising the HCV E1/E2 heterodimer but lacking the T-cell epitope polypeptide.

In some cases, an immunogenic composition of the present disclosure, when administered to an individual in need thereof, induces antibody specific for HCV, where the level of HCV-specific antibody induced is at least equivalent to the level of HCV-specific antibody induced by administration of a control composition comprising the HCV E1/E2 heterodimer but lacking the T-cell epitope polypeptide, or to the level of HCV-specific antibody in the individual before administration of the immunogenic composition. In some cases, an immunogenic composition of the present disclosure, when administered to an individual in need thereof, induces antibody specific for HCV, where the level of HCV-specific antibody induced is at least 10%, at least 15%, at least 20%, at least 25%, at least 30%, at least 40%, at least 50%, at least 75%, at least 100% (or 2-fold), at least 2.5-fold, at least 5-fold, at least 7.5-fold, at least 10-fold, at least 20-fold, at least 50-fold, or at least 100-fold, or more than 100-fold, higher than the level of HCV-specific antibody induced by administration of a control composition comprising the HCV E1/E2 heterodimer but lacking the T-cell epitope polypeptide, or compared the level of HCV-specific antibody in the individual before administration of the immunogenic composition.

An immunogenic composition of the present disclosure, when administered to an individual in need thereof, induces an immune response (e.g., a cellular immune response) in the individual to one or more HCV genotypes. In some cases, an immunogenic composition of the present disclosure, when administered to an individual in need thereof, induces an immune response in the individual to HCV genotype 1. In some cases, an immunogenic composition of the present disclosure, when administered to an individual in need thereof, induces an immune response in the individual to HCV genotype 2. In some cases, an immunogenic composition of the present disclosure, when administered to an individual in need thereof, induces an immune response in the individual to HCV genotype 3. In some cases, an immunogenic composition of the present disclosure, when administered to an individual in need thereof, induces an immune response in the individual to HCV genotype 1 and HCV genotype 3. In some cases, an immunogenic composition of the present disclosure, when administered to an individual in need thereof, induces an immune response in the individual to HCV genotype 1, HCV genotype 2, and HCV genotype 3. In some cases, an immunogenic composition of the present disclosure, when administered to an individual in need thereof, induces an immune response in the individual to HCV genotype 1, HCV genotype 2, HCV genotype 3, and HCV genotype 7. In some cases, an immunogenic composition of the present disclosure, when administered to an individual in need thereof, induces an immune response in the individual to HCV genotype 1, HCV genotype 2, HCV genotype 3, HCB genotype 4, HCV genotype 5, HCV genotype 6, and HCV genotype 7. In some cases, an immunogenic composition of the present disclosure, when administered to an individual in need thereof, induces an immune response in the individual to HCV genotype 1, HCV genotype 2, HCV genotype 3, HCV genotype 4, HCV genotype 5, and HCV genotype 6.

### T-Cell Epitope Polypeptides

A T-cell epitope polypeptide suitable for inclusion in a composition of the present disclosure exhibits one or more of the following features: 1) displays immunodominant epitopes; 2) displays epitopes that are conserved between or among two or more different HCV genotypes; 3) displayed through human leukocyte antigen (HLA) alleles that are common within the human population; 4) good yields by chemical synthesis; and 5) good solubility in a buffered aqueous solution.

In some cases, a T-cell epitope polypeptide suitable for inclusion in a composition of the present disclosure displays 1, 2, 3, 4, 5, or from 5 to 10, different epitopes displayed by an HCV polypeptide other than E1 and E2. In some cases, the epitopes are immunodominant epitopes.

In some cases, a T-cell epitope polypeptide suitable for inclusion in a composition of the present disclosure displays one or more epitopes that are conserved between or among two or more HCV genotypes. For example, in some cases, a T-cell epitope polypeptide suitable for inclusion in a composition of the present disclosure displays 1, 2, 3, 4, 5, or from 5 to 10, epitopes that are conserved between HCV genotype 1 and HCV genotype 3. As another example, in some cases, a T-cell epitope polypeptide suitable for inclusion in a composition of the present disclosure displays 1, 2, 3, 4, 5, or from 5 to 10, epitopes that are conserved between HCV genotype 1 and HCV genotype 2. As another example, in some cases, a T-cell epitope polypeptide suitable for inclusion in a composition of the present disclosure displays 1, 2, 3, 4, 5, or from 5 to 10, epitopes that are conserved among HCV genotypes 1, 2, and 3. As another example, in some cases, a T-cell epitope polypeptide suitable for inclusion in a composition of the present disclosure displays 1, 2, 3, 4, 5, or from 5 to 10, epitopes that are conserved among HCV genotypes 1, 2, 3, and 6.

In some cases, a T-cell epitope polypeptide suitable for inclusion in a composition of the present disclosure is presented through one or multiple HLA alleles common within the human population. Examples of such alleles are described in Example 1.

In some cases, a T-cell epitope polypeptide suitable for inclusion in a composition of the present disclosure can be chemically synthesized using standard methods (e.g., a solid-phase peptide synthesis (SPPS) method), where the yield is at least 50%, at least 55%, at least 60%, at least 70%, at least 80%, at least 85%, at least 90%, at least 95%, at least 96%, at least 97%, at least 98%, or more than 98%. In some cases, the yield is 95%. In some cases, the yield is 96%. In some cases, the yield is 97%.

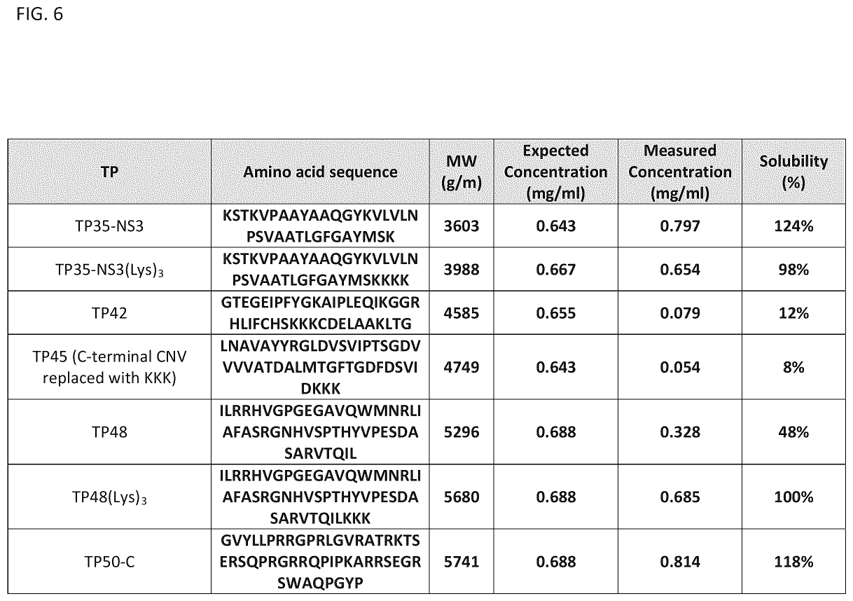

In some cases, a T-cell epitope polypeptide suitable for inclusion in a composition of the present disclosure exhibits solubility in a buffered aqueous solution. As one non-limiting example, a T-cell epitope polypeptide suitable for inclusion in a composition of the present disclosure exhibits solubility of at least 1 mg/ml in the following buffer (“E1E2” buffer): 10 mM sodium citrate, 250 mM NaCl, 1 mM ethylenediaminetetraacetic acid (EDTA), 0.1% non-ionic detergent Tween 80, pH 6.0. For example, the peptides TP35-NS3 and TP50C are soluble in E1E2 buffer at a concentration of at least 1 mg/ml. The peptides TP35-NS3 and TP50C are soluble in E1E2 buffer at a concentration of at least 1 mg/ml, without the need for adding dimethylsulfoxide (DMS). Solubility of various T-cell epitope polypeptides are shown in FIGS. 6 and 12-14. In some cases, a T-cell epitope polypeptide suitable for inclusion in a composition of the present disclosure is soluble in E1E2 buffer at a concentration of at least 25 μg/ml, at least 30 μg/ml, at least 35 g/ml, at least 50 μg/ml, at least 75 μg/ml, at least 100 μg/ml, at least 250 μg/ml, at least 500 g/ml, at least 750 μg/ml, or at least 1 mg/ml. In some cases, a T-cell epitope polypeptide suitable for inclusion in a composition of the present disclosure exhibits good solubility in E1E2 buffer following lyophilization of the T-cell epitope polypeptide; in other words, in some cases, the T-cell epitope polypeptide is lyophilized, then reconstituted in E1E2 buffer, where the T-cell epitope polypeptide exhibits good solubility in the E1E2 buffer following reconstitution.

In some cases, a T-cell epitope polypeptide suitable for inclusion in a composition of the present disclosure exhibits good solubility in water. In some cases, a T-cell epitope polypeptide suitable for inclusion in a composition of the present disclosure is soluble in water at a concentration of at least 25 μg/ml, at least 30 μg/ml, at least 35 μg/ml, at least 50 μg/ml, at least 75 μg/ml, at least 100 μg/ml, at least 250 μg/ml, at least 500 μg/ml, at least 750 μg/ml, or at least 1 mg/ml.

In some cases, T-cell epitopes present in a T-cell epitope polypeptide can be provided in two peptide fragments. For example, for TP35-NS3 or for TP50C, a mixture of two shorter peptides can be used. The peptide fragments can be partially overlapping (e.g., overlapping by from 2 to 10 amino acids), or non-overlapping. The peptide fragments can have a length of from 18 amino acids to 30 amino acids, e.g., can have a length of 18 amino acids (aa), 19 aa, 20 aa, 21 aa, 22 aa, 23 aa, 24 aa, 25 aa, 26 aa, 27 aa, 28 aa, 29 aa, or 30 aa.

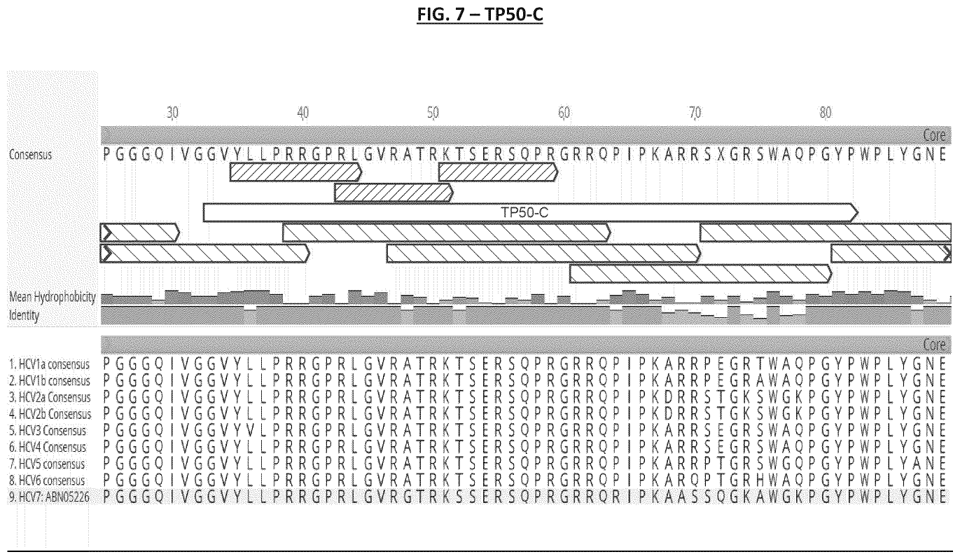

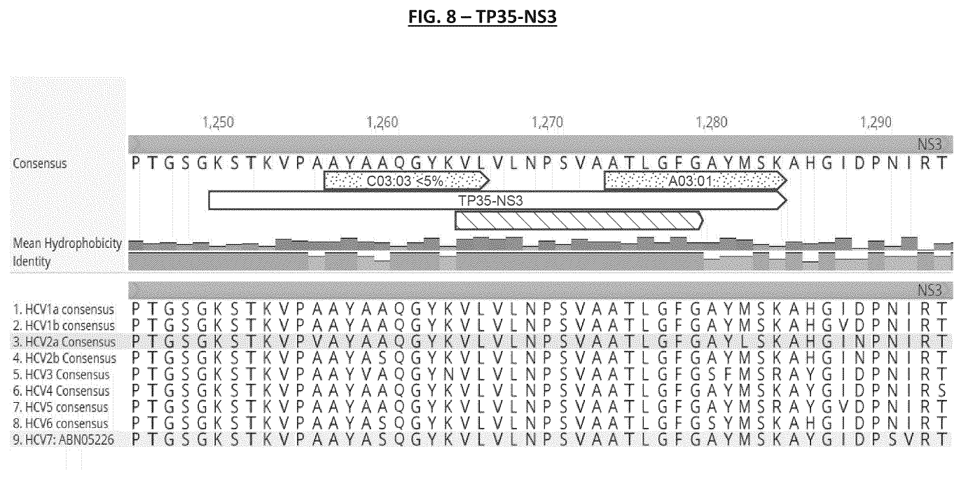

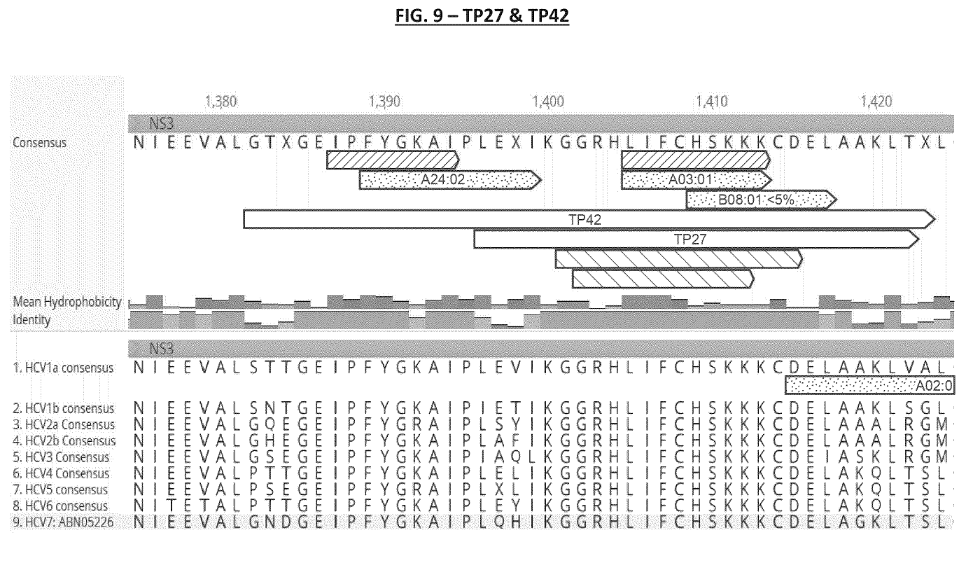

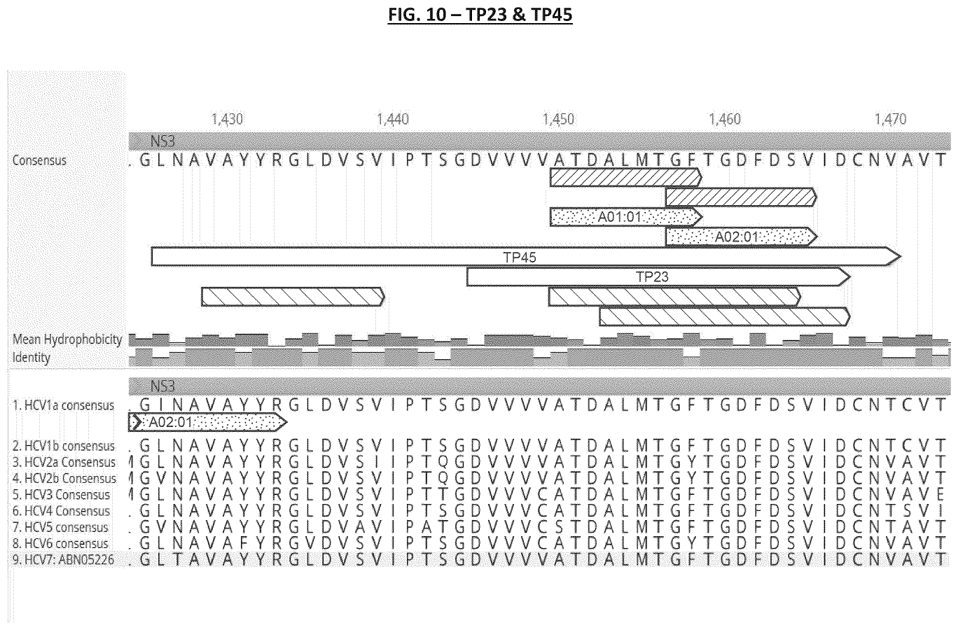

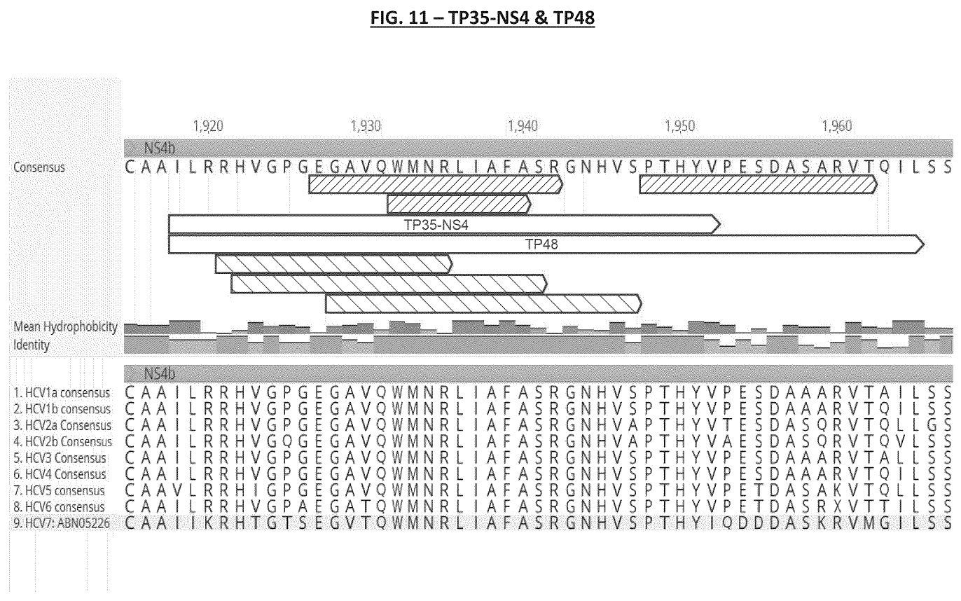

A T-cell epitope polypeptide suitable for inclusion in a composition of the present disclosure can include one or more conservative amino acid substitutions compared to, e.g., the amino acid designated “consensus” in one of FIGS. 7-11, e.g., an amino acid sequence labeled “consensus” and corresponding to TP35-NS3, TP50C, TP23, TP27, TP35-NS4, TP42, TP45, or TP48 (open bars in FIGS. 7-11). Examples of TP35-NS3, TP50C, TP23, TP27, TP35-NS4, TP42, TP45, and TP48 amino acid sequences with conservative amino acid substitutions are provided in FIGS. 7-11.

As described in detail below, a T-cell epitope polypeptide suitable for inclusion in a composition of the present disclosure can include one or more CD8 and/or CD4 epitopes. Examples of CD8 and CD4 epitopes are described below, and depicted in FIGS. 7-11. In FIGS. 7-11, bars with right-slanting hash marks denote conserved MHC class I CD8-specific HCV epitopes; bars with left-slanting hash marks denote conserved MHC class II CD4-specific HCV epitopes; stippled bars denote MHC class I CD8-specifc HCV epitopes with corresponding HLA specificity. Stippled bars with “<5%” indicate the least common HLA alleles (less than 5%) in the U.S. population; the remainder of the stippled bars indicate an HLA frequency of >5% in the U.S. population. The corresponding alleles shown are: HLA-A01:01; HLA-A02:01; HLA-A03:01; HLA-A24:02; HLA-B08:01; and HLA-C03:03.

Those skilled in the art are aware of “anchor” amino acid positions, i.e., that are more likely to be conserved in a particular CD8 epitope (binding to HLA Class I) for various HLA alleles. For example, in a 9-amino acid peptide comprising a CD8 epitope (and binding to HLA class I), residues 2 and 9 are likely to be “anchor” amino acids and are therefore likely conserved, for HLA alleles HLA-A01:01, HLA-A02:01, HLA-A03:01, and HLA-A24:02.

### TP35-NS3

In some cases, a T-cell epitope polypeptide suitable for inclusion in a composition of the present disclosure comprises an amino acid sequence having at least 20% (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100%) amino acid sequence identity to the following amino acid sequence: KSTKVPAAYAAQGYKVLVLNPSVAATLGFGAYMSK (SEQ ID NO:133; also referred to herein as “TP35-NS3”); where the T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the T-cell epitope polypeptide is part of a fusion polypeptide, as described in detail below.

In some cases, a TP35-NS3 T-cell epitope polypeptide suitable for inclusion in a composition of the present disclosure comprises the following amino acid sequence: KSTKVPX₁AYX₂X₃QGYX₄VLVLNPSVAATLGFGX₅X₆X₇SX₈ (SEQ ID NO:134), where X₁ is A or V; X₂ is A or V; X₃ is A or S; X₄ is K or N; X₅ is A or S; X₆ is Y or F; X₇ is M or L; and X₈ is K or R; where the TP35-NS3 T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the T-cell epitope polypeptide has a length of 35 amino acids. In some cases, the T-cell epitope polypeptide is part of a fusion polypeptide, as described in detail below.

In some cases, a TP35-NS3 T-cell epitope polypeptide suitable for inclusion in a composition of the present disclosure comprises the following amino acid sequence: KSTKVPVAYAAQGYKVLVLNPSVAATLGFGAYLSK (SEQ ID NO:135); where the TP35-NS3 T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the T-cell epitope polypeptide has a length of 35 amino acids. In some cases, the T-cell epitope polypeptide is part of a fusion polypeptide, as described in detail below.

In some cases, a TP35-NS3 T-cell epitope polypeptide suitable for inclusion in a composition of the present disclosure comprises the following amino acid sequence: KSTKVPAAYASQGYKVLVLNPSVAATLGFGAYMSK (SEQ ID NO:136); where the TP35-NS3 T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the T-cell epitope polypeptide has a length of 35 amino acids. In some cases, the T-cell epitope polypeptide is part of a fusion polypeptide, as described in detail below.

In some cases, a TP35-NS3 T-cell epitope polypeptide suitable for inclusion in a composition of the present disclosure comprises the following amino acid sequence: KSTKVPAAYVAQGYNVLVLNPSVAATLGFGSFMSR (SEQ ID NO:137); where the TP35-NS3 T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the T-cell epitope polypeptide has a length of 35 amino acids. In some cases, the T-cell epitope polypeptide is part of a fusion polypeptide, as described in detail below.

In some cases, a TP35-NS3 T-cell epitope polypeptide suitable for inclusion in a composition of the present disclosure comprises the following amino acid sequence: KSTKVPAAYAAQGYKVLVLNPSVAATLGFGAYMSR (SEQ ID NO:138); where the TP35-NS3 T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the T-cell epitope polypeptide has a length of 35 amino acids. In some cases, the T-cell epitope polypeptide is part of a fusion polypeptide, as described in detail below.

In some cases, a TP35-NS3 T-cell epitope polypeptide suitable for inclusion in a composition of the present disclosure comprises the following amino acid sequence: KSTKVPAAYASQGYKVLVLNPSVAATLGFGSYMSK (SEQ ID NO:139); where the TP35-NS3 T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the T-cell epitope polypeptide has a length of 35 amino acids. In some cases, the T-cell epitope polypeptide is part of a fusion polypeptide, as described in detail below.

In some cases, a TP35-NS3 T-cell epitope polypeptide suitable for inclusion in a composition of the present disclosure comprises the following amino acid sequence: KSTKVPAAYASQGYKVLVLNPSVAATLGFGAYMSK (SEQ ID NO:136); where the TP35-NS3 T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the T-cell epitope polypeptide has a length of 35 amino acids. In some cases, the T-cell epitope polypeptide is part of a fusion polypeptide, as described in detail below.

A TP35-NS3 T-cell epitope polypeptide suitable for inclusion in a composition of the present disclosure can include a CD8 epitope, e.g., a stretch of 10 contiguous amino acids having the amino acid sequence AYX₁X₂QGYX₃VL (SEQ ID NO:140), where X₁ is A or V; X₂ is A or S; and X₃ is K or N; and/or a stretch of 11 contiguous amino acids having the amino acid sequence ATLGFGX₁X₂X₃SX₄ (SEQ ID NO:141), where X₁ is A or S; X₂ is Y or F; X₃ is M or L; and X₄ is K or R. In some cases, the CD8 epitope is a stretch of 10 contiguous amino acids having the amino acid sequence AYAAQGYKVL (SEQ ID NO:142). In some cases, the CD8 epitope is a stretch of 10 contiguous amino acids having the amino acid sequence AYASQGYKVL (SEQ ID NO:187). In some cases, the CD8 epitope is a stretch of 10 contiguous amino acids having the amino acid sequence AYVAQGYNVL (SEQ ID NO:143). A TP35-NS3 T-cell epitope polypeptide suitable for inclusion in a composition of the present disclosure can include a CD8 epitope, e.g., a stretch of 10 contiguous amino acids having the amino acid sequence AYAAQGYKVL (SEQ ID NO:142) and/or a stretch of 11 contiguous amino acids having the amino acid sequence ATLGFGAYMSK (SEQ ID NO:144). A TP35-NS3 T-cell epitope polypeptide suitable for inclusion in a composition of the present disclosure can include: i) a stretch of 10 contiguous amino acids having the amino acid sequence AYAAQGYKVL (SEQ ID NO:142); and ii) a stretch of 11 contiguous amino acids having the amino acid sequence ATLGFGAYMSK (SEQ ID NO:144).

A TP35-NS3 T-cell epitope polypeptide suitable for inclusion in a composition of the present disclosure can include a CD4 epitope, e.g., a stretch of about 15 contiguous amino acids having the amino acid sequence: VLVLNPSVAATLGFG (SEQ ID NO:145).

A TP35-NS3 T-cell epitope polypeptide suitable for inclusion in a composition of the present disclosure can include: i) a CD4 epitope, e.g., a stretch of about 15 contiguous amino acids having the amino acid sequence: VLVLNPSVAATLGFG (SEQ ID NO:145); ii) a first CD8 epitope, e.g., a stretch of 10 contiguous amino acids having the amino acid sequence AYX₁X₂QGYX₃VL (SEQ ID NO:140), where X₁ is A or V; X₂ is A or S; and X₃ is K or N; and iii) a second CD8 epitope, e.g., a stretch of 11 contiguous amino acids having the amino acid sequence ATLGFGX₁X₂X₃SX₄ (SEQ ID NO:141), where X₁ is A or S; X₂ is Y or F; X₃ is M or L; and X₄ is K or R. A TP35-NS3 T-cell epitope polypeptide suitable for inclusion in a composition of the present disclosure can include: i) a CD4 epitope, e.g., a stretch of about 15 contiguous amino acids having the amino acid sequence: VLVLNPSVAATLGFG (SEQ ID NO:145); ii) a first CD8 epitope, e.g., a stretch of 10 contiguous amino acids having the amino acid sequence AYAAQGYKVL (SEQ ID NO:142); and iii) a second CD8 epitope, e.g., a stretch of 11 contiguous amino acids having the amino acid sequence ATLGFGAYMSK (SEQ ID NO:144).

In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: KSTKVPAAYAAQGYKVLVLNPSVAATLGFGAYMSK (SEQ ID NO:133); and has a length of from 35 amino acids to 40 amino acids (e.g., 35 amino acids (aa), 36 aa, 37 aa, 38 aa, 39 aa, or 40 aa).

In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: KSTKVPAAYAAQGYKVLVLNPSVAATLGFGAYMSK (SEQ ID NO:133); and has a length of 35 amino acids. In some cases, a T-cell epitope polypeptide suitable for inclusion in a composition of the present disclosure comprises the amino acid sequence KSTKVPAAYAAQGYKVLVLNPSVAATLGFGAYMSK (SEQ ID NO:133); and has a length of 35 amino acids.

In some cases, a suitable T-cell epitope polypeptide is a TP35-NS3 polypeptide with from 1 amino acid to 5 amino acids removed from the N-terminus and/or from 1 amino acid to 5 amino acids removed from the C-terminus. Thus, e.g., a suitable T-cell epitope polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: PAAYAAQGYKVLVLNPSVAATLGFGAYMSK (SEQ ID NO:146); and has a length of 30 amino acids. As another example, a suitable T-cell epitope polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: KSTKVPAAYAAQGYKVLVLNPSVAATLGFG (SEQ ID NO:147); and has a length of 30 amino acids.

### TP50-C

In some cases, a T-cell epitope polypeptide suitable for inclusion in a composition of the present disclosure comprises an amino acid sequence having at least 20% (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100%) amino acid sequence identity to the following amino acid sequence: GVYLLPRRGPRLGVRATRKTSERSQPRGRRQPIPKARRSEGRSWAQPGYP (SEQ ID NO:148); also referred to herein as “TP50C”); where the T-cell epitope polypeptide has a length of from 40 amino acids to 50 amino acids (e.g., T-cell epitope polypeptide has a length of 40 amino acids (aa), 41 aa, 42 aa, 43 aa, 44 aa, 45 aa, 46 aa, 47 aa, 48 aa, 49 aa, or 50 aa). In some cases, the T-cell epitope polypeptide is part of a fusion polypeptide, as described in detail below.

In some cases, a TP50-C T-cell epitope polypeptide suitable for inclusion in a composition of the present disclosure comprises the following amino acid sequence: GVYX₁LPRRGPRLGVRX₂TRKX₃SERSQPRGRRQX₄IPKX₅X₆X₇X₈X₉GX₁₀X₁₁WX₁₂X₁₃PGY P (SEQ ID NO:149), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁₁ is S, T, H, or A; X₁₂ is A or G; and X₁₃ is Q or K; where the T-cell epitope polypeptide has a length of from 40 amino acids to 50 amino acids (e.g., the T-cell epitope polypeptide has a length of 40 amino acids (aa), 41 aa, 42 aa, 43 aa, 44 aa, 45 aa, 46 aa, 47 aa, 48 aa, 49 aa, or 50 aa). In some cases, the T-cell epitope polypeptide has a length of 50 amino acids. In some cases, the T-cell epitope polypeptide is part of a fusion polypeptide, as described in detail below.

In some cases, a TP50-C T-cell epitope polypeptide suitable for inclusion in a composition of the present disclosure comprises the following amino acid sequence: GVYX₁LPRRGPRLGVRX₂TRKX₃SERSQPRGRRQX₄IPKX₅X₆X₇X₈X₉GX₁₀X₁₁WX₁₂X₁₃PGY P (SEQ ID NO:149), where X₁ is L or V; X₂ is A; X₃ is T; X₄ is P; X₅ is A or D; X₆ is R; X₇ is R or Q; X₈ is S or P; X₉ is E or T; X₁₀ is R or K; X₁₁ is S, T, H, or A; X₁₂ is A or G; and X₁₃ is Q or K; where the T-cell epitope polypeptide has a length of from 40 amino acids to 50 amino acids (e.g., the T-cell epitope polypeptide has a length of 40 amino acids (aa), 41 aa, 42 aa, 43 aa, 44 aa, 45 aa, 46 aa, 47 aa, 48 aa, 49 aa, or 50 aa). In some cases, the T-cell epitope polypeptide has a length of 50 amino acids. In some cases, the T-cell epitope polypeptide is part of a fusion polypeptide, as described in detail below.

In some cases, a TP50-C T-cell epitope polypeptide suitable for inclusion in a composition of the present disclosure comprises the following amino acid sequence: GVYLLPRRGPRLGVRATRKTSERSQPRGRRQPIPKARRPEGRTWAQPGYP (SEQ ID NO:150); where the T-cell epitope polypeptide has a length of from 40 amino acids to 50 amino acids (e.g., the T-cell epitope polypeptide has a length of 40 amino acids (aa), 41 aa, 42 aa, 43 aa, 44 aa, 45 aa, 46 aa, 47 aa, 48 aa, 49 aa, or 50 aa). In some cases, the T-cell epitope polypeptide has a length of 50 amino acids. In some cases, the T-cell epitope polypeptide is part of a fusion polypeptide, as described in detail below.

In some cases, a TP50-C T-cell epitope polypeptide suitable for inclusion in a composition of the present disclosure comprises the following amino acid sequence: GVYLLPRRGPRLGVRATRKTSERSQPRGRRQPIPKARRPEGRAWAQPGYP (SEQ ID NO:151); where the T-cell epitope polypeptide has a length of from 40 amino acids to 50 amino acids (e.g., the T-cell epitope polypeptide has a length of 40 amino acids (aa), 41 aa, 42 aa, 43 aa, 44 aa, 45 aa, 46 aa, 47 aa, 48 aa, 49 aa, or 50 aa). In some cases, the T-cell epitope polypeptide has a length of 50 amino acids. In some cases, the T-cell epitope polypeptide is part of a fusion polypeptide, as described in detail below.

In some cases, a TP50-C T-cell epitope polypeptide suitable for inclusion in a composition of the present disclosure comprises the following amino acid sequence: GVYLLPRRGPRLGVRATRKTSERSQPRGRRQPIPKDRRSTGKSWGKPGYP (SEQ ID NO:152); where the T-cell epitope polypeptide has a length of from 40 amino acids to 50 amino acids (e.g., the T-cell epitope polypeptide has a length of 40 amino acids (aa), 41 aa, 42 aa, 43 aa, 44 aa, 45 aa, 46 aa, 47 aa, 48 aa, 49 aa, or 50 aa). In some cases, the T-cell epitope polypeptide has a length of 50 amino acids. In some cases, the T-cell epitope polypeptide is part of a fusion polypeptide, as described in detail below.

In some cases, a TP50-C T-cell epitope polypeptide suitable for inclusion in a composition of the present disclosure comprises the following amino acid sequence: GVYLLPRRGPRLGVRATRKTSERSQPRGRRQPIPKDRRSTGKSWGKPGYP (SEQ ID NO:152); where the T-cell epitope polypeptide has a length of from 40 amino acids to 50 amino acids (e.g., the T-cell epitope polypeptide has a length of 40 amino acids (aa), 41 aa, 42 aa, 43 aa, 44 aa, 45 aa, 46 aa, 47 aa, 48 aa, 49 aa, or 50 aa). In some cases, the T-cell epitope polypeptide has a length of 50 amino acids. In some cases, the T-cell epitope polypeptide is part of a fusion polypeptide, as described in detail below.

In some cases, a TP50-C T-cell epitope polypeptide suitable for inclusion in a composition of the present disclosure comprises the following amino acid sequence: GVYVLPRRGPRLGVRATRKTSERSQPRGRRQPIPKARRSEGRSWAQPGYP (SEQ ID NO:153); where the T-cell epitope polypeptide has a length of from 40 amino acids to 50 amino acids (e.g., the T-cell epitope polypeptide has a length of 40 amino acids (aa), 41 aa, 42 aa, 43 aa, 44 aa, 45 aa, 46 aa, 47 aa, 48 aa, 49 aa, or 50 aa). In some cases, the T-cell epitope polypeptide has a length of 50 amino acids. In some cases, the T-cell epitope polypeptide is part of a fusion polypeptide, as described in detail below.

In some cases, a TP50-C T-cell epitope polypeptide suitable for inclusion in a composition of the present disclosure comprises the following amino acid sequence: GVYLLPRRGPRLGVRATRKTSERSQPRGRRQPIPKARRPTGRSWGQPGYP (SEQ ID NO:154); where the T-cell epitope polypeptide has a length of from 40 amino acids to 50 amino acids (e.g., the T-cell epitope polypeptide has a length of 40 amino acids (aa), 41 aa, 42 aa, 43 aa, 44 aa, 45 aa, 46 aa, 47 aa, 48 aa, 49 aa, or 50 aa). In some cases, the T-cell epitope polypeptide has a length of 50 amino acids. In some cases, the T-cell epitope polypeptide is part of a fusion polypeptide, as described in detail below.

In some cases, a TP50-C T-cell epitope polypeptide suitable for inclusion in a composition of the present disclosure comprises the following amino acid sequence: GVYLLPRRGPRLGVRATRKTSERSQPRGRRQPIPKARQPTGRHWAQPGYP (SEQ ID NO:155); where the T-cell epitope polypeptide has a length of from 40 amino acids to 50 amino acids (e.g., the T-cell epitope polypeptide has a length of 40 amino acids (aa), 41 aa, 42 aa, 43 aa, 44 aa, 45 aa, 46 aa, 47 aa, 48 aa, 49 aa, or 50 aa). In some cases, the T-cell epitope polypeptide has a length of 50 amino acids. In some cases, the T-cell epitope polypeptide is part of a fusion polypeptide, as described in detail below.

In some cases, a TP50-C T-cell epitope polypeptide suitable for inclusion in a composition of the present disclosure comprises the following amino acid sequence: GVYLLPRRGPRLGVRTTRKSSERSQPRGRRQRIPKAASSQGKAWGKPGYP (SEQ ID NO:156); where the T-cell epitope polypeptide has a length of from 40 amino acids to 50 amino acids (e.g., the T-cell epitope polypeptide has a length of 40 amino acids (aa), 41 aa, 42 aa, 43 aa, 44 aa, 45 aa, 46 aa, 47 aa, 48 aa, 49 aa, or 50 aa). In some cases, the T-cell epitope polypeptide has a length of 50 amino acids. In some cases, the T-cell epitope polypeptide is part of a fusion polypeptide, as described in detail below.

A TP50-C T-cell epitope polypeptide suitable for inclusion in a composition of the present disclosure can include one or more CD4 epitopes, e.g., one or more of: i) a stretch of about 25 contiguous amino acids having the amino acid sequence: RRGPRLGVRATRKTSERSQPRGRRQ (SEQ ID NO:157) or RRGPRLGVRGTRKSSERSQPRGRRQ (SEQ ID NO:158); ii) a stretch of about 24 contiguous amino acids having the amino acid sequence: RATRKTSERSQPRGRRQPIPKARR (SEQ ID NO:159), or RGTRKSSERSQPRGRRQRIPKAAQ (SEQ ID NO:160), or RATRKTSERSQPRGRRQPIPKARQ (SEQ ID NO:161), or RATRKTSERSQPRGRRQPIPKDRR (SEQ ID NO:162); or iii) a stretch of about 20 contiguous amino acids having the amino acid sequence: RRQPIPKARRSEGRSWAQPG (SEQ ID NO:163), or RRQPIPKARPSEGRTWAQPG (SEQ ID NO:164), or RRQPIPKARRPEGRAWAQPG (SEQ ID NO:165), or RRQPIPKDRRSTGKSWGKPG (SEQ ID NO:166), or RRQPIPKDRRSTGKSWGKPG (SEQ ID NO:167), or RRQPIPKARRPTGRSWGQPG (SEQ ID NO:168), or RRQPIPKARQPTGRHWAQPG (SEQ ID NO:169), or RRQRIPKAASSQGKAWGKPG (SEQ ID NO:170).

In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: GVYLLPRRGPRLGVRATRKTSERSQPRGRRQPIPKARRSEGRSWAQPGYP (SEQ ID NO:148); and has a length of from 50 amino acids to about 60 amino acids.

In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: GVYLLPRRGPRLGVRATRKTSERSQPRGRRQPIPKARRSEGRSWAQPGYP (SEQ ID NO:148); and has a length of 50 amino acids. In some cases, a T-cell epitope polypeptide suitable for inclusion in a composition of the present disclosure comprises the amino acid sequence GVYLLPRRGPRLGVRATRKTSERSQPRGRRQPIPKARRSEGRSWAQPGYP (SEQ ID NO:148); and has a length of 50 amino acids.

### TP23

In some cases, a T-cell epitope polypeptide suitable for inclusion in a composition of the present disclosure comprises an amino acid sequence having at least 20% (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100%) amino acid sequence identity to the following amino acid sequence: DVVVVATDALMTGFTGDFDSVID (SEQ ID NO:186; also referred to herein as “TP23”); where the T-cell epitope polypeptide has a length of from 18 amino acids to 23 amino acids (e.g., T-cell epitope polypeptide has a length of 18 amino acids (aa), 19 aa, 20 aa, 21 aa, 22 aa, or 23 aa). In some cases, the TP23 T-cell epitope polypeptide has a length of 23 amino acids. In some cases, the T-cell epitope polypeptide is part of a fusion polypeptide, as described in detail below.

In some cases, a TP23 T-cell epitope polypeptide suitable for inclusion in a composition of the present disclosure comprises the following amino acid sequence: DVVVX₁X₂TDALMTGX₃TGDFDSVID (SEQ ID NO:171), where X₁ is V or C; X₂ is A or S; and X₃ is F or Y; where the TP23 T-cell epitope polypeptide has a length of from 18 amino acids to 23 amino acids (e.g., T-cell epitope polypeptide has a length of 18 amino acids (aa), 19 aa, 20 aa, 21 aa, 22 aa, or 23 aa). In some cases, the TP23 T-cell epitope polypeptide has a length of 23 amino acids. In some cases, the TP23 T-cell epitope polypeptide is part of a fusion polypeptide, as described in detail below.

In some cases, a TP23 T-cell epitope polypeptide suitable for inclusion in a composition of the present disclosure comprises the following amino acid sequence: DVVVVATDALMTGYTGDFDSVID (SEQ ID NO:172); where the TP23 T-cell epitope polypeptide has a length of from 18 amino acids to 23 amino acids (e.g., T-cell epitope polypeptide has a length of 18 amino acids (aa), 19 aa, 20 aa, 21 aa, 22 aa, or 23 aa). In some cases, the TP23 T-cell epitope polypeptide has a length of 23 amino acids. In some cases, the TP23 T-cell epitope polypeptide is part of a fusion polypeptide, as described in detail below.

In some cases, a TP23 T-cell epitope polypeptide suitable for inclusion in a composition of the present disclosure comprises the following amino acid sequence: DVVVCATDALMTGFTGDFDSVID (SEQ ID NO:173); where the TP23 T-cell epitope polypeptide has a length of from 18 amino acids to 23 amino acids (e.g., T-cell epitope polypeptide has a length of 18 amino acids (aa), 19 aa, 20 aa, 21 aa, 22 aa, or 23 aa). In some cases, the TP23 T-cell epitope polypeptide has a length of 23 amino acids. In some cases, the TP23 T-cell epitope polypeptide is part of a fusion polypeptide, as described in detail below.

In some cases, a TP23 T-cell epitope polypeptide suitable for inclusion in a composition of the present disclosure comprises the following amino acid sequence: DVVVCSTDALMTGFTGDFDSVID (SEQ ID NO:174); where the TP23 T-cell epitope polypeptide has a length of from 18 amino acids to 23 amino acids (e.g., T-cell epitope polypeptide has a length of 18 amino acids (aa), 19 aa, 20 aa, 21 aa, 22 aa, or 23 aa). In some cases, the TP23 T-cell epitope polypeptide has a length of 23 amino acids. In some cases, the TP23 T-cell epitope polypeptide is part of a fusion polypeptide, as described in detail below.

In some cases, a TP23 T-cell epitope polypeptide suitable for inclusion in a composition of the present disclosure comprises the following amino acid sequence: DVVVCATDALMTGYTGDFDSVID (SEQ ID NO:175); where the TP23 T-cell epitope polypeptide has a length of from 18 amino acids to 23 amino acids (e.g., T-cell epitope polypeptide has a length of 18 amino acids (aa), 19 aa, 20 aa, 21 aa, 22 aa, or 23 aa). In some cases, the TP23 T-cell epitope polypeptide has a length of 23 amino acids. In some cases, the TP23 T-cell epitope polypeptide is part of a fusion polypeptide, as described in detail below.

A TP23 T-cell epitope polypeptide suitable for inclusion in a composition of the present disclosure can include one or more CD8 epitopes, e.g., one or both of: i) a stretch of about 9 contiguous amino acids having the amino acid sequence: ATDALMTGF (SEQ ID NO:176), or STDALMTGF (SEQ ID NO:177), or ATDALMTGY (SEQ ID NO:178); or ii) a stretch of about 9 contiguous amino acids having the amino acid sequence GFTGDFDSV (SEQ ID NO:179), or GYTGDFDSV (SEQ ID NO:180).

A TP23 T-cell epitope polypeptide suitable for inclusion in a composition of the present disclosure can include one or more CD4 epitopes, e.g., a stretch of about 15 contiguous amino acids having the amino acid sequence: ATDALMTGFTGDFDS (SEQ ID NO:181), or STDALMTGFTGDFDS (SEQ ID NO:182), or ATDALMTGYTGDFDS (SEQ ID NO:183), or ALMTGFTGDFDSVID (SEQ ID NO:184), or ALMTGYTGDFDSVID (SEQ ID NO:185).

In some cases, a TP23 T-cell epitope polypeptide suitable for inclusion in a composition of the present disclosure includes: a) one or more CD8 epitopes, e.g., one or both of: i) a stretch of about 9 contiguous amino acids having the amino acid sequence: ATDALMTGF (SEQ ID NO:176), or STDALMTGF (SEQ ID NO:177), or ATDALMTGY (SEQ ID NO:178); or ii) a stretch of about 9 contiguous amino acids having the amino acid sequence GFTGDFDSV (SEQ ID NO:179), or GYTGDFDSV (SEQ ID NO:180); and b) one or more CD4 epitopes, e.g., a stretch of about 15 contiguous amino acids having the amino acid sequence: ATDALMTGFTGDFDS (SEQ ID NO:181), or STDALMTGFTGDFDS (SEQ ID NO:182), or ATDALMTGYTGDFDS (SEQ ID NO:183), or ALMTGFTGDFDSVID (SEQ ID NO:184), or ALMTGYTGDFDSVID (SEQ ID NO:185).

In some cases, an immunogenic composition of the present disclosure includes only a T-cell epitope polypeptide comprising an amino acid sequence having at least 20% (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100%) amino acid sequence identity to the following amino acid sequence: DVVVVATDALMTGFTGDFDSVID (SEQ ID NO:186; “TP23”); where the T-cell epitope polypeptide has a length of from 23 amino acids to 30 amino acids (e.g., T-cell epitope polypeptide has a length of 23 amino acids (aa), 24 aa, 25 aa, 26 aa, 27 aa, 28 aa, 29 aa, or 30 aa).

In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: DVVVVATDALMTGFTGDFDSVID (SEQ ID NO:186); and has a length of 23 amino acids. In some cases, a T-cell epitope polypeptide suitable for inclusion in a composition of the present disclosure comprises the amino acid sequence DVVVVATDALMTGFTGDFDSVID (SEQ ID NO:186); and has a length of 23 amino acids.

### TP27

In some cases, a T-cell epitope polypeptide suitable for inclusion in a composition of the present disclosure comprises an amino acid sequence having at least 20% (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100%) amino acid sequence identity to the following amino acid sequence: LEQIKGGRHLIFCHSKKKCDELAAKLT (SEQ ID NO:188; also referred to herein as “TP27”); where the T-cell epitope polypeptide has a length of from 22 amino acids to 27 amino acids (e.g., T-cell epitope polypeptide has a length of 22 amino acids (aa), 23 aa, 24 aa, 25 aa, 26 aa, or 27 aa). In some cases, the T-cell epitope polypeptide is part of a fusion polypeptide, as described in detail below.

In some cases, a TP27 T-cell epitope polypeptide suitable for inclusion in a composition of the present disclosure comprises the following amino acid sequence: X₁X₂X₃X₄KGGRHLIFCHSKKKCDEX₅AX₆X₇LX₈(SEQ ID NO:189), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S; where the T-cell epitope polypeptide has a length of from 22 amino acids to 27 amino acids (e.g., T-cell epitope polypeptide has a length of 22 amino acids (aa), 23 aa, 24 aa, 25 aa, 26 aa, or 27 aa). In some cases, the T-cell epitope polypeptide has a length of 27 amino acids. In some cases, the T-cell epitope polypeptide is part of a fusion polypeptide, as described in detail below.

In some cases, a TP27 T-cell epitope polypeptide suitable for inclusion in a composition of the present disclosure comprises the following amino acid sequence: LEVIKGGRHLIFCHSKKKCDELAAKLV (SEQ ID NO:190); where the T-cell epitope polypeptide has a length of from 22 amino acids to 27 amino acids (e.g., T-cell epitope polypeptide has a length of 22 amino acids (aa), 23 aa, 24 aa, 25 aa, 26 aa, or 27 aa). In some cases, the T-cell epitope polypeptide has a length of 27 amino acids. In some cases, the T-cell epitope polypeptide is part of a fusion polypeptide, as described in detail below.

In some cases, a TP27 T-cell epitope polypeptide suitable for inclusion in a composition of the present disclosure comprises the following amino acid sequence: IETIKGGRHLIFCHSKKKCDELAAKLS (SEQ ID NO:191); where the T-cell epitope polypeptide has a length of from 22 amino acids to 27 amino acids (e.g., T-cell epitope polypeptide has a length of 22 amino acids (aa), 23 aa, 24 aa, 25 aa, 26 aa, or 27 aa). In some cases, the T-cell epitope polypeptide has a length of 27 amino acids. In some cases, the T-cell epitope polypeptide is part of a fusion polypeptide, as described in detail below.

In some cases, a TP27 T-cell epitope polypeptide suitable for inclusion in a composition of the present disclosure comprises the following amino acid sequence: LSYIKGGRHLIFCHSKKKCDELAAALR (SEQ ID NO:192); where the T-cell epitope polypeptide has a length of from 22 amino acids to 27 amino acids (e.g., T-cell epitope polypeptide has a length of 22 amino acids (aa), 23 aa, 24 aa, 25 aa, 26 aa, or 27 aa). In some cases, the T-cell epitope polypeptide has a length of 27 amino acids. In some cases, the T-cell epitope polypeptide is part of a fusion polypeptide, as described in detail below.

In some cases, a TP27 T-cell epitope polypeptide suitable for inclusion in a composition of the present disclosure comprises the following amino acid sequence: LAFIKGGRHLIFCHSKKKCDELAALR (SEQ ID NO:193); where the T-cell epitope polypeptide has a length of from 22 amino acids to 27 amino acids (e.g., T-cell epitope polypeptide has a length of 22 amino acids (aa), 23 aa, 24 aa, 25 aa, 26 aa, or 27 aa). In some cases, the T-cell epitope polypeptide has a length of 27 amino acids. In some cases, the T-cell epitope polypeptide is part of a fusion polypeptide, as described in detail below.

In some cases, a TP27 T-cell epitope polypeptide suitable for inclusion in a composition of the present disclosure comprises the following amino acid sequence: IAQLKGGRHLIFCHSKKKCDEIASKLR (SEQ ID NO:195); where the T-cell epitope polypeptide has a length of from 22 amino acids to 27 amino acids (e.g., T-cell epitope polypeptide has a length of 22 amino acids (aa), 23 aa, 24 aa, 25 aa, 26 aa, or 27 aa). In some cases, the T-cell epitope polypeptide has a length of 27 amino acids. In some cases, the T-cell epitope polypeptide is part of a fusion polypeptide, as described in detail below.

In some cases, a TP27 T-cell epitope polypeptide suitable for inclusion in a composition of the present disclosure comprises the following amino acid sequence: LELIKGGRHLIFCHSKKKCDELAQLT (SEQ ID NO:196); where the T-cell epitope polypeptide has a length of from 22 amino acids to 27 amino acids (e.g., T-cell epitope polypeptide has a length of 22 amino acids (aa), 23 aa, 24 aa, 25 aa, 26 aa, or 27 aa). In some cases, the T-cell epitope polypeptide has a length of 27 amino acids. In some cases, the T-cell epitope polypeptide is part of a fusion polypeptide, as described in detail below.

In some cases, a TP27 T-cell epitope polypeptide suitable for inclusion in a composition of the present disclosure comprises the following amino acid sequence: LXLIKGGRHLIFCHSKKKCDELAKQLT (SEQ ID NO:197); where the T-cell epitope polypeptide has a length of from 22 amino acids to 27 amino acids (e.g., T-cell epitope polypeptide has a length of 22 amino acids (aa), 23 aa, 24 aa, 25 aa, 26 aa, or 27 aa). In some cases, the T-cell epitope polypeptide has a length of 27 amino acids. In some cases, the T-cell epitope polypeptide is part of a fusion polypeptide, as described in detail below.

In some cases, a TP27 T-cell epitope polypeptide suitable for inclusion in a composition of the present disclosure comprises the following amino acid sequence: LEYIKGGRHLIFCHSKKKCDELAKQLT (SEQ ID NO:198); where the T-cell epitope polypeptide has a length of from 22 amino acids to 27 amino acids (e.g., T-cell epitope polypeptide has a length of 22 amino acids (aa), 23 aa, 24 aa, 25 aa, 26 aa, or 27 aa). In some cases, the T-cell epitope polypeptide has a length of 27 amino acids. In some cases, the T-cell epitope polypeptide is part of a fusion polypeptide, as described in detail below.

In some cases, a TP27 T-cell epitope polypeptide suitable for inclusion in a composition of the present disclosure comprises the following amino acid sequence: LQHIKGGRHLIFCHSKKKCDELAGKLT (SEQ ID NO:199); where the T-cell epitope polypeptide has a length of from 22 amino acids to 27 amino acids (e.g., T-cell epitope polypeptide has a length of 22 amino acids (aa), 23 aa, 24 aa, 25 aa, 26 aa, or 27 aa). In some cases, the T-cell epitope polypeptide has a length of 27 amino acids. In some cases, the T-cell epitope polypeptide is part of a fusion polypeptide, as described in detail below.

A TP27 T-cell epitope polypeptide suitable for inclusion in a composition of the present disclosure can include one or more CD8 epitopes, e.g., one or both of: i) a stretch of about 9 contiguous amino acids having the amino acid sequence: LIFCHSKKK (SEQ ID NO:200); and ii) a stretch of about 9 contiguous amino acids having the amino acid sequence: HSKKKCDEL (SEQ ID NO:201). In some cases, a TP27 T-cell epitope polypeptide suitable for inclusion in a composition of the present disclosure includes the following CD8 epitopes: i) a stretch of about 9 contiguous amino acids having the amino acid sequence: LIFCHSKKK (SEQ ID NO:200); and ii) a stretch of about 9 contiguous amino acids having the amino acid sequence: HSKKKCDEL (SEQ ID NO:201).

A TP27 T-cell epitope polypeptide suitable for inclusion in a composition of the present disclosure can include one or more CD4 epitopes, e.g., a stretch of about 15 contiguous amino acids having the amino acid sequence: KGGRHLIFCHSKKKCD (SEQ ID NO:202), or a stretch of about 11 contiguous amino acids having the amino acid sequence: GRHLIFCHSKK (SEQ ID NO:244).

A TP27 T-cell epitope polypeptide suitable for inclusion in a composition of the present disclosure can include: a) the following CD8 epitopes: i) a stretch of about 9 contiguous amino acids having the amino acid sequence: LIFCHSKKK (SEQ ID NO:200); and ii) a stretch of about 9 contiguous amino acids having the amino acid sequence: HSKKKCDEL (SEQ ID NO:201); and b) the following CD4 epitope: a stretch of about 11 contiguous amino acids having the amino acid sequence: GRHLIFCHSKK (SEQ ID NO:244). A TP27 T-cell epitope polypeptide suitable for inclusion in a composition of the present disclosure can include: a) the following CD8 epitopes: i) a stretch of about 9 contiguous amino acids having the amino acid sequence: LIFCHSKKK (SEQ ID NO:200); and ii) a stretch of about 9 contiguous amino acids having the amino acid sequence: HSKKKCDEL (SEQ ID NO:201); and b) the following CD4 epitope: a stretch of about 15 contiguous amino acids having the amino acid sequence: KGGRHLIFCHSKKKCD (SEQ ID NO:202).

In some cases, an immunogenic composition of the present disclosure includes only a T-cell epitope polypeptide comprising an amino acid sequence having at least 20% (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100%) amino acid sequence identity to the following amino acid sequence: LEQIKGGRHLIFCHSKKKCDELAAKLT (SEQ ID NO:188; “TP27”); where the T-cell epitope polypeptide has a length of from 27 amino acids to 34 amino acids (e.g., T-cell epitope polypeptide has a length of 27 amino acids (aa), 28 aa, 29 aa, 30 aa, 31 aa, 32 aa, 33 aa, or 34 aa).

In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: LEQIKGGRHLIFCHSKKKCDELAAKLT (SEQ ID NO:188); and has a length of 27 amino acids. In some cases, a T-cell epitope polypeptide suitable for inclusion in a composition of the present disclosure comprises the amino acid sequence LEQIKGGRHLIFCHSKKKCDELAAKLT (SEQ ID NO:188); and has a length of 27 amino acids.

### TP35-NS4

In some cases, a T-cell epitope polypeptide suitable for inclusion in a composition of the present disclosure comprises an amino acid sequence having at least 20% (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100%) amino acid sequence identity to the following amino acid sequence: ILRRHVGPGEGAVQWMNRLIAFASRGNHVSPTHYV (SEQ ID NO:203; also referred to herein as “TP35-NS4”); where the T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the T-cell epitope polypeptide is part of a fusion polypeptide, as described in detail below.

In some cases, a TP35-NS4 T-cell epitope polypeptide suitable for inclusion in a composition of the present disclosure comprises the following amino acid sequence: X₁X₂X₃RHX₄GX₅X₆EGX₇X₈QWMNRLIAFASRGNHVX₉PTHYX₁₀ (SEQ ID NO:204), where X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; and X₁₀ is V or I; where the TP35-NS4 T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the TP35-NS4 T-cell epitope polypeptide has a length of 35 amino acids. In some cases, the T-cell epitope polypeptide is part of a fusion polypeptide, as described in detail below.

In some cases, a TP35-NS4 T-cell epitope polypeptide suitable for inclusion in a composition of the present disclosure comprises the following amino acid sequence: X₁X₂X₃RHX₄GX₅X₆EGX₇X₈QWMNRLIAFASRGNHVX₉PTHYX₁₀ (SEQ ID NO:204), where X₁ is I or V; X₂ is L; X₃ is R; X₄ is V or I; X₅ is P or Q; X₆ is G or A; X₇ is A; X₈ is V or T; X₉ is S or A; and X₁₀ is V; where the TP35-NS4 T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the TP35-NS4 T-cell epitope polypeptide has a length of 35 amino acids. In some cases, the T-cell epitope polypeptide is part of a fusion polypeptide, as described in detail below.

In some cases, a TP35-NS4 T-cell epitope polypeptide suitable for inclusion in a composition of the present disclosure comprises the following amino acid sequence: ILRRHVGPGEGAVQWMNRLIAFASRGNHVAPTHYV (SEQ ID NO:205); where the TP35-NS4 T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the TP35-NS4 T-cell epitope polypeptide has a length of 35 amino acids. In some cases, the T-cell epitope polypeptide is part of a fusion polypeptide, as described in detail below.

In some cases, a TP35-NS4 T-cell epitope polypeptide suitable for inclusion in a composition of the present disclosure comprises the following amino acid sequence: ILRRHVGQGEGAVQWMNRLIAFASRGNHVAPTHYV (SEQ ID NO:206); where the TP35-NS4 T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the TP35-NS4 T-cell epitope polypeptide has a length of 35 amino acids. In some cases, the T-cell epitope polypeptide is part of a fusion polypeptide, as described in detail below.

In some cases, a TP35-NS4 T-cell epitope polypeptide suitable for inclusion in a composition of the present disclosure comprises the following amino acid sequence: VLRRHIGPGEGAVQWMNRLIAFASRGNHVSPTHYV (SEQ ID NO:207); where the TP35-NS4 T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the TP35-NS4 T-cell epitope polypeptide has a length of 35 amino acids. In some cases, the T-cell epitope polypeptide is part of a fusion polypeptide, as described in detail below.

In some cases, a TP35-NS4 T-cell epitope polypeptide suitable for inclusion in a composition of the present disclosure comprises the following amino acid sequence: ILRRHVGPAEGATQWMNRLIAFASRGNHVSPTHYV (SEQ ID NO:208); where the TP35-NS4 T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the TP35-NS4 T-cell epitope polypeptide has a length of 35 amino acids. In some cases, the T-cell epitope polypeptide is part of a fusion polypeptide, as described in detail below.

In some cases, a TP35-NS4 T-cell epitope polypeptide suitable for inclusion in a composition of the present disclosure comprises the following amino acid sequence: IIKRHTGTSEGVTQWMNRLIAFASRGNHVSPTHYI (SEQ ID NO:209); where the TP35-NS4 T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the TP35-NS4 T-cell epitope polypeptide has a length of 35 amino acids. In some cases, the T-cell epitope polypeptide is part of a fusion polypeptide, as described in detail below.

A TP35-NS4 T-cell epitope polypeptide suitable for inclusion in a composition of the present disclosure can include one or more CD8 epitopes, e.g., a stretch of about 16 contiguous amino acids having the amino acid sequence: EGAVQWMNRLIAFASR (SEQ ID NO:210), or EGATQWMNRLIAFASR (SEQ ID NO:211), or EGVTQWMNRLIAFASR (SEQ ID NO:212); or a stretch of about 9 contiguous amino acids having the amino acid sequence: WMNRLIAFA (SEQ ID NO:213).

A TP35-NS4 T-cell epitope polypeptide suitable for inclusion in a composition of the present disclosure can include one or more CD4 epitopes, e.g., a stretch of about 15 contiguous amino acids having the amino acid sequence: RHVGPGEGAVQWMNR (SEQ ID NO:214), or RHVGQGEGAVQWMNR (SEQ ID NO:215), or RHIGPGEGAVQWMNR (SEQ ID NO:216), or RHVGPAEGATQWMNR (SEQ ID NO:217), or RHTGTSEGVTQWMNR (SEQ ID NO:218); or a stretch of about 20 contiguous amino acids having the amino acid sequence: HVGPGEGAVQWMNRLIAFAS (SEQ ID NO:219), or HVGQGEGAVQWMNRLIAFAS (SEQ ID NO:220), or HIGPGEGAVQWMNRLIAFAS (SEQ ID NO:221), or HVGPAEGATQWMNRLIAFAS (SEQ ID NO:222), or HTGTSEGVTQWMNRLIAFAS (SEQ ID NO:223); or a stretch of about 20 contiguous amino acids having the amino acid sequence: GAVQWMNRLIAFASRGNHVS (SEQ ID NO:224), or GAVQWMNRLIAFASRGNHVA (SEQ ID NO:225), or GATQWMNRLIAFASRGNHVS (SEQ ID NO:226), or GVTQWMNRLIAFASRGNHVS (SEQ ID NO:227).

In some cases, TP35-NS4 T-cell epitope polypeptide suitable for inclusion in a composition of the present disclosure includes: a) one or more CD8 epitopes, e.g., a stretch of about 16 contiguous amino acids having the amino acid sequence: EGAVQWMNRLIAFASR (SEQ ID NO:210), or EGATQWMNRLIAFASR (SEQ ID NO:211), or EGVTQWMNRLIAFASR (SEQ ID NO:212); or a stretch of about 9 contiguous amino acids having the amino acid sequence: WMNRLIAFA (SEQ ID NO:213); and b) one or more CD4 epitopes, e.g., a stretch of about 15 contiguous amino acids having the amino acid sequence: RHVGPGEGAVQWMNR (SEQ ID NO:214), or RHVGQGEGAVQWMNR (SEQ ID NO:215), or RHIGPGEGAVQWMNR (SEQ ID NO:216), or RHVGPAEGATQWMNR (SEQ ID NO:217), or RHTGTSEGVTQWMNR (SEQ ID NO:218); or a stretch of about 20 contiguous amino acids having the amino acid sequence: HVGPGEGAVQWMNRLIAFAS (SEQ ID NO:219), or HVGQGEGAVQWMNRLIAFAS (SEQ ID NO:220), or HIGPGEGAVQWMNRLIAFAS (SEQ ID NO:221), or HVGPAEGATQWMNRLIAFAS (SEQ ID NO:222), or HTGTSEGVTQWMNRLIAFAS (SEQ ID NO:223); or a stretch of about 20 contiguous amino acids having amino acid sequence: GAVQWMNRLIAFASRGNHVS (SEQ ID NO:224), or GAVQWMNRLIAFASRGNHVA (SEQ ID NO:225), or GATQWMNRLIAFASRGNHVS (SEQ ID NO:226), or GVTQWMNRLIAFASRGNHVS (SEQ ID NO:227).

In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: ILRRHVGPGEGAVQWMNRLIAFASRGNHVSPTHYV (SEQ ID NO:203); and has a length of from 35 amino acids to 40 amino acids (e.g., 35 amino acids (aa), 36 aa, 37 aa, 38 aa, 39 aa, or 40 aa).

In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: ILRRHVGPGEGAVQWMNRLIAFASRGNHVSPTHYV (SEQ ID NO:203); and has a length of 35 amino acids. In some cases, a T-cell epitope polypeptide suitable for inclusion in a composition of the present disclosure comprises the amino acid sequence ILRRHVGPGEGAVQWMNRLIAFASRGNHVSPTHYV (SEQ ID NO:203); and has a length of 35 amino acids.

### TP42

In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: GTEGEIPFYGKAIPLEQIKGGRHLIFCHSKKKCDELAAKLTG (SEQ ID NO:228); and has a length of from 33 amino acids to about 50 amino acids (e.g., 33 amino acids (aa), 34 aa, 35 aa, 36 aa, 37 aa, 38 aa, 39 aa, 40 aa, 41 aa, 42 aa, or from 42 aa to 50 aa). In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: GTEGEIPFYGKAIPLEQIKGGRHLIFCHSKKKCDELAAKLTG (SEQ ID NO:228); and has a length of 42 amino acids. In some cases, a T-cell epitope polypeptide suitable for inclusion in a composition of the present disclosure comprises the amino acid sequence GTEGEIPFYGKAIPLEQIKGGRHLIFCHSKKKCDELAAKLTG (SEQ ID NO:228); and has a length of 42 amino acids.

In some cases, a TP42 T-cell epitope polypeptide suitable for inclusion in a composition of the present disclosure comprises the following amino acid sequence: X₁X₂X₃GEIPFYGX₄AIPX₅X₆X₇X₈KGGRHLIFCHSKKKCDEX₉AX₁₀X₁₁LX₁₂X₁₃(K)n (SEQ ID NO:229), where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, or L; X₈ is I or L; X₉ is L or I; X₁₀ is A, K, or S; X₁₁ is K, Q, or A; X₁₂ is T, R, or S; and X₁₃ is G or S, wherein n is an integer from 2 to 10, and where the TP42 T-cell epitope polypeptide has a length of from 34 amino acids to 52 amino acids (e.g., 34 amino acids (aa), 35 aa, 36 aa, 37 aa, 38 aa, 39 aa, 40 aa, 41 aa, 42 aa, 43 aa, 44 aa, 45 aa, 46 aa, 47 aa, 48 aa, 49 aa, 50 aa, 51 aa, or 52 aa. In some cases, a TP42 T-cell epitope polypeptide suitable for inclusion in a composition of the present disclosure comprises the following amino acid sequence: X₁X₂X₃GEIPFYGX₄AIPX₅X₆X₇X₈KGGRHLIFCHSKKKCDEX₉AX₁₀X₁₁LX₁₂X₁₃KKK (SEQ ID NO:230), where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, or L; X₈ is I or L; X₉ is L or I; X₁₀ is A, K, or S; X₁₁ is K, Q, or A; X₁₂ is T, R, or S; and X₁₃ is G or S. In some cases, a TP42 T-cell epitope polypeptide suitable for inclusion in a composition of the present disclosure comprises the following amino acid sequence: X₁X₂X₃GEIPFYGX₄AIPX₅X₆X₇X₈KGGRHLIFCHSKKKCDEX₉AX₁₀X₁₁LX₁₂X₁₃(K)n (SEQ ID NO:229), where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, V, or L; X₈ is I or L; X₉ is L or I; X₁₀ is A, K, or S; X₁₁ is K, Q, or A; X₁₂ is T, R, V, or S; and X₁₃ is G or S, wherein n is an integer from 2 to 10, and where the TP42 T-cell epitope polypeptide has a length of from 34 amino acids to 52 amino acids (e.g., 34 amino acids (aa), 35 aa, 36 aa, 37 aa, 38 aa, 39 aa, 40 aa, 41 aa, 42 aa, 43 aa, 44 aa, 45 aa, 46 aa, 47 aa, 48 aa, 49 aa, 50 aa, 51 aa, or 52 aa. In some cases, a TP42 T-cell epitope polypeptide suitable for inclusion in a composition of the present disclosure comprises the following amino acid sequence: X₁X₂X₃GEIPFYGX₄AIPX₅X₆X₇X₈KGGRHLIFCHSKKKCDEX₉AX₁₀X₁₁LX₁₂X₁₃KKK (SEQ ID NO:230), where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, V, or L; X₈ is I or L; X₉ is L or I; X₁₀ is A, K, or S; X₁₁ is K, Q, or A; X₁₂ is T, R, V, or S; and X₁₃ is G or S. In some cases, the TP42 T-cell epitope polypeptide has a length of 45 amino acids. In some cases, the TP42 T-cell epitope polypeptide comprises the following amino acid sequence: GTEGEIPFYGKAIPLEQIKGGRHLIFCHSKKKCDELAAKLTGKKK (SEQ ID NO:231) and has a length of 45 amino acids.

In some cases, a TP42 T-cell epitope polypeptide suitable for inclusion in a composition of the present disclosure comprises the following amino acid sequence: X₁X₂X₃GEIPFYGX₄AIPX₅X₆X₇X₈KGGRHLIFCHSKKKCDEX₉AX₁₀X₁₁LX₁₂X₁₃ (SEQ ID NO:232), where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, or L; X₈ is I or L; X₉ is L or I; X₁₀ is A, K, or S; X₁₁ is K, Q, or A; X₁₂ is T, R, or S; and X₁₃ is G or S; where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the T-cell epitope polypeptide has a length of 42 amino acids. In some cases, the T-cell epitope polypeptide is part of a fusion polypeptide, as described in detail below.

In some cases, a TP42 T-cell epitope polypeptide suitable for inclusion in a composition of the present disclosure comprises the following amino acid sequence: STTGEIPFYGKAIPLEVIKGGRHLIFCHSKKKCDELAAKLVA (SEQ ID NO:233); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the T-cell epitope polypeptide has a length of 42 amino acids. In some cases, the T-cell epitope polypeptide is part of a fusion polypeptide, as described in detail below.

In some cases, a TP42 T-cell epitope polypeptide suitable for inclusion in a composition of the present disclosure comprises the following amino acid sequence: SNTGEIPFYGKAIPIETIKGGRHLIFCHSKKKCDELAAKLSG (SEQ ID NO:234); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the T-cell epitope polypeptide has a length of 42 amino acids. In some cases, the T-cell epitope polypeptide is part of a fusion polypeptide, as described in detail below.

In some cases, a TP42 T-cell epitope polypeptide suitable for inclusion in a composition of the present disclosure comprises the following amino acid sequence: GQEGEIPFYGRAIPLSYIKGGRHLIFCHSKKKCDELAAALRG (SEQ ID NO:235); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the T-cell epitope polypeptide has a length of 42 amino acids. In some cases, the T-cell epitope polypeptide is part of a fusion polypeptide, as described in detail below.

In some cases, a TP42 T-cell epitope polypeptide suitable for inclusion in a composition of the present disclosure comprises the following amino acid sequence: GHEGEIPFYGKAIPLAFIKGGRHLIFCHSKKKCDELAALRG (SEQ ID NO:236); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the T-cell epitope polypeptide has a length of 42 amino acids. In some cases, the T-cell epitope polypeptide is part of a fusion polypeptide, as described in detail below.

In some cases, a TP42 T-cell epitope polypeptide suitable for inclusion in a composition of the present disclosure comprises the following amino acid sequence: GSEGEIPFYGKAIPIAQLKGGRHLIFCHSKKKCDEIASKLRG (SEQ ID NO:237); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the T-cell epitope polypeptide has a length of 42 amino acids. In some cases, the T-cell epitope polypeptide is part of a fusion polypeptide, as described in detail below.

In some cases, a TP42 T-cell epitope polypeptide suitable for inclusion in a composition of the present disclosure comprises the following amino acid sequence: PTTGEIPFYGKAIPLELIKGGRHLIFCHSKKKCDELAQLTS (SEQ ID NO:238); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the T-cell epitope polypeptide has a length of 42 amino acids. In some cases, the T-cell epitope polypeptide is part of a fusion polypeptide, as described in detail below.

In some cases, a TP42 T-cell epitope polypeptide suitable for inclusion in a composition of the present disclosure comprises the following amino acid sequence: PSEGEIPFYGRAIPLXLIKGGRHLIFCHSKKKCDELAKQLTS (SEQ ID NO:239); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the T-cell epitope polypeptide has a length of 42 amino acids. In some cases, the T-cell epitope polypeptide is part of a fusion polypeptide, as described in detail below.

In some cases, a TP42 T-cell epitope polypeptide suitable for inclusion in a composition of the present disclosure comprises the following amino acid sequence: PTTGEIPFYGKAIPLEYIKGGRHLIFCHSKKKCDELAKQLTS (SEQ ID NO:240); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the T-cell epitope polypeptide has a length of 42 amino acids. In some cases, the T-cell epitope polypeptide is part of a fusion polypeptide, as described in detail below.

In some cases, a TP42 T-cell epitope polypeptide suitable for inclusion in a composition of the present disclosure comprises the following amino acid sequence: GNDGEIPFYGKAIPLQHIKGGRHLIFCHSKKKCDELAGKLTS (SEQ ID NO:241); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the T-cell epitope polypeptide has a length of 42 amino acids. In some cases, the T-cell epitope polypeptide is part of a fusion polypeptide, as described in detail below.

A TP42 T-cell epitope polypeptide suitable for inclusion in a composition of the present disclosure can include one or more CD8 epitopes, e.g., one or more of: i) a stretch of about 11 contiguous amino acids having the amino acid sequence: FYGX₁AIPX₂X₃X₄X₅(SEQ ID NO:242), where X₁ is K or R; X₂ is L or I; X₃ is E, S, A, Q, or V; X₄ is Q, T, Y, F, L, or H; and X₅ is I or L; ii) a stretch of about 9 contiguous amino acids having the amino acid sequence: LIFCHSKKK (SEQ ID NO:200); and iii) a stretch of about 9 contiguous amino acids having the amino acid sequence: HSKKKCDEL (SEQ ID NO:201). In some cases, a TP42 T-cell epitope polypeptide suitable for inclusion in a composition of the present disclosure includes the following CD8 epitopes: i) a stretch of about 11 contiguous amino acids having the amino acid sequence: FYGKAIPLEQI (SEQ ID NO:243); ii) ii) a stretch of about 9 contiguous amino acids having the amino acid sequence: LIFCHSKKK (SEQ ID NO:200); and iii) a stretch of about 9 contiguous amino acids having the amino acid sequence: HSKKKCDEL (SEQ ID NO:201).

A TP42 T-cell epitope polypeptide suitable for inclusion in a composition of the present disclosure can include one or more CD4 epitopes, e.g., a stretch of about 15 contiguous amino acids having the amino acid sequence: KGGRHLIFCHSKKKCD (SEQ ID NO:202), or a stretch of about 11 contiguous amino acids having the amino acid sequence: GRHLIFCHSKK (SEQ ID NO:244).

A TP42 T-cell epitope polypeptide suitable for inclusion in a composition of the present disclosure can include: a) the following CD8 epitopes: i) a stretch of about 11 contiguous amino acids having the amino acid sequence: FYGKAIPLEQI (SEQ ID NO:243); ii) ii) a stretch of about 9 contiguous amino acids having the amino acid sequence: LIFCHSKKK (SEQ ID NO:200); and iii) a stretch of about 9 contiguous amino acids having the amino acid sequence: HSKKKCDEL (SEQ ID NO:201); and b) the following CD4 epitope: a stretch of about 11 contiguous amino acids having the amino acid sequence: GRHLIFCHSKK (SEQ ID NO:244). A TP42 T-cell epitope polypeptide suitable for inclusion in a composition of the present disclosure can include: a) the following CD8 epitopes: i) a stretch of about 11 contiguous amino acids having the amino acid sequence: FYGKAIPLEQI (SEQ ID NO:243); ii) ii) a stretch of about 9 contiguous amino acids having the amino acid sequence: LIFCHSKKK (SEQ ID NO:200); and iii) a stretch of about 9 contiguous amino acids having the amino acid sequence: HSKKKCDEL (SEQ ID NO:201); and b) the following CD4 epitope: a stretch of about 15 contiguous amino acids having the amino acid sequence: KGGRHLIFCHSKKKCD (SEQ ID NO:202).

In some cases, a suitable T-cell epitope polypeptide is a TP42 polypeptide with from 1 amino acid to 5 amino acids removed from the N-terminus and/or from 1 amino acid to 5 amino acids removed from the C-terminus. Thus, e.g., a suitable T-cell epitope polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: IPFYGKAIPLEQIKGGRHLIFCHSKKKCDELAAKLTG (SEQ ID NO:245); and has a length of 37 amino acids. As another example, a suitable T-cell epitope polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: GTEGEIPFYGKAIPLEQIKGGRHLIFCHSKKKCDELA (SEQ ID NO:246); and has a length of 37 amino acids.

### TP45

In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: LNAVAYYRGLDVSVIPTSGDVVVVATDALMTGFTGDFDSVIDCNV (SEQ ID NO:247); and has a length of from 45 amino acids to about 50 amino acids. In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: LNAVAYYRGLDVSVIPTSGDVVVVATDALMTGFTGDFDSVIDCNV (SEQ ID NO:247); and has a length of 45 amino acids. In some cases, a T-cell epitope polypeptide suitable for inclusion in a composition of the present disclosure comprises the amino acid sequence LNAVAYYRGLDVSVIPTSGDVVVVATDALMTGFTGDFDSVIDCNV (SEQ ID NO:247); and has a length of 45 amino acids. In some cases, a T-cell epitope polypeptide suitable for inclusion in a composition of the present disclosure comprises the amino acid sequence LNAVAYYRGLDVSVIPTSGDVVVVATDALMTGFTGDFDSVIDKKK (SEQ ID NO:248); and has a length of 45 amino acids.

In some cases, a TP45 T-cell epitope polypeptide suitable for inclusion in a composition of the present disclosure comprises the following amino acid sequence: X₁X₂AVAX₃YRGX₄DVX₅X₆IPX₇X₈GDVVVX₉X₁₀TDALMTGX₁₁TGDFDSVIDX₁₂X₁₃X₁₄(K)n (SEQ ID NO:249), where X₁ is L or V; X₂ is N or T; X₃ is Y or F; X₄ is L or V; X₅ is S or A; X₆ is V or I; X₇ is T or A; X₈ is S, Q, or T; X₉ is V or C; X₁₀ is A or S; X₁₁ is F or Y; X₁₂ is C or K; X₁₃ is N or K; and X₁₄ is V or K, wherein n is an integer from 2 to 10, and where the TP45 T-cell epitope polypeptide has a length of from 36 amino acids to 55 amino acids. In some cases, a TP45 T-cell epitope polypeptide suitable for inclusion in a composition of the present disclosure comprises the following amino acid sequence: X₁X₂AVAX₃YRGX₄DVX₅X₆IPX₇X₈GDVVVX₉X₁₀TDALMTGX₁₁TGDFDSVIDX₁₂X₁₃X₁₄KKK (SEQ ID NO:250), where X₁ is L or V; X₂ is N or T; X₃ is Y or F; X₄ is L or V; X₅ is S or A; X₆ is V or I; X₇ is T or A; X₈ is S, Q, or T; X₉ is V or C; X₁₀ is A or S; X₁₁ is F or Y; X₁₂ is C or K; X₁₃ is N or K; and X₁₄ is V or K; where the TP45 T-cell epitope polypeptide has a length of 48 amino acids. In some cases, the TP45 T-cell epitope polypeptide comprises the following amino acid sequence: LNAVAYYRGLDVSVIPTSGDVVVVATDALMTGFTGDFDSVIDCNVKKK (SEQ ID NO:251) and has a length of 48 amino acids.

In some cases, a TP45 T-cell epitope polypeptide suitable for inclusion in a composition of the present disclosure comprises the following amino acid sequence: X₁X₂AVAX₃YRGX₄DVX₅X₆IPX₇X₈GDVVVX₉X₁₀TDALMTGX₁₁TGDFDSVIDX₁₂X₁₃X₁₄ (SEQ ID NO:252), where X₁ is L or V; X₂ is N or T; X₃ is Y or F; X₄ is L or V; X₅ is S or A; X₆ is V or I; X₇ is T or A; X₈ is S, Q, or T; X₉ is V or C; X₁₀ is A or S; X₁₁ is F or Y; X₁₂ is C or K; X₁₃ is N or K; and X₁₄ is V or K; where the TP23 T-cell epitope polypeptide has a length of from 18 amino acids to 23 amino acids (e.g., T-cell epitope polypeptide has a length of 18 amino acids (aa), 19 aa, 20 aa, 21 aa, 22 aa, or 23 aa). In some cases, the TP23 T-cell epitope polypeptide has a length of 23 amino acids. In some cases, the TP23 T-cell epitope polypeptide is part of a fusion polypeptide, as described in detail below.

In some cases, a TP45 T-cell epitope polypeptide suitable for inclusion in a composition of the present disclosure comprises the following amino acid sequence: X₁X₂AVAX₃YRGX₄DVX₅X₆IPX₇X₈GDVVVX₉X₁₀TDALMTGX₁₁TGDFDSVIDX₁₂X₁₃X₁₄ (SEQ ID NO:252), where X₁ is L; X₂ is N; X₃ is Y; X₄ is L; X₅ is S; X₆ is V; X₇ is T; X₈ is S, Q, or T; X₉ is V or C; X₁₀ is A; X₁₁ is F or Y; X₁₂ is K; X₁₃ is K; and X₁₄ is K; where the TP23 T-cell epitope polypeptide has a length of from 18 amino acids to 23 amino acids (e.g., T-cell epitope polypeptide has a length of 18 amino acids (aa), 19 aa, 20 aa, 21 aa, 22 aa, or 23 aa). In some cases, the TP23 T-cell epitope polypeptide has a length of 23 amino acids. In some cases, the TP23 T-cell epitope polypeptide is part of a fusion polypeptide, as described in detail below.

In some cases, a suitable T-cell epitope polypeptide is a TP45 polypeptide with from 1 amino acid to 5 amino acids removed from the N-terminus and/or from 1 amino acid to 5 amino acids removed from the C-terminus. Thus, for example, in some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: LNAVAYYRGLDVSVIPTSGDVVVVATDALMTGFTGDFDSVID (SEQ ID NO:253); and has a length of 43 amino acids. For example, in some cases, a T-cell epitope polypeptide suitable for inclusion in a composition of the present disclosure comprises the amino acid sequence LNAVAYYRGLDVSVIPTSGDVVVVATDALMTGFTGDFDSVID (SEQ ID NO:253); and has a length of 43 amino acids.

### TP48

In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: ILRRHVGPGEGAVQWMNRLIAFASRGNHVSPTHYVPESDASARVTQIL (SEQ ID NO:254); and has a length of from 48 amino acids to about 55 amino acids, or has a length of from 40 amino acids to about 48 amino acids (e.g., has a length of 40 amino acids (aa), 41 aa, 42 aa, 43 aa, 44 aa, 45 aa, 46 aa, 47 aa, or 48 aa). In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: ILRRHVGPGEGAVQWMNRLIAFASRGNHVSPTHYVPESDASARVTQIL (SEQ ID NO:254); and has a length of 48 amino acids. In some cases, a T-cell epitope polypeptide suitable for inclusion in a composition of the present disclosure comprises the amino acid sequence ILRRHVGPGEGAVQWMNRLIAFASRGNHVSPTHYVPESDASARVTQIL (SEQ ID NO:254); and has a length of 48 amino acids.

In some cases, a TP48 T-cell epitope polypeptide suitable for inclusion in a composition of the present disclosure comprises the following amino acid sequence: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:255), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, where n is an integer from 2 to 10, and where the TP48 T-cell epitope polypeptide has a length of from 38 amino acids to 58 amino acids. In some cases, a TP48 T-cell epitope polypeptide suitable for inclusion in a composition of the present disclosure comprises the following amino acid sequence: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉LKKK (SEQ ID NO:256), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, where the TP48 T-cell epitope polypeptide has a length of 51 amino acids. In some cases, the TP48 T-cell epitope polypeptide comprises the amino acid sequence: ILRRHVGPGEGAVQWMNRLIAFASRGNHVSPTHYVPESDASARVTQILKKK (SEQ ID NO:257) and has a length of 51 amino acids.

In some cases, a TP48 T-cell epitope polypeptide suitable for inclusion in a composition of the present disclosure comprises the following amino acid sequence: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L (SEQ ID NO:266), where X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V; where the TP48 T-cell epitope polypeptide has a length of from 40 amino acids to about 48 amino acids (e.g., has a length of 40 amino acids (aa), 41 aa, 42 aa, 43 aa, 44 aa, 45 aa, 46 aa, 47 aa, or 48 aa). In some cases, the TP48 T-cell epitope polypeptide has a length of 48 amino acids. In some cases, the T-cell epitope polypeptide is part of a fusion polypeptide, as described in detail below.

In some cases, a TP48 T-cell epitope polypeptide suitable for inclusion in a composition of the present disclosure comprises the following amino acid sequence: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L (SEQ ID NO:266), where X₁ is I or V; X₂ is L; X₃ is R; X₄ is V or I; X₅ is P or Q; X₆ is G or A; X₇ is A; X₈ is V or T; X₉ is S or A; X₁₀ is V; X₁₁ is P, T, or A; X₁₂ is E; X₁₃ is S or T; X₁₄ is S or A; X₁₅ is A, Q, or R; X₁₆ is R or K; X₁₇ is T; X₁₈ is Q, A, or T; and X₁₉ is I, L, or V; where the TP48 T-cell epitope polypeptide has a length of from 40 amino acids to about 48 amino acids (e.g., has a length of 40 amino acids (aa), 41 aa, 42 aa, 43 aa, 44 aa, 45 aa, 46 aa, 47 aa, or 48 aa). In some cases, the TP48 T-cell epitope polypeptide has a length of 48 amino acids. In some cases, the T-cell epitope polypeptide is part of a fusion polypeptide, as described in detail below.

In some cases, a TP48 T-cell epitope polypeptide suitable for inclusion in a composition of the present disclosure comprises the following amino acid sequence: ILRRHVGPGEGAVQWMNRLIAFASRGNHVSPTHYVPESDAAARVTAIL (SEQ ID NO:267); where the TP48 T-cell epitope polypeptide has a length of from 40 amino acids to about 48 amino acids (e.g., has a length of 40 amino acids (aa), 41 aa, 42 aa, 43 aa, 44 aa, 45 aa, 46 aa, 47 aa, or 48 aa). In some cases, the TP48 T-cell epitope polypeptide has a length of 48 amino acids. In some cases, the T-cell epitope polypeptide is part of a fusion polypeptide, as described in detail below.

In some cases, a TP48 T-cell epitope polypeptide suitable for inclusion in a composition of the present disclosure comprises the following amino acid sequence: ILRRHVGPGEGAVQWMNRLIAFASRGNHVSPTHYVPESDAAARVTQIL (SEQ ID NO:268); where the TP48 T-cell epitope polypeptide has a length of from 40 amino acids to about 48 amino acids (e.g., has a length of 40 amino acids (aa), 41 aa, 42 aa, 43 aa, 44 aa, 45 aa, 46 aa, 47 aa, or 48 aa). In some cases, the TP48 T-cell epitope polypeptide has a length of 48 amino acids. In some cases, the T-cell epitope polypeptide is part of a fusion polypeptide, as described in detail below.

In some cases, a TP48 T-cell epitope polypeptide suitable for inclusion in a composition of the present disclosure comprises the following amino acid sequence: ILRRHVGPGEGAVQWMNRLIAFASRGNHVAPTHYVTESDASQRVTQLL (SEQ ID NO:269); where the TP48 T-cell epitope polypeptide has a length of from 40 amino acids to about 48 amino acids (e.g., has a length of 40 amino acids (aa), 41 aa, 42 aa, 43 aa, 44 aa, 45 aa, 46 aa, 47 aa, or 48 aa). In some cases, the TP48 T-cell epitope polypeptide has a length of 48 amino acids. In some cases, the T-cell epitope polypeptide is part of a fusion polypeptide, as described in detail below.

In some cases, a TP48 T-cell epitope polypeptide suitable for inclusion in a composition of the present disclosure comprises the following amino acid sequence: ILRRHVGQGEGAVQWMNRLIAFASRGNHVAPTHYVAESDASQRVTQVL (SEQ ID NO:270); where the TP48 T-cell epitope polypeptide has a length of from 40 amino acids to about 48 amino acids (e.g., has a length of 40 amino acids (aa), 41 aa, 42 aa, 43 aa, 44 aa, 45 aa, 46 aa, 47 aa, or 48 aa). In some cases, the TP48 T-cell epitope polypeptide has a length of 48 amino acids. In some cases, the T-cell epitope polypeptide is part of a fusion polypeptide, as described in detail below.

In some cases, a TP48 T-cell epitope polypeptide suitable for inclusion in a composition of the present disclosure comprises the following amino acid sequence: ILRRHVGPGEGAVQWMNRLIAFASRGNHVSPTHYVPESDAAARVTALL (SEQ ID NO:271); where the TP48 T-cell epitope polypeptide has a length of from 40 amino acids to about 48 amino acids (e.g., has a length of 40 amino acids (aa), 41 aa, 42 aa, 43 aa, 44 aa, 45 aa, 46 aa, 47 aa, or 48 aa). In some cases, the TP48 T-cell epitope polypeptide has a length of 48 amino acids. In some cases, the T-cell epitope polypeptide is part of a fusion polypeptide, as described in detail below.

In some cases, a TP48 T-cell epitope polypeptide suitable for inclusion of the present disclosure comprises the following amino acid sequence: ILRRHVGPGEGAVQWMNRLIAFASRGNHVSPTHYVPESDAAARVTQIL (SEQ ID NO:268); where the TP48 T-cell epitope polypeptide has a length of from 40 amino acids to about 48 amino acids (e.g., has a length of 40 amino acids (aa), 41 aa, 42 aa, 43 aa, 44 aa, 45 aa, 46 aa, 47 aa, or 48 aa). In some cases, the TP48 T-cell epitope polypeptide has a length of 48 amino acids. In some cases, the T-cell epitope polypeptide is part of a fusion polypeptide, as described in detail below.

In some cases, a TP48 T-cell epitope polypeptide suitable for inclusion of the present disclosure comprises the following amino acid sequence: VLRRHIGPGEGAVQWMNRLIAFASRGNHVSPTHYV PETDASAKVTQLL (SEQ ID NO:272); where the TP48 T-cell epitope polypeptide has a length of from 40 amino acids to about 48 amino acids (e.g., has a length of 40 amino acids (aa), 41 aa, 42 aa, 43 aa, 44 aa, 45 aa, 46 aa, 47 aa, or 48 aa). In some cases, the TP48 T-cell epitope polypeptide has a length of 48 amino acids. In some cases, the T-cell epitope polypeptide is part of a fusion polypeptide, as described in detail below.

In some cases, a TP48 T-cell epitope polypeptide suitable for inclusion of the present disclosure comprises the following amino acid sequence: ILRRHVGPAEGATQWMNRLIAFASRGNHVSPTHYVPETDASRXVTTIL (SEQ ID NO:273); where the TP48 T-cell epitope polypeptide has a length of from 40 amino acids to about 48 amino acids (e.g., has a length of 40 amino acids (aa), 41 aa, 42 aa, 43 aa, 44 aa, 45 aa, 46 aa, 47 aa, or 48 aa). In some cases, the TP48 T-cell epitope polypeptide has a length of 48 amino acids. In some cases, the T-cell epitope polypeptide is part of a fusion polypeptide, as described in detail below.

In some cases, a TP48 T-cell epitope polypeptide suitable for inclusion of the present disclosure comprises the following amino acid sequence: IIKRHTGTSEGVTQWMNRLIAFASRGNHVSPTHYIQDDDASKRVMGIL (SEQ ID NO:274); where the TP48 T-cell epitope polypeptide has a length of from 40 amino acids to about 48 amino acids (e.g., has a length of 40 amino acids (aa), 41 aa, 42 aa, 43 aa, 44 aa, 45 aa, 46 aa, 47 aa, or 48 aa). In some cases, the TP48 T-cell epitope polypeptide has a length of 48 amino acids. In some cases, the T-cell epitope polypeptide is part of a fusion polypeptide, as described in detail below.

In some cases, a TP48 T-cell epitope polypeptide suitable for inclusion of the present disclosure comprises the following amino acid sequence: ILRRHVGPAEGATQWMNRLIAFASRGNHVSPTHYVPETDASRXVTTIL (SEQ ID NO:273), where X is R or K; where the TP48 T-cell epitope polypeptide has a length of from 40 amino acids to about 48 amino acids (e.g., has a length of 40 amino acids (aa), 41 aa, 42 aa, 43 aa, 44 aa, 45 aa, 46 aa, 47 aa, or 48 aa). In some cases, the TP48 T-cell epitope polypeptide has a length of 48 amino acids. In some cases, the T-cell epitope polypeptide is part of a fusion polypeptide, as described in detail below.

A TP48 T-cell epitope polypeptide suitable for inclusion in a composition of the present disclosure can include one or more CD8 epitopes, e.g., a stretch of about 16 contiguous amino acids having the amino acid sequence: EGAVQWMNRLIAFASR (SEQ ID NO:210), or EGATQWMNRLIAFASR (SEQ ID NO:211), or EGVTQWMNRLIAFASR (SEQ ID NO:212); or a stretch of about 9 contiguous amino acids having the amino acid sequence: WMNRLIAFA (SEQ ID NO:213); or a stretch of about 15 contiguous amino acids having the amino acid sequence: PTHYX₁X₂X₃X₄DAX₅X₆X₇VX₈ (SEQ ID NO:276), where X₁ is V or I; X₂ is P, T, A, or Q, X₃ is E or D; X₄ is S, T, or D; X₅ is S or A; X₆ is A, Q, R, or K; X₇ is R, K, or X; and X₈ is T or M.

A TP48 T-cell epitope polypeptide suitable for inclusion in a composition of the present disclosure can include one or more CD8 epitopes, e.g., a stretch of about 16 contiguous amino acids having the amino acid sequence: EGAVQWMNRLIAFASR (SEQ ID NO:210), or EGATQWMNRLIAFASR (SEQ ID NO:211), or EGVTQWMNRLIAFASR (SEQ ID NO:212); or a stretch of about 9 contiguous amino acids having the amino acid sequence: WMNRLIAFA (SEQ ID NO:213); or a stretch of about 15 contiguous amino acids having the amino acid sequence: PTHYX₁X₂X₃X₄DAX₅X₆X₇VX₈ (SEQ ID NO:276), where X₁ is V; X₂ is P, T, or A, X₃ is E; X₄ is S or T; X₅ is S or A; X₆ is A, Q, or R; X₇ is R or K; and X₈ is T.

A TP48 T-cell epitope polypeptide suitable for inclusion in a composition of the present disclosure can include one or more CD4 epitopes, e.g., a stretch of about 15 contiguous amino acids having the amino acid sequence: RHVGPGEGAVQWMNR (SEQ ID NO:214), or RHVGQGEGAVQWMNR (SEQ ID NO:215), or RHIGPGEGAVQWMNR (SEQ ID NO:216), or RHVGPAEGATQWMNR (SEQ ID NO:217), or RHTGTSEGVTQWMNR (SEQ ID NO:218); or a stretch of about 20 contiguous amino acids having the amino acid sequence: HVGPGEGAVQWMNRLIAFAS (SEQ ID NO:219), or HVGQGEGAVQWMNRLIAFAS (SEQ ID NO:220), or HIGPGEGAVQWMNRLIAFAS (SEQ ID NO:221), or HVGPAEGATQWMNRLIAFAS (SEQ ID NO:222), or HTGTSEGVTQWMNRLIAFAS (SEQ ID NO:223); or a stretch of about 20 contiguous amino acids having amino acid sequence: GAVQWMNRLIAFASRGNHVS (SEQ ID NO:224), or GAVQWMNRLIAFASRGNHVA (SEQ ID NO:225), or GATQWMNRLIAFASRGNHVS (SEQ ID NO:226), or GVTQWMNRLIAFASRGNHVS (SEQ ID NO:227).

In some cases, TP48 T-cell epitope polypeptide suitable for inclusion in a composition of the present disclosure includes: a) one or more CD8 epitopes, e.g., a stretch of about 16 contiguous amino acids having the amino acid sequence: EGAVQWMNRLIAFASR (SEQ ID NO:210), or EGATQWMNRLIAFASR (SEQ ID NO:211), or EGVTQWMNRLIAFASR (SEQ ID NO:212); or a stretch of about 9 contiguous amino acids having the amino acid sequence: WMNRLIAFA (SEQ ID NO:213); or a stretch of about 15 contiguous amino acids having the amino acid sequence: PTHYX₁X₂X₃X₄DAX₅X₆X₇VX₈ (SEQ ID NO:276), where X₁ is V or I; X₂ is P, T, A, or Q, X₃ is E or D; X₄ is S, T, or D; X₅ is S or A; X₆ is A, Q, R, or K; X₇ is R, K, or X; and X₈ is T or M; or a stretch of about 15 contiguous amino acids having the amino acid sequence: PTHYX₁X₂X₃X₄DAX₅X₆X₇VX₈ (SEQ ID NO:276), where X₁ is V; X₂ is P, T, or A, X₃ is E; X₄ is S or T; X₅ is S or A; X₆ is A, Q, or R; X₇ is R or K; and X₈ is T; and b) one or more CD4 epitopes, e.g., a stretch of about 15 contiguous amino acids having the amino acid sequence: RHVGPGEGAVQWMNR (SEQ ID NO:214), or RHVGQGEGAVQWMNR (SEQ ID NO:215), or RHIGPGEGAVQWMNR (SEQ ID NO:216), or RHVGPAEGATQWMNR (SEQ ID NO:217), or RHTGTSEGVTQWMNR (SEQ ID NO:218); or a stretch of about 20 contiguous amino acids having the amino acid sequence: HVGPGEGAVQWMNRLIAFAS (SEQ ID NO:219), or HVGQGEGAVQWMNRLIAFAS (SEQ ID NO:220), or HIGPGEGAVQWMNRLIAFAS (SEQ ID NO:221), or HVGPAEGATQWMNRLIAFAS (SEQ ID NO:222), or HTGTSEGVTQWMNRLIAFAS (SEQ ID NO:223); or a stretch of about 20 contiguous amino acids havin amino acid sequence: GAVQWMNRLIAFASRGNHVS (SEQ ID NO:224), or GAVQWMNRLIAFASRGNHVA (SEQ ID NO:225), or GATQWMNRLIAFASRGNHVS (SEQ ID NO:226), or GVTQWMNRLIAFASRGNHVS (SEQ ID NO:227).

In some cases, a suitable T-cell epitope polypeptide is a TP48 polypeptide with from 1 amino acid to 5 amino acids removed from the N-terminus and/or from 1 amino acid to 5 amino acids removed from the C-terminus. Thus, e.g., a suitable T-cell epitope polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence:

(SEQ ID NO: 277)
RHVGPGEGAVQWMNRLIAFASRGNHVSPTHYVPESDASARVTQIL;

and has a length of 45 amino acids. As another example, a suitable T-cell epitope polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence:

(SEQ ID NO: 278)
ILRRHVGPGEGAVQWMNRLIAFASRGNHVSPTHYVPESDASARVT;

and has a length of 45 amino acids.

### TP33

In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: HSKKKCDELAAKLTGLGLNAVAYYRGLDVSVIP (SEQ ID NO:279); and has a length of from 33 amino acids to about 37 amino acids (e.g., has a length of 33 amino acids (aa), 34 aa, 35 aa, 36 aa, or 37 aa), or has a length of from 30 amino acids to about 33 amino acids (e.g., has a length of 30 amino acids (aa), 31 aa, 32 aa, or 33 aa). In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: HSKKKCDELAAKLTGLGLNAVAYYRGLDVSVIP (SEQ ID NO:279); and has a length of 33 amino acids. In some cases, a T-cell epitope polypeptide suitable for inclusion in a composition of the present disclosure comprises the amino acid sequence HSKKKCDELAAKLTGLGLNAVAYYRGLDVSVIP (SEQ ID NO:279); and has a length of 33 amino acids. Such T-cell epitope polypeptides are also referred to herein as “TP33” polypeptides. In some cases, a TP33 T-cell epitope polypeptide comprises a CD8 epitope comprising a contiguous stretch of 19 amino acids of one of the following amino acid sequences:

i)
(SEQ ID NO: 280)
DELAAKLTGLGLNAVAYYR;
ii)
(SEQ ID NO: 281)
DELAAKLVALGINAVAYYR;
iii)
(SEQ ID NO: 282)
DELAAKLSGLGLNAVAYYR;
iv)
(SEQ ID NO: 283)
DELAAALRGMGLNAVAYYR;
v)
(SEQ ID NO: 284)
DELAAALRGMGVNAVAYYR;
vi)
(SEQ ID NO: 285)
DELASKLRGMGLNAVAYYR;
vii)
(SEQ ID NO: 286)
DELAAKLRGMGLNAVAYYR,

In some cases, a TP33 T-cell epitope polypeptide suitable for inclusion in a composition of the present disclosure comprises the following amino acid sequence: HSKKKCDELAX₁X₂LX₃X₄X₅GX₆NAVAYYRGLDVSX₇IP (SEQ ID NO:287), where X₁ is A or S; X₂ is K or A; X₃ is V, S, R, or T; X₄ is A or G; X₅ is L or M; X₆ is I, L, or V; and X₇ is V or I; and has a length of from 33 amino acids to about 37 amino acids (e.g., has a length of 33 amino acids (aa), 34 aa, 35 aa, 36 aa, or 37 aa), or has a length of from 30 amino acids to about 33 amino acids (e.g., has a length of 30 amino acids (aa), 31 aa, 32 aa, or 33 aa). In some cases, a TP33 T-cell epitope polypeptide suitable for inclusion in a composition of the present disclosure comprises the following amino acid sequence: HSKKKCDELAX₁X₂LX₃X₄X₅GX₆NAVAYYRGLDVSX₇IP (SEQ ID NO:287), where X₁ is A or S; X₂ is K or A; X₃ is V, S, R, or T; X₄ is A or G; X₅ is L or M; X₆ is I, L, or V; and X₇ is V or I; where the TP33 T-cell epitope polypeptide has a length of 33 amino acids.

In some cases, a TP33 T-cell epitope polypeptide suitable for inclusion in a composition of the present disclosure comprises the following amino acid sequence: HSKKKCDELAAKLX₁X₂X₃GX₄NAVAYYRGLDVSVIP (SEQ ID NO:288), where X₁ is V, S, R, or T; X₂ is A or G; X₃ is L or M; and X₄ is I, L, or V; and has a length of from 33 amino acids to about 37 amino acids (e.g., has a length of 33 amino acids (aa), 34 aa, 35 aa, 36 aa, or 37 aa), or has a length of from 30 amino acids to about 33 amino acids (e.g., has a length of 30 amino acids (aa), 31 aa, 32 aa, or 33 aa). In some cases, a TP33 T-cell epitope polypeptide suitable for inclusion in a composition of the present disclosure comprises the following amino acid sequence: HSKKKCDELAAKLX₁X₂X₃GX₄NAVAYYRGLDVSVIP (SEQ ID NO:288), where X₁ is V, S, R, or T; X₂ is A or G; X₃ is L or M; and X₄ is I, L, or V; where the TP33 T-cell epitope polypeptide has a length of 33 amino acids.

In some cases, a TP33 T-cell epitope polypeptide suitable for inclusion in a composition of the present disclosure comprises the following amino acid sequence: HSKKKCDELAAALRGMGXiNAVAYYRGLDVSX₁IP (SEQ ID NO:289), where X₁ is I, L, or V; and X₂ is V or I; and has a length of from 33 amino acids to about 37 amino acids (e.g., has a length of 33 amino acids (aa), 34 aa, 35 aa, 36 aa, or 37 aa), or has a length of from 30 amino acids to about 33 amino acids (e.g., has a length of 30 amino acids (aa), 31 aa, 32 aa, or 33 aa). In some cases, a TP33 T-cell epitope polypeptide suitable for inclusion in a composition of the present disclosure comprises the following amino acid sequence: HSKKKCDELAAALRGMGXiNAVAYYRGLDVSX₁IP (SEQ ID NO:289), where X₁ is I, L, or V; and X₂ is V or I; where the TP33 T-cell epitope polypeptide has a length of 33 amino acids.

In some cases, a T-cell epitope polypeptide suitable for inclusion in a composition of the present disclosure comprises the amino acid sequence HSKKKCDELAAKLRGMGLNAVAYYRGLDVSVIP (SEQ ID NO:290); and has a length of 33 amino acids.

### TP42-2

In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: KGGRHLIFCHSKKKCDELAAKLTGLGLNAVAYYRGLDVSVIP (SEQ ID NO:291); and has a length of from 42 amino acids to about 46 amino acids (e.g., has a length of 42 amino acids (aa), 43 aa, 44 aa, 45 aa, or 46 aa), or has a length of from 38 amino acids to about 42 amino acids (e.g., has a length of 38 amino acids (aa), 39 aa, 40 aa, 41 aa, or 42 aa). In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: KGGRHLIFCHSKKKCDELAAKLTGLGLNAVAYYRGLDVSVIP (SEQ ID NO:291); and has a length of 42 amino acids. In some cases, a T-cell epitope polypeptide suitable for inclusion in a composition of the present disclosure comprises the amino acid sequence KGGRHLIFCHSKKKCDELAAKLTGLGLNAVAYYRGLDVSVIP (SEQ ID NO:291); and has a length of 42 amino acids. Such T-cell epitope polypeptides are also referred to herein as “TP42-2” polypeptides. In some cases, a TP42-2 T-cell epitope polypeptide comprises a CD8 epitope comprising a contiguous stretch of 19 amino acids of one of the following amino acid sequences: i) DELAAKLTGLGLNAVAYYR (SEQ ID NO:280); ii) DELAAKLVALGINAVAYYR (SEQ ID NO:281); iii) DELAAKLSGLGLNAVAYYR (SEQ ID NO:282); iv) DELAAALRGMGLNAVAYYR (SEQ ID NO:283); v) DELAAALRGMGVNAVAYYR (SEQ ID NO:284); vi) DELASKLRGMGLNAVAYYR (SEQ ID NO:285); vii) DELAAKLRGMGLNAVAYYR (SEQ ID NO:286).

In some cases, a TP42-2 T-cell epitope polypeptide suitable for inclusion in a composition of the present disclosure comprises the following amino acid sequence: KGGRHLIFCHSKKKCDELAX₁X₂LX₃X₄X₅GX₆NAVAYYRGLDVSX₇IP (SEQ ID NO:292), where X₁ is A or S; X₂ is K or A; X₃ is V, S, R, or T; X₄ is A or G; X₅ is L or M; X₆ is I, L, or V; and X₇ is V or I; and has a length of from 42 amino acids to about 46 amino acids (e.g., has a length of 42 amino acids (aa), 43 aa, 44 aa, 45 aa, or 46 aa), or has a length of from 38 amino acids to about 42 amino acids (e.g., has a length of 38 amino acids (aa), 39 aa, 40 aa, 41 aa, or 42 aa). In some cases, a TP42-2 T-cell epitope polypeptide suitable for inclusion in a composition of the present disclosure comprises the following amino acid sequence:

(SEQ ID NO: 292)
KGGRHLIFCHSKKKCDELAX₁X₂LX₃X₄X₅GX₆NAVAYYRGLDVSX₇IP,

where X₁ is A or S; X₂ is K or A; X₃ is V, S, R, or T; X₄ is A or G; X₅ is L or M; X₆ is I, L, or V; and X₇ is V or I; where the TP42-2 T-cell epitope polypeptide has a length of 42 amino acids.

In some cases, a TP42-2 T-cell epitope polypeptide suitable for inclusion in a composition of the present disclosure comprises the following amino acid sequence:

(SEQ ID NO: 293)
KGGRHLIFCHSKKKCDELAAKLX₁X₂X₃GX₄NAVAYYRGLDVSVIP,

where X₁ is V, S, R, or T; X₂ is A or G; X₃ is L or M; and X₄ is I, L, or V; and has a length of from 42 amino acids to about 46 amino acids (e.g., has a length of 42 amino acids (aa), 43 aa, 44 aa, 45 aa, or 46 aa), or has a length of from 38 amino acids to about 42 amino acids (e.g., has a length of 38 amino acids (aa), 39 aa, 40 aa, 41 aa, or 42 aa). In some cases, a TP42-2 T-cell epitope polypeptide suitable for inclusion in a composition of the present disclosure comprises the following amino acid sequence:

(SEQ ID NO: 293)
KGGRHLIFCHSKKKCDELAAKLX₁X₂X₃GX₄NAVAYYRGLDVSVIP,

where X₁ is V, S, R, or T; X₂ is A or G; X₃ is L or M; and X₄ is I, L, or V; where the TP42-2 T-cell epitope polypeptide has a length of 42 amino acids.

In some cases, a TP42-2 T-cell epitope polypeptide suitable for inclusion in a composition of the present disclosure comprises the following amino acid sequence: KGGRHLIFCHSKKKCDELAAALRGMGXiNAVAYYRGLDVSXIIP (SEQ ID NO:294), where X₁ is I, L, or V; and X₂ is V or I; and has a length of from 42 amino acids to about 46 amino acids (e.g., has a length of 42 amino acids (aa), 43 aa, 44 aa, 45 aa, or 46 aa), or has a length of from 38 amino acids to about 42 amino acids (e.g., has a length of 38 amino acids (aa), 39 aa, 40 aa, 41 aa, or 42 aa). In some cases, a TP42-2 T-cell epitope polypeptide suitable for inclusion in a composition of the present disclosure comprises the following amino acid sequence: HSKKKCDELAAALRGMGXiNAVAYYRGLDVSXIIP (SEQ ID NO:289), where X₁ is I, L, or V; and X₂ is V or I; where the TP42-2 T-cell epitope polypeptide has a length of 42 amino acids.

In some cases, a TP42-2 T-cell epitope polypeptide suitable for inclusion in a composition of the present disclosure comprises the amino acid sequence KGGRHLIFCHSKKKCDELAAKLRGMGLNAVAYYRGLDVSVIP (SEQ ID NO:290); and has a length of 42 amino acids.

### Multiple T-Cell Epitope Polypeptides in a Single Polypeptide Chain

The present disclosure provides a T-cell epitope polypeptide comprising 2, 3, 4, or 5 of the aforementioned T-cell epitope polypeptides in a single polypeptide chain. In other words, the present disclosure provides a fusion polypeptide comprising 2, 3, 4, or 5 of the aforementioned T-cell epitope polypeptides in a single polypeptide chain. Such a fusion polypeptide can be synthesized recombinantly, e.g., where a nucleic acid (e.g., a recombinant expression vector) comprising a nucleotide sequence encoding the fusion polypeptide is introduced into a host cell in vitro, generating a genetically modified host cell; and the host cell synthesizes the encoded fusion polypeptide. Suitable host cells include, e.g., prokaryotic host cells (e.g., E. coli); and eukaryotic host cells (e.g., yeast, such as Saccharomyces cerevisiae, Pichia, and the like; mammalian host cells; and insect cells). The present disclosure further provides a nucleic acid comprising a nucleotide sequence encoding a fusion polypeptide of the present disclosure (e.g., a T-cell epitope polypeptide comprising 2, 3, 4, or 5 of the aforementioned T-cell epitope polypeptides in a single polypeptide chain). The nucleic acid can be present in: i) a recombinant expression vector, for production of the fusion polypeptide, e.g., in a cell in vitro); ii) in an RNA, e.g., for administration to an individual; or iii) in a DNA, e.g., for administration to an individual).

For example, in some cases, an immunogenic composition of the present disclosure comprises: a) i) an HCV E1/E2 heterodimeric polypeptide; ii) an HCV E2 polypeptide; or iii) an HCV E1 polypeptide; b) a T-cell epitope comprising, in order from N-terminus to C-terminus: i) a TP35-NS3 polypeptide, as described above (e.g., a polypeptide having the following amino acid sequence: KSTKVPAAYAAQGYKVLVLNPSVAATLGFGAYMSK (SEQ ID NO:133); and having a length of 35 amino acids); and ii) a TP50C polypeptide, as described above (e.g., a polypeptide having the following amino acid sequence: GVYLLPRRGPRLGVRATRKTSERSQPRGRRQPIPKARRSEGRSWAQPGYP (SEQ ID NO:148); and having a length of 50 amino acids. Thus, e.g., in some cases, an immunogenic composition of the present disclosure comprises: a) i) an HCV E1/E2 heterodimeric polypeptide; ii) an HCV E2 polypeptide; or iii) an HCV E1 polypeptide; b) a T-cell epitope comprising a polypeptide having at least 20% (at least 20%, at least 30%, at least 40%, at least 50%, at least 60%, at least 70%, at least 80%, at least 90%, at least 95%, at least 98%, or 100%) amino acid identity to: KSTKVPAAYAAQGYKVLVLNPSVAATLGFGAYMSKGVYLLPRRGPRLGVRATRKTSE RSQPRGRRQPIPKARRSEGRSWAQPGYP (SEQ ID NO:295) and having a length of 85 amino acids.

As another example, in some cases, an immunogenic composition of the present disclosure comprises: a) i) an HCV E1/E2 heterodimeric polypeptide; ii) an HCV E2 polypeptide; or iii) an HCV E1 polypeptide; b) a T-cell epitope comprising, in order from N-terminus to C-terminus: i) a TP50C polypeptide, as described above (e.g., a polypeptide having the following amino acid sequence: GVYLLPRRGPRLGVRATRKTSERSQPRGRRQPIPKARRSEGRSWAQPGYP (SEQ ID NO:148); and having a length of 50 amino acids; and ii) a TP35-NS3 polypeptide, as described above (e.g., a polypeptide having the following amino acid sequence: KSTKVPAAYAAQGYKVLVLNPSVAATLGFGAYMSK (SEQ ID NO:133); and having a length of 35 amino acids). Thus, e.g., in some cases, an immunogenic composition of the present disclosure comprises: a) i) an HCV E1/E2 heterodimeric polypeptide; ii) an HCV E2 polypeptide; or iii) an HCV E1 polypeptide; b) a T-cell epitope comprising a polypeptide having at least 20% (at least 20%, at least 30%, at least 40%, at least 50%, at least 60%, at least 70%, at least 80%, at least 90%, at least 95%, at least 98%, or 100%) amino acid identity to: GVYLLPRRGPRLGVRATRKTSERSQPRGRRQPIPKARRSEGRSWAQPGYPKSTKVPAAY AAQGYKVLVLNPSVAATLGFGAYMSK (SEQ ID NO:296) and having a length of 85 amino acids.

As another example, in some cases, an immunogenic composition of the present disclosure comprises: a) i) an HCV E1/E2 heterodimeric polypeptide; ii) an HCV E2 polypeptide; or iii) an HCV E1 polypeptide; b) a T-cell epitope comprising, in order from N-terminus to C-terminus: i) a TP35-NS3 polypeptide, as described above (e.g., a polypeptide having the following amino acid sequence: KSTKVPAAYAAQGYKVLVLNPSVAATLGFGAYMSK (SEQ ID NO:133); and having a length of 35 amino acids); ii) a TP50C polypeptide, as described above (e.g., a polypeptide having the following amino acid sequence: GVYLLPRRGPRLGVRATRKTSERSQPRGRRQPIPKARRSEGRSWAQPGYP (SEQ ID NO:148); and having a length of 50 amino acids; and iii) a TP23 polypeptide, as described above (e.g., a polypeptide having the following amino acid sequence: DVVVVATDALMTGFTGDFDSVID (SEQ ID NO:186); and having a length of 23 amino acids). Thus, e.g., in some cases, an immunogenic composition of the present disclosure comprises: a) i) an HCV E1/E2 heterodimeric polypeptide; ii) an HCV E2 polypeptide; or iii) an HCV E1 polypeptide; b) a T-cell epitope comprising a polypeptide having at least 20% (at least 20%, at least 30%, at least 40%, at least 50%, at least 60%, at least 70%, at least 80%, at least 90%, at least 95%, at least 98%, or 100%) amino acid identity to: KSTKVPAAYAAQGYKVLVLNPSVAATLGFGAYMSKGVYLLPRRGPRLGVRATRKTSE RSQPRGRRQPIPKARRSEGRSWAQPGYPDVVVVATDALMTGFTGDFDSVID (SEQ ID NO:295) and having a length of 108 amino acids.

As another example, in some cases, an immunogenic composition of the present disclosure comprises: a) i) an HCV E1/E2 heterodimeric polypeptide; ii) an HCV E2 polypeptide; or iii) an HCV E1 polypeptide; b) a T-cell epitope comprising, in order from N-terminus to C-terminus: i) a TP35-NS3 polypeptide, as described above (e.g., a polypeptide having the following amino acid sequence: KSTKVPAAYAAQGYKVLVLNPSVAATLGFGAYMSK (SEQ ID NO:133); and having a length of 35 amino acids); ii) a TP50C polypeptide, as described above (e.g., a polypeptide having the following amino acid sequence: GVYLLPRRGPRLGVRATRKTSERSQPRGRRQPIPKARRSEGRSWAQPGYP (SEQ ID NO:148); and having a length of 50 amino acids; iii) a TP23 polypeptide, as described above (e.g., a polypeptide having the following amino acid sequence: DVVVVATDALMTGFTGDFDSVID (SEQ ID NO:186); and having a length of 23 amino acids); and iv) a TP27 polypeptide, as described above (e.g., a polypeptide having the following amino acid sequence: LEQIKGGRHLIFCHSKKKCDELAAKLT (SEQ ID NO:188); and having a length of 27 amino acids). Thus, e.g., in some cases, an immunogenic composition of the present disclosure comprises: a) i) an HCV E1/E2 heterodimeric polypeptide; ii) an HCV E2 polypeptide; or iii) an HCV E1 polypeptide; b) a T-cell epitope comprising a polypeptide having at least 20% (at least 20%, at least 30%, at least 40%, at least 50%, at least 60%, at least 70%, at least 80%, at least 90%, at least 95%, at least 98%, or 100%) amino acid identity to: KSTKVPAAYAAQGYKVLVLNPSVAATLGFGAYMSKGVYLLPRRGPRLGVRATRKTSE RSQPRGRRQPIPKARRSEGRSWAQPGYPDVVVVATDALMTGFTGDFDSVIDLEQIKGGR HLIFCHSKKKCDELAAKLT SEQ ID NO:298) and having a length of 135 amino acids.

As another example, in some cases, an immunogenic composition of the present disclosure comprises: a) i) an HCV E1/E2 heterodimeric polypeptide; ii) an HCV E2 polypeptide; or iii) an HCV E1 polypeptide; b) a T-cell epitope comprising, in order from N-terminus to C-terminus: i) a TP35-NS3 polypeptide, as described above (e.g., a polypeptide having the following amino acid sequence: KSTKVPAAYAAQGYKVLVLNPSVAATLGFGAYMSK (SEQ ID NO:133); and having a length of 35 amino acids); ii) a TP50C polypeptide, as described above (e.g., a polypeptide having the following amino acid sequence: GVYLLPRRGPRLGVRATRKTSERSQPRGRRQPIPKARRSEGRSWAQPGYP (SEQ ID NO:148); and having a length of 50 amino acids; iii) a TP23 polypeptide, as described above (e.g., a polypeptide having the following amino acid sequence: DVVVVATDALMTGFTGDFDSVID (SEQ ID NO:186); and having a length of 23 amino acids); iv) a TP27 polypeptide, as described above (e.g., a polypeptide having the following amino acid sequence: LEQIKGGRHLIFCHSKKKCDELAAKLT (SEQ ID NO:188); and having a length of 27 amino acids); and v) a TP35-NS4 polypeptide, as described above (e.g., a polypeptide having the following amino acid sequence: ILRRHVGPGEGAVQWMNRLIAFASRGNHVSPTHYV (SEQ ID NO:203); and having a length of 35 amino acids. Thus, e.g., in some cases, an immunogenic composition of the present disclosure comprises: a) i) an HCV E1/E2 heterodimeric polypeptide; ii) an HCV E2 polypeptide; or iii) an HCV E1 polypeptide; b) a T-cell epitope comprising a polypeptide having at least 20% (at least 20%, at least 30%, at least 40%, at least 50%, at least 60%, at least 70%, at least 80%, at least 90%, at least 95%, at least 98%, or 100%) amino acid identity to: KSTKVPAAYAAQGYKVLVLNPSVAATLGFGAYMSKGVYLLPRRGPRLGVRATRKTSE RSQPRGRRQPIPKARRSEGRSWAQPGYPDVVVVATDALMTGFTGDFDSVIDLEQIKGGR HLIFCHSKKKCDELAAKLTILRRHVGPGEGAVQWMNRLIAFASRGNHVSPTHYV (SEQ ID NO:299) and having a length of 170 amino acids.

As another example, in some cases, an immunogenic composition of the present disclosure comprises: a) i) an HCV E1/E2 heterodimeric polypeptide; ii) an HCV E2 polypeptide; or iii) an HCV E1 polypeptide; b) a T-cell epitope comprising, in order from N-terminus to C-terminus: i) a TP35-NS3 polypeptide, as described above (e.g., a polypeptide having the following amino acid sequence: KSTKVPAAYAAQGYKVLVLNPSVAATLGFGAYMSK (SEQ ID NO:133); and having a length of 35 amino acids); ii) a TP50C polypeptide, as described above (e.g., a polypeptide having the following amino acid sequence: GVYLLPRRGPRLGVRATRKTSERSQPRGRRQPIPKARRSEGRSWAQPGYP (SEQ ID NO:148); and having a length of 50 amino acids; iii) a TP42 polypeptide as described above (e.g., a polypeptide having following amino acid sequence: GTEGEIPFYGKAIPLEQIKGGRHLIFCHSKKKCDELAAKLTG (SEQ ID NO:228); and having a length of 42 amino acids); and iv) a TP48 polypeptide as described above (e.g., a polypeptide having following amino acid sequence: ILRRHVGPGEGAVQWMNRLIAFASRGNHVSPTHYVPESDAAARVTQIL (SEQ ID NO:268); and having a length of 48 amino acids).

As another example, in some cases, an immunogenic composition of the present disclosure comprises: a) i) an HCV E1/E2 heterodimeric polypeptide; ii) an HCV E2 polypeptide; or iii) an HCV E1 polypeptide; b) a T-cell epitope polypeptide comprising, in order from N-terminus to C-terminus: i) a TP35-NS3 polypeptide, as described above (e.g., a polypeptide having the following amino acid sequence: KSTKVPAAYAAQGYKVLVLNPSVAATLGFGAYMSK (SEQ ID NO:133); and having a length of 35 amino acids); ii) a TP50C polypeptide, as described above (e.g., a polypeptide having the following amino acid sequence: GVYLLPRRGPRLGVRATRKTSERSQPRGRRQPIPKARRSEGRSWAQPGYP (SEQ ID NO:148); and having a length of 50 amino acids; iii) a TP42 polypeptide as described above (e.g., a polypeptide having following amino acid sequence: GTEGEIPFYGKAIPLEQIKGGRHLIFCHSKKKCDELAAKLTG (SEQ ID NO:228); and having a length of 42 amino acids); iv) a TP48 polypeptide as described above (e.g., a polypeptide having following amino acid sequence: ILRRHVGPGEGAVQWMNRLIAFASRGNHVSPTHYVPESDAAARVTQIL (SEQ ID NO:268); and having a length of 48 amino acids); and v) a TP42-2 polypeptide, as described above (e.g., a polypeptide having the following amino acid sequence: KGGRHLIFCHSKKKCDELAAKLTGLGLNAVAYYRGLDVSVIP (SEQ ID NO:291); and having a length of 42 amino acids.

### Fusion Polypeptides

In some cases, a T-cell epitope polypeptide suitable for inclusion in an immunogenic composition of the present disclosure is present in a fusion polypeptide comprising: a) the T-cell epitope polypeptide; and b) a heterologous fusion partner polypeptide.

Thus, in some cases, an immunogenic composition of the present disclosure includes: a) i) an HCV E1/E2 heterodimer; ii) an HCV E1 polypeptide; or iii) an HCV E2 polypeptide; and b) a fusion polypeptide comprising: i) T-cell epitope polypeptide that comprises one or more T-cell epitopes (e.g., one or more T cell epitopes present in an HCV polypeptide other than an HCV E1 polypeptide or an HCV E2 polypeptide); and ii) a heterologous fusion partner polypeptide.

Suitable heterologous fusion partner polypeptides include, but are not limited to: i) a polypeptide that increases solubility of the T-cell epitope polypeptide in aqueous solution; ii) a polypeptide that facilitates synthesis and/or purification of the T-cell epitope polypeptide; and iii) a polypeptide that presents one or more additional T-cell epitopes other than HCV T-cell epitopes.

As noted above, in some cases, an immunogenic composition of the present disclosure includes: a) i) an HCV E1/E2 heterodimer; ii) an HCV E1 polypeptide; or iii) an HCV E2 polypeptide; and b) a fusion polypeptide comprising: i) T-cell epitope polypeptide that comprises one or more T-cell epitopes (e.g., one or more T cell epitopes present in an HCV polypeptide other than an HCV E1 polypeptide or an HCV E2 polypeptide); and ii) a heterologous fusion partner polypeptide that presents one or more additional T-cell epitopes. In some cases, the heterologous fusion partner polypeptide comprises one or more T cell epitopes present in: a) cholera toxin or toxoid; and/or b) tetanus toxin or toxoid; and/or c) diphtheria toxin or toxoid; and/or d) a meningococcal outer membrane protein.

In some cases, a suitable tetanus toxoid polypeptide comprises the amino acid sequence QYIKANSKFIGIFE (SEQ ID NO:132). In some cases, a suitable tetanus toxoid polypeptide comprises the amino acid sequence QYIKANSKFIGITE (SEQ ID NO:64).

In some cases, a heterologous fusion partner polypeptide can comprise cholera toxin (or toxoid) epitope. In some cases, a suitable heterologous fusion partner polypeptide comprising a cholera toxoid epitope comprises a fragment of cholera toxin-B subunit (CT-B), e.g., a fragment of from 5 amino acids to 25 amino acids, or from 25 amino acids to 50 amino acids, of the following amino acid sequence: MIKLKFGVFF TVLLSSAYAH GTPQNITDLC AEYHNTQIHT LNDKIFSYTE SLAGKREMAI ITFKNGATFQ VEVPGSQHID SQKKAIERMK DTLRIAYLTE AKVEKLCVWN NKTPHAIAAI SMAN (SEQ ID NO:65).

In some cases, a heterologous fusion partner polypeptide can comprise a tetanus toxin (or toxoid) T-cell epitope. In some cases, a suitable heterologous fusion partner polypeptide comprising a tetanus toxin T-cell epitope comprises the amino acid sequence: ILMQYIKANSKFIGI (SEQ ID NO:66); and has a length of from 15 amino acids to 20 amino acids. In some cases, a suitable heterologous fusion partner polypeptide comprising a tetanus toxin T-cell epitope comprises the amino acid sequence: VNNESSE (SEQ ID NO:67). In some cases, a suitable heterologous fusion partner polypeptide comprising a tetanus toxin T-cell epitope comprises the amino acid sequence: PGINGKAIHLVNNESSE (SEQ ID NO:68). In some cases, a suitable heterologous fusion partner polypeptide comprising a tetanus toxin T-cell epitope comprises the amino acid sequence: PNRDIL (SEQ ID NO:69). In some cases, a suitable heterologous fusion partner polypeptide comprising a tetanus toxin T-cell epitope comprises the amino acid sequence: FIGITEL (SEQ ID NO:70). In some cases, a suitable tetanus toxin T-cell epitope comprises the amino acid sequence: SYFPSV (SEQ ID NO:71). In some cases, a suitable heterologous fusion partner polypeptide comprising a tetanus toxin T-cell epitope comprises the amino acid sequence: NSVDDALINSTKIYSYFPSV (SEQ ID NO:72). In some cases, a suitable heterologous fusion partner polypeptide comprising a tetanus toxin T-cell epitope comprises the amino acid sequence: IDKISDVSTIVPYIGPALNI (SEQ ID NO:73).

In some cases, a heterologous fusion partner polypeptide can comprise a diphtheria toxin T-cell epitope In some cases, a suitable heterologous polypeptide comprising a diphtheria toxin T-cell epitope comprises the amino acid sequence: QSIALSSLMVAQAIP (SEQ ID NO:74); and has a length of from 15 amino acids to 20 amino acids. In some cases, a suitable fusion partner heterologous polypeptide comprising a diphtheria toxin T-cell epitope comprises the amino acid sequence: PVFAGANYAAWAVNVAQVI (SEQ ID NO:75). In some cases, a suitable heterologous fusion partner polypeptide comprising a diphtheria toxin T-cell epitope comprises the amino acid sequence: VHHNTEEIVAQSIALSSLMV (SEQ ID NO:76). In some cases, a suitable heterologous fusion partner polypeptide comprising a diphtheria toxin T-cell epitope comprises the amino acid sequence: QSIALSSLMVAQAIPLVGEL (SEQ ID NO:77). In some cases, a suitable heterologous fusion partner polypeptide comprising a diphtheria toxin T-cell epitope comprises the amino acid sequence: VDIGFAAYNFVESIINLFQV (SEQ ID NO:78). In some cases, a suitable heterologous fusion partner polypeptide comprising a diphtheria toxin T-cell epitope comprises the amino acid sequence: QGESGHDIKITAENTPLPIA (SEQ ID NO:79). In some cases, a suitable heterologous fusion partner polypeptide comprising a diphtheria toxin T-cell epitope comprises the amino acid sequence: GVLLPTIPGKLDVNKSKTHI (SEQ ID NO:80). In some cases, a suitable heterologous fusion partner polypeptide comprising a diphtheria toxin T-cell epitope comprises the amino acid sequence of CRM197 (see, e.g., Giannini et al. (1984) Nucl. Acids. Res. 12:4063).

The amino acid sequence of CRM197 is as follows:

(SEQ ID NO: 81)
ADDVVDSSKSFVMENFSSYHGTKPGYVDSIQKGIQKPKSGTQGNYDDDWK
EFYSTDNKYDAAGYSVDNENPLSGKAGGVVKVTYPGLTKVLALKVDNAET
IKKELGLSLTEPLMEQVGTEEFIKRFGDGASRVVLSLPFAEGSSSVEYIN
NWEQAKALSVELEINFETRGKRGQDAMYEYMAQACAGNRVRRSVGSSLSC
INLDWDVIRDKTKTKIESLKEHGPIKNKMSESPNKTVSEEKAKQYLEEFH
QTALEHPELSELKTVTGTNPVFAGANYAAWAVNVAQVIDSETADNLEKTT
AALSILPGIGSVMGIADGAVHENTEEIVAQSIALSSLMVAQAIPLVGELV
DIGFAAYNFVESIINLFQVVHNSYNRPAYSPGHKTQPFLHDGYAVSWNTV
EDSIIRTGFQGESGHDIKITAENTPLPIAGVLLPTIPGKLDVNKSKTHIS
VNGRKIRMRCRAIDGDVTFCRPKSPVYVGNGVHANLHVAFHRSSSEKIHS
NEISSDSIGVLGYQKTVDHTKVNSKLSLFFEIKS.

In some cases, a heterologous fusion partner polypeptide can comprise a tetanus toxin T-cell epitope and a diphtheria toxin T-cell epitope. In some of these cases, the heterologous fusion partner polypeptide can comprise the amino acid sequence: IMQYIKANSKFIGIQSIALSSLMVAQ (SEQ ID NO:82); and can have a length of from 26 amino acids to 30 amino acids.

In some cases, the fusion partner is a poly(Lys) peptide. The fusion partner can be (Lys)n, where n is an integer from 1 to 10. For example, the fusion partner can be (Lys)n, where n is 1, 2, 3, 4, 5, 6, 7, 8, 9, or 10. For example, the fusion partner can be (Lys)n, where n is 3.

TP35-NS3 with Poly(Lysine)

In some cases, a fusion polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: KSTKVPAAYAAQGYKVLVLNPSVAATLGFGAYMSK(K)n (SEQ ID NO:300), where n is 2, 3, 4, 5, 6, 7, 8, 9, or 10 (e.g., where n is 3, or where n is 5); and has a length of from 37 amino acids to about 45 amino acids. In some cases, a fusion polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: KSTKVPAAYAAQGYKVLVLNPSVAATLGFGAYMSKKKK (SEQ ID NO:302); and has a length of from 38 amino acids to about 43 amino acids, where the C-terminal 3 amino acids are KKK. In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: KSTKVPAAYAAQGYKVLVLNPSVAATLGFGAYMSKKKK (SEQ ID NO:302); and has a length of 38 amino acids, where the C-terminal 3 amino acids are KKK. In some cases, a T-cell epitope polypeptide suitable for inclusion in a composition of the present disclosure comprises the amino acid sequence KSTKVPAAYAAQGYKVLVLNPSVAATLGFGAYMSKKKK (SEQ ID NO:302); and has a length of 38 amino acids.

TP42 with Poly(Lysine)

In some cases, a fusion polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence:

(SEQ ID NO: 301)
GTEGEIPFYGKAIPLEQIKGGRHLIFCHSKKKCDELAAKLTG(K)n,

where n is 2, 3, 4, 5, 6, 7, 8, 9, or 10 (e.g., where n is 3, or where n is 5); and has a length of from 44 amino acids to about 52 amino acids. In some cases, a fusion polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: GTEGEIPFYGKAIPLEQIKGGRHLIFCHSKKKCDELAAKLTGKKK (SEQ ID NO:231); and has a length of from 45 amino acids to about 53 amino acids, where the C-terminal 3 amino acids are KKK. In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: GTEGEIPFYGKAIPLEQIKGGRHLIFCHSKKKCDELAAKLTGKKK (SEQ ID NO:231); and has a length of 45 amino acids, where the C-terminal 3 amino acids are KKK. In some cases, a T-cell epitope polypeptide suitable for inclusion in a composition of the present disclosure comprises the amino acid sequence GTEGEIPFYGKAIPLEQIKGGRHLIFCHSKKKCDELAAKLTGKKK (SEQ ID NO:231); and has a length of 45 amino acids.
TP45 with Poly(Lysine)

In some cases, a fusion polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: LNAVAYYRGLDVSVIPTSGDVVVVATDALMTGFTGDFDSVIDCNV(K)n (SEQ ID NO:304), where n is 2, 3, 4, 5, 6, 7, 8, 9, or 10 (e.g., where n is 3, or where n is 5); and has a length of from 47 amino acids to about 57 amino acids. In some cases, a fusion polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: LNAVAYYRGLDVSVIPTSGDVVVVATDALMTGFTGDFDSVIDCNVKKK (SEQ ID NO:251); and has a length of from 48 amino acids to about 53 amino acids, where the C-terminal 3 amino acids are KKK. In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: LNAVAYYRGLDVSVIPTSGDVVVVATDALMTGFTGDFDSVIDCNVKKK (SEQ ID NO:251); and has a length of 48 amino acids, where the C-terminal 3 amino acids are KKK. In some cases, a T-cell epitope polypeptide suitable for inclusion in a composition of the present disclosure comprises the amino acid sequence LNAVAYYRGLDVSVIPTSGDVVVVATDALMTGFTGDFDSVIDCNVKKK (SEQ ID NO:251); and has a length of 48 amino acids.

In some cases, a fusion polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence:

(SEQ ID NO: 305)
LNAVAYYRGLDVSVIPTSGDVVVVATDALMTGFTGDFDSVID(K)n,

where n is 2, 3, 4, 5, 6, 7, 8, 9, or 10 (e.g., where n is 3, or where n is 5); and has a length of from 45 amino acids to about 55 amino acids. In some cases, a fusion polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: LNAVAYYRGLDVSVIPTSGDVVVVATDALMTGFTGDFDSVIDKKK (SEQ ID NO:248); and has a length of from 45 amino acids to about 55 amino acids, where the C-terminal 3 amino acids are KKK. In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: LNAVAYYRGLDVSVIPTSGDVVVVATDALMTGFTGDFDSVIDKKK (SEQ ID NO:248); and has a length of 45 amino acids, where the C-terminal 3 amino acids are KKK. In some cases, a T-cell epitope polypeptide suitable for inclusion in a composition of the present disclosure comprises the amino acid sequence LNAVAYYRGLDVSVIPTSGDVVVVATDALMTGFTGDFDSVIDCNVKKK (SEQ ID NO:251); and has a length of 48 amino acids.
TP48 with Poly(Lysine)

In some cases, a fusion polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: ILRRHVGPGEGAVQWMNRLIAFASRGNHVSPTHYVPESDASARVTQIL(K)n (SEQ ID NO:306), where n is 2, 3, 4, 5, 6, 7, 8, 9, or 10 (e.g., where n is 3, or where n is 5); and has a length of from 50 amino acids to about 58 amino acids. In some cases, a fusion polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: ILRRHVGPGEGAVQWMNRLIAFASRGNHVSPTHYVPESDASARVTQILKKK (SEQ ID NO:257); and has a length of from 51 amino acids to about 58 amino acids, where the C-terminal 3 amino acids are KKK. In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: ILRRHVGPGEGAVQWMNRLIAFASRGNHVSPTHYVPESDASARVTQILKKK (SEQ ID NO:257); and has a length of 51 amino acids, where the C-terminal 3 amino acids are KKK. In some cases, a T-cell epitope polypeptide suitable for inclusion in a composition of the present disclosure comprises the amino acid sequence ILRRHVGPGEGAVQWMNRLIAFASRGNHVSPTHYVPESDASARVTQILKKK (SEQ ID NO:257); and has a length of 51 amino acids.

TP50-C with Poly(Lysine)

In some cases, a fusion polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: GVYLLPRRGPRLGVRATRKTSERSQPRGRRQPIPKARRSEGRSWAQPGYP(K)n (SEQ ID NO:258), where n is 2, 3, 4, 5, 6, 7, 8, 9, or 10 (e.g., where n is 3, or where n is 5); and has a length of from 52 amino acids to about 62 amino acids. In some cases, a fusion polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: GVYLLPRRGPRLGVRATRKTSERSQPRGRRQPIPKARRSEGRSWAQPGYPKKK (SEQ ID NO:259); and has a length of from 53 amino acids to about 58 amino acids, where the C-terminal 3 amino acids are KKK. In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: GVYLLPRRGPRLGVRATRKTSERSQPRGRRQPIPKARRSEGRSWAQPGYPKKK (SEQ ID NO:259); and has a length of 53 amino acids, where the C-terminal 3 amino acids are KKK. In some cases, a T-cell epitope polypeptide suitable for inclusion in a composition of the present disclosure comprises the amino acid sequence GVYLLPRRGPRLGVRATRKTSERSQPRGRRQPIPKARRSEGRSWAQPGYPKKK (SEQ ID NO:259); and has a length of 53 amino acids.

TP23 with Poly(Lysine)

In some cases, a fusion polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: DVVVVATDALMTGFTGDFDSVID(K)n (SEQ ID NO:260), where n is 2, 3, 4, 5, 6, 7, 8, 9, or 10 (e.g., where n is 3, or where n is 5); and has a length of from 25 amino acids to about 33 amino acids. In some cases, a fusion polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: DVVVVATDALMTGFTGDFDSVIDKKK (SEQ ID NO:261); and has a length of from 26 amino acids to about 30 amino acids, where the C-terminal 3 amino acids are KKK. In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: DVVVVATDALMTGFTGDFDSVIDKKK (SEQ ID NO:261); and has a length of 26 amino acids, where the C-terminal 3 amino acids are KKK. In some cases, a T-cell epitope polypeptide suitable for inclusion in a composition of the present disclosure comprises the amino acid sequence DVVVVATDALMTGFTGDFDSVIDKKK (SEQ ID NO:261); and has a length of 26 amino acids.

TP27 with Poly(Lys)

In some cases, a fusion polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: LEQIKGGRHLIFCHSKKKCDELAAKLT(K)n (SEQ ID NO:262), where n is 2, 3, 4, 5, 6, 7, 8, 9, or 10 (e.g., where n is 3, or where n is 5); and has a length of from 30 amino acids to about 37 amino acids. In some cases, a fusion polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: LEQIKGGRHLIFCHSKKKCDELAAKLTKKK (SEQ ID NO:263); and has a length of from 30 amino acids to about 35 amino acids, where the C-terminal 3 amino acids are KKK. In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: LEQIKGGRHLIFCHSKKKCDELAAKLTKKK (SEQ ID NO:263); and has a length of 30 amino acids, where the C-terminal 3 amino acids are KKK. In some cases, a T-cell epitope polypeptide suitable for inclusion in a composition of the present disclosure comprises the amino acid sequence LEQIKGGRHLIFCHSKKKCDELAAKLTKKK (SEQ ID NO:263); and has a length of 30 amino acids.

TP35-NS4 with Poly(Lys)

In some cases, a fusion polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: ILRRHVGPGEGAVQWMNRLIAFASRGNHVSPTHYV(K)n (SEQ ID NO:264), where n is 2, 3, 4, 5, 6, 7, 8, 9, or 10 (e.g., where n is 3, or where n is 5); and has a length of from 38 amino acids to about 48 amino acids. In some cases, a fusion polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: ILRRHVGPGEGAVQWMNRLIAFASRGNHVSPTHYVKKK (SEQ ID NO:265); and has a length of from 38 amino acids to about 45 amino acids, where the C-terminal 3 amino acids are KKK. In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: ILRRHVGPGEGAVQWMNRLIAFASRGNHVSPTHYVKKK (SEQ ID NO:265); and has a length of 38 amino acids, where the C-terminal 3 amino acids are KKK. In some cases, a T-cell epitope polypeptide suitable for inclusion in a composition of the present disclosure comprises the amino acid sequence ILRRHVGPGEGAVQWMNRLIAFASRGNHVSPTHYVKKK (SEQ ID NO:265); and has a length of 38 amino acids.

### Mixtures of Heterologous Polypeptides (T-Cell Epitope Polypeptides)

In some cases, an immunogenic composition of the present disclosure comprises two or more different T-cell epitope polypeptides comprising a T-cell epitope present in an HCV protein other than E1 and E2 (e.g., a mixture of two or more different heterologous polypeptides comprising a T-cell epitope present in an HCV protein other than E1 and E2). In some cases, an immunogenic composition of the present disclosure comprises two different T-cell epitope polypeptides comprising a T-cell epitope present in an HCV protein other than E1 and E2 (e.g., a mixture of two different heterologous polypeptides comprising a T-cell epitope present in an HCV protein other than E1 and E2). In some cases, an immunogenic composition of the present disclosure comprises three different T-cell epitope polypeptides comprising a T-cell epitope present in an HCV protein other than E1 and E2 (e.g., a mixture of three different heterologous polypeptides comprising a T-cell epitope present in an HCV protein other than E1 and E2). In some cases, an immunogenic composition of the present disclosure comprises four different T-cell epitope polypeptides comprising a T-cell epitope present in an HCV protein other than E1 and E2 (e.g., a mixture of four different heterologous polypeptides comprising a T-cell epitope present in an HCV protein other than E1 and E2). In some cases, an immunogenic composition of the present disclosure comprises five different T-cell epitope polypeptides comprising a T-cell epitope present in an HCV protein other than E1 and E2 (e.g., a mixture of five different heterologous polypeptides comprising a T-cell epitope present in an HCV protein other than E1 and E2). In some cases, an immunogenic composition of the present disclosure comprises six different T-cell epitope polypeptides comprising a T-cell epitope present in an HCV protein other than E1 and E2 (e.g., a mixture of six different heterologous polypeptides comprising a T-cell epitope present in an HCV protein other than E1 and E2). In some cases, the immunogenic composition comprises an adjuvant.

For example, in some cases, an immunogenic composition of the present disclosure comprises: a) an HCV E1/E2 heterodimeric polypeptide; b) two or more different heterologous polypeptides comprising a T-cell epitope present in an HCV protein other than E1 and E2; and c) a pharmaceutically acceptable excipient. In some cases, an immunogenic composition of the present disclosure comprises: a) an HCV E2 polypeptide; b) two or more different heterologous polypeptides comprising a T-cell epitope present in an HCV protein other than E1 and E2; and c) a pharmaceutically acceptable excipient. In some cases, an immunogenic composition of the present disclosure comprises: a) an HCV E1 polypeptide; b) two or more different heterologous polypeptides comprising a T-cell epitope present in an HCV protein other than E1 and E2; and c) a pharmaceutically acceptable excipient. In some cases, at least one of the two or more different heterologous polypeptides comprises a fusion partner. In some cases, at least one of the two or more different heterologous polypeptides comprises a C-terminal poly(Lys), e.g., comprises a stretch of 2, 3, 4, 5, 6, 7, 8, 9, or 10 C-terminal Lys. In some cases, the immunogenic composition comprises an adjuvant.

The following compositions, numbered Compositions 1-38, are non-limiting examples. In some cases, any one of Compositions 1-38 does not include: i) an HCV E1/E2 heterodimeric polypeptide; ii) an HCV E2 polypeptide; or iii) an HCV E1 polypeptide.

Composition 1. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: KSTKVPAAYAAQGYKVLVLNPSVAATLGFGAYMSK (SEQ ID NO:133); c) a second T-cell epitope polypeptide comprising an amino acid sequence having at least 20% amino acid sequence identity to: GVYLLPRRGPRLGVRATRKTSERSQPRGRRQPIPKARRSEGRSWAQPGYP (SEQ ID NO:148); and d) a pharmaceutically acceptable excipient. The first T-cell epitope polypeptide can have a length of 35 amino acids; and the second T-cell epitope polypeptide can have a length of 50 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: KSTKVPAAYAAQGYKVLVLNPSVAATLGFGAYMSK (SEQ ID NO:133) and having a length of 35 amino acids; c) a second T-cell epitope polypeptide comprising the following amino acid sequence: GVYLLPRRGPRLGVRATRKTSERSQPRGRRQPIPKARRSEGRSWAQPGYP (SEQ ID NO:148) and having a length of 50 amino acids; and d) a pharmaceutically acceptable excipient. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide.

Composition 2. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: KSTKVPAAYAAQGYKVLVLNPSVAATLGFGAYMSK (SEQ ID NO:133); c) a second T-cell epitope polypeptide comprising an amino acid sequence having at least 20% amino acid sequence identity to: DVVVVATDALMTGFTGDFDSVID (SEQ ID NO:186); and d) a pharmaceutically acceptable excipient. The first T-cell epitope polypeptide can have a length of 35 amino acids; and the second T-cell epitope polypeptide can have a length of 23 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: KSTKVPAAYAAQGYKVLVLNPSVAATLGFGAYMSK (SEQ ID NO:133) and having a length of 35 amino acids; c) a second T-cell epitope polypeptide comprising the following amino acid sequence: DVVVVATDALMTGFTGDFDSVID (SEQ ID NO:186) and having a length of 23 amino acids; and d) a pharmaceutically acceptable excipient. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide.

Composition 3. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: KSTKVPAAYAAQGYKVLVLNPSVAATLGFGAYMSK (SEQ ID NO:133); c) a second T-cell epitope polypeptide comprising an amino acid sequence having at least 20% amino acid sequence identity to: LEQIKGGRHLIFCHSKKKCDELAAKLT (SEQ ID NO:188); and d) a pharmaceutically acceptable excipient. The first T-cell epitope polypeptide can have a length of 35 amino acids; and the second T-cell epitope polypeptide can have a length of 27 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: KSTKVPAAYAAQGYKVLVLNPSVAATLGFGAYMSK (SEQ ID NO:133) and having a length of 35 amino acids; c) a second T-cell epitope polypeptide comprising the following amino acid sequence: LEQIKGGRHLIFCHSKKKCDELAAKLT (SEQ ID NO:188) and having a length of 27 amino acids; and d) a pharmaceutically acceptable excipient. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide.

Composition 4. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: KSTKVPAAYAAQGYKVLVLNPSVAATLGFGAYMSK (SEQ ID NO:133); c) a second T-cell epitope polypeptide comprising an amino acid sequence having at least 20% amino acid sequence identity to: ILRRHVGPGEGAVQWMNRLIAFASRGNHVSPTHYV (SEQ ID NO:203); and d) a pharmaceutically acceptable excipient. The first T-cell epitope polypeptide can have a length of 35 amino acids; and the second T-cell epitope polypeptide can have a length of 35 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: KSTKVPAAYAAQGYKVLVLNPSVAATLGFGAYMSK (SEQ ID NO:133) and having a length of 35 amino acids; c) a second T-cell epitope polypeptide comprising the following amino acid sequence: ILRRHVGPGEGAVQWMNRLIAFASRGNHVSPTHYV (SEQ ID NO:203) and having a length of 35 amino acids; and d) a pharmaceutically acceptable excipient. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide.

Composition 5. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: GVYLLPRRGPRLGVRATRKTSERSQPRGRRQPIPKARRSEGRSWAQPGYP (SEQ ID NO:148); c) a second T-cell epitope polypeptide comprising an amino acid sequence having at least 20% amino acid sequence identity to: DVVVVATDALMTGFTGDFDSVID (SEQ ID NO:186); and d) a pharmaceutically acceptable excipient. The first T-cell epitope polypeptide can have a length of 50 amino acids; and the second T-cell epitope polypeptide can have a length of 23 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: GVYLLPRRGPRLGVRATRKTSERSQPRGRRQPIPKARRSEGRSWAQPGYP (SEQ ID NO:148) and having a length of 50 amino acids; c) a second T-cell epitope polypeptide comprising the following amino acid sequence: DVVVVATDALMTGFTGDFDSVID (SEQ ID NO:186) and having a length of 23 amino acids; and d) a pharmaceutically acceptable excipient. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide.

Composition 6. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: GVYLLPRRGPRLGVRATRKTSERSQPRGRRQPIPKARRSEGRSWAQPGYP (SEQ ID NO:148); c) a second T-cell epitope polypeptide comprising an amino acid sequence having at least 20% amino acid sequence identity to: LEQIKGGRHLIFCHSKKKCDELAAKLT (SEQ ID NO:188); and d) a pharmaceutically acceptable excipient. The first T-cell epitope polypeptide can have a length of 50 amino acids; and the second T-cell epitope polypeptide can have a length of 27 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: GVYLLPRRGPRLGVRATRKTSERSQPRGRRQPIPKARRSEGRSWAQPGYP (SEQ ID NO:148) and having a length of 50 amino acids; c) a second T-cell epitope polypeptide comprising the following amino acid sequence: LEQIKGGRHLIFCHSKKKCDELAAKLT (SEQ ID NO:188) and having a length of 27 amino acids; and d) a pharmaceutically acceptable excipient. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide.

Composition 7. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: GVYLLPRRGPRLGVRATRKTSERSQPRGRRQPIPKARRSEGRSWAQPGYP (SEQ ID NO:148); c) a second T-cell epitope polypeptide comprising an amino acid sequence having at least 20% amino acid sequence identity to: ILRRHVGPGEGAVQWMNRLIAFASRGNHVSPTHYV (SEQ ID NO:203); and d) a pharmaceutically acceptable excipient. The first T-cell epitope polypeptide can have a length of 50 amino acids; and the second T-cell epitope polypeptide can have a length of 35 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: GVYLLPRRGPRLGVRATRKTSERSQPRGRRQPIPKARRSEGRSWAQPGYP (SEQ ID NO:148) and having a length of 50 amino acids; c) a second T-cell epitope polypeptide comprising the following amino acid sequence: ILRRHVGPGEGAVQWMNRLIAFASRGNHVSPTHYV (SEQ ID NO:203) and having a length of 35 amino acids; and d) a pharmaceutically acceptable excipient. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide.

Composition 8. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: DVVVVATDALMTGFTGDFDSVID (SEQ ID NO:186); c) a second T-cell epitope polypeptide comprising an amino acid sequence having at least 20% amino acid sequence identity to: LEQIKGGRHLIFCHSKKKCDELAAKLT (SEQ ID NO:188); and d) a pharmaceutically acceptable excipient. The first T-cell epitope polypeptide can have a length of 23 amino acids; and the second T-cell epitope polypeptide can have a length of 27 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: DVVVVATDALMTGFTGDFDSVID (SEQ ID NO:186) and having a length of 23 amino acids; c) a second T-cell epitope polypeptide comprising the following amino acid sequence: LEQIKGGRHLIFCHSKKKCDELAAKLT (SEQ ID NO:188) and having a length of 27 amino acids; and d) a pharmaceutically acceptable excipient. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide.

Composition 9. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: DVVVVATDALMTGFTGDFDSVID (SEQ ID NO:186); c) a second T-cell epitope polypeptide comprising an amino acid sequence having at least 20% amino acid sequence identity to: ILRRHVGPGEGAVQWMNRLIAFASRGNHVSPTHYV (SEQ ID NO:203); and d) a pharmaceutically acceptable excipient. The first T-cell epitope polypeptide can have a length of 23 amino acids; and the second T-cell epitope polypeptide can have a length of 35 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: DVVVVATDALMTGFTGDFDSVID (SEQ ID NO:186) and having a length of 23 amino acids; c) a second T-cell epitope polypeptide comprising the following amino acid sequence: ILRRHVGPGEGAVQWMNRLIAFASRGNHVSPTHYV (SEQ ID NO:203) and having a length of 35 amino acids; and d) a pharmaceutically acceptable excipient. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide.

Composition 10. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: LEQIKGGRHLIFCHSKKKCDELAAKLT (SEQ ID NO:188); c) a second T-cell epitope polypeptide comprising an amino acid sequence having at least 20% amino acid sequence identity to: ILRRHVGPGEGAVQWMNRLIAFASRGNHVSPTHYV (SEQ ID NO:203); and d) a pharmaceutically acceptable excipient. The first T-cell epitope polypeptide can have a length of 27 amino acids; and the second T-cell epitope polypeptide can have a length of 35 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: LEQIKGGRHLIFCHSKKKCDELAAKLT (SEQ ID NO:188) and having a length of 27 amino acids; c) a second T-cell epitope polypeptide comprising the following amino acid sequence: ILRRHVGPGEGAVQWMNRLIAFASRGNHVSPTHYV (SEQ ID NO:203) and having a length of 35 amino acids; and d) a pharmaceutically acceptable excipient. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide.

Composition 11. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: KSTKVPAAYAAQGYKVLVLNPSVAATLGFGAYMSK (SEQ ID NO:133); c) a second T-cell epitope polypeptide comprising an amino acid sequence having at least 20% amino acid sequence identity to: GVYLLPRRGPRLGVRATRKTSERSQPRGRRQPIPKARRSEGRSWAQPGYP (SEQ ID NO:148); d) a third T-cell epitope polypeptide comprising an amino acid sequence having at least 20% amino acid sequence identity to: DVVVVATDALMTGFTGDFDSVID (SEQ ID NO:186); and e) a pharmaceutically acceptable excipient. The first T-cell epitope polypeptide can have a length of 35 amino acids; the second T-cell epitope polypeptide can have a length of 50 amino acids; and the third T-cell epitope polypeptide can have a length of 23 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: KSTKVPAAYAAQGYKVLVLNPSVAATLGFGAYMSK (SEQ ID NO:133) and having a length of 35 amino acids; c) a second T-cell epitope polypeptide comprising the following amino acid sequence: GVYLLPRRGPRLGVRATRKTSERSQPRGRRQPIPKARRSEGRSWAQPGYP (SEQ ID NO:148) and having a length of 50 amino acids; d) a third T-cell epitope polypeptide comprising an amino acid sequence having at least 20% amino acid sequence identity to: DVVVVATDALMTGFTGDFDSVID (SEQ ID NO:186) and having a length of 23 amino acids; and e) a pharmaceutically acceptable excipient. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; and d) the third T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; and d) the third T-cell epitope polypeptide.

Composition 12. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: KSTKVPAAYAAQGYKVLVLNPSVAATLGFGAYMSK (SEQ ID NO:133); c) a second T-cell epitope polypeptide comprising an amino acid sequence having at least 20% amino acid sequence identity to: GVYLLPRRGPRLGVRATRKTSERSQPRGRRQPIPKARRSEGRSWAQPGYP (SEQ ID NO:148); d) a third T-cell epitope polypeptide comprising an amino acid sequence having at least 20% amino acid sequence identity to: LEQIKGGRHLIFCHSKKKCDELAAKLT (SEQ ID NO:188); and e) a pharmaceutically acceptable excipient. The first T-cell epitope polypeptide can have a length of 35 amino acids; the second T-cell epitope polypeptide can have a length of 50 amino acids; and the third T-cell epitope polypeptide can have a length of 27 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: KSTKVPAAYAAQGYKVLVLNPSVAATLGFGAYMSK (SEQ ID NO:133) and having a length of 35 amino acids; c) a second T-cell epitope polypeptide comprising the following amino acid sequence: GVYLLPRRGPRLGVRATRKTSERSQPRGRRQPIPKARRSEGRSWAQPGYP (SEQ ID NO:148) and having a length of 50 amino acids; d) a third T-cell epitope polypeptide comprising an amino acid sequence having at least 20% amino acid sequence identity to: LEQIKGGRHLIFCHSKKKCDELAAKLT (SEQ ID NO:188) and having a length of 27 amino acids; and e) a pharmaceutically acceptable excipient. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; and d) the third T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; and d) the third T-cell epitope polypeptide.

Composition 13. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: KSTKVPAAYAAQGYKVLVLNPSVAATLGFGAYMSK (SEQ ID NO:133); c) a second T-cell epitope polypeptide comprising an amino acid sequence having at least 20% amino acid sequence identity to: GVYLLPRRGPRLGVRATRKTSERSQPRGRRQPIPKARRSEGRSWAQPGYP (SEQ ID NO:148); d) a third T-cell epitope polypeptide comprising an amino acid sequence having at least 20% amino acid sequence identity to: ILRRHVGPGEGAVQWMNRLIAFASRGNHVSPTHYV (SEQ ID NO:203); and e) a pharmaceutically acceptable excipient. The first T-cell epitope polypeptide can have a length of 35 amino acids; the second T-cell epitope polypeptide can have a length of 50 amino acids; and the third T-cell epitope polypeptide can have a length of 35 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: KSTKVPAAYAAQGYKVLVLNPSVAATLGFGAYMSK (SEQ ID NO:133) and having a length of 35 amino acids; c) a second T-cell epitope polypeptide comprising the following amino acid sequence: GVYLLPRRGPRLGVRATRKTSERSQPRGRRQPIPKARRSEGRSWAQPGYP (SEQ ID NO:148) and having a length of 50 amino acids; d) a third T-cell epitope polypeptide comprising an amino acid sequence having at least 20% amino acid sequence identity to: ILRRHVGPGEGAVQWMNRLIAFASRGNHVSPTHYV (SEQ ID NO:203) and having a length of 35 amino acids; and e) a pharmaceutically acceptable excipient. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; and d) the third T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; and d) the third T-cell epitope polypeptide.

Composition 14. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: GVYLLPRRGPRLGVRATRKTSERSQPRGRRQPIPKARRSEGRSWAQPGYP (SEQ ID NO:148); c) a second T-cell epitope polypeptide comprising an amino acid sequence having at least 20% amino acid sequence identity to: DVVVVATDALMTGFTGDFDSVID (SEQ ID NO:186); d) a third T-cell epitope polypeptide comprising an amino acid sequence having at least 20% amino acid sequence identity to: LEQIKGGRHLIFCHSKKKCDELAAKLT (SEQ ID NO:188); and e) a pharmaceutically acceptable excipient. The first T-cell epitope polypeptide can have a length of 50 amino acids; the second T-cell epitope polypeptide can have a length of 23 amino acids; and the third T-cell epitope polypeptide can have a length of 27 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: GVYLLPRRGPRLGVRATRKTSERSQPRGRRQPIPKARRSEGRSWAQPGYP (SEQ ID NO:148) and having a length of 50 amino acids; c) a second T-cell epitope polypeptide comprising the following amino acid sequence: DVVVVATDALMTGFTGDFDSVID (SEQ ID NO:186) and having a length of 23 amino acids; d) a third T-cell epitope polypeptide comprising an amino acid sequence having at least 20% amino acid sequence identity to: LEQIKGGRHLIFCHSKKKCDELAAKLT (SEQ ID NO:188) and having a length of 27 amino acids; and e) a pharmaceutically acceptable excipient. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; and d) the third T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; and d) the third T-cell epitope polypeptide.

Composition 15. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: GVYLLPRRGPRLGVRATRKTSERSQPRGRRQPIPKARRSEGRSWAQPGYP (SEQ ID NO:148); c) a second T-cell epitope polypeptide comprising an amino acid sequence having at least 20% amino acid sequence identity to: DVVVVATDALMTGFTGDFDSVID (SEQ ID NO:186); d) a third T-cell epitope polypeptide comprising an amino acid sequence having at least 20% amino acid sequence identity to: ILRRHVGPGEGAVQWMNRLIAFASRGNHVSPTHYV (SEQ ID NO:203); and e) a pharmaceutically acceptable excipient. The first T-cell epitope polypeptide can have a length of 50 amino acids; the second T-cell epitope polypeptide can have a length of 23 amino acids; and the third T-cell epitope polypeptide can have a length of 35 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: GVYLLPRRGPRLGVRATRKTSERSQPRGRRQPIPKARRSEGRSWAQPGYP (SEQ ID NO:148) and having a length of 50 amino acids; c) a second T-cell epitope polypeptide comprising the following amino acid sequence: DVVVVATDALMTGFTGDFDSVID (SEQ ID NO:186) and having a length of 23 amino acids; d) a third T-cell epitope polypeptide comprising an amino acid sequence having at least 20% amino acid sequence identity to: ILRRHVGPGEGAVQWMNRLIAFASRGNHVSPTHYV (SEQ ID NO:203) and having a length of 35 amino acids; and e) a pharmaceutically acceptable excipient. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; and d) the third T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; and d) the third T-cell epitope polypeptide.

Composition 16. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: DVVVVATDALMTGFTGDFDSVID (SEQ ID NO:186); c) a second T-cell epitope polypeptide comprising an amino acid sequence having at least 20% amino acid sequence identity to: LEQIKGGRHLIFCHSKKKCDELAAKLT (SEQ ID NO:188); d) a third T-cell epitope polypeptide comprising an amino acid sequence having at least 20% amino acid sequence identity to: ILRRHVGPGEGAVQWMNRLIAFASRGNHVSPTHYV (SEQ ID NO:203); and e) a pharmaceutically acceptable excipient. The first T-cell epitope polypeptide can have a length of 23 amino acids; the second T-cell epitope polypeptide can have a length of 27 amino acids; and the third T-cell epitope polypeptide can have a length of 35 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: DVVVVATDALMTGFTGDFDSVID (SEQ ID NO:186) and having a length of 23 amino acids; c) a second T-cell epitope polypeptide comprising the following amino acid sequence: LEQIKGGRHLIFCHSKKKCDELAAKLT (SEQ ID NO:188) and having a length of 27 amino acids; d) a third T-cell epitope polypeptide comprising an amino acid sequence having at least 20% amino acid sequence identity to: ILRRHVGPGEGAVQWMNRLIAFASRGNHVSPTHYV (SEQ ID NO:203) and having a length of 35 amino acids; and e) a pharmaceutically acceptable excipient. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; and d) the third T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; and d) the third T-cell epitope polypeptide.

Composition 17. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: KSTKVPAAYAAQGYKVLVLNPSVAATLGFGAYMSK (SEQ ID NO:133); c) a second T-cell epitope polypeptide comprising an amino acid sequence having at least 20% amino acid sequence identity to: GVYLLPRRGPRLGVRATRKTSERSQPRGRRQPIPKARRSEGRSWAQPGYP (SEQ ID NO:148); d) a third T-cell epitope polypeptide comprising an amino acid sequence having at least 20% amino acid sequence identity to: DVVVVATDALMTGFTGDFDSVID (SEQ ID NO:186); e) a fourth T-cell epitope polypeptide comprising an amino acid sequence having at least 20% amino acid sequence identity to: LEQIKGGRHLIFCHSKKKCDELAAKLT (SEQ ID NO:188); and f) a pharmaceutically acceptable excipient. The first T-cell epitope polypeptide can have a length of 35 amino acids; the second T-cell epitope polypeptide can have a length of 50 amino acids; the third T-cell epitope polypeptide can have a length of 23 amino acids; and the fourth T-cell epitope polypeptide can have a length of 27 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: KSTKVPAAYAAQGYKVLVLNPSVAATLGFGAYMSK (SEQ ID NO:133) and having a length of 35 amino acids; c) a second T-cell epitope polypeptide comprising the following amino acid sequence: GVYLLPRRGPRLGVRATRKTSERSQPRGRRQPIPKARRSEGRSWAQPGYP (SEQ ID NO:148) and having a length of 50 amino acids; d) a third T-cell epitope polypeptide comprising an amino acid sequence having at least 20% amino acid sequence identity to: DVVVVATDALMTGFTGDFDSVID (SEQ ID NO:186) and having a length of 23 amino acids; e) a fourth T-cell epitope polypeptide comprising an amino acid sequence having at least 20% amino acid sequence identity to: LEQIKGGRHLIFCHSKKKCDELAAKLT (SEQ ID NO:188) and having a length of 27 amino acids; and f) a pharmaceutically acceptable excipient.. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; d) the third T-cell epitope polypeptide; and e) the fourth T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; d) the third T-cell epitope polypeptide; and e) the fourth T-cell epitope polypeptide.

Composition 18. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: KSTKVPAAYAAQGYKVLVLNPSVAATLGFGAYMSK (SEQ ID NO:133); c) a second T-cell epitope polypeptide comprising an amino acid sequence having at least 20% amino acid sequence identity to: GVYLLPRRGPRLGVRATRKTSERSQPRGRRQPIPKARRSEGRSWAQPGYP (SEQ ID NO:148); d) a third T-cell epitope polypeptide comprising an amino acid sequence having at least 20% amino acid sequence identity to: DVVVVATDALMTGFTGDFDSVID (SEQ ID NO:186); e) a fourth T-cell epitope polypeptide comprising an amino acid sequence having at least 20% amino acid sequence identity to: ILRRHVGPGEGAVQWMNRLIAFASRGNHVSPTHYV (SEQ ID NO:203); and f) a pharmaceutically acceptable excipient. The first T-cell epitope polypeptide can have a length of 35 amino acids; the second T-cell epitope polypeptide can have a length of 50 amino acids; the third T-cell epitope polypeptide can have a length of 23 amino acids; and the fourth T-cell epitope polypeptide can have a length of 35 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: KSTKVPAAYAAQGYKVLVLNPSVAATLGFGAYMSK (SEQ ID NO:133) and having a length of 35 amino acids; c) a second T-cell epitope polypeptide comprising the following amino acid sequence: GVYLLPRRGPRLGVRATRKTSERSQPRGRRQPIPKARRSEGRSWAQPGYP (SEQ ID NO:148) and having a length of 50 amino acids; d) a third T-cell epitope polypeptide comprising an amino acid sequence having at least 20% amino acid sequence identity to: DVVVVATDALMTGFTGDFDSVID (SEQ ID NO:186) and having a length of 23 amino acids; e) a fourth T-cell epitope polypeptide comprising an amino acid sequence having at least 20% amino acid sequence identity to: ILRRHVGPGEGAVQWMNRLIAFASRGNHVSPTHYV (SEQ ID NO:203) and having a length of 35 amino acids; and f) a pharmaceutically acceptable excipient.. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; d) the third T-cell epitope polypeptide; and e) the fourth T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; d) the third T-cell epitope polypeptide; and e) the fourth T-cell epitope polypeptide.

Composition 19. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: KSTKVPAAYAAQGYKVLVLNPSVAATLGFGAYMSK (SEQ ID NO:133); c) a second T-cell epitope polypeptide comprising an amino acid sequence having at least 20% amino acid sequence identity to: GVYLLPRRGPRLGVRATRKTSERSQPRGRRQPIPKARRSEGRSWAQPGYP (SEQ ID NO:148); d) a third T-cell epitope polypeptide comprising an amino acid sequence having at least 20% amino acid sequence identity to: LEQIKGGRHLIFCHSKKKCDELAAKLT (SEQ ID NO:188); e) a fourth T-cell epitope polypeptide comprising an amino acid sequence having at least 20% amino acid sequence identity to: ILRRHVGPGEGAVQWMNRLIAFASRGNHVSPTHYV (SEQ ID NO:203); and f) a pharmaceutically acceptable excipient. The first T-cell epitope polypeptide can have a length of 35 amino acids; the second T-cell epitope polypeptide can have a length of 50 amino acids; the third T-cell epitope polypeptide can have a length of 27 amino acids; and the fourth T-cell epitope polypeptide can have a length of 35 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: KSTKVPAAYAAQGYKVLVLNPSVAATLGFGAYMSK (SEQ ID NO:133) and having a length of 35 amino acids; c) a second T-cell epitope polypeptide comprising the following amino acid sequence: GVYLLPRRGPRLGVRATRKTSERSQPRGRRQPIPKARRSEGRSWAQPGYP (SEQ ID NO:148) and having a length of 50 amino acids; d) a third T-cell epitope polypeptide comprising an amino acid sequence having at least 20% amino acid sequence identity to: LEQIKGGRHLIFCHSKKKCDELAAKLT (SEQ ID NO:188) and having a length of 27 amino acids; e) a fourth T-cell epitope polypeptide comprising an amino acid sequence having at least 20% amino acid sequence identity to: ILRRHVGPGEGAVQWMNRLIAFASRGNHVSPTHYV (SEQ ID NO:203) and having a length of 35 amino acids; and f) a pharmaceutically acceptable excipient.. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; d) the third T-cell epitope polypeptide; and e) the fourth T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; d) the third T-cell epitope polypeptide; and e) the fourth T-cell epitope polypeptide.

Composition 20. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: KSTKVPAAYAAQGYKVLVLNPSVAATLGFGAYMSK (SEQ ID NO:133); c) a second T-cell epitope polypeptide comprising an amino acid sequence having at least 20% amino acid sequence identity to: GVYLLPRRGPRLGVRATRKTSERSQPRGRRQPIPKARRSEGRSWAQPGYP (SEQ ID NO:148); d) a third T-cell epitope polypeptide comprising an amino acid sequence having at least 20% amino acid sequence identity to: DVVVVATDALMTGFTGDFDSVID (SEQ ID NO:186); e) a fourth T-cell epitope polypeptide comprising an amino acid sequence having at least 20% amino acid sequence identity to: LEQIKGGRHLIFCHSKKKCDELAAKLT (SEQ ID NO:188); f) a fifth T-cell epitope polypeptide comprising an amino acid sequence having at least 20% amino acid sequence identity to: ILRRHVGPGEGAVQWMNRLIAFASRGNHVSPTHYV (SEQ ID NO:203); and g) a pharmaceutically acceptable excipient. The first T-cell epitope polypeptide can have a length of 35 amino acids; the second T-cell epitope polypeptide can have a length of 50 amino acids; the third T-cell epitope polypeptide can have a length of 23 amino acids; the fourth T-cell epitope polypeptide can have a length of 27 amino acids; and the fifth T-cell epitope polypeptide can have a length of 35 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: KSTKVPAAYAAQGYKVLVLNPSVAATLGFGAYMSK (SEQ ID NO:133) and having a length of 35 amino acids; c) a second T-cell epitope polypeptide comprising the following amino acid sequence: GVYLLPRRGPRLGVRATRKTSERSQPRGRRQPIPKARRSEGRSWAQPGYP (SEQ ID NO:148) and having a length of 50 amino acids; d) a third T-cell epitope polypeptide comprising an amino acid sequence having at least 20% amino acid sequence identity to: DVVVVATDALMTGFTGDFDSVID (SEQ ID NO:186) and having a length of 23 amino acids; e) a fourth T-cell epitope polypeptide comprising an amino acid sequence having at least 20% amino acid sequence identity to: LEQIKGGRHLIFCHSKKKCDELAAKLT (SEQ ID NO:188) and having a length of 27 amino acids; f) a fifth T-cell epitope polypeptide comprising an amino acid sequence having at least 20% amino acid sequence identity to: ILRRHVGPGEGAVQWMNRLIAFASRGNHVSPTHYV (SEQ ID NO:203) and having a length of 35 amino acids; and g) a pharmaceutically acceptable excipient. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; d) the third T-cell epitope polypeptide; e) the fourth T-cell epitope polypeptide; and f) the fifth T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; d) the third T-cell epitope polypeptide; e) the fourth T-cell epitope polypeptide; and f) the fifth T-cell epitope polypeptide.

Composition 21. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: KSTKVPAAYAAQGYKVLVLNPSVAATLGFGAYMSK (SEQ ID NO:133); c) a second T-cell epitope polypeptide comprising an amino acid sequence having at least 20% amino acid sequence identity to: GTEGEIPFYGKAIPLEQIKGGRHLIFCHSKKKCDELAAKLTG(K)n (SEQ ID NO:301), where n is an integer from 2 to 10 (e.g., where n is 2, 3, 4, 5, 6, 7, 8, 9, or 10); and d) a pharmaceutically acceptable excipient. The first T-cell epitope polypeptide can have a length of 35 amino acids; and the second T-cell epitope polypeptide can have a length of from 44 amino acids to 52 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: KSTKVPAAYAAQGYKVLVLNPSVAATLGFGAYMSK (SEQ ID NO:133) and having a length of 35 amino acids; c) a second T-cell epitope polypeptide comprising the following amino acid sequence: GTEGEIPFYGKAIPLEQIKGGRHLIFCHSKKKCDELAAKLTGKKK (SEQ ID NO:231) and has a length of 45 amino acids; and d) a pharmaceutically acceptable excipient. In some cases, the composition comprises an adjuvant. In some cases, at least one of the T cell epitope polypeptides comprises a fusion partner. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide.

Composition 22. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: KSTKVPAAYAAQGYKVLVLNPSVAATLGFGAYMSK (SEQ ID NO:133); c) a second T-cell epitope polypeptide comprising an amino acid sequence having at least 20% amino acid sequence identity to: LNAVAYYRGLDVSVIPTSGDVVVVATDALMTGFTGDFDSVIDCNV(K)n (SEQ ID NO:304), where n is an integer from 2 to 10 (e.g., where n is 2, 3, 4, 5, 6, 7, 8, 9, or 10); and d) a pharmaceutically acceptable excipient. The first T-cell epitope polypeptide can have a length of 35 amino acids; and the second T-cell epitope polypeptide can have a length of from 47 amino acids to 57 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: KSTKVPAAYAAQGYKVLVLNPSVAATLGFGAYMSK (SEQ ID NO:133) and having a length of 35 amino acids; c) a second T-cell epitope polypeptide comprising the following amino acid sequence: LNAVAYYRGLDVSVIPTSGDVVVVATDALMTGFTGDFDSVIDCNVKKK (SEQ ID NO:251) and having a length of 48 amino acids; and d) a pharmaceutically acceptable excipient. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide.

Composition 23. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: KSTKVPAAYAAQGYKVLVLNPSVAATLGFGAYMSK (SEQ ID NO:133); c) a second T-cell epitope polypeptide comprising an amino acid sequence having at least 20% amino acid sequence identity to: ILRRHVGPGEGAVQWMNRLIAFASRGNHVSPTHYVPESDASARVTQIL(Lys)n (SEQ ID NO:308), where n is an integer from 2 to 10 (e.g., where n is 2, 3, 4, 5, 6, 7, 8, 9, or 10); and d) a pharmaceutically acceptable excipient. The first T-cell epitope polypeptide can have a length of 35 amino acids; and the second T-cell epitope polypeptide an have a length of from 50 amino acids to 58 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: KSTKVPAAYAAQGYKVLVLNPSVAATLGFGAYMSK (SEQ ID NO:133) and having a length of 35 amino acids; c) a second T-cell epitope polypeptide comprising the following amino acid sequence: ILRRHVGPGEGAVQWMNRLIAFASRGNHVSPTHYVPESDASARVTQILKKK (SEQ ID NO:257) and having a length of 51 amino acids; and d) a pharmaceutically acceptable excipient. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide.

Composition 24. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: GTEGEIPFYGKAIPLEQIKGGRHLIFCHSKKKCDELAAKLTG(K)n (SEQ ID NO:301), where n is an integer from 2 to 10 (e.g., where n is 2, 3, 4, 5, 6, 7, 8, 9, or 10); c) a second T-cell epitope polypeptide comprising an amino acid sequence having at least 20% amino acid sequence identity to: LNAVAYYRGLDVSVIPTSGDVVVVATDALMTGFTGDFDSVIDCNV(K)n (SEQ ID NO:304), where n is an integer from 2 to 10 (e.g., where n is 2, 3, 4, 5, 6, 7, 8, 9, or 10); and d) a pharmaceutically acceptable excipient. The first T-cell epitope polypeptide can have a length of from 44 amino acids to 52 amino acids; and the second T-cell epitope polypeptide can have a length of from 47 amino acids to 57 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: GTEGEIPFYGKAIPLEQIKGGRHLIFCHSKKKCDELAAKLTGKKK (SEQ ID NO:231) and having a length of 45 amino acids; c) a second T-cell epitope polypeptide comprising the following amino acid sequence: LNAVAYYRGLDVSVIPTSGDVVVVATDALMTGFTGDFDSVIDCNVKKK (SEQ ID NO:251) and having a length of 48 amino acids; and d) a pharmaceutically acceptable excipient. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide.

Composition 25. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: GTEGEIPFYGKAIPLEQIKGGRHLIFCHSKKKCDELAAKLTG(K)n (SEQ ID NO:301), where n is an integer from 2 to 10 (e.g., where n is 2, 3, 4, 5, 6, 7, 8, 9, or 10); c) a second T-cell epitope polypeptide comprising an amino acid sequence having at least 20% amino acid sequence identity to: ILRRHVGPGEGAVQWMNRLIAFASRGNHVSPTHYVPESDASARVTQIL (SEQ ID NO:254); and d) a pharmaceutically acceptable excipient. The first T-cell epitope polypeptide can have a length of from 44 amino acids to 52 amino acids; and the second T-cell epitope polypeptide can have a length of 48 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: GTEGEIPFYGKAIPLEQIKGGRHLIFCHSKKKCDELAAKLTGKKK (SEQ ID NO:231) and having a length of 45 amino acids; c) a second T-cell epitope polypeptide comprising the following amino acid sequence: ILRRHVGPGEGAVQWMNRLIAFASRGNHVSPTHYVPESDASARVTQIL(Lys)n (SEQ ID NO:308), where n is an integer from 2 to 10 (e.g., where n is 2, 3, 4, 5, 6, 7, 8, 9, or 10); and d) a pharmaceutically acceptable excipient. For example, in some cases, the second T-cell epitope polypeptide comprises the following amino acid sequence: ILRRHVGPGEGAVQWMNRLIAFASRGNHVSPTHYVPESDASARVTQILKKK (SEQ ID NO:257) and has a length of 51 amino acids. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide.

Composition 26. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: GTEGEIPFYGKAIPLEQIKGGRHLIFCHSKKKCDELAAKLTG(K)n (SEQ ID NO:301), where n is an integer from 2 to 10 (e.g., where n is 2, 3, 4, 5, 6, 7, 8, 9, or 10); c) a second T-cell epitope polypeptide comprising an amino acid sequence having at least 20% amino acid sequence identity to: GVYLLPRRGPRLGVRATRKTSERSQPRGRRQPIPKARRSEGRSWAQPGYP (SEQ ID NO:148); and d) a pharmaceutically acceptable excipient. The first T-cell epitope polypeptide can have a length of from 44 amino acids to 52 amino acids; and the second T-cell epitope polypeptide can have a length of 50 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: GTEGEIPFYGKAIPLEQIKGGRHLIFCHSKKKCDELAAKLTGKKK (SEQ ID NO:231) and having a length of 45 amino acids; c) a second T-cell epitope polypeptide comprising the following amino acid sequence: GVYLLPRRGPRLGVRATRKTSERSQPRGRRQPIPKARRSEGRSWAQPGYP (SEQ ID NO:148) and having a length of 50 amino acids; and d) a pharmaceutically acceptable excipient. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide.

Composition 27. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: ILRRHVGPGEGAVQWMNRLIAFASRGNHVSPTHYVPESDASARVTQIL(Lys)n (SEQ ID NO:308), where n is an integer from 2 to 10 (e.g., where n is 2, 3, 4, 5, 6, 7, 8, 9, or 10); c) a second T-cell epitope polypeptide comprising an amino acid sequence having at least 20% amino acid sequence identity to: GVYLLPRRGPRLGVRATRKTSERSQPRGRRQPIPKARRSEGRSWAQPGYP (SEQ ID NO:148); and d) a pharmaceutically acceptable excipient. The first T-cell epitope polypeptide can have a length of from 50 amino acids to 58 amino acids s; and the second T-cell epitope polypeptide can have a length of 50 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: ILRRHVGPGEGAVQWMNRLIAFASRGNHVSPTHYVPESDASARVTQILKKK (SEQ ID NO:257) and having a length of 51 amino acids; c) a second T-cell epitope polypeptide comprising the following amino acid sequence: GVYLLPRRGPRLGVRATRKTSERSQPRGRRQPIPKARRSEGRSWAQPGYP (SEQ ID NO:148) and having a length of 50 amino acids; and d) a pharmaceutically acceptable excipient. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide.

Composition 28. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: KSTKVPAAYAAQGYKVLVLNPSVAATLGFGAYMSKKKK (SEQ ID NO:302); c) a second T-cell epitope polypeptide comprising an amino acid sequence having at least 20% amino acid sequence identity to: GTEGEIPFYGKAIPLEQIKGGRHLIFCHSKKKCDELAAKLTG(K)n (SEQ ID NO:301), where n is an integer from 2 to 10 (e.g., where n is 2, 3, 4, 5, 6, 7, 8, 9, or 10); and d) a pharmaceutically acceptable excipient. The first T-cell epitope polypeptide can have a length of 38 amino acids; and the second T-cell epitope polypeptide can have a length of from 44 amino acids to 52 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: KSTKVPAAYAAQGYKVLVLNPSVAATLGFGAYMSKKKK (SEQ ID NO:302) and having a length of 38 amino acids; c) a second T-cell epitope polypeptide comprising the following amino acid sequence: GTEGEIPFYGKAIPLEQIKGGRHLIFCHSKKKCDELAAKLTGKKK (SEQ ID NO:231) and having a length of 45 amino acids; and d) a pharmaceutically acceptable excipient. In some cases, the composition comprises an adjuvant. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide.

Composition 29. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: KSTKVPAAYAAQGYKVLVLNPSVAATLGFGAYMSKKKK (SEQ ID NO:302); c) a second T-cell epitope polypeptide comprising an amino acid sequence having at least 20% amino acid sequence identity to: LNAVAYYRGLDVSVIPTSGDVVVVATDALMTGFTGDFDSVIDCNVKKK (SEQ ID NO:251); and d) a pharmaceutically acceptable excipient. The first T-cell epitope polypeptide can have a length of 38 amino acids; and the second T-cell epitope polypeptide can have a length of 48 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: KSTKVPAAYAAQGYKVLVLNPSVAATLGFGAYMSKKKK (SEQ ID NO:302) and having a length of 38 amino acids; c) a second T-cell epitope polypeptide comprising the following amino acid sequence: LNAVAYYRGLDVSVIPTSGDVVVVATDALMTGFTGDFDSVIDCNVKKK (SEQ ID NO:251) and having a length of 48 amino acids; and d) a pharmaceutically acceptable excipient. In some cases, the composition comprises an adjuvant. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide.

Composition 30. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: KSTKVPAAYAAQGYKVLVLNPSVAATLGFGAYMSKKKK (SEQ ID NO:302); c) a second T-cell epitope polypeptide comprising an amino acid sequence having at least 20% amino acid sequence identity to: ILRRHVGPGEGAVQWMNRLIAFASRGNHVSPTHYVPESDASARVTQILKKK (SEQ ID NO:257); and d) a pharmaceutically acceptable excipient. The first T-cell epitope polypeptide can have a length of 38 amino acids; and the second T-cell epitope polypeptide can have a length of 51 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: KSTKVPAAYAAQGYKVLVLNPSVAATLGFGAYMSKKKK (SEQ ID NO:302) and having a length of 38 amino acids; c) a second T-cell epitope polypeptide comprising the following amino acid sequence: ILRRHVGPGEGAVQWMNRLIAFASRGNHVSPTHYVPESDASARVTQILKKK (SEQ ID NO:257) and having a length of 51 amino acids; and d) a pharmaceutically acceptable excipient. In some cases, the composition comprises an adjuvant. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide.

Composition 31. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: KSTKVPAAYAAQGYKVLVLNPSVAATLGFGAYMSKKKK (SEQ ID NO:302); c) a second T-cell epitope polypeptide comprising an amino acid sequence having at least 20% amino acid sequence identity to: GVYLLPRRGPRLGVRATRKTSERSQPRGRRQPIPKARRSEGRSWAQPGYP (SEQ ID NO:148); and d) a pharmaceutically acceptable excipient. The first T-cell epitope polypeptide can have a length of 38 amino acids; and the second T-cell epitope polypeptide can have a length of 50 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: KSTKVPAAYAAQGYKVLVLNPSVAATLGFGAYMSKKKK (SEQ ID NO:302) and having a length of 38 amino acids; c) a second T-cell epitope polypeptide comprising the following amino acid sequence: GVYLLPRRGPRLGVRATRKTSERSQPRGRRQPIPKARRSEGRSWAQPGYP (SEQ ID NO:148) and having a length of 50 amino acids; and d) a pharmaceutically acceptable excipient. In some cases, the composition comprises an adjuvant. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; and c) the second T-cell epitope polypeptide.

Composition 32. In some cases, an immunogenic composition of the present disclosure comprises 5 different T-cell epitope polypeptides. Thus, in some cases, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: KSTKVPAAYAAQGYKVLVLNPSVAATLGFGAYMSK (SEQ ID NO:133), and having length of from 35 amino acids to 45 amino acids; c) a second T-cell epitope polypeptide comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: GTEGEIPFYGKAIPLEQIKGGRHLIFCHSKKKCDELAAKLTG(K)n (SEQ ID NO:301), where n is an integer from 2 to 10 (e.g., where n is 2, 3, 4, 5, 6, 7, 8, 9, or 10), and having a length of from 44 amino acids to 52 amino acids; d) a third T-cell epitope polypeptide comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: LNAVAYYRGLDVSVIPTSGDVVVVATDALMTGFTGDFDSVIDCNVKKK (SEQ ID NO:251), and having a length of from 48 amino acids to 55 amino acids; e) a fourth T-cell epitope polypeptide comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: ILRRHVGPGEGAVQWMNRLIAFASRGNHVSPTHYVPESDASARVTQIL(Lys)n (SEQ ID NO:308), where n is an integer from 2 to 10 (e.g., where n is 2, 3, 4, 5, 6, 7, 8, 9, or 10), and having a length of from 50 amino acids to 58 amino acids; f) a fifth T-cell epitope polypeptide comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: GVYLLPRRGPRLGVRATRKTSERSQPRGRRQPIPKARRSEGRSWAQPGYP (SEQ ID NO:148), and having a length of from 50 amino acids to 60 amino acids; and g) a pharmaceutically acceptable excipient. In some cases, the composition comprises an adjuvant. For example, in some cases, the fourth T-cell epitope polypeptide comprises the following amino acid sequence: ILRRHVGPGEGAVQWMNRLIAFASRGNHVSPTHYVPESDASARVTQILKKK (SEQ ID NO:257) and has a length of 51 amino acids. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; d) the third T-cell epitope polypeptide; e) the fourth T-cell epitope polypeptide; and f) the fifth T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; d) the third T-cell epitope polypeptide; e) the fourth T-cell epitope polypeptide; and f) the fifth T-cell epitope polypeptide.

Composition 33. In some cases, an immunogenic composition of the present disclosure comprises 5 different T-cell epitope polypeptides. Thus, in some cases, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: KSTKVPAAYAAQGYKVLVLNPSVAATLGFGAYMSKKKK (SEQ ID NO:302), and having a length of from 38 amino acids to 45 amino acids; c) a second T-cell epitope polypeptide comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: GTEGEIPFYGKAIPLEQIKGGRHLIFCHSKKKCDELAAKLTG(K)n (SEQ ID NO:301), where n is an integer from 2 to 10 (e.g., where n is 2, 3, 4, 5, 6, 7, 8, 9, or 10), and having a length of from 44 amino acids to 52 amino acids; d) a third T-cell epitope polypeptide comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: LNAVAYYRGLDVSVIPTSGDVVVVATDALMTGFTGDFDSVIDCNVKKK (SEQ ID NO:251), and having a length of from 48 amino acids to 55 amino acids; e) a fourth T-cell epitope polypeptide comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: ILRRHVGPGEGAVQWMNRLIAFASRGNHVSPTHYVPESDASARVTQILKKK (SEQ ID NO:257), and having a length of from 51 amino acids to 58 amino acids; f) a fifth T-cell epitope polypeptide comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: GVYLLPRRGPRLGVRATRKTSERSQPRGRRQPIPKARRSEGRSWAQPGYP (SEQ ID NO:148), and having a length of from 50 amino acids to 60 amino acids; and g) a pharmaceutically acceptable excipient. In some cases, the composition comprises an adjuvant. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; d) the third T-cell epitope polypeptide; e) the fourth T-cell epitope polypeptide; and f) the fifth T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; d) the third T-cell epitope polypeptide; e) the fourth T-cell epitope polypeptide; and f) the fifth T-cell epitope polypeptide.

Composition 34. In some cases, an immunogenic composition of the present disclosure comprises 5 different T-cell epitope polypeptides. Thus, in some cases, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: KSTKVPAAYAAQGYKVLVLNPSVAATLGFGAYMSK (SEQ ID NO:133), and having length of from 35 amino acids to 45 amino acids; c) a second T-cell epitope polypeptide comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to:

(SEQ ID NO: 301)
GTEGEIPFYGKAIPLEQIKGGRHLIFCHSKKKCDELAAKLTG(K)n,

where n is an integer from 2 to 10 (e.g., where n is 2, 3, 4, 5, 6, 7, 8, 9, or 10), and having a length of from 44 amino acids to 52 amino acids; d) a third T-cell epitope polypeptide comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: LNAVAYYRGLDVSVIPTSGDVVVVATDALMTGFTGDFDSVID (SEQ ID NO:253), and having a length of from 42 amino acids to 52 amino acids; e) a fourth T-cell epitope polypeptide comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: ILRRHVGPGEGAVQWMNRLIAFASRGNHVSPTHYVPESDASARVTQIL(Lys)n (SEQ ID NO:308), where n is an integer from 2 to 10 (e.g., where n is 2, 3, 4, 5, 6, 7, 8, 9, or 10), and having a length of from 50 amino acids to 58 amino acids; f) a fifth T-cell epitope polypeptide comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: GVYLLPRRGPRLGVRATRKTSERSQPRGRRQPIPKARRSEGRSWAQPGYP (SEQ ID NO:148), and having a length of from 50 amino acids to 60 amino acids; and g) a pharmaceutically acceptable excipient. For example, in some cases, the fourth T-cell epitope polypeptide comprises the following amino acid sequence: ILRRHVGPGEGAVQWMNRLIAFASRGNHVSPTHYVPESDASARVTQILKKK (SEQ ID NO:257) and has a length of 51 amino acids. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; d) the third T-cell epitope polypeptide; e) the fourth T-cell epitope polypeptide; and f) the fifth T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; d) the third T-cell epitope polypeptide; e) the fourth T-cell epitope polypeptide; and f) the fifth T-cell epitope polypeptide.

Composition 35. In some cases, an immunogenic composition of the present disclosure comprises 4 different T-cell epitope polypeptides. Thus, in some cases, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: KSTKVPAAYAAQGYKVLVLNPSVAATLGFGAYMSK (SEQ ID NO:133), and having length of from 35 amino acids to 45 amino acids; c) a second T-cell epitope polypeptide comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: GTEGEIPFYGKAIPLEQIKGGRHLIFCHSKKKCDELAAKLTG(K)n (SEQ ID NO:301), where n is an integer from 2 to 10 (e.g., where n is 2, 3, 4, 5, 6, 7, 8, 9, or 10), and having a length of from 44 amino acids to 52 amino acids; d) a third T-cell epitope polypeptide comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: ILRRHVGPGEGAVQWMNRLIAFASRGNHVSPTHYVPESDASARVTQIL(Lys)n (SEQ ID NO:308), where n is an integer from 2 to 10 (e.g., where n is 2, 3, 4, 5, 6, 7, 8, 9, or 10), and having a length of from 50 amino acids to 58 amino acids; e) a fourth T-cell epitope polypeptide comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: GVYLLPRRGPRLGVRATRKTSERSQPRGRRQPIPKARRSEGRSWAQPGYP (SEQ ID NO:148), and having a length of from 50 amino acids to 60 amino acids; and g) a pharmaceutically acceptable excipient. For example, in some cases, the third T-cell epitope polypeptide comprises the following amino acid sequence: ILRRHVGPGEGAVQWMNRLIAFASRGNHVSPTHYVPESDASARVTQILKKK (SEQ ID NO:257) and has a length of 51 amino acids. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; d) the third T-cell epitope polypeptide; and e) the fourth T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; d) the third T-cell epitope polypeptide; and e) the fourth T-cell epitope polypeptide.

Composition 36. In some cases, an immunogenic composition of the present disclosure comprises 4 different T-cell epitope polypeptides. Thus, in some cases, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: KSTKVPAAYAAQGYKVLVLNPSVAATLGFGAYMSK(K)n (SEQ ID NO:300), where n is an integer from 2 to 10 (e.g., where n is 2, 3, 4, 5, 6, 7, 8, 9, or 10) and having length of from 37 amino acids to 45 amino acids; c) a second T-cell epitope polypeptide comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: GTEGEIPFYGKAIPLEQIKGGRHLIFCHSKKKCDELAAKLTG(K)n (SEQ ID NO:301), where n is an integer from 2 to 10 (e.g., where n is 2, 3, 4, 5, 6, 7, 8, 9, or 10), and having a length of from 44 amino acids to 52 amino acids; d) a third T-cell epitope polypeptide comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: ILRRHVGPGEGAVQWMNRLIAFASRGNHVSPTHYVPESDASARVTQILKKK (SEQ ID NO:257), and having a length of from 51 amino acids to 58 amino acids; e) a fourth T-cell epitope polypeptide comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: GVYLLPRRGPRLGVRATRKTSERSQPRGRRQPIPKARRSEGRSWAQPGYP (SEQ ID NO:148), and having a length of from 50 amino acids to 60 amino acids; and g) a pharmaceutically acceptable excipient. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; d) the third T-cell epitope polypeptide; and e) the fourth T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; d) the third T-cell epitope polypeptide; and e) the fourth T-cell epitope polypeptide.

Composition 37. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 80% amino acid sequence identity to: KSTKVPX₁AYX₂X₃QGYX₄VLVLNPSVAATLGFGX₅X₆X₇SX₈ (SEQ ID NO:134), wherein X₁ is A or V; X₂ is A or V; X₃ is A or S; X₄ is K or N; X₅ is A or S; X₆ is Y or F; X₇ is M or L; and X₈ is K or R, where the TP35-NS3 T-cell epitope polypeptide has a length of from 28 amino acids to 35 amino acids; c) a second T-cell epitope polypeptide comprising an amino acid sequence having at least 80% amino acid sequence identity to: GVYX₁LPRRGPRLGVRX₂TRKX₃SERSQPRGRRQX₄IPKX₅X₆X₇X₈X₉GX₁₀X₁₁WX₁₂X₁₃PGY P (SEQ ID NO:149), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁₁ is S, T, H, or A; X₁₂ is A or G; and X₁₃ is Q or K, where the TP50C T-cell epitope polypeptide has a length of from 40 amino acids to 50 amino acids; d) a third T-cell epitope polypeptide comprising an amino acid sequence having at least 80% amino acid sequence identity to: X₁X₂X₃RHX₄GX₅X₆EGX₇X₈QWMNRLIAFASRGNHVXPTHYX X₁₁X₁₂X₁₃DAX₁₄X₁₅X₁₆VX₁₇X₁₈X₁₉L(K)n (SEQ ID NO:255), where X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, where n is an integer from 2 to 10, and wherein the TP48 T-cell epitope polypeptide has a length of from 38 amino acids to 48 amino acids; and e) a pharmaceutically acceptable excipient. For example, in some cases, the immunogenic composition comprises: a) i) an HCV E1/E2 heterodimeric polypeptide; ii) an HCV E2 polypeptide; or iii) an HCV E1 polypeptide; b) a first T-cell epitope polypeptide comprising the amino acid sequence: KSTKVPAAYAAQGYKVLVLNPSVAATLGFGAYMSK (SEQ ID NO:133), and having a length of 35 amino acids; c) a second T-cell epitope polypeptide comprising the amino acid sequence: GVYLLPRRGPRLGVRATRKTSERSQPRGRRQPIPKARRSEGRSWAQPGYP (SEQ ID NO:148), and having a length of 50 amino acids; d) a third T-cell epitope polypeptide comprising the amino acid sequence: ILRRHVGPGEGAVQWMNRLIAFASRGNHVSPTHYVPESDASARVTQILKKK (SEQ ID NO:257) and having a length of 51 amino acids; and e) a pharmaceutically acceptable excipient. In some cases, the composition comprises an adjuvant. In some cases, at least one of the T cell epitope polypeptides comprises a fusion partner. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; and d) the third T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; d) the third T-cell epitope polypeptide; and e) the fourth T-cell epitope polypeptide.

Composition 38. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: KSTKVPAAYAAQGYKVLVLNPSVAATLGFGAYMSK (SEQ ID NO:133); c) a second T-cell epitope polypeptide comprising an amino acid sequence having at least 20% amino acid sequence identity to: GVYLLPRRGPRLGVRATRKTSERSQPRGRRQPIPKARRSEGRSWAQPGYP (SEQ ID NO:148); d) a third T-cell epitope polypeptide comprising an amino acid sequence having at least 20% amino acid sequence identity to: GTEGEIPFYGKAIPLEQIKGGRHLIFCHSKKKCDELAAKLTG (SEQ ID NO:228); e) a fourth T-cell epitope polypeptide comprising an amino acid sequence having at least 20% amino acid sequence identity to: KGGRHLIFCHSKKKCDELAAKLTGLGLNAVAYYRGLDVSVIP (SEQ ID NO:291); f) a fifth T-cell epitope polypeptide comprising an amino acid sequence having at least 20% amino acid sequence identity to: ILRRHVGPGEGAVQWMNRLIAFASRGNHVSPTHYVPESDASARVTQIL (SEQ ID NO:254); and g) a pharmaceutically acceptable excipient. The first T-cell epitope polypeptide can have a length of 35 amino acids; the second T-cell epitope polypeptide can have a length of 50 amino acids; the third T-cell epitope polypeptide can have a length of 42 amino acids; the fourth T-cell epitope polypeptide can have a length of 42 amino acids; and the fifth T-cell epitope polypeptide can have a length of 48 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: KSTKVPAAYAAQGYKVLVLNPSVAATLGFGAYMSK (SEQ ID NO:133) and having a length of 35 amino acids; c) a second T-cell epitope polypeptide comprising the following amino acid sequence: GVYLLPRRGPRLGVRATRKTSERSQPRGRRQPIPKARRSEGRSWAQPGYP (SEQ ID NO:148) and having a length of 50 amino acids; d) a third T-cell epitope polypeptide comprising an amino acid sequence having at least 20% amino acid sequence identity to: GTEGEIPFYGKAIPLEQIKGGRHLIFCHSKKKCDELAAKLTG (SEQ ID NO:228) and having a length of 42 amino acids; e) a fourth T-cell epitope polypeptide comprising an amino acid sequence having at least 20% amino acid sequence identity to: KGGRHLIFCHSKKKCDELAAKLTGLGLNAVAYYRGLDVSVIP (SEQ ID NO:291) and having a length of 42 amino acids; f) a fifth T-cell epitope polypeptide comprising an amino acid sequence having at least 20% amino acid sequence identity to: ILRRHVGPGEGAVQWMNRLIAFASRGNHVSPTHYVPESDASARVTQIL (SEQ ID NO:254) and having a length of 48 amino acids; and g) a pharmaceutically acceptable excipient. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; d) the third T-cell epitope polypeptide; e) the fourth T-cell epitope polypeptide; and f) the fifth T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; d) the third T-cell epitope polypeptide; e) the fourth T-cell epitope polypeptide; and f) the fifth T-cell epitope polypeptide.

Composition 39. As one example, an immunogenic composition of the present disclosure comprises: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: KSTKVPAAYAAQGYKVLVLNPSVAATLGFGAYMSK (SEQ ID NO:133); c) a second T-cell epitope polypeptide comprising an amino acid sequence having at least 20% amino acid sequence identity to: GVYLLPRRGPRLGVRATRKTSERSQPRGRRQPIPKARRSEGRSWAQPGYP (SEQ ID NO:148); d) a third T-cell epitope polypeptide comprising an amino acid sequence having at least 20% amino acid sequence identity to: GTEGEIPFYGKAIPLEQIKGGRHLIFCHSKKKCDELAAKLTG (SEQ ID NO:228); and e) a pharmaceutically acceptable excipient. The first T-cell epitope polypeptide can have a length of 35 amino acids; the second T-cell epitope polypeptide can have a length of 50 amino acids; and the third T-cell epitope polypeptide can have a length of 42 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first T-cell epitope polypeptide comprising the following amino acid sequence: KSTKVPAAYAAQGYKVLVLNPSVAATLGFGAYMSK (SEQ ID NO:133) and having a length of 35 amino acids; c) a second T-cell epitope polypeptide comprising the following amino acid sequence: GVYLLPRRGPRLGVRATRKTSERSQPRGRRQPIPKARRSEGRSWAQPGYP (SEQ ID NO:148) and having a length of 50 amino acids; d) a third T-cell epitope polypeptide comprising an amino acid sequence having at least 20% amino acid sequence identity to: GTEGEIPFYGKAIPLEQIKGGRHLIFCHSKKKCDELAAKLTG (SEQ ID NO:228) and having a length of 42 amino acids; and e) a pharmaceutically acceptable excipient. In some cases, the composition comprises an adjuvant. In some cases, at least one of the T cell epitope polypeptides comprises a fusion partner. In some cases, at least one of the T cell epitope polypeptides comprises a C-terminal poly(Lys), e.g., comprises a stretch of 2, 3, 4, 5, 6, 7, 8, 9, or 10 C-terminal Lys. In some cases, the immunogenic composition does not include any HCV polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; and d) the third T-cell epitope polypeptide. In some cases, the immunogenic composition does not include any polypeptides other than: a) i) an HCV E1/E2 heterodimeric polypeptide; ii) an HCV E2 polypeptide; or iii) an HCV E1 polypeptide; b) the first T-cell epitope polypeptide; c) the second T-cell epitope polypeptide; and d) the third T-cell epitope polypeptide.

### HCV E1/E2 Heterodimers; HCV E2 Polypeptides; HCV E1 Polypeptides

HCV E1/E2 heterodimers suitable for use in an immunogenic composition of the present disclosure include HCV E1/E2 heterodimers comprising wild-type HCV E1 polypeptides; HCV E1/E2 heterodimers comprising wild-type HCV E2 polypeptides; HCV E1/E2 heterodimers comprising variant HCV E1 polypeptides; and HCV E1/E2 heterodimers comprising variant HCV E2 polypeptides. HCV E2 polypeptides suitable for use in an immunogenic composition of the present disclosure include wild-type E2 polypeptides and variant E2 polypeptides. HCV E1 polypeptides suitable for use in an immunogenic composition of the present disclosure include wild-type E1 polypeptides and variant E1 polypeptides.

### E2 Polypeptides

An E2 polypeptide suitable for inclusion in an E1/E2 heterodimer for inclusion in an immunogenic composition of the present disclosure, or for inclusion by itself in an immunogenic composition of the present disclosure, can have a length of from about 200 amino acids (aa) to about 250 aa, from about 250 aa to about 275 aa, from about 275 aa to about 300 aa, from about 300 aa to about 325 aa, from about 325 aa to about 350 aa, or from about 350 aa to about 365 aa. In some cases, an HCV E2 polypeptide suitable for inclusion in an immunogenic composition of the present disclosure is an HCV E2 ectodomain polypeptide. In some cases, an HCV E2 polypeptide suitable for inclusion in an immunogenic composition of the present disclosure is a full-length HCV E2 polypeptide.

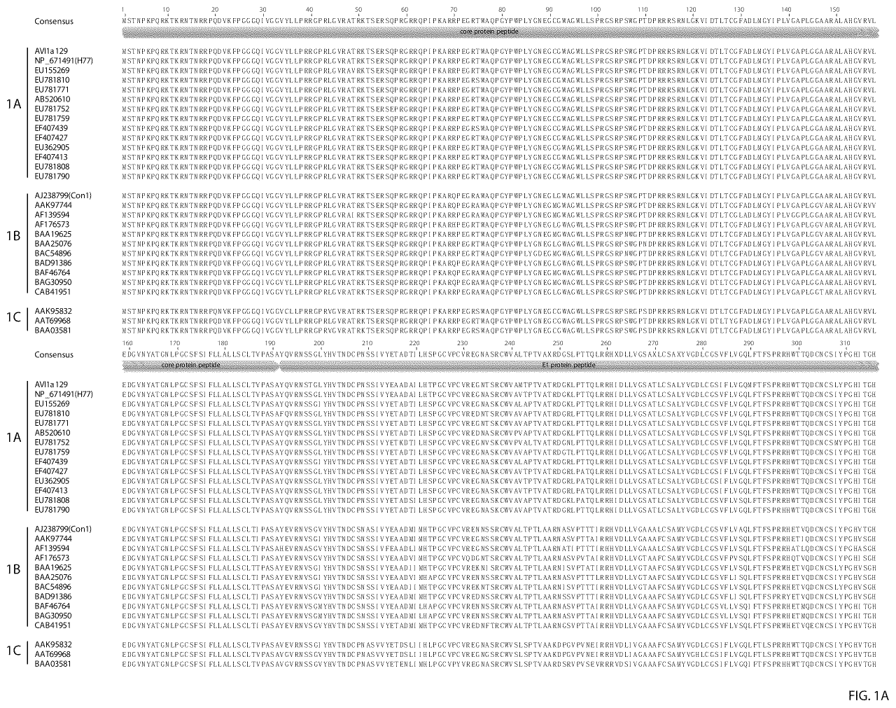

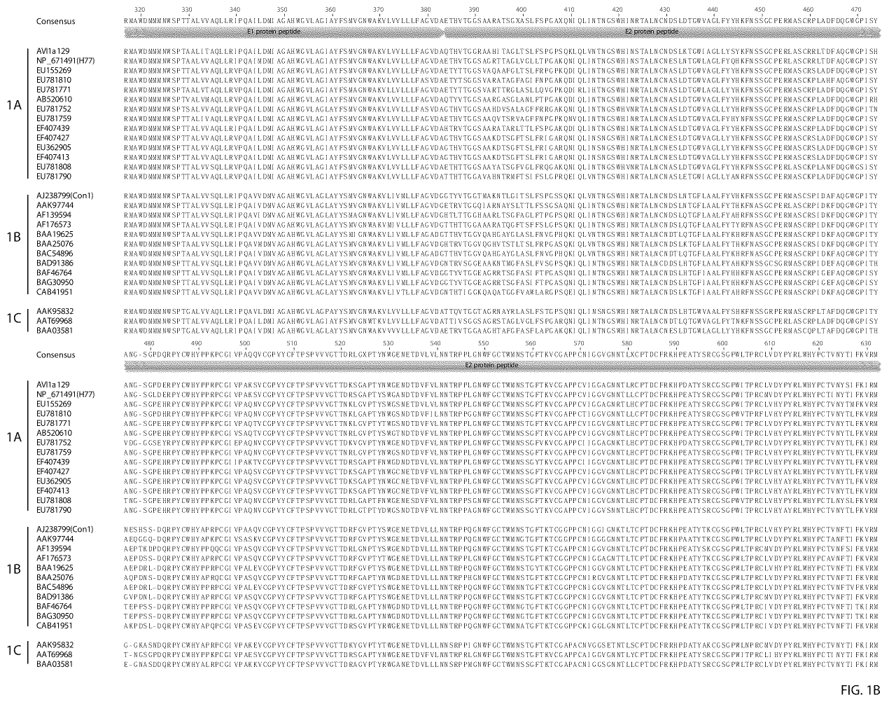

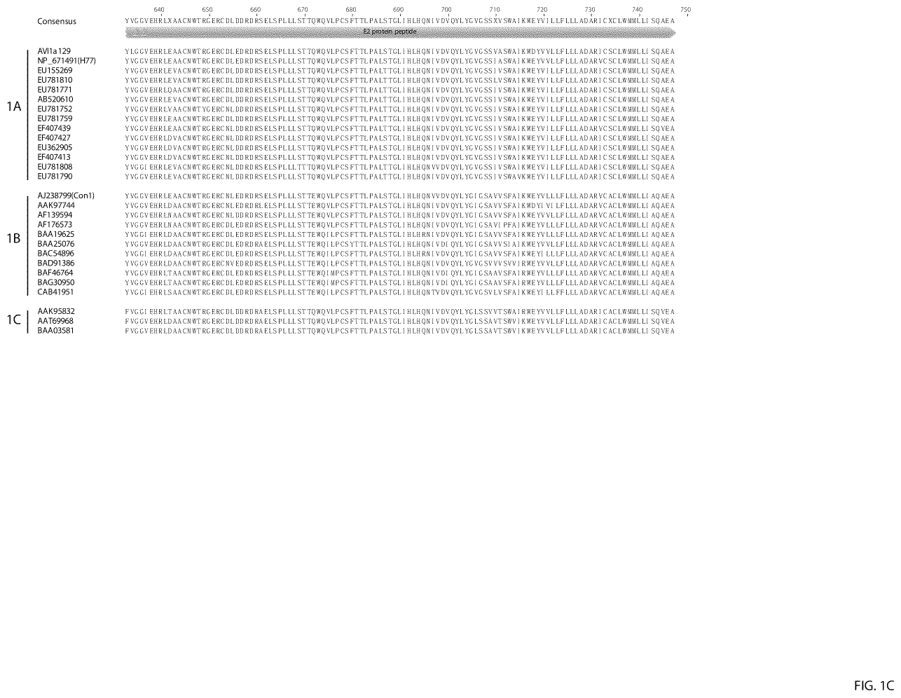

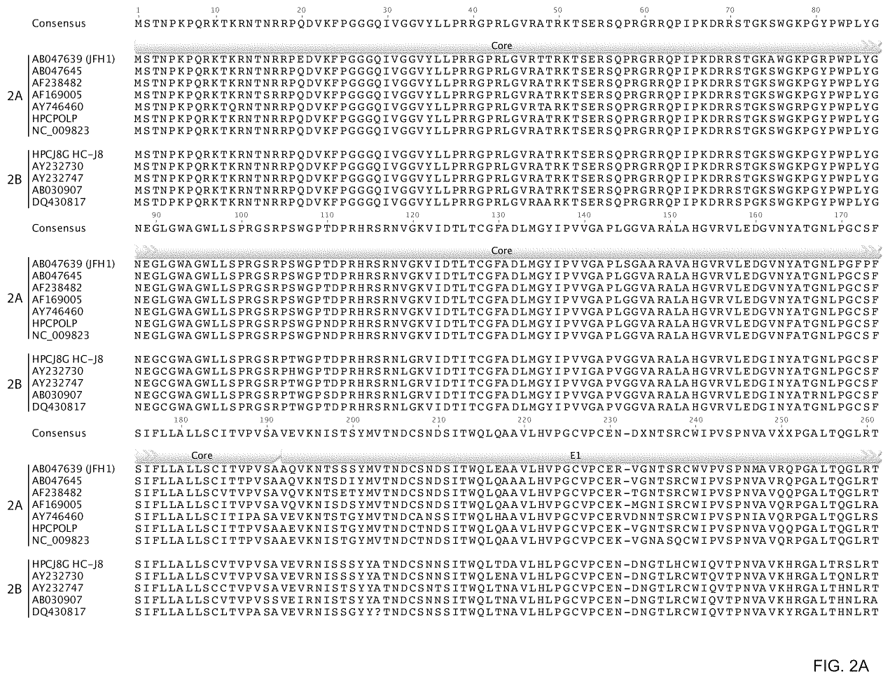

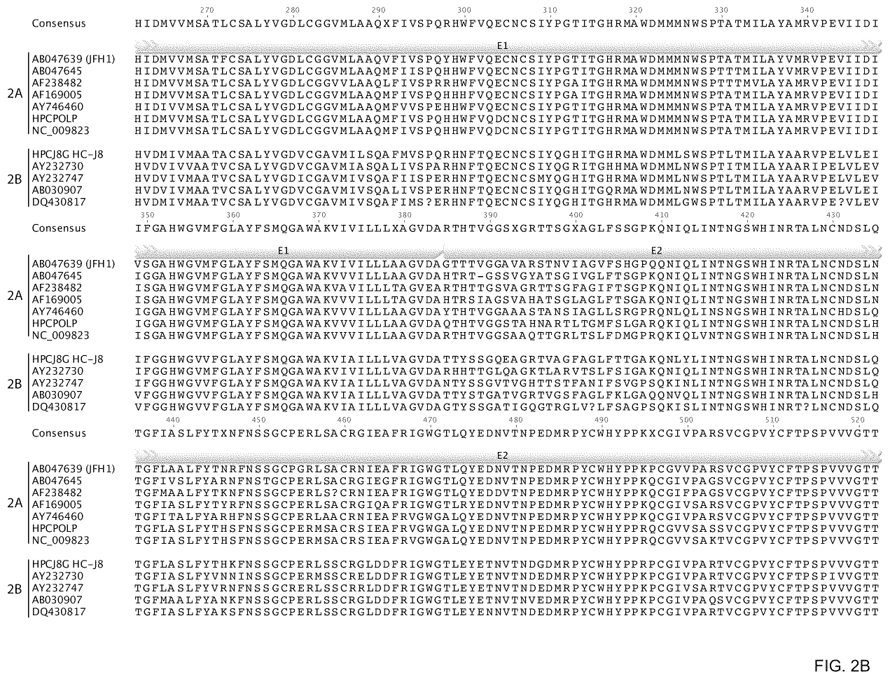

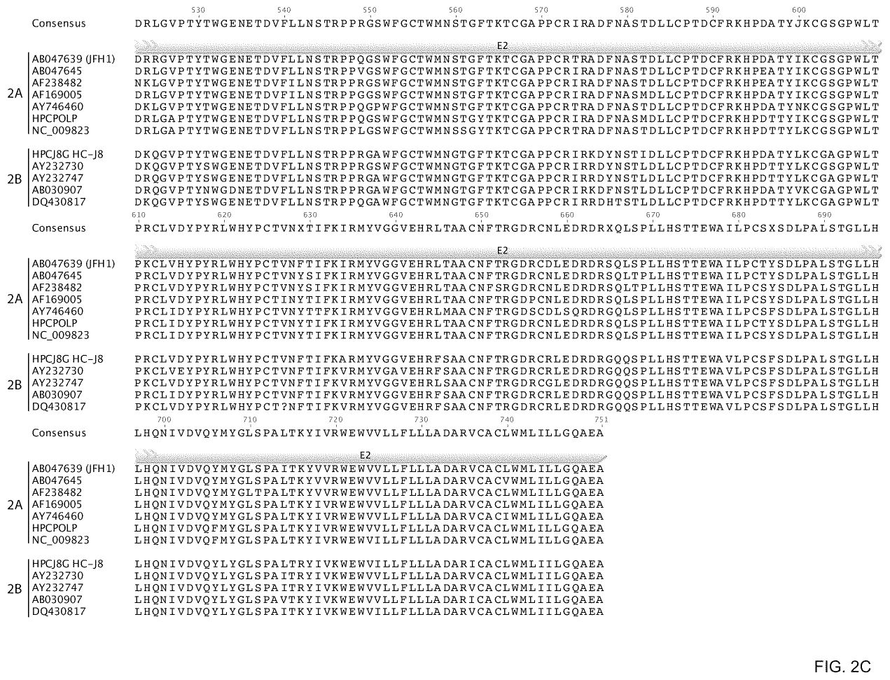

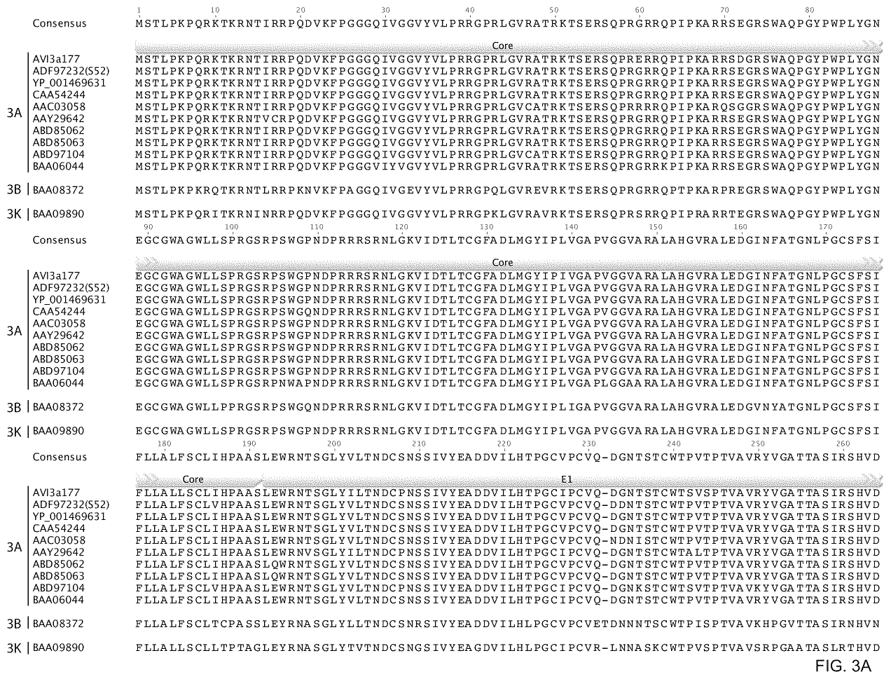

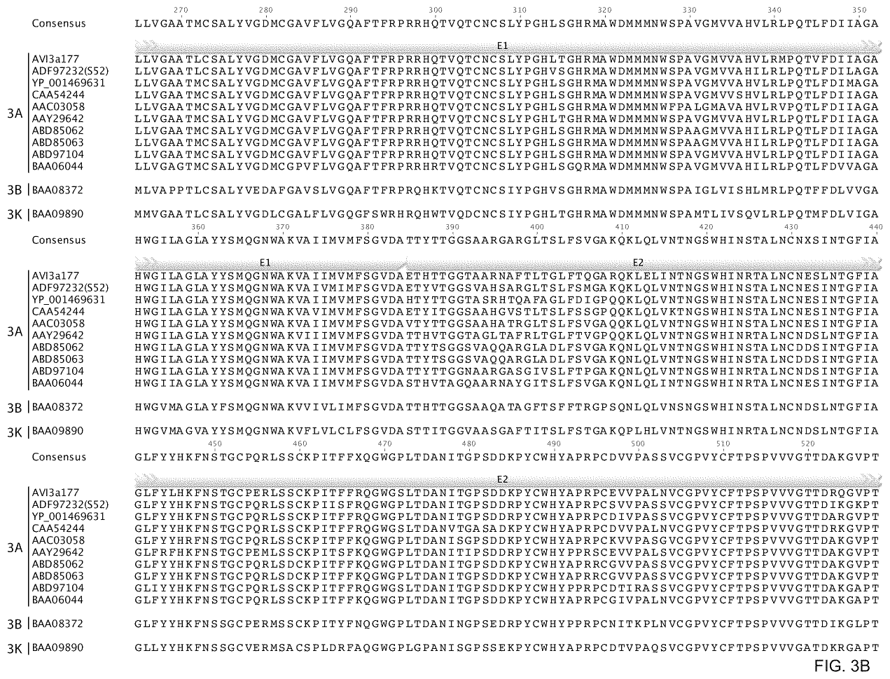

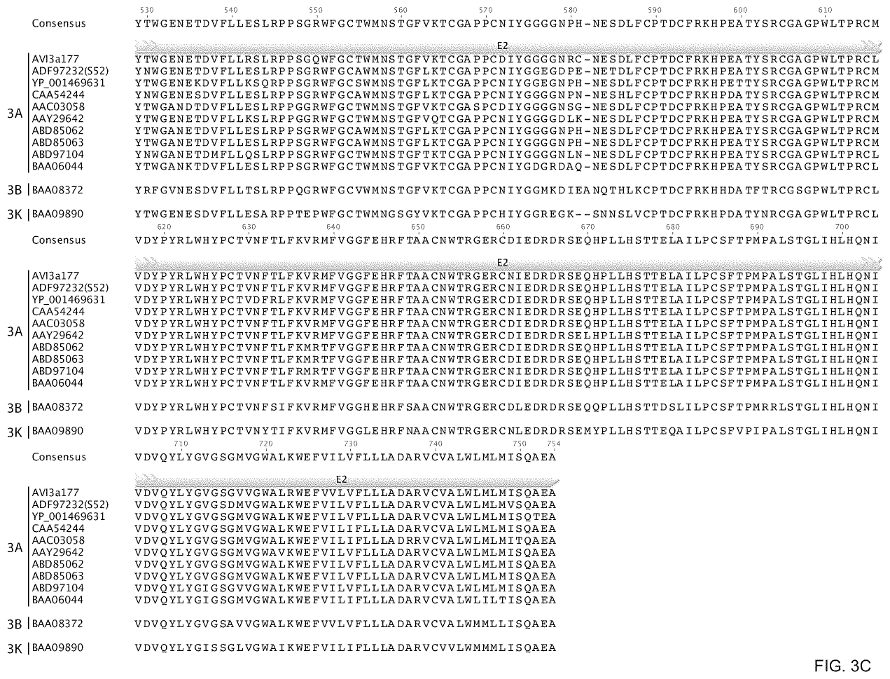

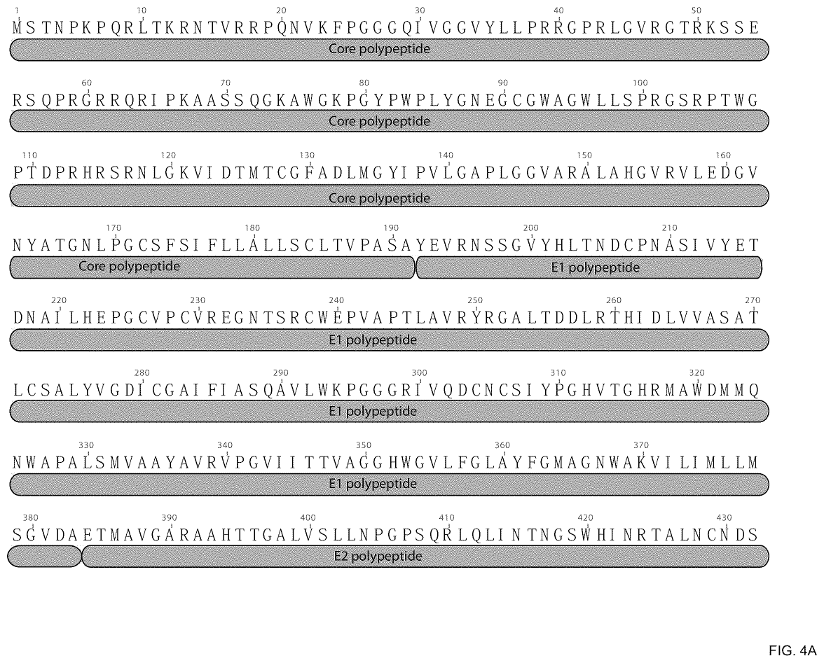

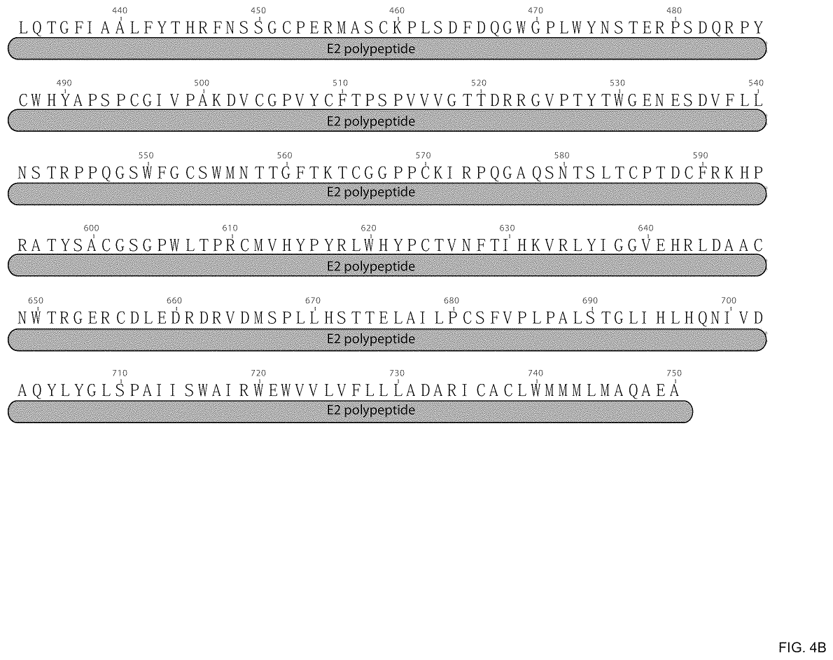

In FIG. 1A-AC, the amino acid sequence of E2 is amino acid 384 to amino acid 746. In FIG. 2A-2B, the amino acid sequence of E2 is amino acid 384 to amino acid 751. In FIG. 3A-3C, the amino acid sequence of E2 is amino acid 385 to amino acid 754. In FIG. 4A-4B, the amino acid sequence of E2 is amino acid 384 to amino acid 750. As used herein, an “E2 polypeptide” includes a precursor E2 protein, including the signal sequence; includes a mature E2 polypeptide which lacks this sequence; and includes an E2 polypeptide with a heterologous signal sequence. An E2 polypeptide can include a C-terminal membrane anchor sequence which occurs at approximately amino acid positions 715-730 and may extend as far as approximately amino acid residue 746 (see, Lin et al., J. Virol. (1994) 68:5063-5073).

In some cases, a E2 polypeptide suitable for inclusion in an immunogenic composition of the present disclosure lacks a portion of its C-terminal region, e.g., from about amino acid 715 to the C-terminus; from about amino acid 625 to the C-terminus; from about amino acid 661 to the C-terminus; from about amino acid 655 to the C-terminus; from about amino acid 500 to the C-terminus, where the amino acid numbering is with reference to the numbering in FIG. 1A-1C. See, e.g., U.S. Pat. No. 6,521,423.

An E2 polypeptide suitable for inclusion in an immunogenic composition of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E2 polypeptide depicted in FIG. 1A-1C, FIG. 2A-2C, FIG. 3A-3C, or FIG. 4A-4B. An E2 polypeptide suitable for inclusion in an immunogenic composition of the present disclosure can comprise an amino acid sequence having at least about 20%, at least about 25%, at least about 30%, at least about 35%, at least about 40%, at least about 45%, at least about 50%, at least about 60%, at least about 70%, or at least about 75%, amino acid sequence identity to an amino acid sequence of an E2 polypeptide depicted in FIG. 1A-IC, FIG. 2A-2C, FIG. 3A-3C, or FIG. 4A-4B.

An E2 polypeptide suitable for inclusion in an immunogenic composition of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E2 polypeptide depicted in FIG. 1A-IC. For example, an E2 polypeptide of genotype 1 can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-746 of an amino acid sequence depicted in FIG. 1A-IC. For example, an E2 polypeptide of genotype 1A can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-746 of an amino acid sequence identified as 1A and depicted in FIG. 1A-1C. For example, an E2 polypeptide of genotype 1B can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-746 of an amino acid sequence identified as 1B and depicted in FIG. 1A-IC. For example, an E2 polypeptide of genotype 1C can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-746 of an amino acid sequence identified as 1C and depicted in FIG. 1A-IC.

An E2 polypeptide suitable for inclusion in an immunogenic composition of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E2 polypeptide depicted in FIG. 2A-2C. For example, an E2 polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-751 of an amino acid sequence depicted in FIG. 2A-2C. For example, an E2 polypeptide of genotype 2A can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-751 of the “consensus” amino acid sequence depicted in FIG. 2A-2C. For example, an E2 polypeptide of genotype 2B can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-751 of the “consensus” amino acid sequence depicted in FIG. 2A-2C.

An E2 polypeptide suitable for inclusion in an immunogenic composition of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E2 polypeptide depicted in FIG. 3A-3C. For example, an E2 polypeptide of genotype 3 can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 385-754 of an amino acid sequence depicted in FIG. 3A-3C. For example, an E2 polypeptide of genotype 3A can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 385-754 of an amino acid sequence identified as 3A and depicted in FIG. 3A-3C. For example, an E2 polypeptide of genotype 3B can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 385-754 of the amino acid sequence identified as 3B and depicted in FIG. 3A-3C. For example, an E2 polypeptide of genotype 3K can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 385-754 of the amino acid sequence identified as 3K and depicted in FIG. 3A-3C.

An E2 polypeptide suitable for inclusion in an immunogenic composition of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the amino acid sequence of the E2 polypeptide depicted in FIG. 4A-4B. For example, an E2 polypeptide of genotype 7A can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-750 of the amino acid sequence depicted in FIG. 4A-4B.

### E1 Polypeptides

An HCV E1 polypeptide suitable for inclusion in an E1/E2 heterodimer for inclusion in an immunogenic composition of the present disclosure, or for inclusion by itself in an immunogenic composition of the present disclosure, can have a length of from about 100 amino acids (aa) to about 150 aa, from about 150 aa to about 175 aa, from about 175 aa to about 195 aa, from about 131 aa to about 175 aa, or from about 175 aa to about 193 aa. In some cases, an HCV E1 polypeptide suitable for inclusion in an E1/E2 heterodimer present in an immunogenic composition of the present disclosure is an HCV E1 ectodomain polypeptide. In some cases, an HCV E1 polypeptide suitable for inclusion in an E1/E2 heterodimer present in an immunogenic composition of the present disclosure is a full-length HCV E1 polypeptide.

In FIG. 1A-1C, the amino acid sequence of E1 is amino acid 192 to amino acid 383. In FIG. 2A-2C, the amino acid sequence of E1 is amino acid 192 to amino acid 383. In FIG. 3A-3C, the amino acid sequence of E1 is amino acid 192 to amino acid 384. In FIG. 4A-4B, the amino acid sequence of E1 is amino acid 192 to amino acid 383. Amino acids at around 170 through approximately 191 serve as a signal sequence for E1. As used herein, “E1 polypeptide” includes a precursor E1 protein, including the signal sequence; includes a mature E1 polypeptide which lacks this sequence; and includes an E1 polypeptide with a heterologous signal sequence. An E1 polypeptide can include a C-terminal membrane anchor sequence which occurs at approximately amino acid positions 360-383 (see, e.g., WO 96/04301). In some cases, a suitable E1 polypeptide lacks a C-terminal portion that includes a transmembrane region. For example, in some cases, a suitable E1 polypeptide lacks the C-terminal portion from amino acid 330 to amino acid 384, or from amino acid 360 to amino acid 384. E1 polypeptides can be an E1 polypeptide of any genotype, subtype or isolate of HCV. E1 polypeptides of genotype 1 and E1 polypeptides of genotype 3 are included in an E1/E2 heterodimer of the present disclosure.

An E1 polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E1 polypeptide depicted in FIG. 1A-1C, FIG. 2A-2C, FIG. 3A-3C, or FIG. 4A-4B.

An E1 polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E1 polypeptide depicted in FIG. 1A-1C. For example, an E1 polypeptide of genotype 1A can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 192-383 of an amino acid sequence identified as 1A and depicted in FIG. 1A-1C. For example, an E1 polypeptide of genotype 1B can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 192-383 of an amino acid sequence identified as 1B and depicted in FIG. 1A-1C. For example, an E1 polypeptide of genotype 1C can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 192-383 of an amino acid sequence identified as 1C and depicted in FIG. 1A-1C.

An E1 polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E1 polypeptide depicted in FIG. 2A-2C. For example, an E1 polypeptide of genotype 2A can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 192-383 of an amino acid sequence identified as 2A and depicted in FIG. 2A-2C. For example, an E1 polypeptide of genotype 2B can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 192-383 of an amino acid sequence identified as 2B and depicted in FIG. 2A-2C.

An E1 polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the consensus E1 polypeptide amino acid sequence depicted in FIG. 3A-3C.

An E1 polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E1 polypeptide depicted in FIG. 4A-4B. For example, an E1 polypeptide of genotype 7A can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 192-383 of the amino acid sequence depicted in FIGS. 4A-4B.

HCV E1 and E2 Polypeptides Comprising Amino Acids from a Proteolytically Cleavable Linker

As described in more detail below, an HCV E1/E2 heterodimer can be generated using a method that involves an HCV E1 or an HCV E2 polypeptide comprising a heterologous proteolytically cleavable linker. Following enzymatic cleavage of the proteolytically cleavable linker, from 1 to 6 (e.g., 1, 2, 3, 4, 5, or 6) heterologous amino acids can remain on the HCV E1 or E2 polypeptide. For example, from 1 to 6 (e.g., 1, 2, 3, 4, 5, or 6) heterologous amino acids can remain at the N-terminus of an HCV E2 polypeptide. As another example, from 1 to 6 (e.g., 1, 2, 3, 4, 5, or 6) heterologous amino acids can remain at the C-terminus of an HCV E2 polypeptide. As another example, from 1 to 6 (e.g., 1, 2, 3, 4, 5, or 6) heterologous amino acids can remain at the N-terminus of an HCV E1 polypeptide. As another example, from 1 to 6 (e.g., 1, 2, 3, 4, 5, or 6) heterologous amino acids can remain at the C-terminus of an HCV E1 polypeptide.

In some cases, amino acids C-terminal to the proteolytic cleavage site in a proteolytically cleavable linker are Gly-Pro, Ser, Gly, or Gly-Ser. Thus, in some cases, a modified HCV E1 polypeptide comprises, appended to the N-terminus of an HCV E1 polypeptide: Gly-Pro, Ser, Gly, or Gly-Ser. In other words, in some cases, a modified HCV E1 polypeptide comprises, in order from N-terminus to C-terminus: a) Gly-Pro, Ser, Gly, or Gly-Ser; and b) an HCV E1 polypeptide.

In some cases, amino acids C-terminal to the proteolytic cleavage site in a proteolytically cleavable linker are Gly-Pro, Ser, Gly, or Gly-Ser. Thus, in some cases, a modified HCV E2 polypeptide comprises, appended to the N-terminus of an HCV E2 polypeptide: Gly-Pro, Ser, Gly, or Gly-Ser. In other words, in some cases, a modified HCV E2 polypeptide comprises, in order from N-terminus to C-terminus: a) Gly-Pro, Ser, Gly, or Gly-Ser; and b) an HCV E2 polypeptide.

In some cases, amino acids N-terminal to the proteolytic cleavage site in a proteolytically cleavable linker are LEVLFQ (SEQ ID NO:83), ENLYYFQ (SEQ ID NO:84), LVPR (SEQ ID NO:85), I(E/D)GR (SEQ ID NO:86), or DDDDK (SEQ ID NO:87). Thus, in some cases, a modified HCV E1 polypeptide comprises, appended to the C-terminus of an HCV E1 polypeptide: LEVLFQ (SEQ ID NO:83), ENLYYFQ (SEQ ID NO:84), LVPR (SEQ ID NO:85), I(E/D)GR (SEQ ID NO:86), or DDDDK (SEQ ID NO:87). In other words, in some cases, a modified HCV E1 polypeptide comprises, in order from N-terminus to C-terminus: a) an HCV E1 polypeptide; and b) LEVLFQ (SEQ ID NO:83), ENLYYFQ (SEQ ID NO:84), LVPR (SEQ ID NO:85), I(E/D)GR (SEQ ID NO:86), or DDDDK (SEQ ID NO:87).

In some cases, amino acids N-terminal to the proteolytic cleavage site in a proteolytically cleavable linker are LEVLFQ (SEQ ID NO:83), ENLYYFQ (SEQ ID NO:84), LVPR (SEQ ID NO:85), I(E/D)GR (SEQ ID NO:86), or DDDDK (SEQ ID NO:87). Thus, in some cases, a modified HCV E2 polypeptide comprises, appended to the C-terminus of an HCV E2 polypeptide: LEVLFQ (SEQ ID NO:83), ENLYYFQ (SEQ ID NO:84), LVPR (SEQ ID NO:85), I(E/D)GR (SEQ ID NO:86), or DDDDK (SEQ ID NO:87). In other words, in some cases, a modified HCV E2 polypeptide comprises, in order from N-terminus to C-terminus: a) an HCV E2 polypeptide; and b) LEVLFQ (SEQ ID NO:83), ENLYYFQ (SEQ ID NO:84), LVPR (SEQ ID NO:85), I(E/D)GR (SEQ ID NO:86), or DDDDK (SEQ ID NO:87).

In some cases, a flexible linker of from 1 to 10 amino acids is interposed between the proteolytically cleavable linker and the HCV E1 or E2 polypeptide. Flexible linkers are intrinsically disordered flexible linker domains or loops that vary in length and can be rich in polar uncharged amino acids. Flexible linkers include, e.g., glycine polymers (G)ₙ, glycine-serine polymers (including, for example, (GS)ₙ, (GSGGS)ₙ (SEQ ID NO:88), (GGSGGS)ₙ (SEQ ID NO:89), and (GGGS)ₙ (SEQ ID NO:90), where n is an integer of at least one, e.g., where n is 1, 2, 3, 4, 5, or 6); glycine-alanine polymers, such as GAGAGAGA and the like; and alanine-serine polymers, e.g., SASASASA and the like. Exemplary linkers can comprise amino acid sequences including, but not limited to, GGSG (SEQ ID NO:91), GGSGG (SEQ ID NO:92), GSGSG (SEQ ID NO:93), GSGGG (SEQ ID NO:94), GGGSG (SEQ ID NO:95), GSSSG (SEQ ID NO:96), and the like.

For example, in some cases, a modified E1 polypeptide comprises, in order from N-terminus to C-terminus: a) Gly-Pro, Ser, Gly, or Gly-Ser; b) a flexible linker of from 1 to 10 amino acids; and c) an HCV E1 polypeptide.

As another example, in some cases, a modified E2 polypeptide comprises, in order from N-terminus to C-terminus: a) Gly-Pro, Ser, Gly, or Gly-Ser; b) a flexible linker of from 1 to 10 amino acids; and c) an HCV E2 polypeptide.

As another in some cases, a modified E1 polypeptide comprises, from N-terminus to C-terminus: a) an HCV E1 polypeptide; b) a flexible linker of from 1 to 10 amino acids; and c) LEVLFQ (SEQ ID NO:83), ENLYYFQ (SEQ ID NO:84), LVPR (SEQ ID NO:85), I(E/D)GR (SEQ ID NO:86), or DDDDK (SEQ ID NO:87).

As another in some cases, a modified E2 polypeptide comprises, from N-terminus to C-terminus: a) an HCV E2 polypeptide; b) a flexible linker of from 1 to 10 amino acids; and c) LEVLFQ (SEQ ID NO:83), ENLYYFQ (SEQ ID NO:84), LVPR (SEQ ID NO:85), I(E/D)GR (SEQ ID NO:86), or DDDDK (SEQ ID NO:87).

E2 with N-Terminal Heterologous Amino Acids

In some cases, an HCV E1/E2 heterodimer suitable for inclusion in an immunogenic composition of the present disclosure comprises a modified HCV E2 polypeptide with from 1 to 6 amino acids from the proteolytically cleavable linker on the N-terminus of the E2 polypeptide. In some cases, an HCV E1/E2 heterodimer suitable for inclusion in an immunogenic composition of the present disclosure comprises: a) an HCV E1 polypeptide; and b) a modified E2 polypeptide comprising, in order from N-terminus to C-terminus: i) from 1 to 6 heterologous amino acids wherein the from 1 to 6 heterologous amino acids are C-terminal to a site of proteolytic cleavage in a proteolytically cleavable linker; and ii) an HCV E2 polypeptide.

Proteolytically cleavable linkers are described elsewhere herein. Following proteolytic cleavage of a precursor polypeptide, as described herein, a modified E2 polypeptide is generated, which modified E2 polypeptide comprises, at its N-terminus, amino acids C-terminal to the protease cleavage site within the proteolytically cleavable linker.

For example, where the proteolytically cleavable linker comprises a PreScission cleavage site (LEVLFQGP; SEQ ID NO:97), where cleavage occurs between the glutamine and the glycine, a modified E2 polypeptide present in a heterodimeric E1/E2 polypeptide suitable for inclusion in an immunogenic composition of the present disclosure comprises, in order from N-terminus to C-terminus: a) Gly-Pro; and b) an HCV E2 polypeptide. As another example, where the proteolytically cleavable linker comprises a TEV cleavage site (ENLYFQS; SEQ ID NO:98), where cleavage occurs between the glutamine and the serine, a modified E2 polypeptide present in a heterodimeric E1/E2 polypeptide suitable for inclusion in an immunogenic composition of the present disclosure comprises, in order from N-terminus to C-terminus: a) Ser; and b) an HCV E2 polypeptide.

As another example, where the proteolytically cleavable linker comprises a TEV cleavage site (ENLYFQG; SEQ ID NO:99), where cleavage occurs between the glutamine and the glycine, a modified E2 polypeptide present in a heterodimeric E1/E2 polypeptide suitable for inclusion in an immunogenic composition of the present disclosure comprises, in order from N-terminus to C-terminus: a) Gly; and b) an HCV E2 polypeptide.

As another example, where the proteolytically cleavable linker comprises a thrombin cleavage site (LVPRGS; SEQ ID NO:100), where cleavage occurs between the arginine and the glycine, a modified E2 polypeptide present in a heterodimeric E1/E2 polypeptide suitable for inclusion in an immunogenic composition of the present disclosure comprises, in order from N-terminus to C-terminus: a) Gly-Ser; and an HCV E2 polypeptide.

As another example, where the proteolytically cleavable linker comprises a Factor Xa cleavage site (I(E/D)GRX, where X is any amino acid except arginine or proline; SEQ ID NO:101), where cleavage occurs between the arginine and the X, a modified E2 polypeptide present in a heterodimeric E1/E2 polypeptide suitable for inclusion in an immunogenic composition of the present disclosure comprises, in order from N-terminus to C-terminus: a) X (where X is any amino acid except arginine or proline); and an HCV E2 polypeptide.

Thus, for example, in some cases, an HCV E1/E2 heterodimer suitable for inclusion in an immunogenic composition of the present disclosure comprises: a) an HCV E1 polypeptide; and b) a modified E2 polypeptide comprising, in order from N-terminus to C-terminus: i) from 1 to 6 heterologous amino acids, wherein the from 1 to 6 (e.g., 1, 2, 3, 4, 5, or 6) heterologous amino acids are C-terminal to a site of proteolytic cleavage in a proteolytically cleavable linker; and ii) an HCV E2 polypeptide. In some cases, the 1 to 6 heterologous amino acids are Gly-Pro. In some cases, the 1 to 6 heterologous amino acids is Ser. In some cases, the 1 to 6 heterologous amino acids is Gly. In some cases, the 1 to 6 heterologous amino acids are Gly-Ser.

As another example, in some cases, an HCV E1/E2 heterodimer suitable for inclusion in an immunogenic composition of the present disclosure comprises: a) an HCV E2 polypeptide; and b) a modified E1 polypeptide comprising, in order from N-terminus to C-terminus: i) from 1 to 6 heterologous amino acids, wherein the from 1 to 6 (e.g., 1, 2, 3, 4, 5, or 6) heterologous amino acids are C-terminal to a site of proteolytic cleavage in a proteolytically cleavable linker; and ii) an HCV E1 polypeptide. In some cases, the 1 to 6 heterologous amino acids are Gly-Pro. In some cases, the 1 to 6 heterologous amino acids is Ser. In some cases, the 1 to 6 heterologous amino acids is Gly. In some cases, the 1 to 6 heterologous amino acids are Gly-Ser. E1 with N-terminal heterologous amino acids

In some cases, an HCV E1/E2 heterodimer suitable for inclusion in an immunogenic composition of the present disclosure comprises a modified HCV E1 polypeptide with from 1 to 6 amino acids from a proteolytically cleavable linker on the N-terminus of the E1 polypeptide. In some cases, an HCV E1/E2 heterodimer suitable for inclusion in an immunogenic composition of the present disclosure comprises: a) an HCV E2 polypeptide; and b) a modified E1 polypeptide comprising, in order from N-terminus to C-terminus: i) from 1 to 6 heterologous amino acids wherein the from 1 to 6 heterologous amino acids are C-terminal to a site of proteolytic cleavage in a proteolytically cleavable linker; and ii) an HCV E1 polypeptide.

Proteolytically cleavable linkers are described elsewhere herein. Following proteolytic cleavage of a precursor polypeptide (e.g., a precursor polypeptide comprising, in order from N-terminus to C-terminus: a) an Fc polypeptide or an HCV E2 polypeptide; b) a proteolytically cleavable linker; and c) an HCV E1 polypeptide), a modified E1 polypeptide is generated, which modified E1 polypeptide comprises, at its N-terminus, amino acids C-terminal to the protease cleavage site within the proteolytically cleavable linker.

For example, where the proteolytically cleavable linker comprises a PreScission cleavage site (LEVLFQGP; SEQ ID NO:97), where cleavage occurs between the glutamine and the glycine, a modified E1 polypeptide present in an HCV E1/E2 heterodimer suitable for inclusion in an immunogenic composition of the present disclosure comprises, in order from N-terminus to C-terminus: a) Gly-Pro; and b) an HCV E1 polypeptide.

As another example, where the proteolytically cleavable linker comprises a TEV cleavage site (ENLYFQS; SEQ ID NO:98), where cleavage occurs between the glutamine and the serine, a modified E1 polypeptide present in an HCV E1/E2 heterodimer suitable for inclusion in an immunogenic composition of the present disclosure comprises, in order from N-terminus to C-terminus: a) Ser; and b) an HCV E1 polypeptide.

As another example, where the proteolytically cleavable linker comprises a TEV cleavage site (ENLYFQG; SEQ ID NO:99), where cleavage occurs between the glutamine and the glycine, a modified E1 polypeptide present in an HCV E1/E2 heterodimer suitable for inclusion in an immunogenic composition of the present disclosure comprises, in order from N-terminus to C-terminus: a) Gly; and b) an HCV E1 polypeptide.

As another example, where the proteolytically cleavable linker comprises a thrombin cleavage site (LVPRGS; SEQ ID NO:100), where cleavage occurs between the arginine and the glycine, a modified E1 polypeptide present in an HCV E1/E2 heterodimer suitable for inclusion in an immunogenic composition of the present disclosure comprises, in order from N-terminus to C-terminus: a) Gly-Ser; and an HCV E1 polypeptide.

As another example, where the proteolytically cleavable linker comprises a Factor Xa cleavage site (I(E/D)GRX, where X is any amino acid except arginine or proline; SEQ ID NO:101), where cleavage occurs between the arginine and the X, a modified E1 polypeptide present in an HCV E1/E2 heterodimer suitable for inclusion in an immunogenic composition of the present disclosure comprises, in order from N-terminus to C-terminus: a) X (where X is any amino acid except arginine or proline); and an HCV E1 polypeptide.

E2 with C-Terminal Heterologous Amino Acids

In some cases, an HCV E1/E2 heterodimer suitable for inclusion in an immunogenic composition of the present disclosure comprises a modified HCV E2 polypeptide with from 1 to 6 amino acids from a proteolytically cleavable linker on the C-terminus of the E2 polypeptide. In some cases, an HCV E1/E2 heterodimer suitable for inclusion in an immunogenic composition of the present disclosure comprises: a) an HCV E1 polypeptide; and b) a modified E2 polypeptide comprising, in order from N-terminus to C-terminus: i) an HCV E2 polypeptide; and ii) from 1 to 6 heterologous amino acids wherein the from 1 to 6 heterologous amino acids are N-terminal to a site of proteolytic cleavage in a proteolytically cleavable linker.

Proteolytically cleavable linkers are described elsewhere herein. Following proteolytic cleavage of a precursor polypeptide (e.g., a precursor polypeptide comprising, in order from N-terminus to C-terminus: a) HCV E2 polypeptide; b) a proteolytically cleavable linker; and c) an Fc polypeptide or an HCV E1 polypeptide), a modified E2 polypeptide is generated, which modified E2 polypeptide comprises, at its C-terminus, amino acids N-terminal to the protease cleavage site within the proteolytically cleavable linker.

For example, where the proteolytically cleavable linker comprises a PreScission cleavage site (LEVLFQGP; SEQ ID NO:97), where cleavage occurs between the glutamine and the glycine, a modified E2 polypeptide present in an HCV E1/E2 heterodimer suitable for inclusion in an immunogenic composition of the present disclosure comprises, in order from N-terminus to C-terminus: a) an HCV E2 polypeptide; and b) LEVLFQ (SEQ ID NO:83).

As another example, where the proteolytically cleavable linker comprises an enterokinase cleavage site (DDDDK; SEQ ID NO:87), where cleavage occurs C-terminal to the Lys, a modified E2 polypeptide present in an HCV E1/E2 heterodimer suitable for inclusion in an immunogenic composition of the present disclosure comprises, in order from N-terminus to C-terminus: a) an HCV E2 polypeptide; and b) DDDDK (SEQ ID NO:87).

As another example, where the proteolytically cleavable linker comprises a TEV cleavage site (ENLYFQG; SEQ ID NO:99), where cleavage occurs between the glutamine and the glycine, a modified E2 polypeptide present in an HCV E1/E2 heterodimer suitable for inclusion in an immunogenic composition of the present disclosure comprises, in order from N-terminus to C-terminus: a) an HCV E2 polypeptide; and b) ENLYFQ.

As another example, where the proteolytically cleavable linker comprises a thrombin cleavage site (LVPRGS; SEQ ID NO:100), where cleavage occurs between the arginine and the glycine, a modified E2 polypeptide present in an HCV E1/E2 heterodimer suitable for inclusion in an immunogenic composition of the present disclosure comprises, in order from N-terminus to C-terminus: a) an HCV E2 polypeptide; and LVPR (SEQ ID NO:85).

As another example, where the proteolytically cleavable linker comprises a Factor Xa cleavage site (I(E/D)GRX, where X is any amino acid except arginine or proline; SEQ ID NO:101), where cleavage occurs between the arginine and the X, a modified E2 polypeptide present in an HCV E1/E2 heterodimer suitable for inclusion in an immunogenic composition of the present disclosure comprises, in order from N-terminus to C-terminus: a) an HCV E2 polypeptide; and I(E/D)GR (SEQ ID NO:86).

E1 with C-Terminal Heterologous Amino Acids

In some cases, an HCV E1/E2 heterodimer suitable for inclusion in an immunogenic composition of the present disclosure comprises a modified HCV E1 polypeptide with from 1 to 6 amino acids from a proteolytically cleavable linker on the C-terminus of the E1 polypeptide. In some cases, an HCV E1/E2 heterodimer suitable for inclusion in an immunogenic composition of the present disclosure comprises: a) an HCV E2 polypeptide; and b) a modified E1 polypeptide comprising, in order from N-terminus to C-terminus: i) an HCV E1 polypeptide; and ii) from 1 to 6 heterologous amino acids wherein the from 1 to 6 heterologous amino acids are N-terminal to a site of proteolytic cleavage in a proteolytically cleavable linker.

Proteolytically cleavable linkers are described elsewhere herein. Following proteolytic cleavage of a precursor polypeptide (e.g., a precursor polypeptide comprising, in order from N-terminus to C-terminus: a) HCV E1 polypeptide; b) a proteolytically cleavable linker; and c) an Fc polypeptide or an HCV E2 polypeptide), a modified E1 polypeptide is generated, which modified E1 polypeptide comprises, at its C-terminus, amino acids N-terminal to the protease cleavage site within the proteolytically cleavable linker.

For example, where the proteolytically cleavable linker comprises a PreScission cleavage site (LEVLFQGP; SEQ ID NO:97), where cleavage occurs between the glutamine and the glycine, a modified E1 polypeptide present in an HCV E1/E2 heterodimer suitable for inclusion in an immunogenic composition of the present disclosure comprises, in order from N-terminus to C-terminus: a) an HCV E1 polypeptide; and b) LEVLFQ (SEQ ID NO:83).

As another example, where the proteolytically cleavable linker comprises an enterokinase cleavage site (DDDDK; SEQ ID NO:87), where cleavage occurs C-terminal to the Lys, a modified E1 polypeptide present in an HCV E1/E2 heterodimer suitable for inclusion in an immunogenic composition of the present disclosure comprises, in order from N-terminus to C-terminus: a) an HCV E1 polypeptide; and b) DDDDK (SEQ ID NO:87).

As another example, where the proteolytically cleavable linker comprises a TEV cleavage site (ENLYFQG; SEQ ID NO:99), where cleavage occurs between the glutamine and the glycine, a modified E1 polypeptide present in an HCV E1/E2 heterodimer suitable for inclusion in an immunogenic composition of the present disclosure comprises, in order from N-terminus to C-terminus: a) an HCV E1 polypeptide; and b) ENLYFQ (SEQ ID NO:102).

As another example, where the proteolytically cleavable linker comprises a thrombin cleavage site (LVPRGS; SEQ ID NO:100), where cleavage occurs between the arginine and the glycine, a modified E1 polypeptide present in an HCV E1/E2 heterodimer suitable for inclusion in an immunogenic composition of the present disclosure comprises, in order from N-terminus to C-terminus: a) an HCV E1 polypeptide; and LVPR (SEQ ID NO:85).

As another example, where the proteolytically cleavable linker comprises a Factor Xa cleavage site (I(E/D)GRX, where X is any amino acid except arginine or proline; SEQ ID NO:101), where cleavage occurs between the arginine and the X, a modified E1 polypeptide present in an HCV E1/E2 heterodimer suitable for inclusion in an immunogenic composition of the present disclosure comprises, in order from N-terminus to C-terminus: a) an HCV E1 polypeptide; and I(E/D)GR (SEQ ID NO:86).

### Additional Polypeptides

In any of the above-described embodiments, one or both of the polypeptide chains of the E1/E2 heterodimer can include one or more additional polypeptides. For example, the E1 polypeptide, the E2 polypeptide, or both the E1 and the E2 polypeptide, can include an affinity tag. Suitable affinity tags include, e.g., immunoglobulin Fc polypeptides, a poly(histidine) tag (e.g., His₆), a maltose binding protein (MBP), a glutathione-S-transferase (GST) polypeptide, calmodulin-binding peptide (CBP), Streptavidin-binding peptide (SBP), Strep-tag II, FLAG (e.g., DYKDDDDK (SEQ ID NO:103), hemagglutinin (HA) (e.g., YPYDVPDYA (SEQ ID NO:104), c-myc T7 ((e.g., EQKLISEEDL; SEQ ID NO:105), Glu-Glu, starch-binding domain (SBD), and Flag-Acidic-Target Tag (FATT), and the like.

In some cases, an E1/E2 heterodimer included in a composition of the present disclosure includes a variant E2 polypeptide. In some cases, the E1 polypeptide or the variant E2 polypeptide can include an Ig Fc polypeptide at the C-terminus of the E1 polypeptide or the variant E2 polypeptide. As another example, in some cases, the E1 polypeptide or the variant E2 polypeptide can include an Ig Fc polypeptide at the N-terminus of the E1 polypeptide or the variant E2 polypeptide. Ig Fc polypeptides are known in the art, and are described elsewhere herein.

### Pharmaceutically Acceptable Excipients

The present disclosure provides an immunogenic composition comprising: a) one or more T-cell epitope polypeptides as described herein; and b) a pharmaceutically acceptable carrier. The present disclosure provides an immunogenic composition comprising: a) one or more T-cell epitope polypeptides as described herein; b) a pharmaceutically acceptable carrier; and c) an adjuvant. The present disclosure provides an immunogenic composition comprising: a) an HCV heterodimeric polypeptide comprising: i) an HCV E1 polypeptide; and ii) an HCV E2 polypeptide; b) a heterologous polypeptide comprising a T-cell epitope present in an HCV protein other than E1 and E2 (“a T-cell epitope polypeptide”); and c) a pharmaceutically acceptable carrier. The present disclosure provides an immunogenic composition comprising: a) an HCV E2 polypeptide; b) a heterologous polypeptide comprising a T-cell epitope present in an HCV protein other than E1 and E2; and c) a pharmaceutically acceptable carrier. The present disclosure provides an immunogenic composition comprising: a) an HCV E1 polypeptide; b) a heterologous polypeptide comprising a T-cell epitope present in an HCV protein other than E1 and E2; and c) a pharmaceutically acceptable carrier. The present disclosure provides an immunogenic composition comprising: a) an HCV E1 polypeptide; b) a heterologous polypeptide comprising a T-cell epitope present in an HCV protein other than E1 and E2; c) a pharmaceutically acceptable carrier; and d) an adjuvant (e.g., an immune-stimulating amount of an adjuvant).

An immunogenic composition of the present disclosure can comprise, in addition to the above-mentioned HCV polypeptides, one or more of: i) a buffer; ii) a salt; iii) a chelating agent; and iv) a non-ionic detergent. Suitable salts include, e.g., sodium citrate; sodium chloride, and the like. For example, the composition can comprise NaCl in a concentration of from about 50 mM to about 500 mM; e.g., an immunogenic composition of the present disclosure can include NaCl in a concentration of from about 50 mM to about 75 mM, from about 75 mM to about 100 mM, from about 100 mM to about 150 mM, from about 150 mM to about 200 mM, from about 200 mM to about 300 mM, from about 300 mM to about 400 mM, or from about 400 mM to about 500 mM. As one non-limiting example, an immunogenic composition of the present disclosure can comprise, in addition to the above-mentioned HCV polypeptides, the following components: 10 mM sodium citrate, 250 mM NaCl, 1 mM ethylenediaminetetraacetic acid (EDTA), 0.1% non-ionic detergent Tween 80, pH 6.0. As another non-limiting example, an immunogenic composition of the present disclosure can comprise, in addition to the above-mentioned HCV polypeptides, the following components: 10 mM sodium citrate, 100 mM NaCl, 1 mM EDTA, 0.1% non-ionic detergent Tween 80, pH 6.0. As another non-limiting example, an immunogenic composition of the present disclosure can comprise, in addition to the above-mentioned HCV polypeptides, the following components: 10 mM sodium citrate, 400 mM NaCl, 1 mM EDTA, 0.1% non-ionic detergent Tween 80, pH 6.0.

In some cases, where an immunogenic composition of the present disclosure includes an HCV E1 polypeptide and an HCV E2 polypeptide, the ratio of HCV E2 polypeptide to HCV E1 polypeptide is in a range of from about 2:1 to 1:1, e.g., from about 2:1 to 1.5:1, or from 1.5:1 to 1:1. In some cases, where an immunogenic composition of the present disclosure includes an HCV E1 polypeptide and a HCV E2 polypeptide, the molar ratio of HCV E2 polypeptide to HCV E1 polypeptide is in a range of from about 1:1 to 1.5:1, from 1.5:1 to 2:1, from 2:1 to 3:1, from 3:1 to 4:1, from 4:1 to 6:1, or from 6:1 to 8:1.

HCV E1 polypeptides, HCV E2 polypeptides, and heterologous polypeptides (e.g., T-cell epitope polypeptide) can be formulated with a pharmaceutically acceptable excipient(s) to generate an immunogenic composition of the present disclosure. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7th ed., Lippincott, Williams, & Wilkins; and Handbook of Pharmaceutical Excipients (2000) A. H. Kibbe et al., eds., 3rd ed. Amer. Pharmaceutical Assoc.

In some embodiments, an HCV E1 polypeptide, an HCV E2 polypeptide (e.g., as an HCV E1/E2 heterodimer), and a heterologous polypeptide (e.g., T-cell epitope polypeptide) are formulated in an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 (TWEEN®20) or polysorbate 80 (TWEEN®80). For example, a formulation of an HCV E1 polypeptide, an HCV E2 polypeptide (e.g., as an HCV E1/E2 heterodimer), and a heterologous polypeptide (e.g., T-cell epitope polypeptide) in an aqueous buffer can include, e.g., from about 0.01% to about 0.05% polysorbate-20 (TWEEN®20) non-ionic detergent. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures. In some cases, the aqueous buffer further includes a non-ionic surfactant. In some cases, the aqueous buffer includes the non-ionic surfactant Triton™X-100, e.g., 0.1% Triton™X-100.

An HCV E1 polypeptide, an HCV E2 polypeptide (e.g., as an HCV E1/E2 heterodimer), and a heterologous polypeptide can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

An immunogenic composition of the present disclosure can include, e.g., pharmaceutical grades of mannitol, lactose, starch, magnesium stearate, sodium saccharin, talcum, cellulose, glucose, sucrose, magnesium, carbonate, and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like.

The concentration of an HCV E1 polypeptide, an HCV E2 polypeptide (e.g., as an HCV E1/E2 heterodimer), and a heterologous polypeptide in a formulation can vary widely (e.g., from less than about 0.1% to at least about 2%, to as much as 20% to 50% or more by weight) and can be selected primarily based on fluid volumes, viscosities, and patient-based factors in accordance with the particular mode of administration selected and the patient's needs.

An immunogenic composition of the present disclosure can be provided in the form of a solution, suspension, tablet, pill, capsule, powder, gel, cream, lotion, ointment, aerosol or the like. It is recognized that oral administration can require protection of the compositions from digestion. This is typically accomplished either by association of the composition with an agent that renders it resistant to acidic and enzymatic hydrolysis or by packaging the composition in an appropriately resistant carrier. Means of protecting from digestion are well known in the art.

An immunogenic composition of the present disclosure can also be provided so as to enhance serum half-life of the polypeptides (an HCV E1 polypeptide, an HCV E2 polypeptide (e.g., as an HCV E1/E2 heterodimer), and a heterologous polypeptide) following administration. For example, where an isolated HCV E1 polypeptide, an HCV E2 polypeptide (e.g., as an HCV E1/E2 heterodimer), and a heterologous polypeptide are formulated for injection, the polypeptides may be provided in a liposome formulation, prepared as a colloid, or other conventional techniques for extending serum half-life. A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028. The preparations may also be provided in controlled release or slow-release forms.

### Adjuvants

An immunogenic composition of the present disclosure can include an adjuvant (e.g., an immunostimulating amount of an adjuvant). An immunogenic composition of the present disclosure can include an immune-stimulating amount of an adjuvant. Examples of known suitable adjuvants that can be used in humans include, but are not necessarily limited to, alum, aluminum phosphate, aluminum hydroxide, MF59 (4.3% w/v squalene, 0.5% w/v Tween 80™, 0.5% w/v Span 85), CpG-containing nucleic acid (where the cytosine is unmethylated), QS21, monophosphoryl lipid A (MPL), 3-Q-desacyl-4^{′}-monophosphoryl lipid A (3DMPL), extracts from Aquilla, immune-stimulating complexes (ISCOMS; complexes of cholesterol, phospholipids, and Quillaja saponins), LT/CT mutants, poly(D,L-lactide-co-glycolide) (PLG) microparticles, Quil A, interleukins, and the like. For experimental animals, one can use Freund's incomplete adjuvant, or Freund's complete adjuvant. Also suitable for use are N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1^{′}-2^{′}-dipalmitoyl-sn-glycero-3-hydroxyphosphoryloxy)-ethylamine (CGP 19835A, referred to as MTP-PE), and RIBI, which contains three components extracted from bacteria: monophosphoryl lipid A, trehalose dimycolate and cell wall skeleton (MPL+TDM+CWS) in a 2% squalene/Tween 80 emulsion. The effectiveness of an adjuvant may be determined by one or more of: i) measuring the amount of antibodies directed against the immunogenic antigen or antigenic epitope thereof; ii) measuring a cytotoxic T lymphocyte response to the antigen; and iii) measuring a helper T cell response to the antigen.

Further exemplary adjuvants to enhance effectiveness of the composition include, but are not limited to: (1) oil-in-water emulsion formulations (with or without other specific immunostimulating agents such as muramyl peptides (see below) or bacterial cell wall components), such as for example (a) MF59™ (see, e.g., WO 90/14837), containing 5% Squalene, 0.5% Tween 80, and 0.5% Span 85 (optionally containing MTP-PE) formulated into submicron particles using a microfluidizer, (b) SAF, containing 10% Squalane, 0.4% Tween 80, 5% pluronic-blocked polymer L121, and thr-MDP either microfluidized into a submicron emulsion or vortexed to generate a larger particle size emulsion, and (c) RIBI™ adjuvant system (RAS), (Ribi Immunochem, Hamilton, Mont.) containing 2% Squalene, 0.2% Tween 80, and one or more bacterial cell wall components such as monophosphorylipid A (MPL), trehalose dimycolate (TDM), and cell wall skeleton (CWS), e.g., MPL+CWS (Detox™); (2) saponin adjuvants, such as QS21 or Stimulon™ (Cambridge Bioscience, Worcester, Mass.; a purified extract of Quillaja saponaria) may be used or particles generated therefrom such as ISCOMs (immunostimulating complexes), which ISCOMS may be devoid of additional detergent e.g. WO 00/07621; (3) Complete Freund's Adjuvant (CFA) and Incomplete Freund's Adjuvant (IFA); (4) cytokines, such as interleukins (e.g. IL-1, IL-2, IL-4, IL-5, IL-6, IL-7, IL-12 (WO99/44636), etc.), interferons (e.g. gamma interferon), macrophage colony stimulating factor (M-CSF), tumor necrosis factor (TNF), etc.; (5) monophosphoryl lipid A (MPL) or 3-O-deacylated MPL (3dMPL) e.g. GB-2220221, EP-A-0689454, optionally in the substantial absence of alum when used with pneumococcal saccharides e.g. WO 00/56358; (6) combinations of 3dMPL with, for example, QS21 and/or oil-in-water emulsions (see, e.g. EP-A-0835318, EP-A-0735898, EP-A-0761231); (7) oligonucleotides comprising a CpG motif containing at least one CG dinucleotide, where the cytosine is unmethylated (see, e.g., WO 96/02555, WO 98/16247, WO 98/18810, WO 98/40100, WO 98/55495, WO 98/37919 and WO 98/52581); (8) a polyoxyethylene ether or a polyoxyethylene ester (see, e.g. WO 99/52549); (9) a polyoxyethylene sorbitan ester surfactant in combination with an octoxynol (WO 01/21207) or a polyoxyethylene alkyl ether or ester surfactant in combination with at least one additional non-ionic surfactant such as an octoxynol (WO 01/21152); (10) a saponin and an immunostimulatory oligonucleotide (e.g. a CpG oligonucleotide) (WO 00/62800); (11) an immunostimulant and a particle of metal salt (see, e.g. WO 00/23105); (12) a saponin and an oil-in-water emulsion (see e.g. WO 99/11241); (13) a saponin (e.g. QS21)+3dMPL+IM2 (optionally including a sterol) (see, e.g. WO 98/57659); (14) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1^{′}-2^{′}-dipalmitoyl-sn-glycero-3-hydroxyphosphoryloxy)-ethylamine MTP-PE), etc. Also suitable for use is Matrix-M™; Matrix-M™ is an adjuvant that comprises 40 nm nanoparticles comprising Quillaja saponins, cholesterol, and phospholipid. Adjuvants suitable for administration to a human are of particular interest. In some cases, the adjuvant is one that enhances a CD4+T helper response to the immunogen. Also suitable for use is a poly inosine:cytosine (poly I:C) nucleic acid. Poly I:C is a synthetic double-stranded RNA Also suitable for use is a cyclic dinucleotide activator of the STING pathway. Examples of suitable cyclic dinucleotide adjuvants include, but are not limited to: 1) bis-(3′,5^{′})-cyclic dimeric adenosine monophosphate (c-di-AMP); 2) bis-(3′,5^{′})-cyclic dimeric guanosine monophosphate (c-di-GMP); and bis-(3′,5^{′})-cyclic dimeric inosine monosphosphate (c-di-IMP). Also suitable for use is poly(I:C).

QS21 has the following structure:

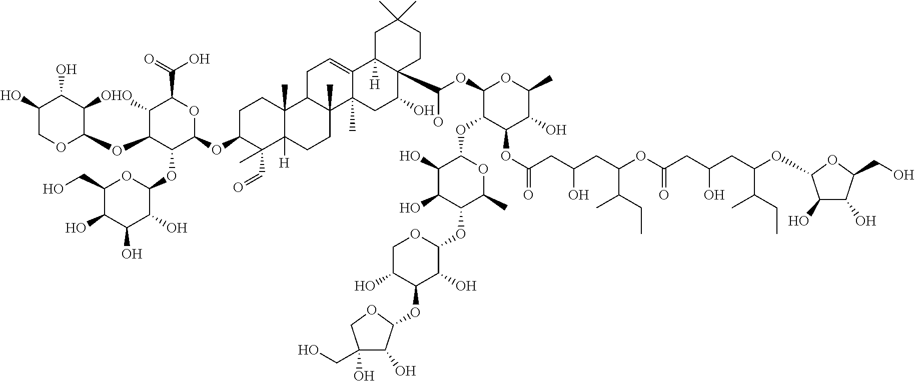

MPL has the following structure:

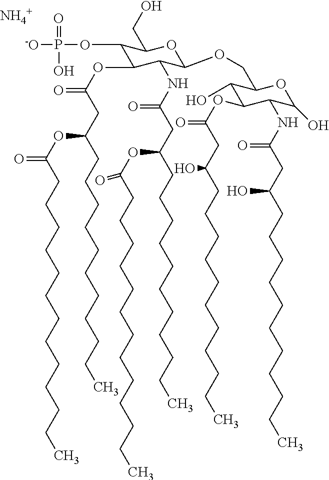

In some instances, the adjuvant is MF59, with or without a CpG-containing oligonucleotide. In other instances, the adjuvant is alum, with or without a CpG-containing oligonucleotide. In other instances, the adjuvant is poly(D,L-lactide-co-glycolide, with or without a CpG-containing oligonucleotide. In other instances, the adjuvant is MPL, with or without a CpG-containing oligonucleotide. In some cases, the adjuvant is Matrix-M™, with or without a CpG-containing oligonucleotide. In some cases, the adjuvant is keyhole limpet hemocyanin. In some cases, the adjuvant is alum. In some cases, the adjuvant is aluminum phosphate. In some cases, the adjuvant is aluminum hydroxide. In some cases, the adjuvant is alum+MPL. In some cases, the adjuvant is MF59. In some cases, the adjuvant is alum+MF59. In some cases, the adjuvant is AS01. AS01 contains QS-21 Stimulon® adjuvant, MPL, and liposomes. In some cases, the adjuvant comprises QS21 and MPL in a liposomal formulation. In some cases, the adjuvant is AS03. A dose of AS03 contains: 10.69 mg squalene; 11.86 mg DL-α-tocopherol; and 4.86 mg polysorbate-80. In some cases, the adjuvant comprises aluminum hydroxide and MPL. In some cases, the adjuvant is AS04. AS04 comprises aluminum hydroxide and MPL. In some cases, the adjuvant is AS15. AS15 is a combination of QS-21 Stimulon® adjuvant, monophosphoryl lipid A, and CpG7909 (an oligonucleotide of the sequence 5^{′}-TCGTCGTTTTGTCGTTTTGTCGTT-3^{′}; (SEQ ID NO:106), in a liposomal formulation. In some instances, the adjuvant is a cyclic dinucleotide (CDN). Suitable CDNs are described below.

In some cases, the adjuvant is selected from the group consisting of an aluminum salt, RIBI, a toll-like receptor agonist, AS01, AS02, AS03, AS04, AS05, a CpG-oligodeoxynucleotide, MF-59, Montanide ISA-51 VG, Montanide ISA-720, Quil A, QS21, a synthetic saponin, an immunostimulatory complex, stearyl tyrosine, a virus-like particle, a reconstituted influenza virosome, a cytokine, mast cell activator compound 48/80, a liposome, a muramyl dipeptide, SAF-1, and combinations thereof. In some cases, the adjuvant is selected from the group consisting of an aluminum salt, alum, PHAD, a CDN, AS01, AS04, a CpG oligodeoxynucleotide, MF59, and combinations of two or more of the foregoing.

In some cases, the adjuvant is a disaccharide synthetic lipid compound, e.g., as described in U.S. Pat. No. 9,518,078. A disaccharide synthetic lipid compound can be a phosphorylated hexaacyl disaccharide (PHAD).

In some cases, the adjuvant is a PHAD of the following structure:

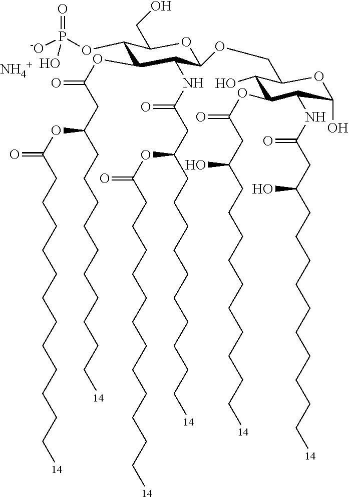

In some cases, a disaccharide synthetic lipid compound is a compound referred to as MPLA-B in U.S. Pat. No. 9,518,078; and has the following structure:

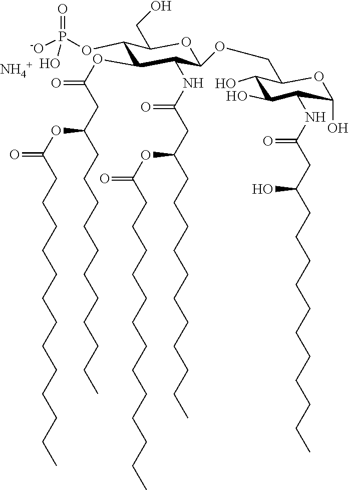

In some cases, a disaccharide synthetic lipid compound is a compound referred to as MPLA-D in U.S. Pat. No. 9,518,078; and has the following structure:

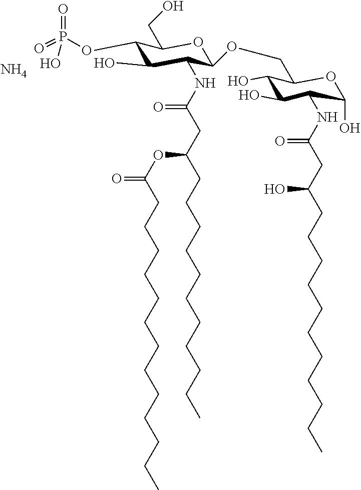

In some cases, a disaccharide synthetic lipid compound is a compound referred to as MPLA-C in U.S. Pat. No. 9,518,078; and has the following structure:

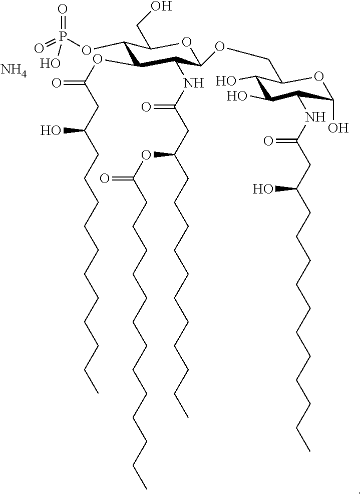

In some cases, the adjuvant is PHAD (also referred to as a glucopyranosyl lipid adjuvant (GLA)). In some cases, the adjuvant is a combination of alum and a PHAD.

### Cyclic Dinucleotides

In some cases, an immunogenic composition of the present disclosure comprises a cyclic dinucleotide (CDN).

In some cases, a CDN suitable for use in an immunogenic composition of the present disclosure is of Formula (I):

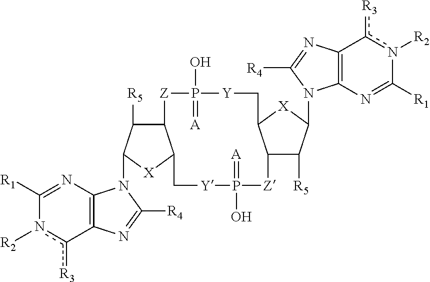

wherein:
A is S or O;
X is S, N, O, CH₂;
Y, Y′ is NH, CH₂, O;
Z, Z′ is NH, CH₂, O;
R1 represents hydrogen or NH₂ which may be substituted;
R2 is hydrogen or absent;
R3 represents NH₂, O, OH, H, or a halogen;
R4 represents hydrogen, halogen, or a straight or branched C₁-C₆ alkyl group which may optionally be substituted;
R5 represents hydrogen, OH or a straight or branched C₁-C₆ alkyl chain or C₁-C₆ straight or branched alkoxy chain which may optionally be substituted;
[symbol] is a single or double bond;
or conjugates thereof, and salts or solvates thereof. See, e.g., US 2008/0286296.

In formula (I), the purine residue is in some cases a guanine (G), adenine (A), xanthine or hypoxanthine (X), or inosine (I) residue. The compound can have identical purine residues, e.g. c-diGMP, c-diAMP, c-diIMP, or c-dXMP, or can contain different purine residues, e.g. c-GpAp, c-GpIp, c-GpXp, c-ApIp, c-ApXp, or c-IpXp. Further, R5 is in some cases an OH group. In addition, X is in some cases an oxygen atom. In one embodiment, Y, Y′, Z, and Z′ are an oxygen atom, O. Thus, in one embodiment, the compound of formula (I) is a cyclic bis(3^{′}-5^{′})diguanylic acid (c-diGMP) or conjugates thereof or a cyclic bis(3^{′}-5^{′})diadenylic acid (c-diAMP) or conjugates thereof, or salts or solvates thereof. In one embodiment, the compound of formula (I) is cyclic Bis(3^{′}-5^{′})adenylic acid, which is also referred to as c-di-AMP; or the pegylated conjugate. With the term “which may be substituted” is meant the substitution with a straight or branched C1-C6 alkyl group or a straight or branched C1-C6 alkoxy group and/or with a halogen, hydroxyl group or carboxyl group.

In some cases, a CDN suitable for use in an immunogenic composition of the present disclosure is selected from the group consisting of cyclic di-adenosine monophosphate (c-di-AMP), cyclic di-guanosine monophosphate (c-di-GMP), and cyclic guanosine monophosphate-adenosine monophosphate (cGAMP). In some cases, a CDN suitable for use in an immunogenic composition of the present disclosure is cGAMP (2^{′}-3^{′}-cyclic GMP-AMP) or cGAMP (3^{′}-3^{′}-cyclic GMP-AMP). In some cases, a CDN suitable for use in an immunogenic composition of the present disclosure is cGAMP (2^{′}-3^{′}-cyclic GMP-AMP). In some cases, a CDN suitable for use in an immunogenic composition of the present disclosure is cGAMP (3^{′}-3^{′}-cyclic GMP-AMP).

In some cases, a CDN suitable for use in an immunogenic composition of the present disclosure is of Formula (II):

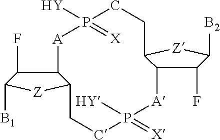

where:
A, C, A′ and C′ are independently selected from NH, O, and S;
X, Y, X′, and Y′ are independently selected from O or S;
Z and Z′ are independently selected from O, S, NH, and CH₂; and
B₁ and B₂ are independently a purine selected from:

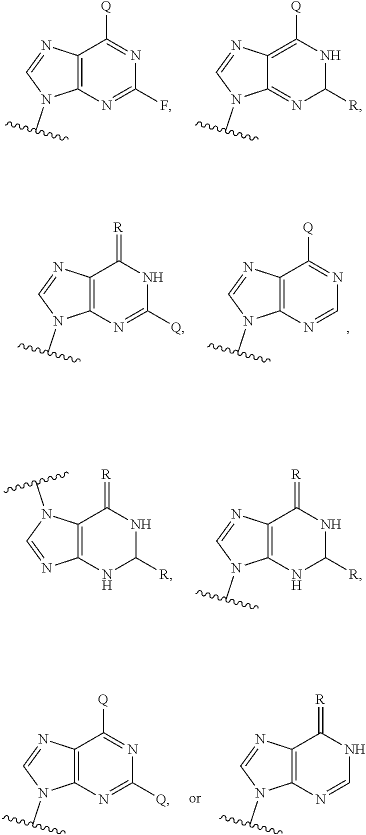

where:
Q is hydrogen or NH₂;
Nitrogen is optionally substituted with a C₁-C₆ alkyl or a C₁-C₆ acyl group; and
R is O or S.

In some cases, a CDN suitable for inclusion in an immunogenic composition of the present disclosure is a fluorinated CND. In some cases, the fluorinated CDN is 2^{′}-F-c-diGMP having the following structure:

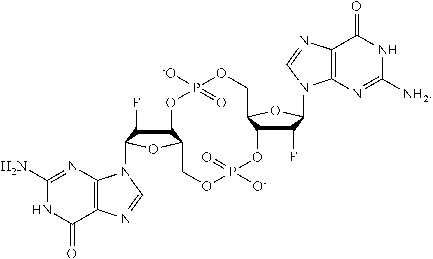

### Archaeal Glycolipid

In some cases, an immunogenic composition of the present disclosure comprises an archaeosome. For example, in some cases, an immunogenic composition of the present disclosure comprises an archaeosome comprising at least one polar synthetic lipid, where the at least one polar synthetic lipid comprises at least one carbohydrate or anionic group linked by covalent bonding to at least one free hydroxyl group of an archaeal core lipid.

An archaeal lipid suitable for use in an immunogenic composition of the present disclosure comprises a polar lipid based on a 2, 3-dialkylglycerol skeleton. These 2, 3-dialkylglycerol groups are isoprenoid and the simplest molecules are derivatives or 2,3-dibiphytanyl-O-sn-glycerol (archeol); for instance, two isoprenoid units of 20 carbons joined at positions sn-2 and sn-3 of glycerol. These alkyl chains are generally saturated; nevertheless, some forms have double bonds in different positions. These lipids have one or two groups of polar head, which may be different with units 2, 3-sn-glycerol joined by C40 alkyl components which are also isoprenoid molecules. For instance, calarcheol (so called because it is the predominant form in some thermophile archaebacteria), has two C40 isoprenoid units bonded from positions 2 to 3^{′} and from position 3 to 2′.

In some cases, an archaeal adjuvant suitable for use in an immunogenic composition of the present disclosure comprises multivalent cations in association with aggregates of a plurality of spherical archaeal polar lipid structures containing aqueous compartments (e.g., an “AMVAD structure”), where the archaeal polar lipid is a total polar lipids extract or archaetidyl glycerophosphate-O-methyl, obtained from an archaeal species. The multivalent cations can be divalent or trivalent cations. The multivalent cations can be divalent Ca²⁺ or Mg²⁺, or trivalent Al³⁺. The Ca²⁺ can be provided as CaCl₂. The Al³⁺ can be provided as AlCl₃ or AlK(SO₄)₂. In some cases, the total polar lipids extract from an archaeal species is mixed with neutral lipids from the archaeal species. See, e.g., U.S. Patent Publication No. 2013/0195932.

In some cases, lipids suitable for use in an immunogenic composition of the present disclosure comprises 1,2-di-O-hexadecyl-sn-glycero-3-phosphatidylcholine and 1,2-di-O-phytanyl-sn-glycero-3-phosphatidylethanolamine. In some cases, the 1,2-di-O-hexadecyl-sn-glycero-3-phosphatidylcholine and 1,2-di-O-phytanyl-sn-glycero-3-phosphatidylethanolamine form uniformly sized particles; for example, the particles can comprise: liposomes, nanoliposomes, niosomes, microspheres, nanospheres, nanoparticles, micelles or archaeosomes.

In some cases, an archaeosome comprises at least one polar synthetic lipid, where the at least one polar synthetic lipid comprises at least one carbohydrate or anionic group linked by covalent bonding to at least one free hydroxyl group of an archaeal core lipid. In some cases, the archaeal core lipid is archaeol (2,3-di-O-diphytanyl-sn-glycerol). In some cases, the archaeal core lipid is caldarchaeol (2,2′,3,3^{′}-tetra-O-dibiphytanyl-sn-diglycerol). In some cases, the the carbohydrate group is selected from the group consisting of: β-D-Glc-(1,6)-β-D-Glc-; β-D-Glc-(1,6)-α-D-Glc-; α-D-Glc-(1,6)-β-D-Glc-; β-D-Glc-(1,4)-β-D-Glc-; α-D-Glc-(1,4)-β-D-Glc-; p-D-Gal-(1,4)-β-D-Glc-; α-D-Gal-(1,6)-β-D-Glc-; β-D-Glc-(1,6)-β-D-Glc-(1,6)-β-D-Glc-; α-D-Glc-(1,4)-α-D-Glc-(1,4)-β-D-Glc-; α-D-Man-(1,2)-α-D-Man-(1,2)-α-D-Man-; and α-D-Man-(1,2)-α-D-Man-(1,2)-α-D-Man-(1,2)-α-D-Man-. In some cases, the carbohydrate group comprises two or three β-D-Glc-units in (1,6) linkage. In some cases, the carbohydrate group is a Galactose-Glucose (gal-glc) group. In some cases, the anionic group is selected from the group consisting of phosphoserine, phosphoethanolamine, phosphoinositol and phosphoglycerol. In some cases, the at least one anionic lipid is selected from the group consisting of archaetidylglycerol, archaetidylglycerolphosphate-methyl, archaetidylserine, and archaetidylinositol. In some cases, the archaeosome comprises at least one conventional lipid. In some cases, the at least one conventional lipid is selected from a group consisting of phosphatidylglycerol, phosphatidylserine, sulfoquinovosyl diacylglycerol (SQDG), and cholesterol. In some cases, the at least one conventional lipid comprises cholesterol, and wherein cholesterol is present in an amount of between 10 and 45 mol % of the total lipid composition. In some cases, the phosphatidylglycerol is present in an amount of between 20 and 80 mol % of the lipid composition. In some cases, the phosphatidylserine is present in an amount of between 10 and 30 mol % of the lipid composition. In some cases, the at least one polar synthetic lipid comprises at least one synthetic immunoactive glycolipid and at least one anionic lipid, and the archaeosome further comprises at least one stabilizing lipid. In some cases, the at least one polar synthetic lipid comprises caldarchaeol having one carbohydrate head group and one anionic head group. In some cases, the carbohydrate head group comprises gentiobiose and the anionic head group comprises phosphoinositol. In some cases, the at least one polar synthetic lipid comprises a first caldarchaeol having two carbohydrate head groups and a second caldarchaeol having two anionic head groups, and wherein the at least one stabilizing lipid is the first and/or second caldarchaeol. In some cases, the at least one polar synthetic lipid comprises gentiotriose-archaeol and wherein the at least one stabilizing lipid comprises cholesterol and at least one of phosphatidylethanolamine, archaetidylglycerol, archaetidylserine or archaetidylglycerolphosphate-methyl.

Caldarchaeol is also known as dibiphytanyldiglycerol tetraether. Two glycerol units are linked together by two strains that consist of two phytanes linked together to form a linear chain of 32 carbon atoms. Caldarchaeol has the following structure:

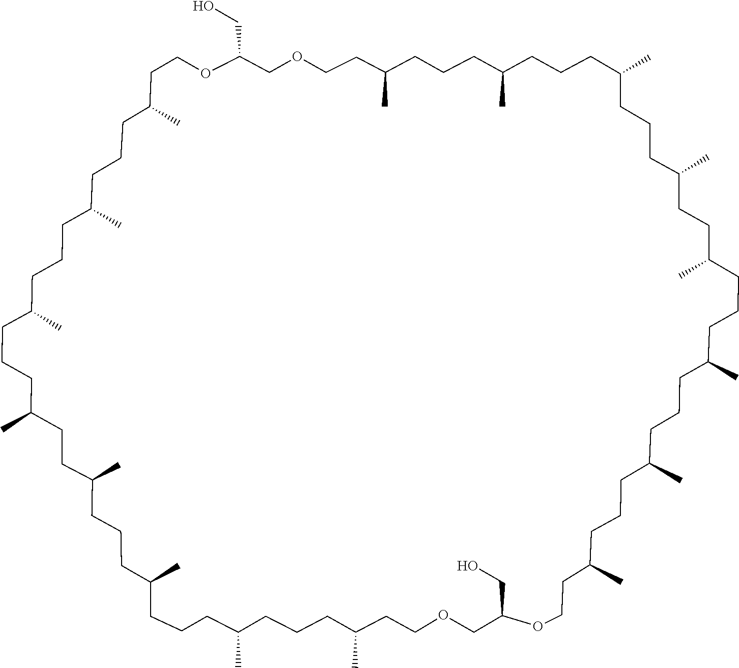 [SMILES: C[C@@H]1CCC[C@H](C)CCC[C@H](C)CC[C@@H](C)CCC[C@@H](C)CCC[C@@H](C)CCC[C@@H](C)CCO[C@H](CO)COCC[C@H](C)CCC[C@H](C)CCC[C@H](C)CCC[C@H](C)CC[C@@H](C)CCC[C@@H](C)CCC[C@@H](C)CCC[C@@H](C)CCO[C@H](CO)COCC[C@H](C)CCC1]

Archaeal lipids can be obtained from any archaea of the phyla Euryarchaeota, Crenarchaeota, Korarchaeota, or, Nanoarchaea. Archaeal lipids can be obtained from any archaea of the genus Thermococcus, Sulfolobus, Halobacterium, Methanococus, Ferroglobus, Therroplasma, Archacoglobus, Haloquadratun, or Halorubrum. Suitable sources of archaeal lipids include, but are not limited to, Thermus aquaticus, Thermus thermophilus; Methanobrevibacter smithii; Thermoplasma acidophilum; a Sulfolobus species, e.g. Sulfolobus acidocaldarius, Sulfolobus solfataricus, Sulfolobus islandicus, Sulfolobus tokodaii, etc.; a Pyrobaculum species, e.g. Pyrobaculum islandicum or Pyrobaculum aerophilum; a Methanococcus species, e.g., Methanocaldococcus vulcanius, Methanocaldococcus jannaschii, Methanococcus acolicus, Methanococcus voltae; or a Halobacterium species such as Halobacterium salinarum; Methanopyrus kandleri; Methanobacterium espanolae; Methanosphaera stadtmanae; Methanosarcina mazei; Natronobacterium magadii; etc.

Total polar lipids (TPL) can be extracted from archaea and collected as the acetone-insoluble fraction. Choquet et al. (1994) Appl. Microbiol. Biotechnol. 42:375; Bligh and Dyer (1959) Can. J. Biochem. Physiol. 37:911. The polar lipids consist of regularly branched, and usually fully saturated, phytanyl chains of 20, 25, or 40 carbon length, with the 20 and 40 being most common. Archaeosomes can be prepared by hydrating TPL in a buffer (e.g., phosphate-buffered saline). The TPL-buffer solution can be sonicated (e.g., at 60 Hz for 10 min).

TPL can be extracted from archaea by stirring the cells (which may be lyophilized) with chloroform-methanol (2:1, v/v) for 1 hour at room temperature. The suspension is passed through a sintered glass filter, and the residue reextracted for an additional hour. Combined filtrates are evaporated, taken up in chloroform-methanol-water (60:30:4.5, v/v/v), and passed through Sephadex G-25 for removal of nonlipid contaminations. Langworthy et al. (1977) J. Bacteriol. 130:1326.

The mean diameter of archaeosomes in an archaeosomal formulation can range from about 50 nm to 600 nm, e.g., from 50 nm to 100 nm, from 100 nm to 150 nm, from 150 nm to 200 nm, from 200 nm to 250 nm, from 250 nm to 300 nm, from 300 nm to 400 nm, from 400 nm to 450 nm, from 450 nm to 500 nm, from 500 nm to 550 nm, or from 550 nm to 600 nm.

### Containers

The present disclosure provides a container comprising an immunogenic composition of the present disclosure. The container can be sterile. The immunogenic composition can be sterile. The immunogenic composition can be suitable for administration to a human subject; e.g., the immunogenic composition can be free of pyrogens, allergens, or other substances that may be harmful to a human subject. Suitable containers include unit-dose containers, multi-dose sealed containers, ampules, vials, syringes, and the like. In some cases, the container is a syringe.

### Methods of Making an Hcv E1/E2 Heterodimer, and for Making a T-Cell Epitope Polypeptide

An HCV E1/E2 heterodimer, an HCV E2 polypeptide, an HCV E1 polypeptide, and a T-cell epitope polypeptide, suitable for inclusion in an immunogenic composition of the present disclosure, can be generated using standard methods for producing a polypeptide in a host cell.

An HCV E1/E2 heterodimer, an HCV E2 polypeptide, an HCV E1 polypeptide, and a T-cell epitope polypeptide, suitable for inclusion in an immunogenic composition of the present disclosure, can be produced using any suitable method, including recombinant and non-recombinant methods (e.g., chemical synthesis). An HCV E1/E2 heterodimer, an HCV E1 polypeptide, and a T-cell epitope polypeptide, suitable for inclusion in an immunogenic composition of the present disclosure, can be generated using standard methods for producing a polypeptide in a host cell.

Where a polypeptide is chemically synthesized, the synthesis may proceed via liquid phase or solid-phase. Solid-phase peptide synthesis (SPPS) allows the incorporation of unnatural amino acids and/or peptide/protein backbone modification. Various forms of SPPS, such as Fmoc and Boc, are available for synthesizing polypeptides. Details of the chemical synthesis are known in the art (e.g., Ganesan A. 2006 Mini Rev. Med Chem. 6:3-10; Camarero J A et al. 2005 Protein Pept Lett. 12:723-8).

Where a polypeptide is produced using recombinant techniques, the polypeptide may be produced as an intracellular protein or as a secreted protein, using any suitable construct and any suitable host cell, which can be a prokaryotic or eukaryotic cell, such as a bacterial (e.g., Escherichia coli) cell or a yeast host cell, respectively. Other examples of eukaryotic cells that may be used as host cells include insect cells, mammalian cells, filamentous fungi, and plant cells. Suitable yeast cells include, e.g., Saccharomyces cerevisiae and Pichia (e.g., Pichia pastoris).

In some cases, the heterologous polypeptide is produced separately from (e.g., in a separate host cell) from the HCV E1/E2 heterodimer. In some cases, the heterologous polypeptide is produced in a first host cell; and the HCV E1/E2 heterodimer is produced in a second host cell. Once the HCV E1/E2 heterodimer and the heterologous polypeptide are separately produced, they can be combined, together with a pharmaceutically acceptable excipient, to generate an immunogenic composition of the present disclosure. In some cases, both the HCV E1/E2 heterodimer and the heterologous polypeptide are purified before being combined to generate an immunogenic composition. For example, both the HCV E1/E2 heterodimer and the heterologous polypeptide can be at least 80%, at least 85%, at least 90%, at least 95%, at least 98%, at least 99%, or more than 99%, pure, e.g., free from other polypeptides, other macromolecules, etc.

### Methods of Making a T-Cell Epitope Polypeptide

A T-cell epitope polypeptide present in an immunogenic composition of the present disclosure can be generated using any known method for making a polypeptide. A T-cell epitope polypeptide can be chemically synthesized. A T-cell epitope polypeptide can be chemically synthesized via liquid phase or solid-phase. Solid-phase peptide synthesis (SPPS) allows the incorporation of unnatural amino acids and/or peptide/protein backbone modification. Various forms of SPPS, such as Fmoc and Boc, are available for synthesizing polypeptides. Details of the chemical synthesis are known in the art (e.g., Ganesan A. 2006 Mini Rev. Med Chem. 6:3-10 and Camarero J A et al. 2005 Protein Pept Lett. 12:723-8).

In some cases, a nucleic acid (e.g., a recombinant expression vector) comprising a nucleotide sequence encoding the T-cell epitope polypeptide is introduced into a host cell, generating a genetically modified (recombinant) host cell, where the recombinant expression vector provides for expression of the T-cell epitope polypeptide in the genetically modified host cell.

In some cases, the T-cell epitope polypeptide is produced as a fusion polypeptide comprising: a) the T-cell epitope polypeptide; and b) a fusion partner, where the fusion partner is an affinity tag. Suitable affinity tags include, e.g., immunoglobulin Fc polypeptides, a poly(histidine) tag (e.g., His₆), a maltose binding protein (MBP), a glutathione-S-transferase (GST) polypeptide, calmodulin-binding peptide (CBP), Streptavidin-binding peptide (SBP), Strep-tag II, FLAG (e.g., DYKDDDDK (SEQ ID NO:103), hemagglutinin (HA) (e.g., YPYDVPDYA (SEQ ID NO:104), c-myc T7 ((e.g., EQKLISEEDL; SEQ ID NO:105), Glu-Glu, starch-binding domain (SBD), and Flag-Acidic-Target Tag (FATT), and the like.

In some cases, the T-cell epitope polypeptide is produced as a fusion polypeptide comprising: a) the heterologous polypeptide; and b) a fusion partner (e.g., where the fusion partner is an Ig Fc polypeptide). In some cases, a proteolytically cleavable linker is interposed between the T-cell epitope polypeptide and the fusion partner, such that the fusion polypeptide comprises: a) the heterologous polypeptide; b) the proteolytically cleavable linker; and c) the fusion partner (e.g., Ig Fc polypeptide).

The proteolytically cleavable linker can include a protease recognition sequence recognized by a protease selected from the group consisting of alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase, gastricsin, gelatinase, Gly-X carboxypeptidase, glycyl endopeptidase, human rhinovirus 3C protease, hypodermin C, IgA-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, V8, venombin A, venombin AB, and Xaa-pro aminopeptidase.

For example, the proteolytically cleavable linker can comprise a matrix metalloproteinase cleavage site, e.g., a cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). For example, the cleavage sequence of MMP-9 is Pro-X-X-Hy (wherein, X represents an arbitrary residue; Hy, a hydrophobic residue), e.g., Pro-X-X-Hy-(Ser/Thr), e.g., Pro-Leu/Gln-Gly-Met-Thr-Ser (SEQ ID NO:107) or Pro-Leu/Gln-Gly-Met-Thr (SEQ ID NO:108). Another example of a protease cleavage site is a plasminogen activator cleavage site, e.g., a uPA or a tissue plasminogen activator (tPA) cleavage site. In some cases, the cleavage site is a furin cleavage site. Specific examples of cleavage sequences of uPA and tPA include sequences comprising Val-Gly-Arg. Another example of a protease cleavage site that can be included in a proteolytically cleavable linker is a tobacco etch virus (TEV) protease cleavage site, e.g., ENLYTQS (SEQ ID NO:109), where the protease cleaves between the glutamine and the serine. Another example of a protease cleavage site that can be included in a proteolytically cleavable linker is an enterokinase cleavage site, e.g., DDDDK (SEQ ID NO:87), where cleavage occurs after the lysine residue. Another example of a protease cleavage site that can be included in a proteolytically cleavable linker is a thrombin cleavage site, e.g., LVPR (SEQ ID NO:85). Additional suitable linkers comprising protease cleavage sites include linkers comprising one or more of the following amino acid sequences: LEVLFQGP (SEQ ID NO:97), cleaved by PreScission protease (a fusion protein comprising human rhinovirus 3C protease and glutathione-S-transferase; Walker et al. (1994) Biotechnol. 12:601); a thrombin cleavage site, e.g., CGLVPAGSGP (SEQ ID NO:110); SLLKSRMVPNFN (SEQ ID NO:111) or SLLIARRMPNFN (SEQ ID NO:112), cleaved by cathepsin B; SKLVQASASGVN (SEQ ID NO:113) or SSYLKASDAPDN (SEQ ID NO:114), cleaved by an Epstein-Barr virus protease; RPKPQQFFGLMN (SEQ ID NO:115) cleaved by MMP-3 (stromelysin); SLRPLALWRSFN (SEQ ID NO:116) cleaved by MMP-7 (matrilysin); SPQGIAGQRNFN (SEQ ID NO:117) cleaved by MMP-9; DVDERDVRGFASFL SEQ ID NO:118) cleaved by a thermolysin-like MMP; SLPLGLWAPNFN (SEQ ID NO:119) cleaved by matrix metalloproteinase 2(MMP-2); SLLIFRSWANFN (SEQ ID NO:120) cleaved by cathespin L; SGVVIATVIVIT (SEQ ID NO:121) cleaved by cathepsin D; SLGPQGIWGQFN (SEQ ID NO:122) cleaved by matrix metalloproteinase 1(MMP-1); KKSPGRVVGGSV (SEQ ID NO:123) cleaved by urokinase-type plasminogen activator; PQGLLGAPGILG (SEQ ID NO:124) cleaved by membrane type 1 matrixmetalloproteinase (MT-MMP); HGPEGLRVGFYESDVMGRGHARLVHVEEPHT (SEQ ID NO:125) cleaved by stromelysin 3 (or MMP-11), thermolysin, fibroblast collagenase and stromelysin-1; GPQGLAGQRGIV (SEQ ID NO:126) cleaved by matrix metalloproteinase 13 (collagenase-3); GGSGQRGRKALE (SEQ ID NO:127) cleaved by tissue-type plasminogen activator(tPA); SLSALLSSDIFN (SEQ ID NO:128) cleaved by human prostate-specific antigen; SLPRFKIIGGFN (SEQ ID NO:129) cleaved by kallikrein (hK3); SLLGIAVPGNFN (SEQ ID NO:130) cleaved by neutrophil elastase; and FFKNIVTPRTPP (SEQ ID NO:131) cleaved by calpain (calcium activated neutral protease).

Depending on the proteolytically cleavable linker, a heterologous polypeptide (a T-cell epitope polypeptide) can comprise, at its N-terminus or at its C-terminus, from 1 to 6 additional amino acids that are N-terminal or C-terminal to the cleavage site of the proteolytically cleavable linker. The following are non-limiting examples. In some cases, a heterologous polypeptide (a T-cell epitope polypeptide) is a modified T-cell epitope polypeptide that comprises from 1 to 6 additional amino acids at the N-terminus of the modified T-cell epitope polypeptide, where the from 1 to 6 additional amino acids are Gly-Pro, Ser, Gly, or Gly-Ser. In some cases, a heterologous polypeptide (a T-cell epitope polypeptide) is a modified T-cell epitope polypeptide that comprises from 1 to 6 additional amino acids at the C-terminus of the modified T-cell epitope polypeptide, where the from 1 to 6 additional amino acids are LEVLFQ (SEQ ID NO:83), ENLYYFQ (SEQ ID NO:84), LVPR (SEQ ID NO:85), I(E/D)GR (SEQ ID NO:86), or DDDDK (SEQ ID NO:87).

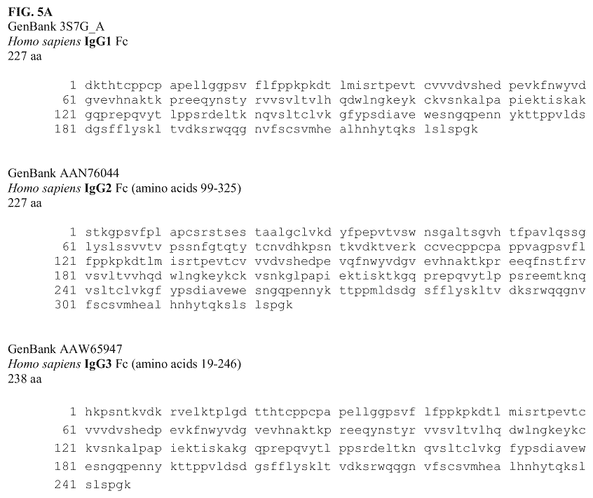

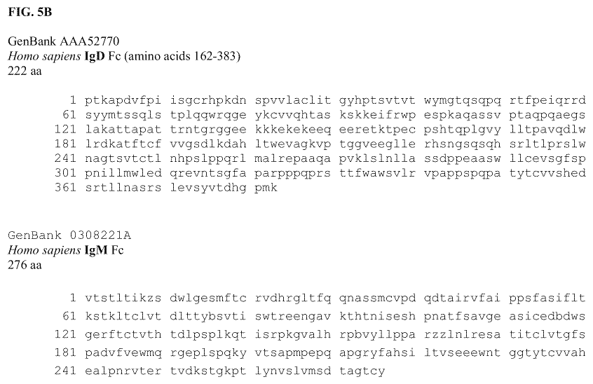

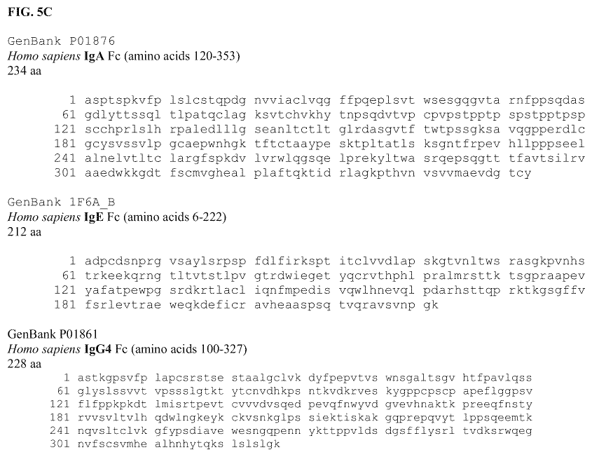

The Fc region can be a human IgG1 Fc, a human IgG2 Fc, a human IgG3 Fc, a human IgG4 Fc, etc. In some cases, the Fc region comprises an amino acid sequence having at least about 70%, at least about 75%, at least about 80%, at least about 85%, at least about 90%, at least about 95%, at least about 98%, at least about 99%, or 100%, amino acid sequence identity to an amino acid sequence of an Fc region depicted in FIG. 5A-5C. In some cases, the Fc region comprises an amino acid sequence having at least about 70%, at least about 75%, at least about 80%, at least about 85%, at least about 90%, at least about 95%, at least about 98%, at least about 99%, or 100%, amino acid sequence identity to the human IgG1 Fc polypeptide depicted in FIG. 5A. In some cases, the Fc region comprises an amino acid sequence having at least about 70%, at least about 75%, at least about 80%, at least about 85%, at least about 90%, at least about 95%, at least about 98%, at least about 99%, or 100%, amino acid sequence identity to the human IgG2 Fc polypeptide depicted in FIG. 5A; e.g., the Fc region comprises an amino acid sequence having at least about 70%, at least about 75%, at least about 80%, at least about 85%, at least about 90%, at least about 95%, at least about 98%, at least about 99%, or 100%, amino acid sequence identity to amino acids 99-325 of the human IgG2 Fc polypeptide depicted in FIG. 5A. In some cases, the Fc region comprises an amino acid sequence having at least about 70%, at least about 75%, at least about 80%, at least about 85%, at least about 90%, at least about 95%, at least about 98%, at least about 99%, or 100%, amino acid sequence identity to the human IgG3 Fc polypeptide depicted in FIG. 5A; e.g., the Fc region comprises an amino acid sequence having at least about 70%, at least about 75%, at least about 80%, at least about 85%, at least about 90%, at least about 95%, at least about 98%, at least about 99%, or 100%, amino acid sequence identity to amino acids 19-246 of the human IgG3 Fc polypeptide depicted in FIG. 5A.

Suitable expression vectors include, but are not limited to, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, herpes simplex virus, human immunodeficiency virus-based lentivirus vectors, murine leukemia virus (MVL)-based gamma retrovirus vectors, and the like), P1-based artificial chromosomes, yeast plasmids, yeast artificial chromosomes, and any other vectors specific for specific hosts of interest (such as Escherichia coli, mammalian cells, insect cells, or yeast cells).

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like.

Suitable prokaryotic cells include, but are not limited to, any of a variety of laboratory strains of Escherichia coli, Lactobacillus sp., Salmonella sp., Shigella sp., and the like. See, e.g., Carrier et al. (1992) J. Immunol. 148:1176-1181; U.S. Pat. No. 6,447,784; and Sizemore et al. (1995) Science 270:299-302.

Suitable eukaryotic host cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, and the like. Suitable yeast cells include, e.g., Saccharomyces cerevisiae and Pichia (e.g., Pichia pastoris).

Suitable insect cells include, e.g., Spodoptera frugiperda cells, e.g., Sf9 cells; Spodoptera frugiperda Sf-21 cells; Trichoplusia ni cells (e.g., Tn-368 cells; High-Five™ BTI-TN5B1-4 cells); etc.

Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, MRC4 fibroblast cells, and the like.

Methods for introduction of nucleic acids into host cells include, for example, transformation, electroporation, conjugation, calcium phosphate methods and the like. The method for transfer can be selected so as to provide for stable expression of the introduced polypeptide-encoding nucleic acid. The polypeptide-encoding nucleic acid can be provided as an inheritable episomal element (e.g., a plasmid) or can be genomically integrated.

In some cases, the T-cell epitope polypeptide is produced in a genetically modified host cell; and the heterologous polypeptide is purified from one or more of the cell culture medium and a cell lysate made from the genetically modified host cell. Methods of purifying a polypeptide from cell culture medium and/or a cell lysate are known in the art and include, e.g., affinity chromatography, size exclusion chromatography,

In some cases, where the T-cell epitope polypeptide is a fusion protein comprising the T-cell epitope polypeptide and a fusion partner, the fusion protein is purified on an affinity column comprising an antibody specific for the fusion partner, or other affinity partner that binds the fusion partner, immobilized on an insoluble support. In some cases, where the T-cell epitope polypeptide is a fusion protein comprising the T-cell epitope polypeptide and a fusion partner, and where the fusion partner is an Ig Fc polypeptide, the fusion protein can be purified on a Protein A column (i.e., affinity chromatography using Protein A immobilized on an insoluble support).

In some cases, where the T-cell epitope polypeptide is a fusion protein comprising the heterologous polypeptide and a fusion partner, and where the fusion partner is an Ig Fc polypeptide, and where a proteolytically cleavable linker is interposed between the Ig Fc and the T-cell epitope polypeptide, the fusion protein can be purified on a Protein A column (i.e., affinity chromatography using Protein A immobilized on an insoluble support). The fusion protein can be immobilized on the Protein A column; and an enzyme that cleaves a proteolytic cleavage site in the proteolytically cleavable linker is applied to the column comprising the immobilized fusion protein; the enzyme releases the heterologous polypeptide from the Protein A column.

### Methods of Making an HCV E1/E2 Heterodimer

An HCV E1/E2 heterodimer can be produced using any suitable method, including recombinant and non-recombinant methods (e.g., chemical synthesis).

Where a polypeptide is chemically synthesized, the synthesis may proceed via liquid phase or solid-phase. Solid-phase peptide synthesis (SPPS) allows the incorporation of unnatural amino acids and/or peptide/protein backbone modification. Various forms of SPPS, such as Fmoc and Boc, are available for synthesizing polypeptides. Details of the chemical synthesis are known in the art (e.g., Ganesan A. 2006 Mini Rev. Med Chem. 6:3-10 and Camarero J A et al. 2005 Protein Pept Lett. 12:723-8).

Where a polypeptide is produced using recombinant techniques, the polypeptide may be produced as an intracellular protein or as a secreted protein, using any suitable construct and any suitable host cell, which can be a prokaryotic or eukaryotic cell, such as a bacterial (e.g., Escherichia coli) cell or a yeast host cell, respectively. Other examples of eukaryotic cells that may be used as host cells include insect cells, mammalian cells, filamentous fungi, and plant cells. Suitable yeast cells include, e.g., Saccharomyces cerevisiae and Pichia (e.g., Pichia pastoris).

Suitable mammalian cells include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, MRC5 cells (ATCC No. CCL-171), and the like. Where mammalian host cells are used, such host cells may include human cells (e.g., HeLa, 293, H9 and Jurkat cells); mouse cells (e.g., NIH3T3, L cells, and C127 cells); primate cells (e.g., Cos 1, Cos 7 and CV1); MRC4 cells; and hamster cells (e.g., Chinese hamster ovary (CHO) cells).

A variety of host-vector systems suitable for the expression of a polypeptide may be employed according to standard procedures known in the art. See, e.g., Sambrook et al., 1989 Current Protocols in Molecular Biology Cold Spring Harbor Press, New York; Ausubel et al. 1995 Current Protocols in Molecular Biology, Eds. Wiley and Sons; “Protein Expression: A Practical Approach” (1999) S. J. Higgins and B. D. James, eds., Oxford University Press; “Protein Expression in Mammalian Cells: Methods and Protocols (Methods in Molecular Biology)” (2012) James L. Hartley, ed., Humana Press; and “Production of Recombinant Proteins” (2005) Gerd Gellisen, ed., Wiley-VCH. Methods for introduction of nucleic acids into host cells include, for example, transformation, electroporation, conjugation, calcium phosphate methods and the like. The method for transfer can be selected so as to provide for stable expression of the introduced polypeptide-encoding nucleic acid. The polypeptide-encoding nucleic acid can be provided as an inheritable episomal element (e.g., a plasmid) or can be genomically integrated. A variety of appropriate vectors for use in production of a peptide of interest are available commercially.

Suitable expression vectors include, but are not limited to, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, herpes simplex virus, HIV-based lentivirus vectors, murine leukemia virus (MVL)-based gamma retrovirus vectors, and the like), P1-based artificial chromosomes, yeast plasmids, yeast artificial chromosomes, and any other vectors specific for specific hosts of interest (such as E. coli, mammalian cells, insect cells, or yeast cells).

An E1 polypeptide, an E2 polypeptide, or an E1/E2 heterodimer can be produced by introducing a recombinant expression vector comprising a nucleotide sequence encoding the E1 polypeptide, E2 polypeptide, or E1/E2 heterodimer into an appropriate host cell, where the host cell produces the encoded E1 polypeptide, E2 polypeptide, or E1/E2 heterodimer. In the expression vector, a polynucleotide comprising a nucleotide sequence(s) encoding the E1 polypeptide, E2 polypeptide, or E1/E2 heterodimer is linked to a regulatory sequence as appropriate to obtain the desired expression properties. These regulatory sequences can include promoters, enhancers, terminators, operators, repressors, and inducers. The promoters can be regulated or constitutive. Expression vectors generally have convenient restriction sites located near the promoter sequence to provide for the insertion of nucleic acid sequences encoding a protein of interest. A selectable marker operative in the expression host cell may be present.

In some cases, the E1/E2 heterodimer is encoded in a recombinant expression vector suitable for expression in a eukaryotic host cell (e.g., an insect cell; a yeast cell; a mammalian host cell, such as CHO cells, HeLa cells, 293 cells, MRC5 cells, etc.). In some cases, a recombinant expression vector comprises a nucleotide sequence encoding E1 and E2 polypeptides (which may be wild-type or variant) as a single polypeptide chain; the recombinant expression vector is introduced into a eukaryotic host cell to generate a genetically modified host cell. In some cases, E1 and E2 polypeptides are initially produced as a single polypeptide chain, which is cleaved in the endoplasmic reticulum (ER) of the genetically modified host cell to produce separate E1 and E2 polypeptides. The separate E1 and E2 polypeptides can form a heterodimer (e.g., a non-covalently linked heterodimer) in the ER. The E1/E2 heterodimer can be isolated from the genetically modified host cell by, e.g., lysis using a non-ionic detergent, or using a freeze-thaw method. See, e.g., Frey et al. (2010) Vaccine 28:6367. The E1/E2 heterodimer can be purified from a cell lysate and/or cell culture medium using any of a variety of methods, including size exclusion chromatography, affinity chromatography, and the like, or combinations of such methods. In some cases, the E1/E2 heterodimer is purified from cell lysate and/or cell culture medium using Galanthus nivalis (GNA) lectin affinity chromatography. In some cases, the E1/E2 heterodimer is purified from a cell lysate. In some cases, the E1/E2 heterodimer is secreted from a cell and is purified from the cell culture medium. Suitable methods that can be used for purifying an E1/E2 heterodimer are described in, e.g., U.S. Pat. Nos. 6,121,020; 6,274,148; and Mazzocca et al. (2005) J. Biol. Chem. 280:11329. For example, in some cases, an E1/E2 heterodimer can be prepared in a method comprising cell disruption and debris removal by microfiltration, followed by purification using three subsequent chromatographic steps: lectin affinity chromatography, hydroxyapatite chromatography, and ion exchange chromatography.

Alternatively, the E1 and E2 polypeptides can be encoded on separate recombinant expression vectors; and produced in a cell (e.g., the same host cell or separate host cells) as separate polypeptides.

If full-length E1 and E2 polypeptides are expressed in a eukaryotic host cell, the E1 and E2 polypeptides remain bound to the endoplasmic reticulum (ER) membrane as asialoglycoproteins. If the E1 and E2 polypeptides have C-terminal truncations, such that the C-terminal transmembrane regions are removed, the truncated polypeptides are secreted and can acquire complex glycans such as sialic acid. Removal of approximately amino acids 660-746 of E2, or amino acids 715-746 of E2, and removal of approximately amino acids 330-383 of E1, results in secretion of E2 and E1 from a eukaryotic host cell. If E1 and E2 are co-expressed in 9the same eukaryotic host cell as full-length polypeptides, they remain in the lumen of the ER as a heterodimer.

In some cases, an E2 polypeptide suitable for use in an E1/E2 heterodimer lacks a transmembrane region. For example, in some cases, an E2 polypeptide suitable for use in an E1/E2 heterodimer, comprises amino acids 384-659, and lacks amino acids 660-746 of a naturally-occurring E2 polypeptide; and may be referred to as “E2 ectodomain polypeptide.” For example, in some cases, an E2 polypeptide suitable for use in an E1/E2 heterodimer comprises amino acids 384-659, lacks amino acids 660-746 of a naturally-occurring E2 polypeptide, and has a length of 276 amino acids.

In some cases, an E1 polypeptide suitable for use in an E1/E2 heterodimer lacks a transmembrane region. For example, in some cases, an E1 polypeptide suitable for use in an E1/E2 heterodimer comprises amino acids 191-329, and lacks amino acids 330-383 of a naturally-occurring E1 polypeptide; and may be referred to as an “E1 ectodomain polypeptide.” For example, in some cases, an E1 polypeptide suitable for use in an E1/E2 heterodimer comprises amino acids 191-329, lacks amino acids 330-383 of a naturally-occurring E1 polypeptide, and has a length of 139 amino acids.

After production in a host cell, an E1 polypeptide, an E2 polypeptide, or an E1/E2 heterodimer (e.g., as separate polypeptides or as a heterodimer) can be purified from the host cell. Methods of purification of recombinantly produced polypeptides from a host cell are known in the art and include, e.g., detergent lysis (e.g., with a non-ionic detergent) or freeze-thaw lysis, followed by one or more of size exclusion column chromatography, high performance liquid chromatography, affinity chromatography, and the like.

In some cases, an E1/E2 heterodimer suitable for inclusion in an immunogenic composition of the present disclosure is produced by purifying an E1/E2 heterodimer on an affinity column, where the E1 or E2 polypeptide comprises an Ig Fc polypeptide linked to the E1 or E2 polypeptide via a proteolytically cleavable linker. For example, the method can comprise: A) culturing a genetically modified eukaryotic host cell that is genetically modified with a nucleic acid (e.g., recombinant expression vector) comprising a nucleotide sequence encoding an E1/E2 polypeptide that comprises, in order from N-terminus to C-terminus: a) a signal peptide that directs the E1/E2 polypeptide to the ER following translation of the E1/E2 polypeptide; b) an HCV E1 polypeptide; c) an Ig Fc region; d) a proteolytically cleavable linker; and e) an HCV E2 polypeptide); B) contacting a lysate of the cultured genetically modified eukaryotic host cell with a solid support comprising an Ig Fc binding moiety, generating an immobilized heterodimer comprising the HCV E1 polypeptide and a fusion polypeptide comprising: a) the Ig Fc; b) the proteolytically cleavable linker; and c) the E2 polypeptide; C) contacting the immobilized heterodimer with an enzyme that cleaves the proteolytically cleavable linker, thereby releasing the heterodimer; and D) collecting the released heterodimer.

In some cases, an E1/E2 heterodimer is produced using a method comprising: A) culturing a genetically modified eukaryotic host cell that is genetically modified with a nucleic acid (e.g., recombinant expression vector) comprising a nucleotide sequence encoding an E1/E2 polypeptide that comprises, in order from N-terminus to C-terminus: a) a signal peptide that directs the E1/E2 polypeptide to the ER following translation of the E1/E2 polypeptide; b) an HCV E1 polypeptide; c) an Ig Fc region; d) a proteolytically cleavable linker; and e) an HCV E2 polypeptide; B) contacting a lysate of the cultured genetically modified eukaryotic host cell with a solid support comprising an Ig Fc binding moiety, generating an immobilized heterodimer comprising the HCV E1 polypeptide and a fusion polypeptide comprising: a) the Ig Fc; b) the proteolytically cleavable linker; and c) the E2 polypeptide; C) contacting the immobilized heterodimer with an enzyme that cleaves the proteolytically cleavable linker, thereby releasing the heterodimer; and D) collecting the released heterodimer.

In some cases, an E1/E2 heterodimer is produced using a method comprising: A) culturing a genetically modified eukaryotic host cell that is genetically modified with a nucleic acid (e.g., recombinant expression vector) comprising a nucleotide sequence encoding an E1/E2 polypeptide that comprises, in order from N-terminus to C-terminus: a) a signal peptide that directs the E1/E2 polypeptide to the ER following translation of the E1/E2 polypeptide; b) an HCV E1 polypeptide; c) an Ig Fc region; d) a proteolytically cleavable linker comprising the amino acid sequence LEVLFQGP (SEQ ID NO:97), and having a length of from 8 amino acids to 15 amino acids; and e) an HCV E2 polypeptide; B) contacting a lysate of the cultured genetically modified eukaryotic host cell with a solid support comprising an Ig Fc binding moiety, generating an immobilized heterodimer comprising the HCV E1 polypeptide and a fusion polypeptide comprising: a) the Ig Fc; b) the proteolytically cleavable linker; and c) the E2 polypeptide; C) contacting the immobilized heterodimer with an enzyme (e.g., a rhinovirus 3C protease) that cleaves the proteolytically cleavable linker, thereby releasing the heterodimer; and D) collecting the released heterodimer.

In some cases, an E1/E2 heterodimer is produced using a method comprising: A) culturing a genetically modified eukaryotic host cell that is genetically modified with a nucleic acid (e.g., recombinant expression vector) comprising a nucleotide sequence encoding an E1/E2 polypeptide that comprises, in order from N-terminus to C-terminus: a) a signal peptide that directs the E1/E2 polypeptide to the ER following translation of the E1/E2 polypeptide; b) an HCV E1 polypeptide; c) an Ig Fc region; d) a proteolytically cleavable linker comprising the amino acid sequence LEVLFQGP (SEQ ID NO:97), and having a length of from 8 amino acids to 15 amino acids; and e) an HCV E2 polypeptide; B) contacting a lysate of the cultured genetically modified eukaryotic host cell with a solid support comprising an Ig Fc binding moiety, generating an immobilized heterodimer comprising the HCV E1 polypeptide and a fusion polypeptide comprising: a) the Ig Fc; b) the proteolytically cleavable linker; and c) the E2 polypeptide; C) contacting the immobilized heterodimer with an enzyme (e.g., a fusion polypeptide comprising a glutathione-S-transferase and a human rhinovirus 3C protease (GST-HRV3C protease)) that cleaves the proteolytically cleavable linker, thereby releasing the E1E2 heterodimer; and D) collecting the released E1E2 heterodimer. In some cases, a solution comprising the released E1E2 heterodimer is applied to glutathione immobilized on a solid support, to remove the GST-HRV3C protease. For example, a solution comprising the released heterodimer can be applied to a glutathione-Sepharose 4B column, where the GST-HRV3C binds to the glutathione-Sepharose 4B; the flow-through (unbound material) comprises the released E1E2 heterodimer. In some cases, the released E1E2 heterodimer is further subjected to hydroxyapatite chromatography. Hydroxyapatite chromatography can be carried out as described in, e.g., Mazzocca et al. (2005) J. Biol. Chem. 280:11329.

Suitable Ig Fc binding moieties include, but are not limited to, Protein A (Graille et al. (2000) Proc. Natl. Acad. Sci. USA 97:5399); Protein G (Sjöbring et al. (1991) J. Biol. Chem. 266:399); and a Protein A/G fusion polypeptide (Eliasson et al. (1988) J. Biol. Chem. 263:4323).

The Ig Fc binding moiety can be immobilized onto a solid support, where the solid support can be of any of a variety of forms, e.g., a bead, a magnetic bead, a plate, and the like. The solid support can be made of any of a variety of materials, including, but not limited to, polystyrene, agarose, polyesters, polyethylene, and the like.

As an alternative to Fc, an affinity tag such as, e.g., polyhistidine (e.g., (His)₆), glutathione-S-transferase (GST), calmodulin-binding peptide (CBP), Streptavidin-binding peptide (SBP), Strep-tag II, FLAG (e.g., DYKDDDDK (SEQ ID NO:103), hemagglutinin (HA) (e.g., YPYDVPDYA (SEQ ID NO:104), c-myc T7 ((e.g., EQKLISEEDL; SEQ ID NO:105), Glu-Glu, and the like, can be used. (Wood D. 2014. Current Opinion in Structural Biology 26 54-61; Kimple M E et al. 2013. Current Protocols in Protein Science 9.9.1-9.9.23). Other suitable affinity tags include, e.g., starch-binding domain (SBD); and Flag-Acidic-Target Tag (FATT). See, e.g., Wood D. 2014. Current Opinion in Structural Biology 26 54-61).

One or more additional purification steps can be carried out. For example, a solution comprising the released heterodimer, produced as described above, can be subjected to size exclusion chromatography, hydroxyapatite chromatography, and the like. Hydroxyapatite chromatography can be carried out as described in, e.g., Mazzocca et al. (2005) J. Biol. Chem. 280:11329.

An E1/E2 heterodimer can be purified such that the E1/E2 heterodimer is at least 60% pure, at least 65% pure, at least 70% pure, at least 75% pure, at least 80% pure, at least 85% pure, at least 90% pure, at least 95% pure, at least 98% pure, at least 99% pure, or greater than 99% pure.

### Nucleic Acid Immunogenic Compositions

The present disclosure provides nucleic acid compositions comprising: a) one or more nucleic acids comprising a nucleotide sequence(s) encoding polypeptides (e.g., HCV E1/E2; HCV E1; HCV E2; a T-cell epitope polypeptide) as described above. The present disclosure provides an immunogenic composition comprising: a) a nucleic acid (e.g., a recombinant viral expression vector(s)) comprising nucleotide sequence(s) encoding one or more of: an HCV E1/E2 heterodimer; an HCV E1 polypeptide; an HCV E2 polypeptide; and a T-cell epitope polypeptide, where such polypeptides are described above. The polypeptides can be encoded in the same nucleic acid, or on separate nucleic acids. For example, where the nucleic acid(s) are recombinant expression vectors, the polypeptides can be encoded in the same or separate recombinant expression vectors.

In some cases, the nucleic acid(s) is/are DNA. In some cases, the nucleic acid(s) is/are RNA. In some cases, the nucleic acid(s) is/are present in expression vector(s), generating recombinant expression vector(s) comprising the nucleic acid(s). In some cases, the recombinant expression vector(s) is/are recombinant bacterial vectors. In some cases, the recombinant expression vector(s) is/are recombinant viral vector(s). In some cases, the recombinant viral vector(s) are packaged into viral particles. In some cases, the nucleic acid(s) are present in bacteria (e.g., non-pathogenic bacteria (e.g., attenuated bacteria) suitable for delivery of nucleic acids to an individual). Where the recombinant expression vector is a bacterial vector or a viral vector, the vector is suitably attenuated so as not to cause significant pathology in an individual.

In some cases, the nucleic acid is present in an expression vector. Suitable expression vectors include, but are not limited to, a replication-defective adenovirus vector; a replication-defective vaccinia virus vector; a lentivirus vector (e.g., a self-inactivating lentivirus vector); a retroviral vector (e.g., a self-inactivating retroviral vector); an adeno-associated virus vector; and the like. In some cases, the vector is a modified vaccinia Ankara (MVA) vector, or an MVA-based vector (see, e.g., Verheust et al. (2012) Vaccine 30:2623). In some cases, the vector is a replication-defective adenovirus vector. In some cases, the vector is a replication-defective adenovirus 6 (Ad6) vector. In some cases, the vector is a replication-defective simian adenovirus vector (e.g., ChAd3). Suitable viral vectors are described in, e.g., Zhou et al. (2012) Invest. Ophthalmol. Vis. Sci. 53:2804; Swadling et al. (2014) Sci. Transl. Med. 6:261ra153; and Choi and Chang (2013) Clin. Exp. Vaccine Res. 2:97. In many cases, the recombinant viral vectors are packaged into viral particles; and the viral particles are formulated in an immunogenic composition along with a pharmaceutically acceptable carrier. Suitable pharmaceutically acceptable carriers are described above.

In some cases, an immunogenic composition of the present disclosure comprises: a) a recombinant viral vector comprising nucleotide sequences encoding one or more of: an HCV E1/E2 heterodimer; an HCV E1 polypeptide; an HCV E2 polypeptide; and a T-cell epitope polypeptide; and b) a pharmaceutically acceptable excipient. In some cases, an immunogenic composition of the present disclosure comprises: a) a recombinant viral vector comprising nucleotide sequences encoding: an HCV E1/E2 heterodimer; and a T-cell epitope polypeptide; and b) a pharmaceutically acceptable excipient. In some cases, an immunogenic composition of the present disclosure comprises: a) a recombinant viral vector comprising nucleotide sequences encoding: an HCV E1 polypeptide; and a T-cell epitope polypeptide; and b) a pharmaceutically acceptable excipient. In some cases, an immunogenic composition of the present disclosure comprises: a) a recombinant viral vector comprising nucleotide sequences encoding: an HCV E2 polypeptide; and a T-cell epitope polypeptide; and b) a pharmaceutically acceptable excipient.

In some cases, the present disclosure provides: a) a first immunogenic composition comprising: i) a recombinant viral vector comprising nucleotide sequences encoding one or more of: an HCV E1/E2 heterodimer; an HCV E1 polypeptide; an HCV E2 polypeptide; and a T-cell epitope polypeptide; and ii) a pharmaceutically acceptable excipient; and b) a second immunogenic composition comprising: i) a recombinant viral vector comprising nucleotide sequences encoding one or more of: an HCV E1/E2 heterodimer; an HCV E1 polypeptide; an HCV E2 polypeptide; and a T-cell epitope polypeptide; and ii) a pharmaceutically acceptable excipient.

In some cases, the present disclosure provides: a) a first immunogenic composition comprising: i) a first recombinant viral vector comprising nucleotide sequences encoding one or more of: an HCV E1/E2 heterodimer; an HCV E1 polypeptide; an HCV E2 polypeptide; and a T-cell epitope polypeptide; and ii) a pharmaceutically acceptable excipient; and b) a second immunogenic composition comprising: i) a second recombinant viral vector comprising nucleotide sequences encoding one or more of: an HCV E1/E2 heterodimer; an HCV E1 polypeptide; an HCV E2 polypeptide; and a T-cell epitope polypeptide; and ii) a pharmaceutically acceptable excipient. In some cases, the first recombinant viral vector is a replication-defective adenovirus-based recombinant viral vector; and the second recombinant viral vector is an MVA-based recombinant viral vector. In some cases, the first recombinant viral vector is a chimpanzee adenovirus-based recombinant viral vector; and the second recombinant viral vector is an MVA-based recombinant viral vector.

In some cases, the nucleic acid(s) are present in bacteria (e.g., non-pathogenic bacteria suitable for delivery of nucleic acids to an individual). In some cases, the nucleic acid(s) are present in recombinant expression vector(s) present in bacteria (e.g., non-pathogenic bacteria suitable for delivery of nucleic acids to an individual). Thus, the present disclosure provides an immunogenic composition comprising a non-pathogenic, bacterium that harbors a nucleic acid(s) comprising nucleotide sequences encoding one or more of: an HCV E1/E2 heterodimer; an HCV E1 polypeptide; an HCV E2 polypeptide; and a T-cell epitope polypeptide, where such polypeptides are described above. The present disclosure provides an immunogenic composition comprising a non-pathogenic bacterium that harbors a recombinant expression vector(s) comprising nucleotide sequences encoding one or more of: an HCV E1/E2 heterodimer; an HCV E1 polypeptide; an HCV E2 polypeptide; and a T-cell epitope polypeptide, where such polypeptides are described above. In some cases, the bacteria are live. In some cases, the bacteria are live attenuated bacteria. In some cases, the bacteria are killed.

Bacteria suitable for delivery of nucleic acid(s) (which nucleic acid(s) may be present in expression vector(s)) include, but are not limited to, Lactobacillus; Lactococcus (e.g., Lactococcus lactis); Salmonella, e.g., attenuated, non-pathogenic Salmonella, e.g., Salmonella enterica serovar Typhi, Salmonella enterica serovar Typhimurium; non-pathogenic strains of Francisella; non-pathogenic strains of Escherichia coli; non-pathogenic strains of Bordetella pertussis; non-pathogenic strains of Listeria; non-pathogenic strains of Shigella; non-pathogenic strains of Vibrio (e.g., Vibrio cholera); Streptococcus gordonii; non-pathogenic strains of Yersinia enterocolitica; non-pathogenic strains of Shigella flexneri; non-pathogenic strains of Pseudomonas aeruginosa; non-pathogenic strains of Bacillus subtilis; and the like.

In some cases, one or more virulence genes in the bacterium is all or partially deleted. For example, for Salmonella enterica serovar Typhi and Salmonella enterica serovar Typhimurium, an aroA, aroC, and aroD mutation can be made. Other mutations that can attenuate pathogenicity affect biosynthesis of the nucleotides adenine (pur) and guanine (guaBA), and outer membrane proteins C and F (ompC, ompF), as well as expression of the cAMP receptor (cya/crp), the conversion of UDP-galactose to UDP-glucose (galE), DNA recombination and repair (recA, recBC), and regulation of virulence genes (phoP, phoQ). For Listeria monocytogenes, attenuation can be achieved with auxotrophic mutants, or deletion of virulence factors such as the genes actA and internalin B (intB).

### Methods of Inducing an Immune Response to Hcv

The present disclosure provides a method of inducing an immune response (e.g., a protective immune response) to at least one HCV genotype in a mammalian subject. In some cases, a method of the present disclosure for inducing an immune response in an individual to at least one HCV genotype comprises administering an immunogenic composition of the present disclosure, where the immunogenic composition comprises polypeptides (e.g., HCV E1/E2; HCV E1; HCV E2; T-cell epitope polypeptide). In some cases, a method of the present disclosure for inducing an immune response in an individual to at least one HCV genotype comprises administering an immunogenic composition of the present disclosure, where the immunogenic composition comprises one or more nucleic acids comprising nucleotide sequences encoding polypeptides (e.g., HCV E1/E2; HCV E1; HCV E2; T-cell epitope polypeptide).

### Administering an Immunogenic Composition Comprising Polypeptides

In some cases, the methods comprise administering to an individual in need thereof an effective amount of an immunogenic composition of the present disclosure, where the immunogenic composition comprises: a) an HCV E1/E2 heterodimer; b) a T-cell epitope polypeptide comprising a T-cell epitope present in an HCV polypeptide other than E1 and E2; and c) a pharmaceutically acceptable excipient; or where the immunogenic composition comprises: a) an HCV E2 polypeptide; b) a T-cell epitope polypeptide comprising a T-cell epitope present in an HCV polypeptide other than E1 and E2; and c) a pharmaceutically acceptable excipient; or where the immunogenic composition comprises: a) an HCV E1 polypeptide; b) a T-cell epitope polypeptide comprising a T-cell epitope present in an HCV polypeptide other than E1 and E2; and c) a pharmaceutically acceptable excipient.

### Administering an Immunogenic Composition Comprising Nucleic Acid(s)

In some cases, a method of the present disclosure for inducing an immune response to HCV in an individual comprises administering to the individual an effective amount of a nucleic acid(s) comprising nucleotide sequences encoding: 1) an HCV E1/E2 heterodimer and a T-cell epitope polypeptide; 2) an HCV E2 polypeptide and a T-cell epitope polypeptide; or 3) an HCV E1 polypeptide and a T-cell epitope polypeptide. The polypeptides can be encoded in the same nucleic acid, or on separate nucleic acids. For example, where the nucleic acid(s) are recombinant expression vectors, the polypeptides can be encoded in the same or separate recombinant expression vectors.

In some cases, the nucleic acid(s) is/are DNA. In some cases, the nucleic acid(s) is/are RNA. In some cases, the nucleic acid(s) is/are present in expression vector(s) such that a recombinant expression vector(s) comprising the nucleic acid(s) are administered. In some cases, the recombinant expression vector(s) is/are recombinant viral vector(s). In some cases, the recombinant viral vector(s) are packaged into viral particles. In some cases, the nucleic acid(s) are present in bacteria (e.g., non-pathogenic bacteria (e.g., attenuated bacteria) suitable for delivery of nucleic acids to an individual).

In some cases, the nucleic acid is present in an expression vector, thereby generating a recombinant expression vector. Suitable expression vectors include, but are not limited to, a replication-defective adenovirus vector; a replication-defective vaccinia virus vector; a lentivirus vector (e.g., a self-inactivating lentivirus vector); a retroviral vector (e.g., a self-inactivating retroviral vector); an adeno-associated virus vector; and the like. In some cases, the vector is a modified vaccinia Ankara (MVA) vector, or an MVA-based vector (see, e.g., Verheust et al. (2012) Vaccine 30:2623). In some cases, the vector is a replication-defective adenovirus vector. In some cases, the vector is a replication-defective adenovirus 6 (Ad6) vector. In some cases, the vector is a replication-defective simian adenovirus vector (e.g., ChAd3). Suitable viral vectors are described in, e.g., Zhou et al. (2012) Invest. Ophthalmol. Vis. Sci. 53:2804; Swadling et al. (2014) Sci. Transl. Med. 6:261ra153; and Choi and Chang (2013) Clin. Exp. Vaccine Res. 2:97. In many cases, the recombinant viral vectors are packaged into viral particles; and the viral particles are formulated in an immunogenic composition along with a pharmaceutically acceptable carrier.

In some cases, an HCV E1/E2 heterodimer is encoded by nucleotide sequences present in a first recombinant viral vector, e.g., an adenovirus vector, a vaccinia virus vector, an MVA vector or MVA-based vector; and a T-cell epitope polypeptide is encoded by nucleotide sequences present in a second recombinant viral vector, e.g., an adenovirus vector, a vaccinia virus vector, an MVA vector or MVA-based vector.

In some cases, a prime-boost vaccine protocol is used. In some cases, a first (priming) immunogenic composition is administered, where the first immunogenic composition comprises a recombinant viral vector comprising nucleotide sequences encoding one or more of: a) an HCV E1/E2 heterodimer; b) an HCV E2 polypeptide; c) an HCV E1 polypeptide; and d) a T-cell epitope polypeptide; and, after a time, a second (booster) immunogenic composition is administered, where the second immunogenic composition comprises a recombinant viral vector comprising nucleotide sequences encoding one or more of: a) an HCV E1/E2 heterodimer; b) an HCV E2 polypeptide; c) an HCV E1 polypeptide; and d) a T-cell epitope polypeptide. In some cases, the first recombinant viral vector and the second recombinant viral vector are the same. In some cases, the first recombinant viral vector and the second recombinant viral vector are different. For example, in some cases, the first recombinant viral vector is a vaccinia-based recombinant viral vector; and the second recombinant viral vector is an adenovirus-based recombinant viral vector. In general, the recombinant viral vectors are packaged into viral particles. A second immunogenic composition can be administered at a time period of from 1 day to 1 year following administration of the first immunogenic composition. For example, a second immunogenic composition can be administered at a time period of from 1 day to 1 week, from 1 week to 2 weeks, from 2 weeks to 1 month, from 1 month to 2 months, from 2 months to 6 months, or from 6 months to 1 year following administration of the first immunogenic composition.

For example, in some cases, a first (priming) vaccine comprising a recombinant adenovirus (e.g., Ad6 or chimpanzee Ad (e.g., ChAd3)) that comprises a nucleotide sequence encoding an HCV E1/E2 heterodimer is followed by a second (booster) vaccine comprising a recombinant MVA vector that comprises a nucleotide sequence encoding a T-cell epitope polypeptide. Other prime-boost protocols can be used. For example, multiple primes and/or multiple boosts can be administered.

In some cases, a first (priming) immunogenic composition is administered, where the first immunogenic composition comprises one or more of: a) an HCV E1/E2 heterodimer; b) an HCV E2 polypeptide; c) an HCV E1 polypeptide; and d) a T-cell epitope polypeptide, as described above; and a second (boosting) immunogenic composition is administered, where the second immunogenic composition comprises a recombinant viral vector comprising nucleotide sequence(s) encoding one or more of: a) an HCV E1/E2 heterodimer; b) an HCV E2 polypeptide; c) an HCV E1 polypeptide; and d) a T-cell epitope polypeptide, as described above.

In some cases, a first (priming) immunogenic composition is administered, where the first immunogenic composition comprises a recombinant viral vector comprising nucleotide sequence(s) encoding one or more of: a) an HCV E1/E2 heterodimer; b) an HCV E2 polypeptide; c) an HCV E1 polypeptide; and d) a T-cell epitope polypeptide, as described above; and a second (boosting) immunogenic composition is administered, where the second immunogenic composition comprises one or more of: a) an HCV E1/E2 heterodimer; b) an HCV E2 polypeptide; c) an HCV E1 polypeptide; and d) a T-cell epitope polypeptide, as described above.

In some cases, a co-immunization regimen is carried out, in which a polypeptide(s) per se is administered substantially concomitantly with a nucleic acid(s) encoding the polypeptide(s). For example, in some cases, a method of the present disclosure for inducing an immune response to an HCV polypeptide can comprise administering: a) a first immunogenic composition of the present disclosure, as described above, where the immunogenic composition comprises: i) an HCV E1/E2 heterodimer; ii) a T-cell epitope polypeptide; and iii) a pharmaceutically acceptable carrier; or i) an HCV E1 polypeptide; ii) a T-cell epitope polypeptide; and iii) a pharmaceutically acceptable carrier; or i) an HCV E2 polypeptide; ii) a T-cell epitope polypeptide; and iii) a pharmaceutically acceptable carrier; and b) a second immunogenic composition of the present disclosure, as described above, where the immunogenic composition comprises: i) one or more nucleic acids comprising nucleotide sequence encoding one or more of: an HCV E1/E2 heterodimer, an HCV E1 polypeptide, an HCV E2 polypeptide, and a T-cell epitope polypeptide; and ii) a pharmaceutically acceptable carrier. In some cases, the first and the second immunogenic compositions are in a single formulation. In some cases, the first and the second immunogenic compositions are in separate formulations. In some cases, the first and the second immunogenic compositions are administered via the same route of administration. In some cases, the first and the second immunogenic compositions are administered via different routes of administration. In some cases, the first and the second immunogenic compositions are in separate formulations that are administered substantially simultaneously, e.g., within 1 minute, within 1 minute to 5 minutes, within 5 minutes to 15 minutes, or within 15 minutes to 30 minutes, of one another. In some cases, the first and the second immunogenic compositions are administered multiple times to an individual.

In some cases, a co-immunization regimen is carried out, in which a polypeptide(s) per se is administered substantially concomitantly with a nucleic acid(s) encoding the polypeptide(s). For example, in some cases, a method of the present disclosure for inducing an immune response to an HCV polypeptide can comprise administering: a) a first immunogenic composition of the present disclosure, as described above, where the immunogenic composition comprises: i) an HCV E1/E2 heterodimer; ii) a T-cell epitope polypeptide; and iii) a pharmaceutically acceptable carrier; and b) a second immunogenic composition of the present disclosure, as described above, where the immunogenic composition comprises: i) one or more nucleic acids comprising nucleotide sequences encoding an HCV E1/E2 heterodimer and a T-cell epitope polypeptide; and ii) a pharmaceutically acceptable carrier. In some cases, the first and the second immunogenic compositions are in a single formulation. In some cases, the first and the second immunogenic compositions are in separate formulations. In some cases, the first and the second immunogenic compositions are administered via the same route of administration. In some cases, the first and the second immunogenic compositions are administered via different routes of administration. In some cases, the first and the second immunogenic compositions are in separate formulations that are administered substantially simultaneously, e.g., within 1 minute, within 1 minute to 5 minutes, within 5 minutes to 15 minutes, or within 15 minutes to 30 minutes, of one another. In some cases, the first and the second immunogenic compositions are administered multiple times to an individual. In some cases, the one or more nucleic acids are recombinant viral vectors.

In some cases, a method of the present disclosure for inducing an immune response to HCV in an individual comprises administering to the individual an effective amount of a nucleic acid(s) comprising nucleotide sequences encoding 1) an HCV E1/E2 heterodimer and a T-cell epitope polypeptide; 2) an HCV E2 polypeptide and a T-cell epitope polypeptide; or 3) an HCV E1 polypeptide and a T-cell epitope polypeptide. In some cases, the nucleic acid is an RNA comprising nucleotide sequences encoding a polypeptide of the present disclosure (e.g., an HCV E1/E2 heterodimer; an HCV E1 polypeptide; an HCV E2 polypeptide; a T-cell epitope polypeptide, as described herein. See, e.g., Weiner (2013) Molec. Therapy 21:506; and Ulmer et al. (2012) Vaccine 30:4414. In some cases, an RNA (e.g., a single mRNA molecule; or 2 mRNA molecules; or 3 mRNA molecules) comprising nucleotide sequences encoding a polypeptide of the present disclosure is formulated with a liposome. In some cases, an RNA (e.g., a single mRNA molecule; or 2 mRNA molecules) comprising nucleotide sequences encoding a polypeptide of the present disclosure is complexed with protamine. In some cases, an RNA (e.g., a single mRNA molecule; or 2 mRNA molecules) comprising nucleotide sequences encoding a polypeptide of the present disclosure is complexed with 1,2-dioleoyl-3-trimethylammonium-propane/1,2-dioleoyl-sn-glycero-3-phosphoethanolamine (DOTAP/DOPE).

In some cases, the nucleic acid(s) are present in bacteria (e.g., non-pathogenic bacteria suitable for delivery of nucleic acids to an individual). In some cases, the nucleic acid(s) are present in recombinant expression vector(s) present in bacteria (e.g., non-pathogenic bacteria suitable for delivery of nucleic acids to an individual). In some cases, the bacteria are live. In some cases, the bacteria are live attenuated bacteria. In some cases, the bacteria are killed. Bacteria suitable for delivery of nucleic acid(s) (which may be present in expression vectors) include, but are not limited to, Lactobacillus; Lactococcus (e.g., Lactococcus lactis); Salmonella, e.g., attenuated, non-pathogenic Salmonella, e.g., Salmonella enterica serovar Typhi, Salmonella enterica serovar Typhimurium; non-pathogenic strains of Escherichia coli; non-pathogenic strains of Bordetella pertussis; non-pathogenic strains of Listeria; non-pathogenic strains of Shigella; non-pathogenic strains of Vibrio (e.g., Vibrio cholera); Streptococcus gordonii; non-pathogenic strains of Yersinia enterocolitica; non-pathogenic strains of Shigella flexneri; non-pathogenic strains of Pseudomonas aeruginosa; non-pathogenic strains of Bacillus subtilis; and the like. In some cases, one or more virulence genes in the bacterium is all or partially deleted. For example, for Salmonella enterica serovar Typhi and Salmonella enterica serovar Typhimurium, an aroA, aroC, and aroD mutation can be made. Other mutations that can attenuate pathogenicity affect biosynthesis of the nucleotides adenine (pur) and guanine (guaBA), and outer membrane proteins C and F (ompC, ompF), as well as expression of the cAMP receptor (cya/crp), the conversion of UDP-galactose to UDP-glucose (galE), DNA recombination and repair (recA, recBC), and regulation of virulence genes (phoP, phoQ). For Listeria monocytogenes, attenuation can be achieved with auxotrophic mutants, or deletion of virulence factors such as the genes actA and internalin B (intB).

### General Considerations

An immunogenic composition of the present disclosure is generally administered to a human subject who: i) has an HCV infection; or ii) is at risk of acquiring an HCV infection (e.g., is at greater risk than the general population of acquiring an HCV infection); or iii) is naïve with respect to HCV infection, so as to prevent or at least partially arrest the development of disease and its complications. An amount adequate to accomplish this is defined as a “therapeutically effective dose” or a “therapeutically effective amount.” “Prophylactic” use of a subject immunogenic composition generally refers to administration to an individual who has not been infected with HCV (e.g., a “naïve” individual). “Therapeutic” use of a subject immunogenic composition can refer to “prophylactic” use (administration to an individual who has not been infected with HCV) and/or to administration to an individual who has an HCV infection. A “therapeutically effective amount” of an immunogenic composition of the present disclosure, can be an amount that, when administered in one or more doses to an individual who is not infected with HCV, is effective to induce an immune response in the individual to HCV. A “therapeutically effective amount” of an immunogenic composition of the present disclosure, can be an amount that, when administered in one or more doses to an individual who is infected with HCV, is effective to enhance an immune response in the individual to HCV.

Amounts effective for therapeutic use will depend on, e.g., the manner of administration, the weight and general state of health of the patient, and the judgment of the prescribing physician. Single or multiple doses of a subject immunogenic composition can be administered depending on the dosage and frequency required and tolerated by the patient, and route of administration.

In some cases, an effective amount of an immunogenic composition of the present disclosure is an amount that, when administered to an individual in one or more doses, is effective to induce an antibody response (e.g., a neutralizing antibody response) to HCV in the individual. For example, antibody to HCV (e.g., extracellular HCV), and/or to an HCV-infected cell, can be induced.

An effective amount of an immunogenic composition of the present disclosure can be an amount that, when administered to an individual in one or more doses, is effective to induce a neutralizing antibody response to HCV of a variety of genotypes (e.g., genotype 1; genotype 3; etc.). A neutralizing antibody response reduces binding of HCV to one or more host receptors for HCV and inhibits entry of HCV into a cell.

In some cases, an effective amount (e.g., a therapeutically effective amount) of an immunogenic composition of the present disclosure is an amount that, when administered to an individual in one or more doses, is effective to induce a cytotoxic T lymphocyte (CTL) response to HCV. For example, a CTL response to an HCV-infected cell can be induced.

In some cases, an effective amount (e.g., a therapeutically effective amount) of an immunogenic composition of the present disclosure is an amount that, when administered to an individual in one or more doses, is effective to induce a helper T lymphocyte (e.g., CD4⁺ T cell) to HCV in an individual.

In some cases, an effective amount (e.g., a therapeutically effective amount) of an immunogenic composition of the present disclosure is an amount that, when administered to an individual in one or more doses, is effective to induce an antibody response (e.g., a neutralizing antibody response) and/or a CTL response and/or a helper T cell response to HCV genotype 1. In some cases, an effective amount (e.g., a therapeutically effective amount) of an immunogenic composition of the present disclosure is an amount that, when administered to an individual in one or more doses, is effective to induce an antibody response (e.g., a neutralizing antibody response) and/or a CTL response and/or a helper T cell response to HCV genotype 3. In some cases, an effective amount (e.g., a therapeutically effective amount) of an immunogenic composition of the present disclosure is an amount that, when administered to an individual in one or more doses, is effective to induce an antibody response (e.g., a neutralizing antibody response) and/or a CTL response and/or a helper T cell response to HCV genotype 1 and HCV genotype 3. In some cases, an effective amount (e.g., a therapeutically effective amount) of an immunogenic composition of the present disclosure is an amount that, when administered to an individual in one or more doses, is effective to induce an antibody response (e.g., a neutralizing antibody response) and/or a CTL response and/or a helper T cell response to HCV of any genotype.

An immunogenic composition of the present disclosure is generally administered in an amount effective to elicit an immune response, e.g., a humoral immune response (e.g., an antibody response) and/or a CTL response, in the mammalian subject. Effective amounts of E1/E2, E1, or E2 polypeptides for immunization will vary, and can generally range from about 1 μg to 100 μg per 70 kg patient, e.g., from about 5 μg/70 kg to about 50 pg/70 kg. Substantially higher dosages (e.g. 10 mg to 100 mg or more) of an HCV E1/E2, E1, or E2 polypeptide may be suitable in oral, nasal, or topical administration routes. In some cases, a dose of an immunogenic composition of the present disclosure comprises an HCV E1/E2 heterodimer in an amount of from 4 μg to 100 μg. For example, in some cases, a dose of an immunogenic composition of the present disclosure comprises an HCV E1/E2 heterodimer in an amount of from 4 μg to 5 μg, from 5 μg to 10 μg, from 10 μg to 15 μg, from 15 μg to 20 μg, from 20 μg to 25 μg, from 25 g to 30 μg, from 30 μg to 40 μg, from 40 μg to 50 μg, from 50 μg to 60 μg, from 60 μg to 70 μg, from 70 μg to 80 μg, from 80 μg to 90 μg, or from 90 μg to 100 μg. In some cases, a dose of an immunogenic composition of the present disclosure comprises an HCV E1/E2 heterodimer in an amount of from about 100 μg to about 200 μg.

In some cases, the amount of T-cell epitope polypeptide in a given dose ranges from 0.5 g to 125 μg; e.g., a suitable dose of T-cell epitope polypeptide ranges from 0.5 μg to 1 μg, from 1 μg to 5 μg, from 5 μg to 10 μg, from 10 μg to 15 μg, from 15 μg to 20 μg, from 20 μg to 25 μg, from 25 μg to 30 μg, from 30 μg to 40 μg, from 40 μg to 50 μg, from 50 μg to 60 μg, from 60 g to 75 μg, from 75 μg to 100 μg, or from 100 μg to 125 μg. In some cases, where two or more different T-cell epitope polypeptides are included in an immunogenic composition, a dose will include from 0.5 μg to 125 μg of each T-cell epitope polypeptide. In some cases, where two (or more) different T-cell epitope polypeptides are included in an immunogenic composition, a dose will include from 1 μg to 10 μg (e.g., 1 μg, 2 μg, 3 μg, 4 μg, 5 μg, 6 μg, 7 μg, 8 μg, 9 μg, or 10 g) of each T-cell epitope polypeptide. As one non-limiting example, where an immunogenic composition of the present disclosure includes 2 or more different T-cell epitope polypeptides, the composition can include 1 μg of each different T-cell epitope polypeptide. As another non-limiting example, where an immunogenic composition of the present disclosure includes 2 or more different T-cell epitope polypeptides, the composition can include 3 μg of each different T-cell epitope polypeptide. As another non-limiting example, where an immunogenic composition of the present disclosure includes 2 or more different T-cell epitope polypeptides, the composition can include 6 μg of each different T-cell epitope polypeptide.

In some cases, a single dose of an immunogenic composition of the present disclosure comprises: a) an HCV E1/E2 heterodimer in an amount of from about 10 μg to about 15 μg (e.g., g, 11 μg, 12 μg, 13 μg, 14 μg, or 15 μg E1/E2 heterodimer); and b) two or more different T-cell epitope polypeptides in an amount of from about 5 μg each different T-cell epitope polypeptide to about 15 μg each different T-cell epitope polypeptide (e.g., 5 μg, 6 μg, 7 μg, 8 μg, 9 μg, 10 μg, 11 μg, 12 μg, 13 μg, 14 μg, or 15 μg each different T-cell epitope polypeptide. As one non-limiting example, in some cases, a single dose of an immunogenic composition of the present disclosure comprises: a) 15 μg E1/E2 heterodimer; and b) 45 μg T-cell epitope polypeptides (9 μg each of TP35-NS3, TP50C, TP23, TP27, and TP35-NS4).

In some cases, dose of an immunogenic composition of the present disclosure comprises an E1/E2 heterodimer a T-cell epitope polypeptide, where the molar ratio of T-cell epitope polypeptide to E1/E2 heterodimer in the composition is from about 0.1:1 to about 25:1. For example, the molar ratio of T-cell epitope polypeptide to E1/E2 heterodimer in an immunogenic composition of the present disclosure is from about 0.1:1 to about 0.2:1, from about 0.2:1 to 0.3:1, from about 0.3:1 to about 0.4:1, from about 0.4:1 to about 0.5:1, or from about 0.5:1 to 1:1. Where multiple different T-cell epitope polypeptides are present in an immunogenic composition of the present disclosure the molar ratio of the different T-cell epitope polypeptides to E1/E2 heterodimer can each be independently from about 0.1:1 to about 0.2:1, from about 0.2:1 to 0.3:1, from about 0.3:1 to about 0.4:1, from about 0.4:1 to about 0.5:1, or from about 0.5:1 to 1:1. As another example, the molar ratio of any given T-cell epitope polypeptide to E1/E2 heterodimer is from 1:1 to 25:1, e.g., from 1:1 to 5:1, from 5:1 to 10:1, from 10:1 to 15:1, from 15:1 to 20:1, or from 20:1 to 25:1. In some cases, the molar ratio of a T-cell epitope polypeptide to E1/E2 heterodimer in an immunogenic composition of the present disclosure is from about 10:1 to 20:1. In some cases, the molar ratio of a T-cell epitope polypeptide to E1/E2 heterodimer in an immunogenic composition of the present disclosure is from about 10:1 to 15:1. In some cases, the molar ratio of a T-cell epitope polypeptide to E1/E2 heterodimer in a composition of the present disclosure is 15:1.

The initial administration can be followed by booster immunization of the same immunogenic composition or a different immunogenic composition. In some instances, a subject method of inducing an immune response involves an initial administration of an immunogenic composition of the present disclosure, followed by at least one booster, and in some instances involves two or more (e.g., three, four, or five) boosters. The interval between an initial administration and a booster, or between a give booster and a subsequent booster, can be from about 1 week to about 12 weeks, e.g., from about 1 week to about 2 weeks, from about 2 weeks to about 4 weeks, from about 4 weeks to about 6 weeks, from about 6 weeks to about 8 weeks, from about 8 weeks to about 10 weeks, or from about 10 weeks to about 12 weeks. The interval between an initial administration and a booster, or between a give booster and a subsequent booster, can be from 4 months to 6 months, or from 6 months to 1 year.

In general, immunization can be accomplished by administration of an immunogenic composition of the present disclosure by any suitable route, including administration of the composition orally, nasally, nasopharyngeally, parenterally, enterically, gastrically, topically, transdermally, subcutaneously, intramuscularly, in tablet, solid, powdered, liquid, aerosol form, locally or systemically, with or without added excipients. Actual methods for preparing parenterally administrable compositions will be known or apparent to those skilled in the art and are described in more detail in such publications as Remington's Pharmaceutical Science, 15th ed., Mack Publishing Company, Easton, Pa. (1980). In some instances, immunization is accomplished by intramuscular injection of an immunogenic composition of the present disclosure.

### Individuals Suitable for Administration

Individuals who are suitable for administration with an immunogenic composition of the present disclosure include immunologically naïve individuals (e.g., individuals who have not been infected with HCV and/or who have not been administered with an HCV vaccine). Individuals suitable for administration include humans.

Individuals who are suitable for administration with an immunogenic composition of the present disclosure composition of the present disclosure include individuals who are at greater risk than the general population of becoming infected with HCV, where such individuals include, e.g., intravenous drug users; individuals who are the recipients, or the prospective recipients, of blood or blood products from another (donor) individual(s); individuals who are the recipients, or the prospective recipients, of non-autologous cells, tissues, or organs from another (donor) individual; health care workers; emergency medical and non-medical personnel (e.g., first responders; fire fighters; emergency medical team personnel; etc.) and the like.

Individuals who are suitable for administration with an immunogenic composition of the present disclosure composition of the present disclosure include individuals who recently became exposed to HCV or who recently became infected with HCV. For example, a subject immunogenic composition can be administered to an individual within from about 24 hours to about 48 hours, from about 48 hours to about 1 week, or from about 1 week to about 4 weeks, following possible or suspected exposure to HCV or following infection with HCV.

Individuals who are suitable for administration with an immunogenic composition of the present disclosure composition of the present disclosure include individuals who were previously infected with HCV, who were treated for HCV, and who were cured.

Individuals who are suitable for administration with an immunogenic composition of the present disclosure composition of the present disclosure include individuals who have been diagnosed as having an HCV infection, and include chronically infected individuals. In some cases, an individual who has been diagnosed as having an HCV infection is treated with an anti-viral agent and an immunogenic composition of the present disclosure. Suitable anti-viral agents for treating HCV infection include, e.g., ribavirin (1-β-D-ribofuranosyl-1H-1,2,4-triazole-3-carboxamide); interferon-alpha (IFN-α) (where “IFN-α” includes IFN-α2a; IFN-α2b; IFN-α that is conjugated with poly(ethylene glycol) (“pegylated IFN-α), where the pegylated IFN-α can be pegylated IFN-α2a or pegylated IFN-α 2b); an HCV NS3 protease inhibitor (e.g., boceprevir; telaprevir); and an HCV NS5 protease inhibitor.

In some cases, an individual who has been diagnosed as having an HCV infection is treated with, e.g.: 1) IFN-α+ribavirin; and an immunogenic composition of the present disclosure; 2) IFN-α+ribavirin+an HCV protease inhibitor (e.g., boceprevir or telaprevir); and an immunogenic composition of the present disclosure; 3) Harvoni; and an immunogenic composition of the present disclosure; 4) an inhibitor of HCV NS5B; and an immunogenic composition of the present disclosure; 5) an inhibitor of HCV NS5A; and an immunogenic composition of the present disclosure; or 6) an inhibitor of HCV NS5B+an inhibitor of HCV NS5A; and an immunogenic composition of the present disclosure. Suitable anti-viral agents for treating HCV infection include Sovaldi (Sofosbuvir; a nucleotide analog that functions as an NS5B polymerase inhibitor), alone or in combination with pegylated IFN-α and ribavirin; and Harvoni. Harvoni is a formulation comprising 90 mg ledipasvir and 400 mg sofosbuvir. Ledipasvir is an inhibitor of HCV NS5A.

### Examples of Non-Limiting Aspects of the Disclosure

### Aspects Set A

Aspects, including embodiments, of the present subject matter described above may be beneficial alone or in combination, with one or more other aspects or embodiments. Without limiting the foregoing description, certain non-limiting aspects of the disclosure numbered 1-27 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. An immunogenic composition comprising:
a) one or more T-cell epitope polypeptides selected from:
i) a TP35-NS3 T-cell epitope polypeptide comprising an amino acid sequence having at least 80% (at least 80%, at least 90%, at least 95%, at least 98%, at least 99%, or 100%) amino acid sequence identity to:
KSTKVPX₁AYX₂X₃QGYX₄VLVLNPSVAATLGFGX₅X₆X₇SX₈ (SEQ ID NO:134), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids; and
ii) a TP50C T-cell epitope polypeptide comprising an amino acid sequence having at least 80% (at least 80%, at least 90%, at least 95%, at least 98%, at least 99%, or 100%) amino acid sequence identity to:
GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:149), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁₁ is S, T, H, or A; X₁₂ is A or G; and X₁₃ is Q or K, wherein the TP50C T-cell epitope polypeptide has a length of from 40 amino acids to 50 amino acids;
iii) a TP23 T-cell epitope polypeptide comprising an amino acid sequence having at least 80% (at least 80%, at least 90%, at least 95%, at least 98%, at least 99%, or 100%) amino acid sequence identity to: DVVVX₁X₂TDALMTGX₃TGDFDSVID (SEQ ID NO:171), wherein X₁ is V or C; X₂ is A or S; and X₃ is F or Y, wherein the TP23 T-cell epitope polypeptide has a length of from 18 amino acids to 23 amino acids;
iv) a TP27 T-cell epitope polypeptide comprising an amino acid sequence having at least 80% (at least 80%, at least 90%, at least 95%, at least 98%, at least 99%, or 100%) amino acid sequence identity to: X₁X₂X₃X₄KGGRHLIFCHSKKKCDEX₅AX₆X₇LX₈(SEQ ID NO:189), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids;
a TP35-NS4 T-cell epitope polypeptide comprising an amino acid sequence having at least 80% (at least 80%, at least 90%, at least 95%, at least 98%, at least 99%, or 100%) amino acid sequence identity to: X₁X₂X₃RHX₄GX₅X₆EGX₇X₈QWMNRLIAFASRGNHVX₉PTHYX₁₀ (SEQ ID NO:204), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; and X₁₀ is V or I, wherein the TP35-NS4 T-cell epitope polypeptide has a length of from 28 amino acids to 35 amino acids;
vi) a TP42 T-cell epitope polypeptide comprising an amino acid sequence having at least 80% (at least 80%, at least 90%, at least 95%, at least 98%, at least 99%, or 100%) amino acid sequence identity to: X₁X₂X₃GEIPFYGX₄AIPX₅X₆X₇X₈KGGRHLIFCHSKKKCDEX₉AX₁₀X₁₁LX₁₂X₁₃(K)n (SEQ ID NO:229), where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, or L; X₈ is I or L; X₉ is L or I; X₁₀ is A, K, or S; X₁₁ is K, Q, or A; X₁₂ is T, R, or S; and X₁₃ is G or S, wherein n is an integer from 2 to 10, and wherein the TP42 T-cell epitope polypeptide has a length of from 34 amino acids to 52 amino acids;
vii) a TP45 T-cell epitope polypeptide comprising an amino acid sequence having at least 80% (at least 80%, at least 90%, at least 95%, at least 98%, at least 99%, or 100%) amino acid sequence identity to: X₁X₂AVAX₃YRGX₄DVX₅X₆IPX₇X₈GDVVVX₉X₁₀TDALMTGX₁₁TGDFDSVIDX₁₂X₁₃X₁₄(K)n (SEQ ID NO:249), wherein X₁ is L or V; X₂ is N or T; X₃ is Y or F; X₄ is L or V; X₅ is S or A; X₆ is V or I; X₇ is T or A; X₈ is S, Q, or T; X₉ is V or C; X₁₀ is A or S; X₁₁ is F or Y; X₁₂ is C or K; X₁₃ is N or K; and X₁₄ is V or K, wherein n is an integer from 2 to 10, and wherein the TP45 T-cell epitope polypeptide has a length of from 36 amino acids to 55 amino acids; and
viii) a TP48 T-cell epitope polypeptide comprising an amino acid sequence having at least 80% (at least 80%, at least 90%, at least 95%, at least 98%, at least 99%, or 100%) amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:255), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acids; and
b) an immune-stimulating amount of an adjuvant.

Aspect 2. The immunogenic composition of aspect 1, wherein the one or more T-cell epitope polypeptides comprises a heterologous fusion partner.

Aspect 3. The immunogenic composition of aspect 2, wherein the heterologous fusion partner is (Lys)ₙ, wherein n is an integer from 2 to 10.

Aspect 4. The immunogenic composition of aspect 3, wherein n is 3, and wherein the fusion partner is at the C-terminus of the T-cell epitope polypeptide.

Aspect 5. The immunogenic composition of any one of aspects 1-4, wherein the composition comprises 2, 3, 4, or 5 different T-cell epitope polypeptides.

Aspect 6. The immunogenic composition of any one of aspects 1-5, wherein one or more T-cell epitope polypeptides comprise:
a) a TP35-NS3 T-cell epitope polypeptide comprising an amino acid sequence having at least 80% (at least 80%, at least 90%, at least 95%, at least 98%, at least 99%, or 100%) amino acid sequence identity to:
KSTKVPAAYAAQGYKVLVLNPSVAATLGFGAYMSK (SEQ ID NO:133), wherein the TP35-NS3 T-cell epitope polypeptide has a length of from 28 amino acids to 35 amino acids; and
b) a TP50C T-cell epitope polypeptide comprising an amino acid sequence having at least 80% (at least 80%, at least 90%, at least 95%, at least 98%, at least 99%, or 100%) amino acid sequence identity to:
GVYLLPRRGPRLGVRATRKTSERSQPRGRRQPIPKARRSEGRSWAQPGYP (SEQ ID NO:148), wherein the TP50C T-cell epitope polypeptide has a length of from 40 amino acids to 50 amino acids.

Aspect 7. The immunogenic composition of any one of aspects 1-5, wherein the one or more T-cell epitope polypeptides comprise:
a) a TP35-NS3 T-cell epitope polypeptide comprising an amino acid sequence having at least 80% (at least 80%, at least 90%, at least 95%, at least 98%, at least 99%, or 100%) amino acid sequence identity to:
KSTKVPAAYAAQGYKVLVLNPSVAATLGFGAYMSK (SEQ ID NO:133), wherein the T-cell epitope polypeptide has a length of from 28 amino acids to 35 amino acids; and
b) a TP50C T-cell epitope polypeptide comprising an amino acid sequence having at least 80% (at least 80%, at least 90%, at least 95%, at least 98%, at least 99%, or 100%) amino acid sequence identity to:
GVYLLPRRGPRLGVRATRKTSERSQPRGRRQPIPKARRSEGRSWAQPGYP (SEQ ID NO:148), wherein the T-cell epitope polypeptide has a length of from 40 amino acids to 50 amino acids; and
c) one or more T-cell epitope polypeptides selected from:
   i) a TP23 T-cell epitope polypeptide comprising an amino acid sequence having at least 80% (at least 80%, at least 90%, at least 95%, at least 98%, at least 99%, or 100%) amino acid sequence identity to: DVVVVATDALMTGFTGDFDSVID (SEQ ID NO:186), wherein the TP23 T-cell epitope polypeptide has a length of from 18 amino acids to 23 amino acids;
   ii) a TP27 T-cell epitope polypeptide comprising an amino acid sequence having at least 80% (at least 80%, at least 90%, at least 95%, at least 98%, at least 99%, or 100%) amino acid sequence identity to: LEQIKGGRHLIFCHSKKKCDELAAKLT (SEQ ID NO:188), wherein the TP27 T-cell epitope polypeptide has a length of from 22 amino acids to 27 amino acids; and
   iii) a TP35-NS4 T-cell epitope polypeptide comprising an amino acid sequence having at least 80% (at least 80%, at least 90%, at least 95%, at least 98%, at least 99%, or 100%) amino acid sequence identity to: ILRRHVGPGEGAVQWMNRLIAFASRGNHVSPTHYV (SEQ ID NO:203), wherein the TP35-NS4 T-cell epitope polypeptide has a length of from 28 amino acids to 35 amino acids.

Aspect 8. The immunogenic composition of any one of aspects 1-5, wherein the one or more T-cell epitope polypeptides comprise:
a) a TP35-NS3 T-cell epitope polypeptide comprising an amino acid sequence having at least 80% (at least 80%, at least 90%, at least 95%, at least 98%, at least 99%, or 100%) amino acid sequence identity to:
KSTKVPAAYAAQGYKVLVLNPSVAATLGFGAYMSK (SEQ ID NO:133), wherein the T-cell epitope polypeptide has a length of from 28 amino acids to 35 amino acids; and
b) a TP50C T-cell epitope polypeptide comprising an amino acid sequence having at least 80% (at least 80%, at least 90%, at least 95%, at least 98%, at least 99%, or 100%) amino acid sequence identity to:
GVYLLPRRGPRLGVRATRKTSERSQPRGRRQPIPKARRSEGRSWAQPGYP (SEQ ID NO:148), wherein the T-cell epitope polypeptide has a length of from 40 amino acids to 50 amino acids; and
c) a TP23 T-cell epitope polypeptide comprising an amino acid sequence having at least 80% (at least 80%, at least 90%, at least 95%, at least 98%, at least 99%, or 100%) amino acid sequence identity to: DVVVVATDALMTGFTGDFDSVID (SEQ ID NO:186), wherein the TP23 T-cell epitope polypeptide has a length of from 18 amino acids to 23 amino acids;
d) a TP27 T-cell epitope polypeptide comprising an amino acid sequence having at least 80% (at least 80%, at least 90%, at least 95%, at least 98%, at least 99%, or 100%) amino acid sequence identity to: LEQIKGGRHLIFCHSKKKCDELAAKLT (SEQ ID NO:188), wherein the TP27 T-cell epitope polypeptide has a length of from 22 amino acids to 27 amino acids; and
e) a TP35-NS4 T-cell epitope polypeptide comprising an amino acid sequence having at least 80% (at least 80%, at least 90%, at least 95%, at least 98%, at least 99%, or 100%) amino acid sequence identity to: ILRRHVGPGEGAVQWMNRLIAFASRGNHVSPTHYV (SEQ ID NO:203), wherein the TP35-NS4 T-cell epitope polypeptide has a length of from 28 amino acids to 35 amino acids.

Aspect 9. The immunogenic composition of aspect 2, wherein heterologous fusion partner is one or more of:
a) cholera toxin or toxoid;
b) tetanus toxin or toxoid; and/or
c) diphtheria toxin or toxoid; and/or
d) CRM197.

Aspect 10. The immunogenic composition of any one of aspects 1-9, wherein the composition comprises a polypeptide comprising one or more T cell epitopes present in:
a) cholera toxin or toxoid; and/or
b) tetanus toxin or toxoid; and/or
c) diphtheria toxin or toxoid; and/or
d) CRM197.

Aspect 11. The immunogenic composition of any one of aspects 1-10, further comprising a hepatitis C virus (HCV) E1/E2 heterodimeric polypeptide comprising:
i) an HCV E1 polypeptide; and
ii) an HCV E2 polypeptide;

Aspect 12. The immunogenic composition of aspect 11, wherein the HCV E1 polypeptide comprises an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to an E1 polypeptide depicted in FIGS. 1A-IC, FIGS. 2A-2C, FIGS. 3A-3C, and FIGS. 4A-4B.

Aspect 13. The immunogenic composition of aspect 11, wherein the HCV E2 polypeptide comprises an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to an E2 polypeptide depicted in one of FIGS. 1A-IC, FIGS. 2A-2C, FIGS. 3A-3C, and FIGS. 4A-4B.

Aspect 14. The immunogenic composition of any one of aspects 11-13, wherein the E2 polypeptide and/or the E1 polypeptide lacks a C-terminal transmembrane domain.

Aspect 15. The immunogenic composition of any one of aspects 11-14, wherein the HCV E2 polypeptide and the HCV E1 polypeptide are derived from an HCV of the same genotype.

Aspect 16. The immunogenic composition of any one of aspects 11-15, wherein the HCV E2 polypeptide and the HCV E1 polypeptide are derived from an HCV of different genotypes.

Aspect 17. The immunogenic composition of any one of aspects 11-16, wherein the HCV E1/E2 heterodimeric polypeptide comprises:
a) an HCV E1 polypeptide; and
b) a modified E2 polypeptide comprising, in order from N-terminus to C-terminus:
i) from 1 to 6 heterologous amino acids, wherein the from 1 to 6 heterologous amino acids are C-terminal to a site of proteolytic cleavage in a proteolytically cleavable linker; and
ii) an HCV E2 polypeptide; or
a) an HCV E2 polypeptide; and
b) a modified E1 polypeptide comprising, in order from N-terminus to C-terminus:
i) from 1 to 6 heterologous amino acids, wherein the from 1 to 6 heterologous amino acids are C-terminal to a site of proteolytic cleavage in a proteolytically cleavable linker; and
ii) an HCV E1 polypeptide; or
a) an HCV E1 polypeptide; and

b) a modified E2 polypeptide comprising, in order from N-terminus to C-terminus:

i) an HCV E2 polypeptide; and

ii) from 1 to 6 heterologous amino acids, wherein the from 1 to 6 heterologous amino acids are N-terminal to a site of proteolytic cleavage in a proteolytically cleavable linker; or
a) an HCV E2 polypeptide; and
b) a modified E1 polypeptide comprising, in order from N-terminus to C-terminus:
   i) an HCV E1 polypeptide; and
   ii) from 1 to 6 heterologous amino acids, wherein the from 1 to 6 heterologous amino acids are N-terminal to a site of proteolytic cleavage in a proteolytically cleavable linker.

Aspect 18. The immunogenic composition of aspect 17, wherein:
a) the from 1 to 6 heterologous amino acids at the N-terminus of the modified E2 polypeptide or the modified E1 polypeptide are Gly-Pro, Ser, Gly, or Gly-Ser; or
b) the from 1 to 6 heterologous amino acids at the C-terminus of the modified E2 polypeptide or the modified E1 polypeptide are LEVLFQ (SEQ ID NO:83), ENLYYFQ (SEQ ID NO:84), LVPR (SEQ ID NO:85), I(E/D)GR (SEQ ID NO:86), or DDDDK (SEQ ID NO:87).

Aspect 19. The immunogenic composition of any one of aspects 1-18, wherein the adjuvant comprises: MF59; alum; poly(DL-lactide co-glycolide); a CpG oligonucleotide; a suspension of liposomes comprising 3^{′}-O-desacyl-4^{′}-monophosphoryl lipid A (MPL) and Quillaja saponaria 21 (QS21); a mixture of alum and MPL; a phosphorylated hexaacyl disaccharide (PHAD); a mixture of alum and PHAD; or a cyclic dinucleotide.

Aspect 20. The immunogenic composition of any one of aspects 1-18, wherein the adjuvant comprises MF59.

Aspect 21. The immunogenic composition of any one of aspects 1-18, wherein the adjuvant comprises alum.

Aspect 22. A method of inducing an immune response in an individual to a hepatitis C virus (HCV) polypeptide, the method comprising administering to the individual:
a) an effective amount of the immunogenic composition of any one of aspects 1-21; or
b) one or more nucleic acids comprising nucleotide sequences encoding the T-cell epitope polypeptide(s) and/or the HCV E1/E2 heterodimeric polypeptide.

Aspect 23. The method of aspect 22, wherein said administering is by intramuscular administration.

Aspect 24. The method of aspect 22, wherein said administering is by subcutaneous administration.

Aspect 25. A container comprising the immunogenic composition of any one of aspects 1-21.

Aspect 26. The container of aspect 25, wherein the container and the composition are sterile.

Aspect 27. The container of aspect 25 or aspect 26, wherein the container is a syringe.

### Aspects Set B

Aspects, including embodiments, of the present subject matter described above may be beneficial alone or in combination, with one or more other aspects or embodiments. Without limiting the foregoing description, certain non-limiting aspects of the disclosure numbered 1-30 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. An immunogenic composition comprising:
a) one or more T-cell epitope polypeptides selected from:
i) a TP35-NS3 T-cell epitope polypeptide comprising an amino acid sequence having at least 80%, at least 90%, at least 95%, at least 98%, at least 99%, or 100%, amino acid sequence identity to:
KSTKVPX₁AYX₂X₃QGYX₄VLVLNPSVAATLGFGX₅X₆X₇SX₈ (SEQ ID NO:134), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids; and
ii) a TP50C T-cell epitope polypeptide comprising an amino acid sequence having at least 80%, at least 90%, at least 95%, at least 98%, at least 99%, or 100%, amino acid sequence identity to:
GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:149), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁₁ is S, T, H, or A; X₁₂ is A or G; and X₁₃ is Q or K, wherein the TP50C T-cell epitope polypeptide has a length of from 40 amino acids to 50 amino acids;
iii) a TP23 T-cell epitope polypeptide comprising an amino acid sequence having at least 80%, at least 90%, at least 95%, at least 98%, at least 99%, or 100%, amino acid sequence identity to: DVVVX₁X₂TDALMTGX₃TGDFDSVID (SEQ ID NO:171), wherein X₁ is V or C; X₂ is A or S; and X₃ is F or Y, wherein the TP23 T-cell epitope polypeptide has a length of from 18 amino acids to 23 amino acids;
iv) a TP27 T-cell epitope polypeptide comprising an amino acid sequence having at least 80%, at least 90%, at least 95%, at least 98%, at least 99%, or 100%, amino acid sequence identity to: X₁X₂X₃X₄KGGRHLIFCHSKKKCDEX₅AX₆X₇LX₈(SEQ ID NO:189), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids;
v) a TP35-NS4 T-cell epitope polypeptide comprising an amino acid sequence having at least 80%, at least 90%, at least 95%, at least 98%, at least 99%, or 100%, amino acid sequence identity to: X₁X₂X₃RHX₄GX₅X₆EGX₇X₈QWMNRLIAFASRGNHVX₉PTHYX₁₀ (SEQ ID NO:204), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; and X₁₀ is V or I, wherein the TP35-NS4 T-cell epitope polypeptide has a length of from 28 amino acids to 35 amino acids;
vi) a TP42 T-cell epitope polypeptide comprising an amino acid sequence having at least 80%, at least 90%, at least 95%, at least 98%, at least 99%, or 100%, amino acid sequence identity to: X₁X₂X₃GEIPFYGX₄AIPX₅X₆X₇X₈KGGRHLIFCHSKKKCDEX₉AX₁₀X₁₁LX₁₂X₁₃(K)n (SEQ ID NO:229), where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, or L; X₈ is I or L; X₉ is L or I; X₁₀ is A, K, or S; X₁₁ is K, Q, or A; X₁₂ is T, R, or S; and X₁₃ is G or S, wherein n is an integer from 2 to 10, and wherein the TP42 T-cell epitope polypeptide has a length of from 34 amino acids to 52 amino acids;
vii) a TP45 T-cell epitope polypeptide comprising an amino acid sequence having at least 80%, at least 90%, at least 95%, at least 98%, at least 99%, or 100%, amino acid sequence identity to: X₁X₂AVAX₃YRGX₄DVX₅X₆IPX₇X₈GDVVVX₉X₁₀TDALMTGX₁₁TGDFDSVIDX₁₂X₁₃X₁₄(K)n (SEQ ID NO:249), wherein X₁ is L or V; X₂ is N or T; X₃ is Y or F; X₄ is L or V; X₅ is S or A; X₆ is V or I; X₇ is T or A; X₈ is S, Q, or T; X₉ is V or C; X₁₀ is A or S; X₁₁ is F or Y; X₁₂ is C or K; X₁₃ is N or K; and X₁₄ is V or K, wherein n is an integer from 2 to 10, and wherein the TP45 T-cell epitope polypeptide has a length of from 36 amino acids to 55 amino acids; and
viii) a TP48 T-cell epitope polypeptide comprising an amino acid sequence having at least 80%, at least 90%, at least 95%, at least 98%, at least 99%, or 100%, amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:255), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acids;
ix) a TP33 T-cell epitope polypeptide comprising an amino acid sequence having at least 80%, at least 90%, at least 95%, at least 98%, at least 99%, or 100%, amino acid sequence identity to: HSKKKCDELAX₁X₂LX₃X₄X₅GX₆NAVAYYRGLDVSX₇IP (SEQ ID NO:287), where X₁ is A or S; X₂ is K or A; X₃ is V, S, R, or T; X₄ is A or G; X₅ is L or M; X₆ is I, L, or V; and X₇ is V or I; wherein the TP33 T-cell epitope polypeptide has a length of from 30 amino acids to 36 amino acids;
x) a TP42-2 T-cell epitope polypeptide comprising an amino acid sequence having at least 80%, at least 90%, at least 95%, at least 98%, at least 99%, or 100%, amino acid sequence identity to: KGGRHLIFCHSKKKCDELAX₁X₂LX₃X₄X₅GX₆NAVAYYRGLDVSX₇IP (SEQ ID NO:292), where X₁ is A or S; X₂ is K or A; X₃ is V, S, R, or T; X₄ is A or G; X₅ is L or M; X₆ is I, L, or V; and X₇ is V or I; wherein the TP42-2 T-cell epitope polypeptide has a length of from 38 amino acids to 46 amino acids; and
b) an immune-stimulating amount of an adjuvant.

Aspect 2. The immunogenic composition of aspect 1, wherein the one or more T-cell epitope polypeptides comprises a heterologous fusion partner.

Aspect 3. The immunogenic composition of aspect 2, wherein the heterologous fusion partner is (Lys)ₙ, wherein n is an integer from 2 to 10.

Aspect 4. The immunogenic composition of aspect 3, wherein n is 3, and wherein the fusion partner is at the C-terminus of the T-cell epitope polypeptide.

Aspect 5. The immunogenic composition of any one of aspects 1-4, wherein the composition comprises 2, 3, 4, or 5 different T-cell epitope polypeptides.

Aspect 6. The immunogenic composition of any one of aspects 1-5, wherein one or more T-cell epitope polypeptides comprise:
a) a TP35-NS3 T-cell epitope polypeptide comprising an amino acid sequence having at least 80%, at least 90%, at least 95%, at least 98%, at least 99%, or 100%, amino acid sequence identity to:
KSTKVPAAYAAQGYKVLVLNPSVAATLGFGAYMSK (SEQ ID NO:133), wherein the TP35-NS3 T-cell epitope polypeptide has a length of from 28 amino acids to 35 amino acids; and
b) a TP50C T-cell epitope polypeptide comprising an amino acid sequence having at least 80%, at least 90%, at least 95%, at least 98%, at least 99%, or 100%, amino acid sequence identity to:
GVYLLPRRGPRLGVRATRKTSERSQPRGRRQPIPKARRSEGRSWAQPGYP (SEQ ID NO:148), wherein the TP50C T-cell epitope polypeptide has a length of from 40 amino acids to 50 amino acids.

Aspect 7. The immunogenic composition of any one of aspects 1-5, wherein the one or more T-cell epitope polypeptides comprise:
a) a TP35-NS3 T-cell epitope polypeptide comprising an amino acid sequence having at least 80%, at least 90%, at least 95%, at least 98%, at least 99%, or 100%, amino acid sequence identity to:
KSTKVPAAYAAQGYKVLVLNPSVAATLGFGAYMSK (SEQ ID NO:133), wherein the T-cell epitope polypeptide has a length of from 28 amino acids to 35 amino acids; and
b) a TP50C T-cell epitope polypeptide comprising an amino acid sequence having at least 80%, at least 90%, at least 95%, at least 98%, at least 99%, or 100%, amino acid sequence identity to:
GVYLLPRRGPRLGVRATRKTSERSQPRGRRQPIPKARRSEGRSWAQPGYP (SEQ ID NO:148), wherein the T-cell epitope polypeptide has a length of from 40 amino acids to 50 amino acids; and
c) one or more T-cell epitope polypeptides selected from:
   i) a TP23 T-cell epitope polypeptide comprising an amino acid sequence having at least 80%, at least 90%, at least 95%, at least 98%, at least 99%, or 100%, amino acid sequence identity to: DVVVVATDALMTGFTGDFDSVID (SEQ ID NO:186), wherein the TP23 T-cell epitope polypeptide has a length of from 18 amino acids to 23 amino acids;
   ii) a TP27 T-cell epitope polypeptide comprising an amino acid sequence having at least 80%, at least 90%, at least 95%, at least 98%, at least 99%, or 100%, amino acid sequence identity to: LEQIKGGRHLIFCHSKKKCDELAAKLT (SEQ ID NO:188), wherein the TP27 T-cell epitope polypeptide has a length of from 22 amino acids to 27 amino acids; and
   iii) a TP35-NS4 T-cell epitope polypeptide comprising an amino acid sequence having at least 80%, at least 90%, at least 95%, at least 98%, at least 99%, or 100%, amino acid sequence identity to: ILRRHVGPGEGAVQWMNRLIAFASRGNHVSPTHYV (SEQ ID NO:203), wherein the TP35-NS4 T-cell epitope polypeptide has a length of from 28 amino acids to 35 amino acids.

Aspect 8. The immunogenic composition of any one of aspects 1-5, wherein the one or more T-cell epitope polypeptides comprise:
a) a TP35-NS3 T-cell epitope polypeptide comprising an amino acid sequence having at least 80%, at least 90%, at least 95%, at least 98%, at least 99%, or 100%, amino acid sequence identity to:
KSTKVPAAYAAQGYKVLVLNPSVAATLGFGAYMSK (SEQ ID NO:133), wherein the T-cell epitope polypeptide has a length of from 28 amino acids to 35 amino acids; and
b) a TP50C T-cell epitope polypeptide comprising an amino acid sequence having at least 80%, at least 90%, at least 95%, at least 98%, at least 99%, or 100%, amino acid sequence identity to:
GVYLLPRRGPRLGVRATRKTSERSQPRGRRQPIPKARRSEGRSWAQPGYP (SEQ ID NO:148), wherein the T-cell epitope polypeptide has a length of from 40 amino acids to 50 amino acids; and
c) a TP23 T-cell epitope polypeptide comprising an amino acid sequence having at least 80%, at least 90%, at least 95%, at least 98%, at least 99%, or 100%, amino acid sequence identity to: DVVVVATDALMTGFTGDFDSVID (SEQ ID NO:186), wherein the TP23 T-cell epitope polypeptide has a length of from 18 amino acids to 23 amino acids;
d) a TP27 T-cell epitope polypeptide comprising an amino acid sequence having at least 80%, at least 90%, at least 95%, at least 98%, at least 99%, or 100%, amino acid sequence identity to: LEQIKGGRHLIFCHSKKKCDELAAKLT (SEQ ID NO:188), wherein the TP27 T-cell epitope polypeptide has a length of from 22 amino acids to 27 amino acids; and
e) a TP35-NS4 T-cell epitope polypeptide comprising an amino acid sequence having at least 80%, at least 90%, at least 95%, at least 98%, at least 99%, or 100%, amino acid sequence identity to: ILRRHVGPGEGAVQWMNRLIAFASRGNHVSPTHYV (SEQ ID NO:203), wherein the TP35-NS4 T-cell epitope polypeptide has a length of from 28 amino acids to 35 amino acids.

Aspect 9. The immunogenic composition of any one of aspects 1-5, wherein the one or more T-cell epitope polypeptides comprise:
a) a TP35-NS3 T-cell epitope polypeptide comprising an amino acid sequence having at least 80%, at least 90%, at least 95%, at least 98%, at least 99%, or 100%, amino acid sequence identity to:
KSTKVPAAYAAQGYKVLVLNPSVAATLGFGAYMSK (SEQ ID NO:133), wherein the T-cell epitope polypeptide has a length of from 28 amino acids to 35 amino acids;
b) a TP50C T-cell epitope polypeptide comprising an amino acid sequence having at least 80%, at least 90%, at least 95%, at least 98%, at least 99%, or 100%, amino acid sequence identity to:
GVYLLPRRGPRLGVRATRKTSERSQPRGRRQPIPKARRSEGRSWAQPGYP (SEQ ID NO:148), wherein the T-cell epitope polypeptide has a length of from 40 amino acids to 50 amino acids;
c) a TP42 T-cell epitope polypeptide comprising an amino acid sequence having at least 80%, at least 90%, at least 95%, at least 98%, at least 99%, or 100%, amino acid sequence identity to: GTEGEIPFYGKAIPLEQIKGGRHLIFCHSKKKCDELAAKLTG (SEQ ID NO:228), wherein the T-cell epitope polypeptide has a length of from 38 amino acids to 45 amino acids; and
d) a TP48 T-cell epitope polypeptide comprising an amino acid sequence having at least 80%, at least 90%, at least 95%, at least 98%, at least 99%, or 100%, amino acid sequence identity to: ILRRHVGPGEGAVQWMNRLIAFASRGNHVSPTHYVPESDAAARVTQIL (SEQ ID NO:268), wherein the T-cell epitope polypeptide has a length of from 45 amino acids to 55 amino acids.

Aspect 10. The immunogenic composition of any one of aspects 1-5, wherein the one or more T-cell epitope polypeptides comprise:
a) a TP35-NS3 T-cell epitope polypeptide comprising an amino acid sequence having at least 80%, at least 90%, at least 95%, at least 98%, at least 99%, or 100%, amino acid sequence identity to:
KSTKVPAAYAAQGYKVLVLNPSVAATLGFGAYMSK (SEQ ID NO:133), wherein the TP35-NS3 T-cell epitope polypeptide has a length of from 28 amino acids to 35 amino acids; and
b) a TP50-C T-cell epitope polypeptide comprising an amino acid sequence having at least 80%, at least 90%, at least 95%, at least 98%, at least 99%, or 100%, amino acid sequence identity to:
GVYLLPRRGPRLGVRATRKTSERSQPRGRRQPIPKARRSEGRSWAQPGYP (SEQ ID NO:148), wherein the TP50-C T-cell epitope polypeptide has a length of from 40 amino acids to 50 amino acids;
c) a TP42 T-cell epitope polypeptide comprising an amino acid sequence having at least 80%, at least 90%, at least 95%, at least 98%, at least 99%, or 100%, amino acid sequence identity to: GTEGEIPFYGKAIPLEQIKGGRHLIFCHSKKKCDELAAKLTG (SEQ ID NO:228), wherein the TP42 T-cell epitope polypeptide has a length of from 38 amino acids to 45 amino acids;
d) a TP48 T-cell epitope polypeptide comprising an amino acid sequence having at least 80%, at least 90%, at least 95%, at least 98%, at least 99%, or 100%, amino acid sequence identity to: ILRRHVGPGEGAVQWMNRLIAFASRGNHVSPTHYVPESDAAARVTQIL (SEQ ID NO:268), wherein the T-cell epitope polypeptide has a length of from 45 amino acids to 55 amino acids; and
e) a TP42-2 T-cell epitope polypeptide comprising an amino acid sequence having at least 80%, at least 90%, at least 95%, at least 98%, at least 99%, or 100%, amino acid sequence identity to: KGGRHLIFCHSKKKCDELAAKLTGLGLNAVAYYRGLDVSVIP (SEQ ID NO:291), wherein the TP42-2 T-cell epitope polypeptide has a length of from 38 amino acids to 45 amino acids.

Aspect 11. The immunogenic composition of any one of aspects 1-5, wherein the one or more T-cell epitope polypeptides comprise:
a) a TP35-NS3 T-cell epitope polypeptide comprising an amino acid sequence having at least 80%, at least 90%, at least 95%, at least 98%, at least 99%, or 100%, amino acid sequence identity to:
KSTKVPAAYAAQGYKVLVLNPSVAATLGFGAYMSK (SEQ ID NO:133), wherein the T-cell epitope polypeptide has a length of from 28 amino acids to 35 amino acids;
b) a TP50C T-cell epitope polypeptide comprising an amino acid sequence having at least 80%, at least 90%, at least 95%, at least 98%, at least 99%, or 100%, amino acid sequence identity to:
GVYLLPRRGPRLGVRATRKTSERSQPRGRRQPIPKARRSEGRSWAQPGYP (SEQ ID NO:148), wherein the T-cell epitope polypeptide has a length of from 40 amino acids to 50 amino acids; and
c) a TP42 T-cell epitope polypeptide comprising an amino acid sequence having at least 80%, at least 90%, at least 95%, at least 98%, at least 99%, or 100%, amino acid sequence identity to: GTEGEIPFYGKAIPLEQIKGGRHLIFCHSKKKCDELAAKLTG (SEQ ID NO:228), wherein the T-cell epitope polypeptide has a length of from 38 amino acids to 45 amino acids.

Aspect 12. The immunogenic composition of aspect 2, wherein heterologous fusion partner is one or more of:
a) cholera toxin or toxoid;
b) tetanus toxin or toxoid; and
c) diphtheria toxin or toxoid; and
d) CRM197.

Aspect 13. The immunogenic composition of any one of aspects 1-12, wherein the composition comprises a polypeptide comprising one or more T cell epitopes present in:
a) cholera toxin or toxoid; and/or
b) tetanus toxin or toxoid; and/or
c) diphtheria toxin or toxoid; and/or
d) CRM197.

Aspect 14. The immunogenic composition of any one of aspects 1-13, further comprising a hepatitis C virus (HCV) E1/E2 heterodimeric polypeptide comprising: i) an HCV E1 polypeptide; and ii) an HCV E2 polypeptide;

Aspect 15. The immunogenic composition of aspect 14, wherein the HCV E1 polypeptide comprises an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to an E1 polypeptide depicted in FIG. 1A-IC, FIG. 2A-2C, FIG. 3A-3C, and FIG. 4A-4B.

Aspect 16. The immunogenic composition of aspect 14, wherein the HCV E2 polypeptide comprises an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to an E2 polypeptide depicted in one of FIG. 1A-IC, FIG. 2A-2C, FIG. 3A-3C, and FIG. 4A-4B.

Aspect 17. The immunogenic composition of any one of aspects 14-16, wherein the E2 polypeptide and/or the E1 polypeptide lacks a C-terminal transmembrane domain.

Aspect 18. The immunogenic composition of any one of aspects 14-17, wherein the HCV E2 polypeptide and the HCV E1 polypeptide are derived from an HCV of the same genotype.

Aspect 19. The immunogenic composition of any one of aspects 14-18, wherein the HCV E2 polypeptide and the HCV E1 polypeptide are derived from an HCV of different genotypes.

Aspect 20. The immunogenic composition of any one of aspects 14-19, wherein the HCV E1/E2 heterodimeric polypeptide comprises:
a) an HCV E1 polypeptide; and
b) a modified E2 polypeptide comprising, in order from N-terminus to C-terminus: i) from 1 to 6 heterologous amino acids, wherein the from 1 to 6 heterologous amino acids are C-terminal to a site of proteolytic cleavage in a proteolytically cleavable linker; and ii) an HCV E2 polypeptide; or
a) an HCV E2 polypeptide; and
b) a modified E1 polypeptide comprising, in order from N-terminus to C-terminus: i) from 1 to 6 heterologous amino acids, wherein the from 1 to 6 heterologous amino acids are C-terminal to a site of proteolytic cleavage in a proteolytically cleavable linker; and ii) an HCV E1 polypeptide; or
a) an HCV E1 polypeptide; and
b) a modified E2 polypeptide comprising, in order from N-terminus to C-terminus: i) an HCV E2 polypeptide; and ii) from 1 to 6 heterologous amino acids, wherein the from 1 to 6 heterologous amino acids are N-terminal to a site of proteolytic cleavage in a proteolytically cleavable linker; or
a) an HCV E2 polypeptide; and
b) a modified E1 polypeptide comprising, in order from N-terminus to C-terminus: i) an HCV E1 polypeptide; and ii) from 1 to 6 heterologous amino acids, wherein the from 1 to 6 heterologous amino acids are N-terminal to a site of proteolytic cleavage in a proteolytically cleavable linker.

Aspect 21. The immunogenic composition of aspect 20, wherein:
a) the from 1 to 6 heterologous amino acids at the N-terminus of the modified E2 polypeptide or the modified E1 polypeptide are Gly-Pro, Ser, Gly, or Gly-Ser; or
b) the from 1 to 6 heterologous amino acids at the C-terminus of the modified E2 polypeptide or the modified E1 polypeptide are LEVLFQ (SEQ ID NO:83), ENLYYFQ (SEQ ID NO:84), LVPR (SEQ ID NO:85), I(E/D)GR (SEQ ID NO:86), or DDDDK (SEQ ID NO:87).

Aspect 22. The immunogenic composition of any one of aspects 1-21, wherein the adjuvant comprises MF59; alum; poly(DL-lactide co-glycolide); a CpG oligonucleotide; a suspension of liposomes comprising 3^{′}-O-desacyl-4^{′}-monophosphoryl lipid A (MPL) and Quillaja saponaria 21 (QS21); a mixture of alum and MPL; a phosphorylated hexaacyl disaccharide (PHAD); a mixture of alum and PHAD; or a cyclic dinucleotide.

Aspect 23. The immunogenic composition of any one of aspects 1-21, wherein the adjuvant comprises MF59.

Aspect 24. The immunogenic composition of any one of aspects 1-21, wherein the adjuvant comprises alum.

Aspect 25. A method of inducing an immune response in an individual to a hepatitis C virus (HCV) polypeptide, the method comprising administering to the individual:
a) an effective amount of the immunogenic composition of any one of aspects 1-24; or
b) one or more nucleic acids comprising nucleotide sequences encoding the T-cell epitope polypeptide(s) and/or the HCV E1/E2 heterodimeric polypeptide.

Aspect 26. The method of aspect 25, wherein said administering is by intramuscular administration.

Aspect 27. The method of aspect 25, wherein said administering is by subcutaneous administration.

Aspect 28. A container comprising the immunogenic composition of any one of aspects 1-24.

Aspect 29. The container of aspect 28, wherein the container and the composition are sterile.

Aspect 30. The container of aspect 28 or aspect 29, wherein the container is a syringe.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

All reported CD8 T cell epitopes until Jul. 12, 2013 were extracted from the Immune Epitope Database and Analysis Resource (IDEB). These CD8 epitopes were narrowed down through an alternative search performed based on the common HLA MHC class-I Alleles in North America. According to the latest demographic statistics from 2013, the population of USA as the representative of the North America comprises of different ethnic groups including White (64%), Black (12%), Hispanic (16%), Asian or Pacific Islanders (5%), and others including Natives, Alaskans, and etc. (3%) (https://en(dot)wikipedia(dot)org/wiki/Race_and_ethnicity_in_the_United_States).

The first 30 common MHC-I alleles (frequency>˜ 6-7%) for each of the first four common populations in USA were used in a search for any HCV epitopes that were reported for any of these individual alleles, which comprised of A*01:01, A*02:03, A*02:06, A*02:07, A*03:01, A*11:01, A*23:01, A*24:02, A*25:01, A*26:01, A*29:02, A*30:01, A*30:02, A*31:01, A*32:01, A*33:03, A*34:02, A*68:01, A*68:02, A*74:01, B*07:02, B*08:01, B*14:02, B*15:01, B*15:02, B*15:03, B*18:01, B*35:01, B*38:02, B*40:01, B*40:02, B*42:01, B*44:01, B*44:03, B*45:01, B*46:01, B*49:01, B*51:01, B*52:01, B*53:01, B*54:01, B*55:02, B*57:01, B*58:01, C*01:02, C*02:02, C*03:03, C*03:04, C*04:01, C*05:01, C*06:02, C*07:01, C*07:02, C*08:01, C*08:02, C*14:02, and C*16:01 (https://www.proimmune.com/ecommerce/page.php?page=MHC_alleles). Using IDEB database until Jul. 12, 2013, a total of 106 MHC-I CD8 T cell epitopes that were restricted to each of the afore-mentioned alleles were found. The sequence of each epitope was searched and entered in an alignment including sequences from HCV genotypes 1a, 1b, and 3 as the genotypes of interest for a vaccine that will be potentially used in North America. Out of 106 epitopes, 64 were located and annotated on the sequences of HCV genotypes of 1a, 1b, and 3. In the case of mismatch of 1-3 amino acids in the sequence of the epitope against the HCV sequences, the epitope was still included in the analysis. The excluded 42 epitopes were consisting of those that were located on structural regions of HCV polyprotein (core, E1, E2, and P7), were repeats of other epitopes, or were not found.

### Example 2

T-cell epitope polypeptides TP35-NS3, TP35-NS3(Lys)₃, TP42, TP45 (with C-terminal CNV replaced with KKK), TP45(Lys)₃, TP48, TP48(Lys)₃, TP50C, TP23, TP23(Lys)₃, TP27, TP27(Lys)₃, TP35-NS4, and TP35-NS4(Lys)₃, were synthesized using solid-phase peptide synthesis. The solubility of these peptides was tested under various conditions and using various protocols.

Protocol 1 The solubility of the T-cell epitope polypeptides in E1E2 buffer was determined. E1E2 buffer: 10 mM sodium citrate, 250 mM NaCl, 1 mM ethylenediaminetetraacetic acid (EDTA), 0.1% non-ionic detergent Tween 80, pH 6.0. Lyophilized T-cell epitope polypeptides were individually added to E1E2 buffer at a final concentration of 1 mg T-cell epitope polypeptide per ml E1E2 buffer, to generate T-cell epitope polypeptide/E1E2 solutions. The T-cell epitope polypeptide/E1E2 solutions were vortexed for 3 seconds. The T-cell epitope polypeptide/E1E2 solutions were then centrifuged at 10,000×g for 30 seconds. As precipitation was visible in the TP42, TP45-3+KKK, and TP48 solutions, 0.5 ml of E1E2 buffer was added and the solutions were vortexed and centrifuged again. After centrifugation, the concentration of peptides was measured using Nanodrop (Thermo Fisher).

The results are shown in FIG. 6. Solubility values shown in FIG. 6 are estimates based on A280 nm reading. As shown in FIG. 6, TP35-NS3, TP35-NS3-(Lys)₃, TP48-(Lys)₃, and TP50-C exhibit good solubility in E1E2 buffer. In contrast, TP42 and TP45 (with C-terminal CNV replaced with KKK) exhibited much lower solubility in E1E2 buffer than TP35-NS3, TP35-NS3-(Lys)₃, TP48-(Lys)₃, and TP50-C.

Protocol 2 All T-cell epitope peptides were lyophilized. E1E2 buffer was added to the lyophilized peptides to a concentration of 1 mg peptide/ml E1E2 buffer, to generate reconstituted T-cell epitope peptides (“TPs”). The reconstituted TPs were vortexed for 3 seconds, then incubated at room temperature for 30 minutes. After the 30-minute incubation period, all reconstituted TPs were centrifuged at 10,000×g for 30 seconds. Following centrifugation, the concentration of the TPs in the supernatant was measured using Nanodrop (Thermo Fisher).

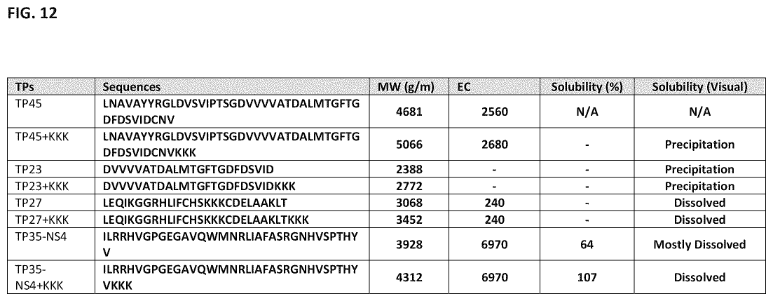

The results are shown in FIG. 12. EC—extinction coefficient. EC was used to calculate the TP concentration.

Protocol 3 TP42, TP45(Lys)₃, and TP48 were lyophilized. The lyophilized peptides were reconstituted in: i) E1E2 buffer; or ii) E1E2 buffer without Tween; or iii) water. The reconstituted peptide solutions were vigorously vortexed for 20 seconds. After vortexing, the peptide solutions were incubated at room temperature with continuous shaking at 300 rpm for 2 hours 30 minutes. Afterwards, peptide solutions were centrifuged at 5000×g for 1 minute. After centrifugation, the concentration of the peptides in the supernatant was measured.

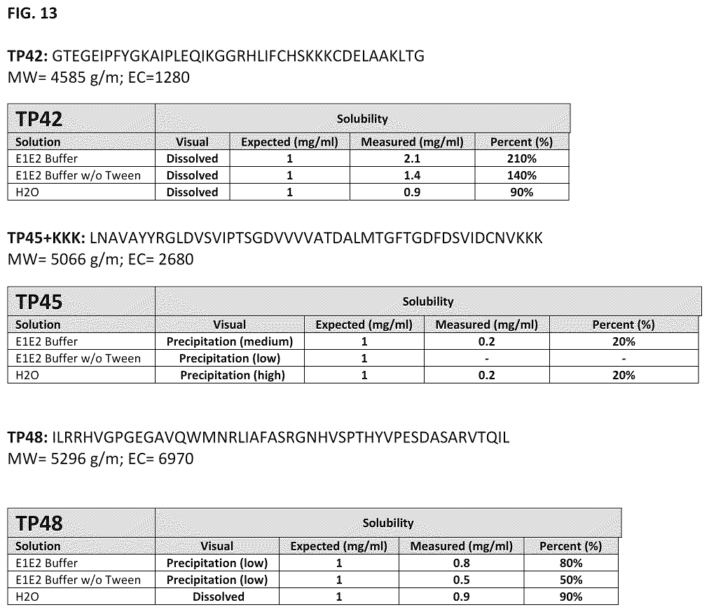

The results are shown in FIG. 13. The results indicate that TP42 and TP48 showed good solubility.

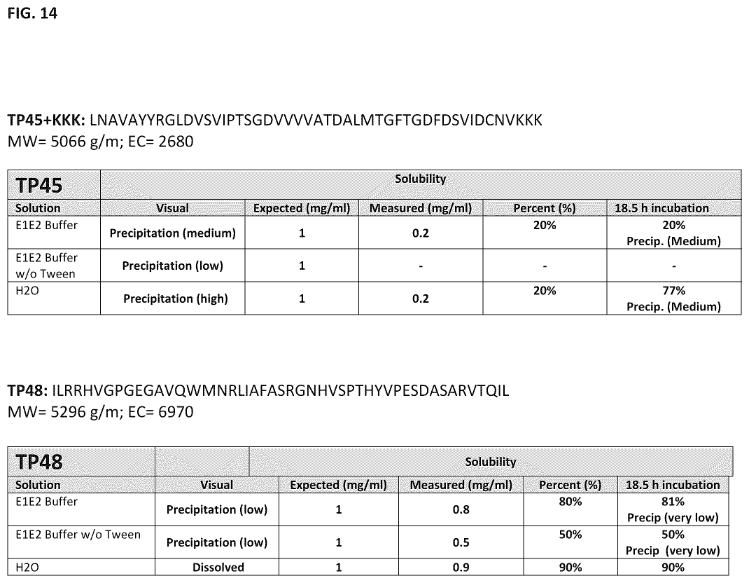

Protocol 4 TP45(Lys)₃, and TP48 were lyophilized. The lyophilized peptides were reconstituted in: i) E1E2 buffer; or ii) E1E2 buffer without Tween; or iii) water. The reconstituted peptide solutions were vigorously vortexed for 20 seconds. After vortexing, the peptide solutions were incubated at room temperature with continuous shaking at 300 rpm for 2 hours 30 minutes. Afterwards, peptide solutions were centrifuged at 5000×g for 1 minute. The results are shown in FIG. 14.

### Example 3: Epitope-Based Design of TPs

Reported HCV MHC-I and MHC-H epitopes that are conserved for 5 genotypes of HCV: A further analysis was done to extend the conservancy of epitopes among 5 genotypes of HCV. First, all of the amino acid sequences for different HCV genotypes were extracted from NCBI. This step resulted in 526, 302, 24, 97, 36, 31, 6, 61, and 3 sequences for HCV genotypes 1a, 1b, 2a, 2b, 3, 4, 5, 6, and 7, respectively. Next, consensus sequences were built for each of the 5 genotypes of HCV1a, HCV-1b, HCV-2a, HCV-2b, and HCV-3. These consensus sequences were populated in one Genious alignment document.

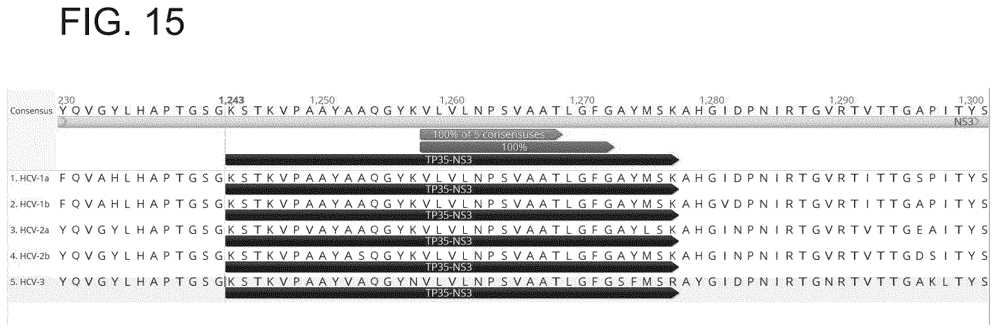

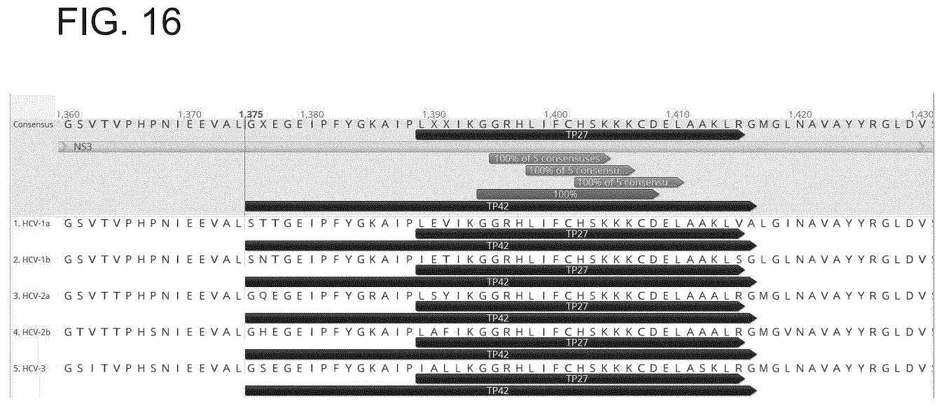

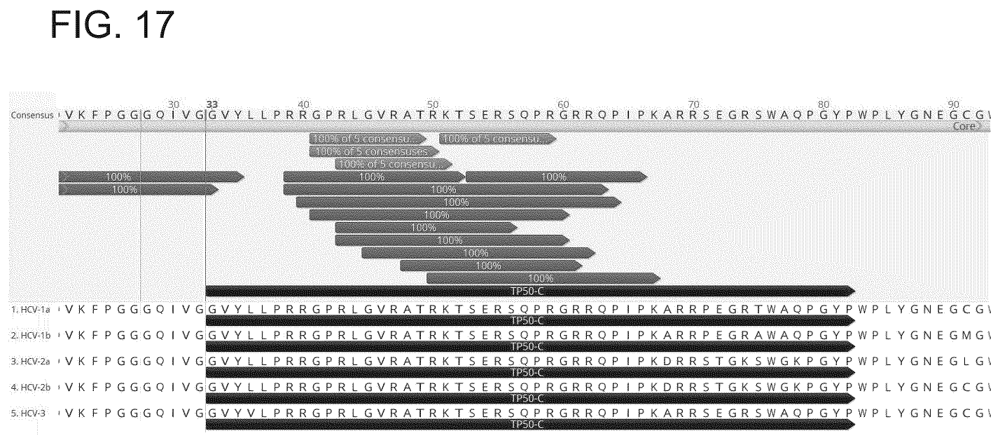

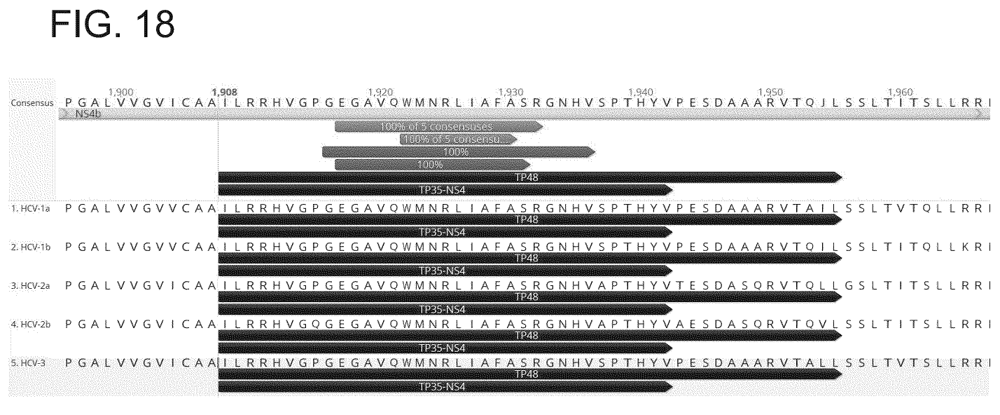

Using IEDB software, all of the reported HCV MHC-I and MHC-II epitopes were extracted from database, which resulted in 744 MHC-I and 679 MHC-II epitopes. Next, all those epitopes were checked for conservancy against 5 consensus sequences. This resulted in 18 and 22 epitopes for MHC-I and MHC-II, respectively. Of those, 10 of MHC-I and 14 of MHC-II were located on the candidate TPs (TP50-C, TP35-NS3, TP42, and TP48). These epitopes are depicted in FIG. 15-18.

Predicted HCV MHC-I and MHC-II epitopes for designed 4 TPs. In a parallel analysis, potential predicted epitopes were searched for in the region of our already designed 4 TPs. To do this, the sequences of 4 TPs were checked against 87 common (a sum of the first 63 common HLAs in the USA population and the common HLAs recommended by IEDB). This analysis provided a list of 81,417 possible epitopes with different scores based on IC50 and percentile rank (depending on the applicable analysis method). To choose the high affinity epitopes, the following methods and thresholds were used according to the method that was available for each pair of epitope-allele and based on IEDB recommendation.

In 54 allele the ANN method was used to select epitopes with high affinity (an IC50<50 nm). This resulted in 82 allele-epitope pairs that included 69 unique epitopes and 23 unique alleles paired with each other. Using Consensus method for the same 54 alleles at Percentile Rank of <1%, 93 allele-epitope pairs selected that included 66 unique epitopes and 40 unique alleles paired with each other. Combining ANN and Consensus methods resulted in 104 unique epitopes that were paired with 42 unique alleles which resulted in 130 epitope-pair to be used for Population Coverage Analysis.

Of the 33 remaining epitopes, NetMHCpan method was used to select the epitopes with high affinity for 32 epitopes and SMM method was used for one epitope. A cutoff of a percentile rank of <1% and IC50<50 nm was used in NetMHCpan method. This resulted in 11 epitope-allele pairs that included 8 unique epitopes and 7 unique alleles that paired with each other. In the case of SMM method, using SMM percentile rank of <1% resulted in 1 epitope-allele pair. Both were combined and entered into Genious.

By pooling all the predicted epitopes and removing the repeated ones, a total of 104 epitopes selected for population coverage analysis which were predicted to be potentially recognized by 50 HLAs at high affinity, according to the cutoff recommended by each method.

### Population Coverage Analysis Based on MHC-I Epitopes

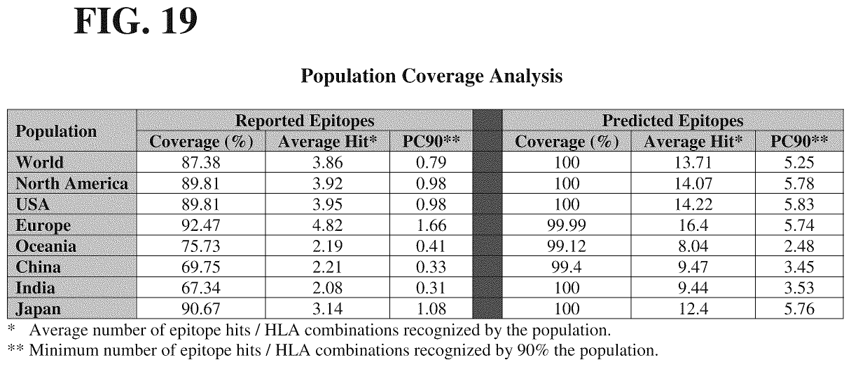

Population coverage analysis was done for two groups of MHC-I epitope-HLA pairs. First, for the 10 reported MHC-I epitope-HLA pairs and second for the 104 predicted epitope-HLA pairs. The results are depicted in FIG. 19.

### Memory Immune Response to TPs in Mice

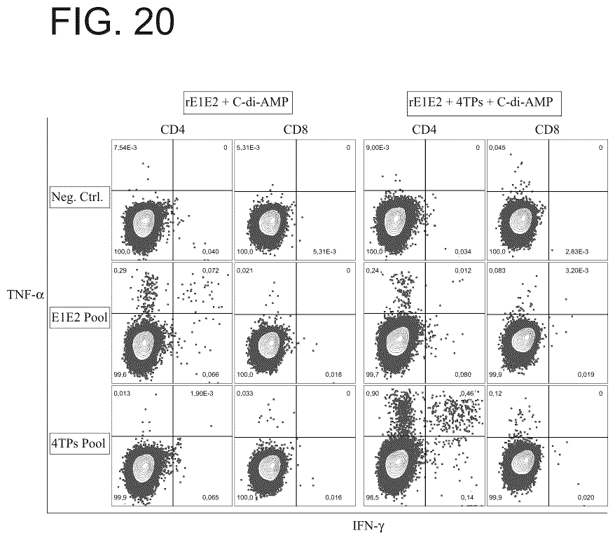

To check the solubility of a mixture of all 4 TPs (including TP50-C, TP35-NS3, TP42, and TP48) and the rE1E2 component of the vaccine, each TP was solubilized separately, as described above, and mixed with each other. The mixture of all 4 TPs was then further mixed with rE1E2. In the final solution of 4 TPS and rE1E2 no visible precipitation observed. The mixture was used to immunize mice. After three immunization, the mice were sacrificed, the spleens were harvested, splenocytes were isolated and memory immune response was investigated via an in vitro assay by detecting polyfunctional T cells expressing IFN-γ and TNF-α in an intracellular cytokine assay protocol by flow cytometry. The results demonstrated a CD4 (but not CD8) T cell response following to re-stimulation of the splenocytes by a pool of peptides that spans the whole length of all 4 TPs (FIG. 20). The lack of a CD8 T cell response could be due to the lack of enough number of matching epitope-HLA pair in mice in comparison to human, since the TPs were basically designed based on the reported HCV epitopes according to human HLAs.

### TP Analysis

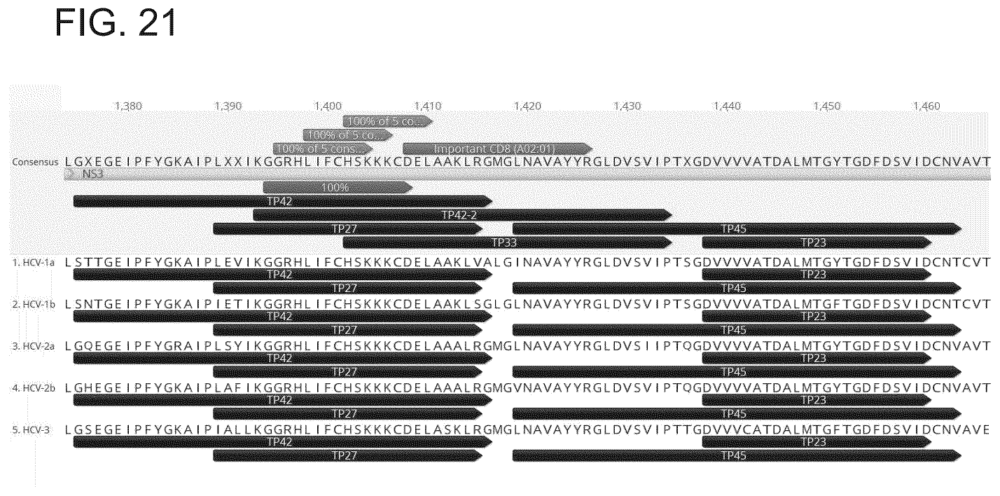

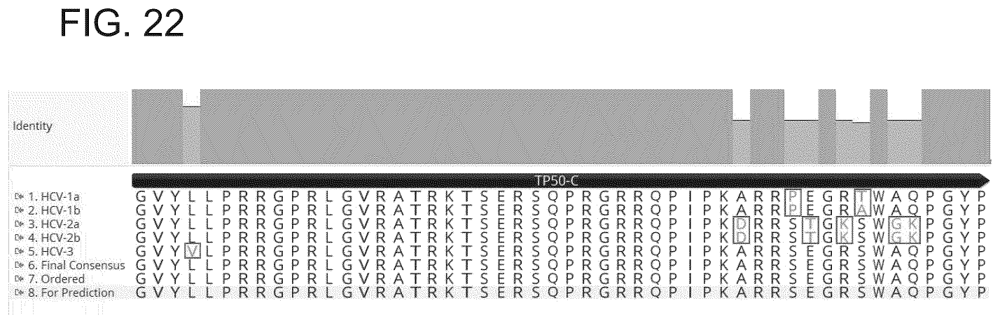

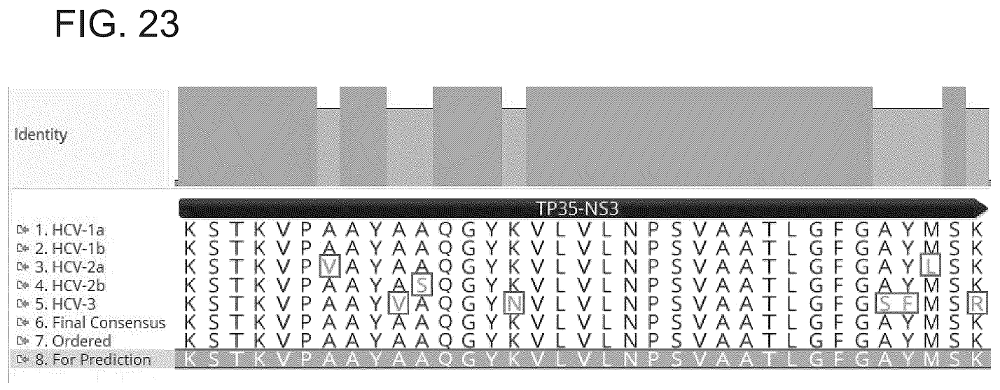

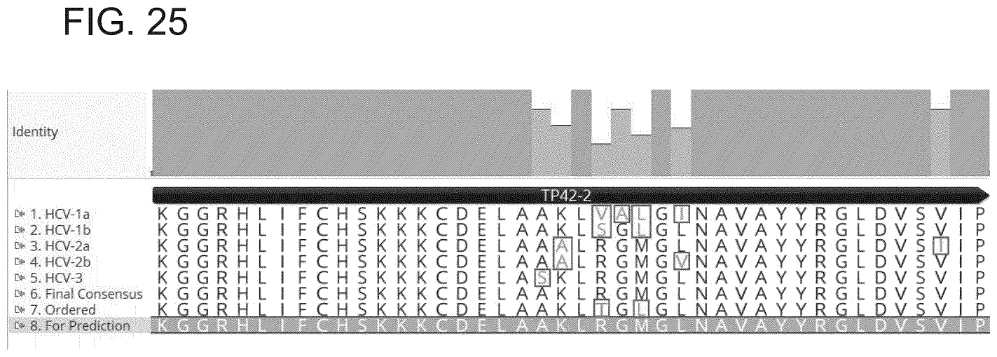

FIG. 21 depicts amino acid sequences of TP42, TP42-2, TP27, TP45, TP33, and TP23. FIG. 22-26 provide amino acid sequence alignments for TP50-C, TP35-NS3, TP42, TP42-2, and TP48, respectively. The “final consensus” sequences represent the consensus sequences for each of the TPs according to the most recent analysis. The “ordered” sequences represents the TP sequences that have been manufactured and tested for solubility. The “for prediction” sequences represent the TP sequences that were used in prediction analysis, to provide the predicted epitopes using IEDB software and its Databank.

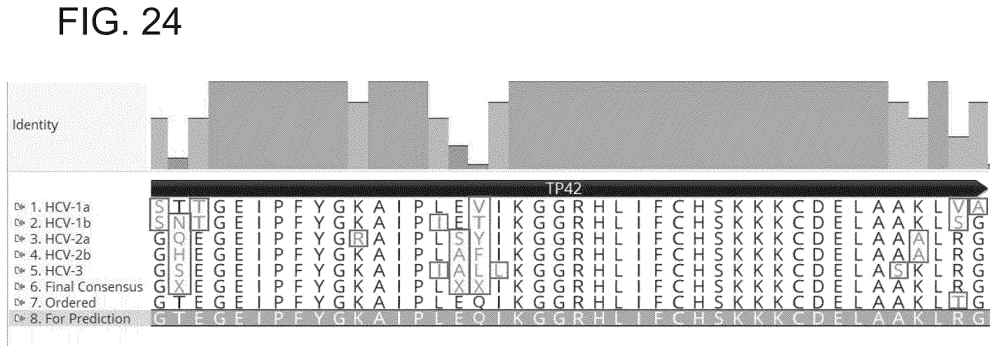

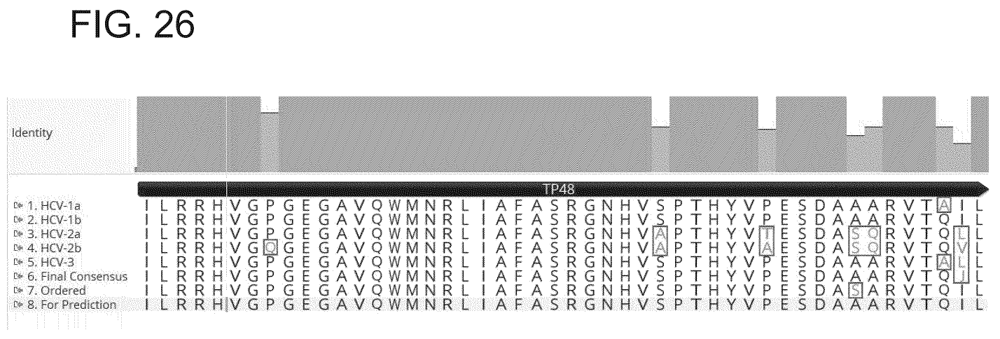

The Geneious software assigns letter “J” to any position of a consensus sequence, where the alternative amino acid could be “I” or “L” and assigns “X” where the alternative amino acid could be more than 3 different amino acids. Thus, in FIG. 26, a “J” indicates that the amino acid can be Ile or Leu. In FIG. 24, the “X” in position 2 can be any of Asn, Gln, His, Ser, or Thr.

### Solubility of TP33 and TP42-2

TP33 and TP42-2 were reconstituted in a solution at 0.5 mg/mL. The solutions were vortexed vigorously for 20 seconds. After vortexing, the solutions were incubated at room temperature with continuous shaking at 300 rpm for 2 hours and 30 minutes. After incubation, the solutions were centrifuged at 5000×g for 1 minute, and evaluated for solubility, both visually and by Nanodrop.

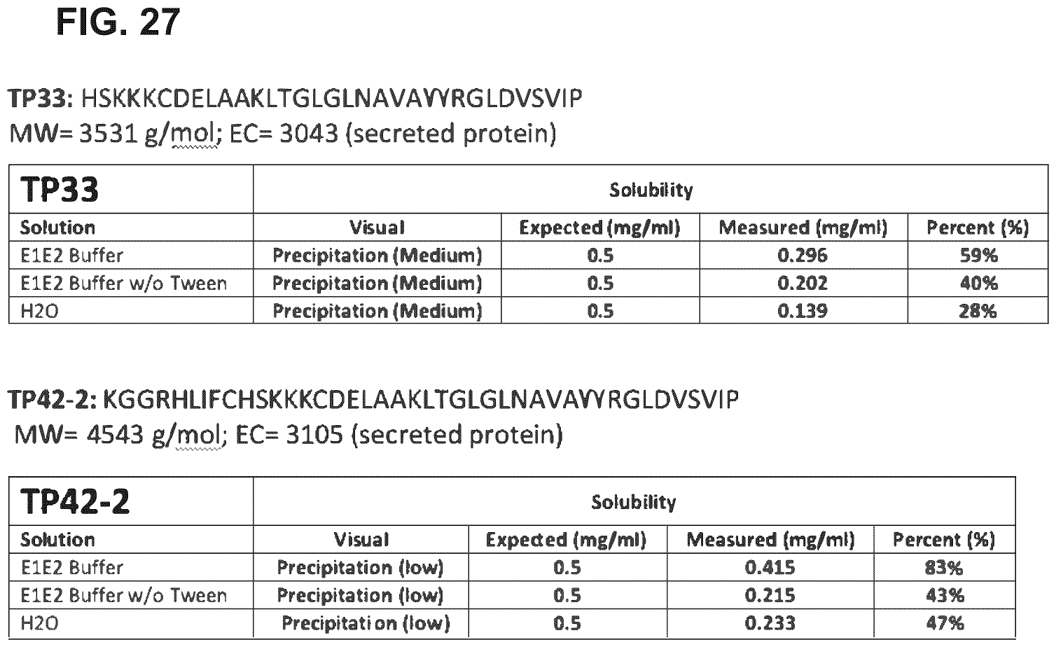

The results are depicted in FIG. 27.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. An immunogenic composition comprising:
a) one or more T-cell epitope polypeptides selected from:
i) a TP35-NS3 T-cell epitope polypeptide comprising an amino acid sequence having at least 80% amino acid sequence identity to:
KSTKVPX₁AYX₂X₃QGYX₄VLVLNPSVAATLGFGX₅X₆X₇SX₈ (SEQ ID NO:134), wherein X₁ is A or V; X₂ is A or V; X₃ is A or S; X₄ is K or N; X₅ is A or S; X₆ is Y or F; X₇ is M or L; and X₅ is K or R, wherein the TP35-NS3 T-cell epitope polypeptide has a length of from 28 amino acids to 35 amino acids; and
ii) a TP50C T-cell epitope polypeptide comprising an amino acid sequence having at least 80% amino acid sequence identity to:
GVYX₁LPRRGPRLGVRX₂TRKX₃SERSQPRGRRQX₄PKX₅X₆X₇X₈X₉GX₁₀X₁₁WX₁₂X₁₃PGYP (SEQ ID NO:149), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁₁ is S, T, H, or A; X₁₂ is A or G; and X₁₃ is Q or K, wherein the TP50C T-cell epitope polypeptide has a length of from 40 amino acids to 50 amino acids;
iii) a TP23 T-cell epitope polypeptide comprising an amino acid sequence having at least 80% amino acid sequence identity to: DVVVX₁X₂TDALMTGX₃TGDFDSVID (SEQ ID NO:171), wherein X₁ is V or C; X₂ is A or S; and X₃ is F or Y, wherein the TP23 T-cell epitope polypeptide has a length of from 18 amino acids to 23 amino acids;
iv) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:189), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids;
v) a TP35-NS4 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀ (SEQ ID NO:204), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; and X₁₀ is V or I, wherein the TP35-NS4 T-cell epitope polypeptide has a length of from 28 amino acids to 35 amino acids;
vi) a TP42 T-cell epitope polypeptide comprising an amino acid sequence having at least 80% amino acid sequence identity to: X₁X₂X₃GEIPFYGX₄AIPX₅X₆X₇X₈KGGRHLIFCHSKKKCDEX₉AX₁₀X₁₁LX₁₂X₁₃(K)n (SEQ ID NO:229), where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, or L; X₈ is I or L; X₉ is L or I; X₁₀ is A, K, or S; X₁₁ is K, Q, or A; X₁₂ is T, R, or S; and X₁₃ is G or S, wherein n is an integer from 2 to 10, and wherein the TP42 T-cell epitope polypeptide has a length of from 34 amino acids to 52 amino acids;
vii) a TP45 T-cell epitope polypeptide comprising an amino acid sequence having at least 80% amino acid sequence identity to: X₁X₂AVAX₃YRGX₄DVX₅X₆IPX₇X₈GDVVVX₉X₁₀TDALMTGX₁₁TGDFDSVIDX₁₂X₁₃X₁₄(K)n (SEQ ID NO:249), wherein X₁ is L or V; X₂ is N or T; X₃ is Y or F; X₄ is L or V; X₅ is S or A; X₆ is V or I; X₇ is T or A; X₈ is S, Q, or T; X₉ is V or C; X₁₀ is A or S; X₁₁ is F or Y; X₁₂ is C or K; X₁₃ is N or K; and X₁₄ is V or K, wherein n is an integer from 2 to 10, and wherein the TP45 T-cell epitope polypeptide has a length of from 36 amino acids to 55 amino acids; and
viii) a TP48 T-cell epitope polypeptide comprising an amino acid sequence having at least 80% amino acid sequence identity to: X₁X₂X₃RHX₄GX₅X₆EGX₇X₅QWMNRLIAFASRGNHVX₉PTHYX₁₀Xu₁₁X₁₂X₁₃DAX₁₄X₁₅X₁₆VX₁₇X₁₈X₁₉L(K)n (SEQ ID NO:255), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acids;
ix) a TP33 T-cell epitope polypeptide comprising an amino acid sequence having at least 80% amino acid sequence identity to: HSKKKCDELAX₁X₂LX₃X₄X₅GX₆NAVAYYRGLDVSX₇IP (SEQ ID NO:287), where X₁ is A or S; X₂ is K or A; X₃ is V, S, R, or T; X₄ is A or G; X₅ is L or M; X₆ is I, L, or V; and X₇ is V or I; wherein the TP33 T-cell epitope polypeptide has a length of from 30 amino acids to 36 amino acids;
x) a TP42-2 T-cell epitope polypeptide comprising an amino acid sequence having at least 80% amino acid sequence identity to: KGGRHLIFCHSKKKCDELAX₁X₂LX₃X₄X₅GX₆NAVAYYRGLDVSX₇IP (SEQ ID NO:292), where X₁ is A or S; X₂ is K or A; X₃ is V, S, R, or T; X₄ is A or G; X₅ is L or M; X₆ is I, L, or V; and X₇ is V or I; wherein the TP42-2 T-cell epitope polypeptide has a length of from 38 amino acids to 46 amino acids; and
b) an immune-stimulating amount of an adjuvant.

2.-24. (canceled)

25. A method of inducing an immune response in an individual to a hepatitis C virus (HCV) polypeptide, the method comprising administering to the individual:
a) an effective amount of the immunogenic composition of claim 1; or
b) one or more nucleic acids comprising nucleotide sequences encoding the T-cell epitope polypeptide(s) and/or the HCV E1/E2 heterodimeric polypeptide.

26.-30. (canceled)

31. A composition comprising one or more nucleic acids selected from the group consisting of:
i) a nucleic acid comprising a nucleotide sequence encoding a TP35-NS3 T-cell epitope polypeptide comprising an amino acid sequence having at least 80% amino acid sequence identity to:
KSTKVPX₁AYX₂X₃QGYX₄VLVLNPSVAATLGFGX₅X₆X₇SX₈ (SEQ ID NO:134), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids; and
ii) a nucleic acid comprising a nucleotide sequence encoding a TP50C T-cell epitope polypeptide comprising an amino acid sequence having at least 80% amino acid sequence identity to:
GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:149), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁₁ is S, T, H, or A; X₁₂ is A or G; and X₁₃ is Q or K, wherein the TP50C T-cell epitope polypeptide has a length of from 40 amino acids to 50 amino acids;
iii) a nucleic acid comprising a nucleotide sequence encoding a TP23 T-cell epitope polypeptide comprising an amino acid sequence having at least 80% amino acid sequence identity to: DVVVX₁X₂TDALMTGX₃TGDFDSVID (SEQ ID NO:171), wherein X₁ is V or C; X₂ is A or S; and X₃ is F or Y, wherein the TP23 T-cell epitope polypeptide has a length of from 18 amino acids to 23 amino acids;
iv) a nucleic acid comprising a nucleotide sequence encoding a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:189), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids;
v) a nucleic acid comprising a nucleotide sequence encoding a TP35-NS4 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀ (SEQ ID NO:204), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; and X₁₀ is V or I, wherein the TP35-NS4 T-cell epitope polypeptide has a length of from 28 amino acids to 35 amino acids;
vi) a nucleic acid comprising a nucleotide sequence encoding a TP42 T-cell epitope polypeptide comprising an amino acid sequence having at least 80% amino acid sequence identity to: X₁X₂X₃GEIPFYGX₄AIPX₅X₆X₇X₈KGGRHLIFCHSKKKCDEX₉AX₁₀X₁₁LX₁₂X₁₃(K)n (SEQ ID NO:229), where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, or L; X₈ is I or L; X₉ is L or I; X₁₀ is A, K, or S; Xu is K, Q, or A; X₁₂ is T, R, or S; and X₁₃ is G or S, wherein n is an integer from 2 to 10, and wherein the TP42 T-cell epitope polypeptide has a length of from 34 amino acids to 52 amino acids;
vii) a nucleic acid comprising a nucleotide sequence encoding a TP45 T-cell epitope polypeptide comprising an amino acid sequence having at least 80% amino acid sequence identity to: X₁X₂AVAX₃YRGX₄DVX₅X₆IPX₇X₈GDVVVX₉X₁₀TDALMTGX₁₁TGDFDSVIDX₁₂X₁₃X₁₄(K)n (SEQ ID NO:249), wherein X₁ is L or V; X₂ is N or T; X₃ is Y or F; X₄ is L or V; X₅ is S or A; X₆ is V or I; X₇ is T or A; X₈ is S, Q, or T; X₉ is V or C; X₁₀ is A or S; X₁₁ is F or Y; X₁₂ is C or K; X₁₃ is N or K; and X₁₄ is V or K, wherein n is an integer from 2 to 10, and wherein the TP45 T-cell epitope polypeptide has a length of from 36 amino acids to 55 amino acids; and
viii) a nucleic acid comprising a nucleotide sequence encoding a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:255), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acids;
ix) a nucleic acid comprising a nucleotide sequence encoding a TP33 T-cell epitope polypeptide comprising an amino acid sequence having at least 80% amino acid sequence identity to: HSKKKCDELAX₁X₂LX₃X₄X₅GX₆NAVAYYRGLDVSX₇IP (SEQ ID NO:287), where X₁ is A or S; X₂ is K or A; X₃ is V, S, R, or T; X₄ is A or G; X₅ is L or M; X₆ is I, L, or V; and X₇ is V or I; wherein the TP33 T-cell epitope polypeptide has a length of from 30 amino acids to 36 amino acids;
x) a nucleic acid comprising a nucleotide sequence encoding a TP42-2 T-cell epitope polypeptide comprising an amino acid sequence having at least 80% amino acid sequence identity to: KGGRHLIFCHSKKKCDELAX₁X₂LX₃X₄X₅GX₆NAVAYYRGLDVSX₇IP (SEQ ID NO:292), where X₁ is A or S; X₂ is K or A; X₃ is V, S, R, or T; X₄ is A or G; X₅ is L or M; X₆ is I, L, or V; and X₇ is V or I; wherein the TP42-2 T-cell epitope polypeptide has a length of from 38 amino acids to 46 amino acids;
xi) a TP42 T-cell epitope polypeptide comprising an amino acid sequence having at least 80% amino acid sequence identity to: X₁X₂X₃GEIPFYGX₄AIPX₅X₆X₇X₈KGGRHLIFCHSKKKCDEX₉AX₁₀X₁₁LX₁₂X₁₃ (SEQ ID NO:232), where X₁ is G, P, or S; X₂ is T, N, Q, H, or S; X₃ is E, T, or D; X₄ is K or R; X₅ is L or I; X₆ is E, A, S, or Q; X₇ is Q T, Y, F, or L; X₈ is I or L; X₉ is L or I; X₁₀ is A, K, or S; X₁₁ is K, Q, or A; X₁₂ is T, R, or S; and X₁₃ is G or S, and wherein the TP42 T-cell epitope polypeptide has a length of from 33 amino acids to 42 amino acids;
xii) a TP45 T-cell epitope polypeptide comprising an amino acid sequence having at least 80% amino acid sequence identity to: X₁X₂AVAX₃YRGX₄DVX₅X₆IPX₇X₈GDVVVX₉X₁₀TDALMTGX₁₁TGDFDSVIDX₁₂X₁₃X₁₄ (SEQ ID NO:252), where X₁ is L or V; X₂ is N or T; X₃ is Y or F; X₄ is L or V; X₅ is S or A; X₆ is V or I; X₇ is T or A; X₈ is S, Q, or T; X₉ is V or C; X₁₀ is A or S; X₁₁ is F or Y; X₁₂ is C or K; X₁₃ is N or K; and X₁₄ is V or K, and wherein the TP45 T-cell epitope polypeptide has a length of from 36 amino acids to 55 amino acids; and
xiii a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L (SEQ ID NO:266), where X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, and wherein the TP48 T-cell epitope polypeptide has a length of from 38 amino acids to 58 amino acids;.

32. The composition of claim 31, wherein the one or more nucleic acids are one or more mRNA molecules.

33. The composition of claim 31, wherein the one or more mRNA molecules are formulated with a liposome.

34. The composition of claim 31, wherein the composition comprises one or more nucleic acids comprising nucleotide sequences encoding a hepatitis C virus (HCV) E1 polypeptide and/or an HCV E2 polypeptide.

35. (canceled)

36. (canceled)

37. A method of inducing an immune response in an individual, the method comprising administering to the individual a composition of claim 31.

38. The method of claim 35, wherein said administering comprises:
a) administering a priming dose comprising the composition; and
b) administering a boosting dose comprising the composition.

39. A nucleic acid comprising a nucleotide sequence encoding a fusion polypeptide comprising from 2 to 10 polypeptides selected from the group consisting of:
i) a TP35-NS3 T-cell epitope polypeptide comprising an amino acid sequence having at least 80% amino acid sequence identity to:
KSTKVPX₁AYX₂X₃QGYX₄VLVLNPSVAATLGFGX₅X₆X₇SX₈ (SEQ ID NO:134), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids; and
ii) a TP50C T-cell epitope polypeptide comprising an amino acid sequence having at least 80% amino acid sequence identity to:
GVYX₁LPRRGPRLGVRX₂TRKX₃SERSQPRGRRQX₄IPKX₅X₆X₇X₅X₉GX₁₀X₁₁WX₁₂X₁₃PGYP (SEQ ID NO:149), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁₁ is S, T, H, or A; X₁₂ is A or G; and X₁₃ is Q or K, wherein the TP50C T-cell epitope polypeptide has a length of from 40 amino acids to 50 amino acids;
iii) a TP23 T-cell epitope polypeptide comprising an amino acid sequence having at least 80% amino acid sequence identity to: DVVVX₁X₂TDALMTGX₃TGDFDSVID (SEQ ID NO:171), wherein X₁ is V or C; X₂ is A or S; and X₃ is F or Y, wherein the TP23 T-cell epitope polypeptide has a length of from 18 amino acids to 23 amino acids;
iv) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:189), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids;
v) a TP35-NS4 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀ (SEQ ID NO:204), wherein X₁ is I or V; X₂ is L or I; X₃ is R or K; X₄ is V, I, or T; X₅ is P, Q, or T; X₆is G, A, or S; X₇is A or V; X₈ is V or T; X₉ is S or A; and X₁₀ is V or I, wherein the TP35-NS4 T-cell epitope polypeptide has a length of from 28 amino acids to 35 amino acids;
vi) a TP42 T-cell epitope polypeptide comprising an amino acid sequence having at least 80% amino acid sequence identity to: X₁X₂X₃GEIPFYGX₄AIPX₅X₆X₇X₈KGGRHLIFCHSKKKCDEX₉AX₁₀X₁₁LX₁₂X₁₃(K)n (SEQ ID NO:229), where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, or L; X₈ is I or L; X₉ is L or I; X₁₀ is A, K, or S; X₁₁ is K, Q, or A; X₁₂ is T, R, or S; and X₁₃ is G or S, wherein n is an integer from 2 to 10, and wherein the TP42 T-cell epitope polypeptide has a length of from 34 amino acids to 52 amino acids;
vii) a TP45 T-cell epitope polypeptide comprising an amino acid sequence having at least 80% amino acid sequence identity to: X₁X₂AVAX₃YRGX₄DVX₅X₆IPX₇X₈GDVVVX₉X₁₀TDALMTGX₁₁TGDFDSVIDX₁₂X₁₃X₁₄(K)n (SEQ ID NO:249), wherein X₁ is L or V; X₂ is N or T; X₃ is Y or F; X₄ is L or V; X₅ is S or A; X₆ is V or I; X₇ is T or A; X₈ is S, Q, or T; X₉ is V or C; X₁₀ is A or S; X₁₁ is F or Y; X₁₂ is C or K; X₁₃ is N or K; and X₁₄ is V or K, wherein n is an integer from 2 to 10, and wherein the TP45 T-cell epitope polypeptide has a length of from 36 amino acids to 55 amino acids; and
viii) a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:255), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acids;
ix) a TP33 T-cell epitope polypeptide comprising an amino acid sequence having at least 80% amino acid sequence identity to: HSKKKCDELAX₁X₂LX₃X₄X₅GX₆NAVAYYRGLDVSX₇IP (SEQ ID NO:287), where X₁ is A or S; X₂ is K or A; X₃ is V, S, R, or T; X₄ is A or G; X₅ is L or M; X₆ is I, L, or V; and X₇ is V or I; wherein the TP33 T-cell epitope polypeptide has a length of from 30 amino acids to 36 amino acids;
x) a TP42-2 T-cell epitope polypeptide comprising an amino acid sequence having at least 80% amino acid sequence identity to: KGGRHLIFCHSKKKCDELAX₁X₂LX₃X₄X₅GX₆NAVAYYRGLDVSX₇IP (SEQ ID NO:292), where X₁ is A or S; X₂ is K or A; X₃ is V, S, R, or T; X₄ is A or G; X₅ is L or M; X₆ is I, L, or V; and X₇ is V or I; wherein the TP42-2 T-cell epitope polypeptide has a length of from 38 amino acids to 46 amino acids;
xi) a TP42 T-cell epitope polypeptide comprising an amino acid sequence having at least 80% amino acid sequence identity to: X₁X₂X₃GEIPFYGX₄AIPX₅X₆X₇X₈KGGRHLIFCHSKKKCDEX₉AX₁₀X₁₁LX₁₂X₁₃ (SEQ ID NO:232), where X₁ is G, P, or S; X₂ is T, N, Q, H, or S; X₃ is E, T, or D; X₄ is K or R; X₅ is L or I; X₆ is E, A, S, or Q; X₇ is Q T, Y, F, or L; X₈ is I or L; X₉ is L or I; X₁₀ is A, K, or S; X₁₁ is K, Q, or A; X₁₂ is T, R, or S; and X₁₃ is G or S, and wherein the TP42 T-cell epitope polypeptide has a length of from 33 amino acids to 42 amino acids;
xii) a TP45 T-cell epitope polypeptide comprising an amino acid sequence having at least 80% amino acid sequence identity to: X₁X₂AVAX₃YRGX₄DVX₅X₆IPX₇X₈GDVVVX₉X₁₀TDALMTGX₁₁TGDFDSVIDX₁₂X₁₃X₁₄ (SEQ ID NO:252), where X₁ is L or V; X₂ is N or T; X₃ is Y or F; X₄ is L or V; X₅ is S or A; X₆ is V or I; X₇ is T or A; X₈ is S, Q, or T; X₉ is V or C; X₁₀ is A or S; X₁₁ is F or Y; X₁₂ is C or K; X₁₃ is N or K; and X₁₄ is V or K, and wherein the TP45 T-cell epitope polypeptide has a length of from 36 amino acids to 55 amino acids; and
xiii a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L (SEQ ID NO:266), where X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, and wherein the TP48 T-cell epitope polypeptide has a length of from 38 amino acids to 58 amino acids.

40. The composition of claim 39, wherein the nucleic acid is an mRNA

41. The composition of claim 40, wherein the mRNA is formulated with a liposome.

42. (canceled)

43. (canceled)

44. A method of inducing an immune response in an individual, the method comprising administering to the individual a composition of claim 39.

45. The method of claim 39, wherein said administering comprises:
a) administering a priming dose comprising the composition; and
b) administering a boosting dose comprising the composition.

46. The immunogenic composition of claim 1, wherein the composition comprises 2, 3, 4, or 5 different T-cell epitope polypeptides.

47. The immunogenic composition of claim 46, wherein the composition comprises:
(a) a TP50C T-cell epitope polypeptide comprising an amino acid sequence having at least 80% amino acid sequence identity to:
GVYLLPRRGPRLGVRATRKTSERSQPRGRRQPIPKARRSEGRSWAQPGYP (SEQ ID NO:148), wherein the TP50C T-cell epitope polypeptide has a length of from 40 amino acids to 50 amino acids; and
(b) a TP48 T-cell epitope polypeptide comprising an amino acid sequence having at least 80% amino acid sequence identity to: ILRRHVGPGEGAVQWMNRLIAFASRGNHVSPTHYVPESDAAARVTQIL (SEQ ID NO:268), wherein the T-cell epitope polypeptide has a length of from 45 amino acids to 55 amino acids.

48. The immunogenic composition of claim 47, wherein the composition further comprises:
(c) a TP35-NS4 T-cell epitope polypeptide comprising an amino acid sequence having at least 80% amino acid sequence identity to: ILRRHVGPGEGAVQWMNRLIAFASRGNHVSPTHYV (SEQ ID NO:203), wherein the TP35-NS4 T-cell epitope polypeptide has a length of from 28 amino acids to 35 amino acids.

49. The immunogenic composition of claim 47, wherein the composition further comprises:
(c) a TP27 T-cell epitope polypeptide comprising an amino acid sequence having at least 80% amino acid sequence identity to: LEQIKGGRHLIFCHSKKKCDELAAKLT (SEQ ID NO:188), wherein the TP27 T-cell epitope polypeptide has a length of from 22 amino acids to 27 amino acids.
